(19) United States
(12) Patent Application Publication
(10) Pub. No.: US 2022/0378773 A1
(43) Pub. Date: Dec. 1, 2022

(54) METHODS FOR TREATING LEUKEMIA AND USE OF A LEUKEMIC STEM CELL SIGNATURE TO PREDICT CLINICAL SENSITIVITY TO THERAPIES

(71) Applicant: CELGENE CORPORATION, Summit, NJ (US)

(72) Inventors: Michael POURDEHNAD, San Francisco, CA (US); Kyle James MACBETH, San Francisco, CA (US); Daniel Weston PIERCE, Belmont, CA (US); Remco Gerard LOOS, Sevilla (ES); Jinhong FAN, deceased (US)

(73) Assignee: CELGENE CORPORATION, Summit, NJ (US)

(21) Appl. No.: 17/772,099

(22) PCT Filed: Oct. 27, 2020
(86) PCT No.: PCT/US2020/057483

§ 371 Date: Apr. 26, 2022

Related U.S. Application Data
(60) Provisional application No. 62/927,052, filed on Oct. 28, 2019.

(51) Int. Cl. A61K 31/454; G01N 33/574; A61P 35/02

(52) U.S. Cl. CPC A61K 31/454; G01N 33/57426; A61P 35/02

(57) ABSTRACT

Provided herein are methods of using certain biomarkers, such as gene sets (e.g., a leukemic stem cell (LSC) signature), in predicting and monitoring clinical sensitivity and therapeutic response to certain compounds in patients having various diseases and disorders, such as cancer (e.g, lymphoma, multiple myeloma (MM), and leukemia, such as acute myeloid leukemia (AML)). Also provided herein are methods of treating diseases using the treatment compounds.

### CROSS-REFERENCE TO RELATED APPLICATIONS

This application claims the benefit of U.S. Provisional Patent Application No. 62/927,052, filed Oct. 28, 2019, which is incorporated by reference herein in its entirety.

### FIELD

Provided herein, in some embodiments, are methods of using certain biomarkers, such as gene sets (e.g., a leukemic stem cell (LSC) signature), in predicting and monitoring clinical sensitivity and therapeutic response to certain compounds in patients having various diseases and disorders, such as cancer (e.g., lymphoma, multiple myeloma (MM), and leukemia, such as acute myeloid leukemia (AML)). Also provided herein, in certain embodiments, are methods of treating diseases using the treatment compounds.

### BACKGROUND

Cancer is characterized primarily by an increase in the number of abnormal cells derived from a given normal tissue, invasion of adjacent tissues by these abnormal cells, or lymphatic or blood-borne spread of malignant cells to regional lymph nodes and to distant sites (metastasis). In general, cancer is divided into solid cancer and hematologic cancer. Examples of solid cancer include, but are not limited to, melanoma, adrenal carcinoma, breast carcinoma, renal cell cancer, pancreatic carcinoma, and small cell lung carcinoma (SCLC), etc.

Blood cancer generally includes three main types: lymphoma, leukemia, and myeloma. Lymphoma refers to cancers that originate in the lymphatic system. Lymphoma includes, but is not limited to, Hodgkin's lymphoma, non-Hodgkin's lymphoma (NHL), diffuse large B-cell lymphoma (DLBCL), and peripheral T-cell lymphomas (PTCL), etc. Leukemia refers to malignant neoplasms of the blood-forming tissues. Acute leukemia involves predominantly undifferentiated cell populations, whereas chronic leukemia involves more mature cell forms. Acute leukemia is divided into acute lymphoblastic leukemia (ALL) and acute myeloblastic leukemia (AML) types. Chronic leukemia is divided into chronic lymphocytic leukemia (CLL) or chronic myelocytic leukemia (CIVIL). Myeloma is a cancer of plasma cells in the bone marrow. Because myeloma frequently occurs at many sites in the bone marrow, it is often referred to as multiple myeloma (MM).

A tremendous demand therefore for new methods, treatments and compositions that can be used to treat patients with cancer including but not limited to, lymphoma (e.g., NHL), MM, leukemia (e.g., AML), and solid cancer. A number of studies have been conducted with the aim of providing compounds that can safely and effectively be used to treat cancers. For example, we have recently identified certain compounds (e.g., Compound D) useful to treat cancer including but not limited to, leukemia (e.g., AML). However, there is a need to develop efficient, sensitive, and accurate methods to detect, quantify, and characterize the pharmacodynamic activity of these compounds. The present invention satisfies these and other needs.

### SUMMARY OF THE INVENTION

Provided herein are methods of identifying a subject having acute myeloid leukemia who is likely to be responsive to a treatment comprising a compound or predicting the responsiveness of a subject having or suspected of having AML to a treatment comprising the compound. Also provide herein are methods of treating a subject having AML with a compound.

In one aspect, provided herein is a method of identifying a subject having acute myeloid leukemia (AML) who is likely to be responsive to a treatment comprising a compound or predicting the responsiveness of a subject having or suspected of having AML to a treatment comprising the compound, comprising:

i. providing a sample from the subject;
ii. measuring gene expression level of one or more genes in the sample;
iii. calculating a leukemic stem cell (LSC) signature score for the sample based on the gene expression level of the one or more genes; and
iv. identifying the subject as being likely to be responsive to the treatment comprising the compound if the level of the LSC signature score is higher than a reference level thereof, and the compound is 2-(4-chlorophenyl)-N-((2-(2,6-dioxopiperidin-3-yl)-1-oxoisoindolin-5-yl)methyl)-2,2-difluoroacetamide (Compound D), which has the following structure:

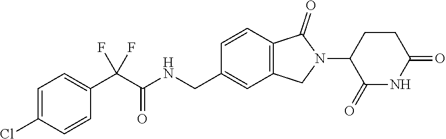 [SMILES: O=C1CCC(N2Cc3cc(CNC(=O)C(F)(F)c4ccc(Cl)cc4)ccc3C2=O)C(=O)N1]

or a stereoisomer or mixture of stereoisomers, isotopologue, pharmaceutically acceptable salt, tautomer, solvate, hydrate, co-crystal, clathrate, or polymorph thereof.

In another aspect, provided herein is a method of treating a subject having AML with a compound, comprising:

(a) identifying the subject having AML that may be responsive to the treatment comprising the compound, comprising:
i. providing a sample from the subject;
ii. measuring gene expression level of one or more genes in the sample;
iii. calculating a leukemic stem cell (LSC) signature score for the sample based on the gene expression level of the one or more genes; and
iv. identifying the subject as being likely to be responsive to the treatment comprising the compound if the level of the LSC signature score is higher than a reference level thereof, and
(b) administering the subject a therapeutically effective amount of the compound if the subject is identified as being likely to be responsive to the treatment comprising the compound, and the compound is Compound D, or a stereoisomer or mixture of stereoisomers, isotopologue, pharmaceutically acceptable salt, tautomer, solvate, hydrate, co-crystal, clathrate, or polymorph thereof.

In certain embodiments, the LSC signature score is calculated as the weighted sum of the expression level of the one or more genes.

In certain embodiments, the reference level is the median LSC signature score in a population.

In certain embodiments, the reference level is a pre-determined LSC signature score level.

In certain embodiments, the LSC signature score that is higher than the reference level thereof suggests that the subject has resistant and/or refractory AML.

In certain embodiments, the one or more genes are selected from the group consisting of

(a) CD34, SPINK2, LAPTM48, HOXA5, GUCY1A3, SHANK3, ANGPT1, ARHGAP22, LOC284422, MYCN, MAMDC2, PRSSL1, KIAA0125, GPSM1, HOXA9, MMRN1, FSCN1, DNMT38, HOXA6, AIF1L, SOCS2, CDK6, FAM69B, NGFRAP1, C3orf54, CPXM1, TNFRSF4, ZBTB46, DPYSL3, NYNRIN, COL24A1, FAM30A, C10orf140, SPNS2, GPR56, AKR1C3, FLT3, TFPI, KCNK17, EPDR1, C1orf150, BIVM, H2AFY2, VWF, EMP1, RAGE, ATP8B4, GATA2, SLC25A37, SGK, LOC652694, ITPR3, LOC654103, CXCR4, FCRL3, RBM38, LILRA5, IL18RAP, CCDC109B, ISG20, MTSS1, CECR1, ADAM19, FCGR2A, AIM2, NPL, IL10RA, CTSL1, GNLY, CKAP4, ADM, KLRB1, SLC15A3, FGR, FCRLA, IL2RB, CXCL16, SLC4A1, GZMH, FLJ22662, LOC647506, GIMAP4, JAZF1, CTSH, GZMA, CHST15, AQP9, CD247, BCL6, SLC7A7, E2F2, LOC647450, GZMB, LOC652493, HBM, CD14, ALAS2, HBB, LOC642113, AHSP, FCN1, CD48, HBA2, and HBA1, or
(b) CD34, SPINK2, LAPTM48, HOXA5, GUCY1A3, SHANK3, ANGPT1, ARHGAP22, LOC284422, MYCN, MAMDC2, PRSSL1, KIAA0125, GPSM1, HOXA9, MMRN1, FSCN1, DNMT38, HOXA6, AIF1L, SOCS2, CDK6, FAM69B, NGFRAP1, C3orf54, CPXM1, TNFRSF4, ZBTB46, DPYSL3, NYNRIN, COL24A1, FAM30A, C10orf140, SPNS2, GPR56, AKR1C3, FLT3, TFPI, KCNK17, EPDR1, C1orf150, BIVM, H2AFY2, VWF, EMP1, RAGE, ATP8B4, and GATA2.

In certain embodiments, the one or more genes are selected from the group consisting of AKR1C3, ARHGAP22, CD34, CDK6, CPXM1, DNMT3B, DPYSL3, EMP1, GPR56, KIAA0125, LAPTM4B, MMRN1, NGFRAP1, NYNRIN, SMIM24, SOCS2, and ZBTB46.

In certain embodiments, the LSC signature score is based on the gene expression levels of AKR1C3, ARHGAP22, CD34, CDK6, CPXM1, DNMT3B, DPYSL3, EMP1, GPR56, KIAA0125, LAPTM4B, MMRN1, NGFRAP1, NYNRIN, SMIM24, SOCS2, and ZBTB46.

In certain embodiments, the LSC signature score is calculated as follows: (expression level of DNMT3B×weight of DNMTT3B)+(expression level of ZBTB46×weight of ZBTB46)+(expression level of NYNRIN×weight of NYNRIN)+(expression level of ARHGAP22×weight of ARHGAP22)+(expression level of LAPTM4B×weight of LAPTM4B)+(expression level of MMRN1×weight of MMRN1)+(expression level of DPYSL3×weight of DPYSL3)+(expression level of KIAA0125×weight of KIAA0125)+(expression level of CDK6×weight of CDK6)+(expression level of CPXM1×weight of CPXM1)+(expression level of SOCS2×weight of SOCS2)+(expression level of SMIM24×weight of SMIM24)+(expression level of EMP1×weight of EMP1)+(expression level of NGFRAP1×weight of NGFRAP1)+(expression level of CD34×weight of CD34)+(expression level of AKR1C3×weight of AKR1C3)+(expression level of GPR56×weight of GPR56); and the weight of DNMTT3B is in a range from 0.08 to 0.09, the weight of ZBTB46 is in a range from −0.03 to −0.04, the weight of NYNRIN is in a range from −0.008 to 0.009, the weight of ARHGAP22 is in a range from −0.015 to 0.01, the weight of LAPTM4B is in a range from −0.006 to 0.005, the weight of MMRN1 is in a range from 0.02 to 0.03, the weight of DPYSL3 is in a range from 0.02 to 0.03, the weight of KIAA0125 is in a range from 0.01 to 0.02, the weight of CDK6 is in a range from −0.08 to −0.07, the weight of CPXM1 is in a range from −0.02 to −0.03, the weight of SOCS2 is in a range from 0.02 to 0.03, the weight of SMIM24 is in a range from −0.02 to −0.03, the weight of EMP1 is in a range from 0.014 to 0.02, the weight of NGFRAP1 is in a range from 0.04 to 0.05, the weight of CD34 is in a range from 0.03 to 0.04, the weight of AKR1C3 is in a range from −0.04 to −0.05, and the weight of GPR56 is in a range from 0.04 to 0.055.

In certain embodiments, the LSC signature score is calculated as follows: (expression level of DNMT3B×0.0874)+(expression level of ZBTB46×−0.0347)+(expression level of NYNRIN×0.00865)+(expression level of ARHGAP22×−0.0138)+(expression level of LAPTM4B×0.00582)+(expression level of MMRN1×0.0258)+(expression level of DPYSL3×0.0284)+(expression level of KIAA0125×0.0196)+(expression level of CDK6×−0.0704)+(expression level of CPXM1×−0.0258)+(expression level of SOCS2×0.0271)+(expression level of SMIM24×−0.0226)+(expression level of EMP1×0.0146)+(expression level of NGFRAP1×0.0465)+(expression level of CD34×0.0338)+(expression level of AKR1C3×−0.0402)+(expression level of GPR56×0.0501).

In certain embodiments, the reference level is 0.1, 0.2, 0.3, 0.4, 0.5, 0.6, 0.7, 0.8. 0.9, 1.0, 1.1, 1.2, 1.3, 1.4, 1.5, 1.6, 1.7, 1.8, 1.9, or 2.

In certain embodiments, the LSC signature score is based on the gene expression levels of TNFRSF4, SLC4A1, SLC7A7, and AIM2.

In certain embodiments, the LSC signature score is calculated as follows: (expression level of TNFRSF4×weight of TNFRSF4)+(expression level of SLC4A1×weight of SLC4A1)+(expression level of SLC7A7×weight of SLC7A7)+(expression level of AIM2×weight of AIM2); and the weight of TNFRSF4 is in a range from −1.5 to −1, the weight of SLC4A1 is in a range from 13 to 14, the weight of SLC7A7 is in a range from −4 to −3, the weight of AIM2 is in a range from −3 to −4.

In certain embodiments, the LSC signature score is calculated as follows: (expression level of TNFRSF4×−1.13)+(expression level of SLC4A1×13.59)+(expression level of SLC7A7×−3.57)+(expression level of AIM2×−3.04).

In certain embodiments, the reference level is in a range from −50 to 115, from −45 to 110, from −40 to 105, from −37 to 100, from −30 to 95, from −25 to 90, from −20 to 85, from −15 to 80, from −10 to 75, from −5 to 70, from 0 to 65, from 5 to 60, from 10 to 55, from 15 to 50, from 20 to 45, from 25 to 40, or from 30 to 35.

In certain embodiments, the LSC signature score is based on the gene expression levels of SLC4A1, SLC7A7, and AIM2.

In certain embodiments, the LSC signature score is calculated as follows: (expression level of SLC4A1×weight of SLC4A1)+(expression level of SLC7A7×weight of SLC7A7)+(expression level of AIM2×weight of AIM2); and the weight of SLC4A1 is in a range from 11 to 15, the weight of SLC7A7 is in a range from −5.5 to −1.5, the weight of AIM2 is in a range from −5 to −1.

In certain embodiments, the LSC signature score is calculated as follows:

is calculated as follows: (expression level of SLC4A1×13.59)+(expression level of SLC7A7×−3.57)+(expression level of AIM2×−3.04).

In certain embodiments, the reference level is in a range from −65 to 110, from −60 to 105, from −55 to 100, from −49 to 93, from −45 to 90, from −40 to 85, from −35 to 80, from −30 to 75, from −25 to 70, from −20 to 65, from −15 to 60, from −10 to 55, from −5 to 50, from 0 to 45, from 5 to 40, from 10 to 35, from 15 to 30, from 20 to 35, or from 25 to 30.

In another aspect, provided herein is a method of identifying a subject having AML, who is likely to be responsive to a treatment comprising a compound or predicting the responsiveness of a subject having or suspected of having AML to a treatment comprising the compound, comprising:

i. providing a sample from the subject;
ii. administering the compound to the sample;
iii. measuring the proportion of one or more types of cells;
iv. identifying the subject as being likely to be responsive to the treatment comprising the compound if the proportion of the one or more types of cells differentiates from a reference proportion of the cells, and the compound is Compound D, or a stereoisomer or mixture of stereoisomers, isotopologue, pharmaceutically acceptable salt, tautomer, solvate, hydrate, co-crystal, clathrate, or polymorph thereof.

In another aspect, provided herein is a method of treating a subject having AML with a compound, comprising:

(a) identifying the subject having AML that may be responsive to the treatment comprising the compound, comprising:
i. providing a sample from the subject;
ii. administering the compound to the sample;
iii. measuring the proportion of one or more types of cells;
iv. identifying the subject as being likely to be responsive to the treatment comprising the compound if the proportion of the one or more types of cells differentiates from a reference proportion of the cells, and
(b) administering to the subject a therapeutically effective amount of the compound if the subject is identified as being likely to be responsive to the treatment comprising the compound, and the compound is Compound D, or a stereoisomer or mixture of stereoisomers, isotopologue, pharmaceutically acceptable salt, tautomer, solvate, hydrate, co-crystal, clathrate, or polymorph thereof.

In certain embodiments, the reference proportion of a type of cells is the proportion of the type of cells in the sample prior to administering the compound.

In certain embodiments, the reference proportion of a type of cells is a pre-determined proportion.

In certain embodiments, the method comprising measuring the proportion of primitive cells and/or the proportion differentiated leukemia cells.

In certain embodiments, a reduction of the proportion of primitive cells and/or an increase of the proportion of differentiated leukemia cells as compared to their respective proportions prior to administering the compound indicates that the subject is likely to be responsive to the treatment comprising the compound.

In certain embodiments, the method comprising measuring the proportion of CD34+, CD15+ cells, CD14+ cells, and/or CD11b+ cells.

In certain embodiments, the method comprising measuring the proportion of CD34+ cells, and a reduction of the proportion of CD34+ cells as compared to the proportion of CD34+ cells prior to administering the compound indicates the subject is likely to be responsive to the treatment comprising the compound.

In certain embodiments, the method comprising measuring the proportion of CD15+ cells and/or CD14+ cells, and an increase of the proportion of CD15+ cells and/or CD14+ cells as compared to the proportion of CD15+ cells and/or CD14+ cells prior to administering the compound indicates the subject is likely to be responsive to the treatment comprising the compound.

In certain embodiments, the AML is refractory or resistant.

In certain embodiments, the AML is resistant to treatment using one or more agents selected from the group consisting of daunorubicin, cytarabine (ara-C), and gemtuzumab ozogamicin, or resistant to chemotherapies.

### BRIEF DESCRIPTION OF THE FIGURES

FIGS. 1A-1C depicts Compound D-mediated degradation of GSPT1 in acute myeloid cells in vitro. FIG. 1A shows the degradation of GSPT1 as assessed by flow cytometric analysis using anti-GSPT-1 conjugated antibody binding to GSPT1, measured by MFI of the Alexa flour 647 fluorophore, following in vitro incubation of Compound D with indicated AML patient samples of varying LSC17 score for 4 hours. FIG. 1B shows the degradation of GSPT1 as assessed by flow cytometric analysis using anti-GSPT-1 conjugated antibody binding to GSPT1, measured by MFI of the Alexa flour 647 fluorophore, following in vitro incubation of Compound D with indicated AML patient samples of varying LSC17 score for 24 hours. Results are expressed as a percentage of vehicle control (1.0 equivalent to 100%). FIG. 1C shows degradation of GSPT1 following 100 nM Compound D incubation for 24 hours as assessed for AML patient samples receiving high and low LSC17 scores, with results presented as mean with error bars representing standard error of the mean. GSPT1=G1 to S phase transition protein 1; ID=identification; LSC17=leukemia stem cell 17-gene signature; MFI=median fluorescence intensity; nd=not determined.

FIGS. 2A-2C depict Compound D-mediated induction of apoptosis of primary acute myeloid leukemia blasts in vitro. FIG. 2A shows representative flow cytometry dot plots for determining apoptotic cells (top panels show apoptosis as assessed by FSC/SSC gated cells; bottom panels show apoptosis as assessed by AnnexinV staining following vehicle (0 nM) or 100 nM Compound D treatment). FIG. 2B shows the percent of Annexin V+ cells (apoptotic) assessed for 9 AML patient samples following incubation with Compound D (0, 3, 30, or 100 nM). Samples were grouped by high and low LSC17 scores. FIG. 2C shows total cell number assessed for 9 AML patient samples following incubation with Compound D (0, 3, 30, or 100 nM). Samples were grouped by high and low LSC17 scores. Data is displayed as group mean with error bars representing standard error of the mean. The P value denotes statistical comparison between LSC17 high and LSC17 low groups. FSC=forward scatter; LSC17=leukemia stem cell 17-gene signature; SSC=side scatter; 7AAD=7-aminoactinomycin D.

FIG. 3 depicts Compound D-mediated inhibition of colony forming leukemia progenitors. The number of colonies formed at 24 hours was assessed per 100,000 cells for 9 AML patient samples following incubation with Compound D (0, 3, 30, or 100 nM) with colony reduction in the samples that formed colonies assessed as a percent of vehicle control when samples were grouped by high and low LSC17 scores. Results are displayed as group mean with error bars representing standard error of the mean. ID=identification; LSC17=leukemia stem cell 17-gene signature.

FIG. 4 depicts effects of Compound D on acute myeloid leukemia patient 110500 and patient 90191 xenografts. Percentage of AML cells or AML cells with different markers (CD34+ or CD15+) from right femur (RF) or from left femur plus tibia bone marrow (BM) post treatments with different doses of Compound D are plotted. The P values denote statistical comparison to vehicle control of the same bone marrow source. AML=acute myeloid leukemia; Bid=twice daily; BM=bone marrow (non-injected); Qd=daily; RF=right femur (AML-cell injected).

FIG. 5 depicts responsiveness to Compound D and changes of different types of cells in samples with high LSC17 signature scores. Percentage of AML cells or percentage of CD34+ cells, and absolute cell number for AML or CD34+ cells in RF or BM from 3 AML samples with high LSC17 scores are shown. Round symbols represent data from vehicle control mice and square symbols represent Compound D-treated mice. The P-values denote statistical comparison between Compound D-treated and vehicle control. AML=acute myeloid leukemia; BM=bone marrow (non-injected); LSC17=leukemic stem cell 17-gene signature; RF=right femur (AML-cell injected).

FIG. 6 depicts responsiveness to Compound D and changes of different types of cells in samples with low LSC17 signature scores. Percentage of AML cells or percentage of CD34+ cells, and absolute cell number for AML or CD34+ cells in the RF or BM from three AML samples with low LSC17 scores are shown. Round symbols represent data from vehicle control mice and square symbols represent Compound D-treated mice. The P-values denote statistical comparison between Compound D-treated and vehicle control. AML=acute myeloid leukemia; BM=bone marrow (non-injected); LSC17=leukemic stem cell 17-gene signature; RF=right femur (AML-cell injected).

FIGS. 7A-7E depict secondary transplant following Compound D treatment of primary transplanted mice. FIG. 7A shows percentage of engrafted AML cells from bone marrow (RF or BM) of secondary mice injected with cells of AML patient sample 110590 isolated from bone marrow of 2.5 mg/kg Compound D-dosed primary mice. Limiting dilution assay (LDA) analysis (bottom panel) shows a 13.3-fold reduction in leukemic stem cells (LSCs) following Compound D dosing compared to vehicle (bottom panel). FIG. 7B shows percentage of engrafted AML cells from bone marrow (RF or BM) of secondary mice injected with cells of AML patient sample 120860 isolated from bone marrow of 2.5 mg/kg Compound D-dosed primary mice. Limiting dilution assay (LDA) analysis (bottom panel) shows no difference in LSC frequency. FIG. 7C shows percentage of engrafted AML cells from bone marrow (RF) of secondary mice injected with cells of AML patient sample 100348 isolated from bone marrow of 2.5 mg/kg Compound D-dosed primary mice. FIG. 7D shows percentage of CD45+ cells in the isolated bone marrow from primary-treated mice for each patient sample. The total cells and total AML cells injected into each secondary mouse (without mouse cell depletion) are also shown. FIG. 7E shows percentage of AML graft in secondary mice receiving cells of AML patient sample 110102 (top) or AML patient sample 0590 (bottom) from vehicle- or Compound D-treated primary xenografted mice are shown with each symbol representing a single secondary transplanted mouse. AML=acute myeloid leukemia; BM=bone marrow (non-injected); ID=identification; K=thousand; M=million; RF=right femur (AML cell injected).

FIGS. 8A-8B depict representative flow cytometry analysis of cord blood xenograft. FIG. 8A shows representative gating strategies for identification of cell populations isolated from cord blood-xenografted vehicle. FIG. 8B shows representative gating strategies for identification of cell populations isolated from Compound D-treated mouse bone marrow. CD45+ cells were gated (GlyA-CD45+, left column) and further subgated to determine CD38 and CD34 expression (second column from left) or CD19 and CD33 expression (middle column). GlyA-CD45+ and GlyA-CD45+CD33+ cells were subgated to determine expression of CD14 and CD15 (second from right and far right columns, respectively). BM=bone marrow (non-injected); GlyA=glycophorin A; RF=right femur (acute myeloid leukemia cell injected).

FIGS. 9A-9D depict effects of Compound D on cord blood graft populations. Percentages or absolute cell numbers of each of the sub cell types were plotted for CB1 and CB2 engrafted cells. FIG. 9A shows percentages or absolute cell numbers of CB1 and CB2 engrafted cells. FIG. 9B shows percentages or absolute cell numbers of CD19+ or CD33+ cells. FIG. 9C shows percentages or absolute cell numbers of CD15+ or CD14+ cells in CD45+ grafts. FIG. 9D shows percentages or absolute cell numbers of GlyA+ cells. Round symbols represent data from vehicle-control treated mice, and square symbols represent data from Compound D-treated mice. The P values denote statistical comparison of Compound D-treated versus control. BM=bone marrow (non-injected); CB=cord blood; GlyA=glycophorin A; RF=right femur (AML cell-injected).

FIGS. 10A-10D depict effects of Compound D on CD34+ and CD34+/CD38-primitive cells. Percentages or absolute cell numbers from each of the sub cell types were plotted for RF or BM for CB1 and CB2 engrafted cells. FIG. 10A shows percentages or absolute cell numbers of CD34+ cells. FIG. 10B shows percentages or absolute cell numbers of CD34+/CD38− cells. FIG. 10C shows percentages or absolute cell numbers of CD34+/CD19+ cells. FIG. 10D shows percentages or absolute cell numbers of CD34+/CD33+ cells. Round symbols represent data from vehicle-control treated mice, and square symbols represent data from Compound D-treated mice. The P values denote statistical comparison of Compound D-treated versus control. BM=bone marrow (non-injected); CB=cord blood; RF=right femur (AML cell-injected).

FIG. 11 depicts effects of Compound D on acute myeloid leukemia graft in NOD/SCID mice. Human CD45+/CD33+ AML engraftment in the injected femur (RF, top panel) and non-injected bones (BM, bottom panel) of Compound D (square) or vehicle control (circle) treated mice are summarized. Each symbol indicates the engraftment level in each treated mouse and bars indicate the median values of each treated group. AML=acute myeloid leukemia; BM=bone marrow (non-injected bones); ns=not significant; RF=right femur (injected bone). * p<0.05; ** p<0.01; *** p<0.001; **** p<0.0001.

FIGS. 12A-12E depict phenotypic profiles induced by Compound D administration. FIG. 12A shows representative flow cytometry analysis of cell surface markers CD15, CD14, CD34, and CD38 on leukemic cells in AML graft, after Compound D or vehicle treatment. FIG. 12B shows representative flow cytometry analysis of cell surface markers CD15, CD14, CD34, CD11b and CD38 on leukemic cells in AML graft, after Compound D or vehicle treatment. FIG. 12C shows the percentage of CD34+ cells from the AML graft after Compound D (square) or vehicle control (circle) treatment. FIG. 12D shows the percentage of CD15+ cells from the AML graft after Compound D (square) or vehicle control (circle) treatment. FIG. 12E shows the percentage of CD14+ cells from the AML graft after Compound D (square) or vehicle control (circle) treatment. Samples were grouped into 3 categories: increase (top panel), decrease (middle panel) and no change (bottom panel) of CD34+, CD15+, and CD14+ cells. Each symbol indicates the percentage of the corresponding population in AML graft of each treated mouse. The percentage in the bracket after each patient number is the relative reduction by Compound D treatment to indicate the responsiveness of each sample to the drug. Bars indicate the mean values. AML=acute myeloid leukemia; BM=bone marrow (non-injected bones); ns=not significant; RF=right femur (injected bones). *p<0.05; **p<0.01; ***p<0.001; ****p<0.0001.

FIG. 13 depicts heterogeneous responses to Compound D in primary acute myeloid leukemia graft and correlation to LSC17 scores. The effect of Compound D on acute myeloid leukemia (AML) graft was presented as percentage of AML reduction to vehicle control treatment. Each symbol represents the relative reduction of median AML engraftment for each patient sample and long horizontal bars indicate the mean values of each group with shorter horizontal bars indicating standard error of the mean (SEM). Samples were summarized in total (solid circle) and were grouped into high LSC17 (square) and low LSC17 scores (triangle). BM=bone marrow (non-injected bones); LSC=leukemic stem cell; RF=right femur (injected bones).

FIGS. 14A-14C depict LSC4 gene signature and LSC3 gene signature and their predictiveness for responsiveness to Compound D treatment. Gene expression profiles were generated by RNA-Seq from the primary cells of each patient sample. FIG. 14A shows 4-gene score (LSC4) identified out of 89 LSC associated gene set. The solid curve represents % reduction by the drug in the experiments, and the dashed curve represents the % reduction predicted by 4-gene scores. FIG. 14B shows discretizing the predictions by a median threshold to either “response” or “no response” and an association between the scores and % reduction (r=0.87, p=0.02). The solid curve represents % reduction by the drug in the experiments, and the dashed curve represents the prediction of % reduction by 4-gene scores. FIG. 14C shows 3-gene score (LSC3) identified among about 46 LSC⁻ gene set may predict responsiveness to Compound D, with very similar results to the LSC4 gene signature described above. The solid curve represents % reduction by the drug in the experiments, and the dashed curve represents the prediction of % reduction by 3-gene scores.

FIGS. 15A-15D depict clinical characteristics of patients and responsiveness of grafts to Compound D. FIG. 15A depicts that samples were characterized for their responses to Compound D based on their profiles of de novo vs secondary/relapse. FIG. 15B depicts that samples were characterized for their responses to Compound D based on their profiles of adverse vs intermediate prognosis. FIG. 15C depicts that samples were characterized for their responses to Compound D based on their profiles of cytogenetically normal vs abnormal karyotypes. FIG. 15D depicts that samples were characterized for their responses to Compound D based on their profiles of Flt3-ITD vs wild-type Flt3 in cytogenetically normal AML. Each symbol represents the relative reduction of median AML engraftment for each patient sample and bars indicate the median values. AML=acute myeloid leukemia; BM=bone marrow (non-injected bones); CN-AML=cytogenetically normal acute myeloid leukemia; Flt3-ITD=fms-like tyrosine kinase 3-internal tandem duplication; RF=right femur (injected bones).

FIGS. 16A-16D depict Compound D induces in vitro acute myeloid leukemia apoptosis through GSPT1 reduction. Acute myeloid leukemia cells were cultured in vitro in the medium supplemented with growth factors, at different Compound D concentrations. FIG. 16A shows GSPT1 degradation in primary leukemic cells at 24 hours of exposure to Compound D. FIG. 16B show induction of apoptosis in leukemic cells. FIG. 16C shows decrease of live cells upon treatment with Compound D. FIG. 16D shows colony-forming leukemic progenitors were reduced by Compound D. GSPT1=G1 to S phase transition protein 1.

FIG. 17 depicts induction of apoptosis and cell death by Compound D treatment. Apoptosis and cell death in the mice treated with Compound D was assessed by staining cells with propidium iodide. Each symbol indicates the percentage of PI+ events in individual mouse treated with vehicle (circle) or Compound D (square). Bars indicate the median values. BM=bone marrow (non-injected bones); PI=propidium iodide; RF=right femur (injected bones). *p<0.05; **p<0.01; ***p<0.001; ****p<0.0001.

FIGS. 18A-18B depict Compound D treatment degrades GSPT1 in acute myeloid leukemia graft. Intracellular flow cytometry (FACS) was performed to measure the expression of GSPT1 after 3 doses of Compound D treatment to the mice bearing AML. FIG. 18A shows mean fluorescence intensity of GSPT1 in CD33+ AML cells harvested from the injected RF (upper panel) and non-injected BM (lower panel) of mice treated with vehicle (circle) and Compound D (square). Each symbol indicates the data from individual mouse treated with vehicle (circle) or Compound D (square) and bars indicate the median values. Numbers above the data points are p values between Compound D treated vs controls. FIG. 18B shows relative GSPT1 reduction by Compound D. Each bar indicates the percentage of median GSPT1 MFI by Compound D treatment relative to vehicle control. The percentage in the bracket after each patient number is the relative reduction by 4 weeks of Compound D treatment to indicate the responsiveness of each sample to the drug. AML=acute myeloid leukemia; BM=bone marrow (non-injected bones); GSPT1=G1 to S phase transition protein 1; MFI=mean fluorescence intensity; PI=propidium iodide; RF right femur (injected bones).

FIG. 19 depicts representative secondary transplantation limiting dilution assay (confidence interval plot of leukemic stem cell frequencies). Solid lines indicate the mean estimation of LSC frequencies and the dotted lines indicate the lower and upper range of LSC frequency estimation in vehicle control (grey dotted) or Compound D (black dotted) primary mice. Each individual symbol indicates the log fraction of non-responding related to each cell dose. LSC=leukemic stem cell; Veh=vehicle.

### DETAILED DESCRIPTION OF THE INVENTION

Certain compounds provided herein including Compound D are cereblon E3 ligase modulators. For example, Compound D causes degradation of the translation termination factor G1 to S phase transition protein 1 (GSPT1) and leads to integrated stress response, unfolded protein response (UPR) activation, and apoptosis in acute myeloid leukemia (AML) cells. Compound D is in clinical development for relapsed and refractory AML.

Refractoriness to induction chemotherapy and relapse after achievement of remission are the main obstacles to cure AML. After standard induction chemotherapy, patients are assigned to different post-remission strategies on the basis of cytogenetic and molecular abnormalities that broadly define adverse, intermediate and favorable risk categories. However, some patients do not respond to induction therapy and another subset will eventually relapse despite the lack of adverse risk factors. There is an urgent need for better biomarkers to identify these high-risk patients, and treatments for this group of patients.

To develop predictive and/or prognostic biomarkers related to stemness, Ng et al. (Ng S W et al. Nature. 2016; 540(7633): 433-37) generated a 17-gene score using functional leukemia stem cell populations (LSC17 score). More details on the method of generating the LSC17 score are described in Section 6.1. As shown by Ng et al., patients with high LSC17 scores had poor outcomes with current treatments including allogeneic stem cell transplantation.

Surprisingly in the present studies described in Section 6.2, AML samples with high LSC17 scores were more sensitive to Compound D treatment in comparison to samples with low LSC17 scores. Furthermore, in the studies described in Section 6.3, a majority of samples with high LSC17 scores responded well to Compound D, and AML was eradicated in the mouse bone marrow in more than half of them.

The unexpected observations provided herein indicate that Compound D can be used to treat AML patients whose diseases are more aggressive in the context of primary induction therapy, and/or patients having refractory AML resistant to conventional treatments such as chemotherapies.

Furthermore, as shown in Section 6, the present disclosure also identifies cell surface markers or changes thereof useful for predicting responsiveness to a treatment compound (e.g., Compound D, or a stereoisomer or a mixture of stereoisomers, tautomer, pharmaceutically acceptable salt, solvate, isotopologue, prodrug, hydrate, co-crystal, clathrate, or a polymorph thereof).

### 5.1. Definitions

As used herein, the term “cancer” includes, but is not limited to, solid cancer and hematological cancer. The term “cancer” refers to disease of tissues or organs, including but not limited to, cancers of the bladder, bone, blood, brain, breast, cervix, chest, colon, endometrium, esophagus, eye, head, kidney, liver, lymph nodes, lung, mouth, neck, ovaries, pancreas, prostate, rectum, skin, stomach, testis, throat, and uterus. Specific cancers include, but are not limited to, advanced malignancy, amyloidosis, neuroblastoma, meningioma, hemangiopericytoma, multiple brain metastase, glioblastoma multiforme, glioblastoma, brain stem glioma, poor prognosis malignant brain tumor, malignant glioma, recurrent malignant glioma, anaplastic astrocytoma, anaplastic oligodendroglioma, neuroendocrine tumor, rectal adenocarcinoma, unresectable colorectal carcinoma, metastatic hepatocellular carcinoma, Kaposi's sarcoma, karotype acute myeloblastic leukemia, Hodgkin's lymphoma, non-Hodgkin's lymphoma, cutaneous T-Cell lymphoma, cutaneous B-Cell lymphoma, diffuse large B-Cell lymphoma, low grade follicular lymphoma, malignant melanoma, malignant mesothelioma, malignant pleural effusion mesothelioma syndrome, peritoneal carcinoma, papillary serous carcinoma, gynecologic sarcoma, soft tissue sarcoma, scleroderma, cutaneous vasculitis, Langerhans cell histiocytosis, leiomyosarcoma, fibrodysplasia ossificans progressive, hormone refractory prostate cancer, resected high-risk soft tissue sarcoma, unrescectable hepatocellular carcinoma, Waldenstrom's macroglobulinemia, smoldering myeloma, indolent myeloma, fallopian tube cancer, androgen independent prostate cancer, androgen dependent stage IV non-metastatic prostate cancer, hormone-insensitive prostate cancer, chemotherapy-insensitive prostate cancer, papillary thyroid carcinoma, follicular thyroid carcinoma, medullary thyroid carcinoma, and leiomyoma.

As used herein, “hematological cancer” includes myeloma, lymphoma, and leukemia. In one embodiment, the myeloma is multiple myeloma. In some embodiments, the leukemia is, for example, acute myelogenous leukemia (AML), acute lymphocytic leukemia (ALL), adult T-cell leukemia, chronic lymphocytic leukemia (CLL), hairy cell leukemia, myelodysplasia, myeloproliferative disorders, chronic myelogenous leukemia (CIVIL), myelodysplastic syndrome (MDS), human lymphotropic virus-type 1 (HTLV-1) leukemia, mastocytosis, or B-cell acute lymphoblastic leukemia. In some embodiments, the lymphoma is, for example, diffuse large B-cell lymphoma (DLBCL), B-cell immunoblastic lymphoma, small non-cleaved cell lymphoma, human lymphotropic virus-type 1 (HTLV-1) leukemia/lymphoma, adult T-cell lymphoma, peripheral T-cell lymphoma (PTCL), cutaneous T-cell lymphoma (CTCL), mantle cell lymphoma (MCL), Hodgkin's lymphoma (HL), non-Hodgkin's lymphoma (NHL), AIDS-related lymphoma, follicular lymphoma, small lymphocytic lymphoma, T-cell/histiocyte rich large B-cell lymphoma, transformed lymphoma, primary mediastinal (thymic) large B-cell lymphoma, splenic marginal zone lymphoma, Richter's transformation, nodal marginal zone lymphoma, or ALK-positive large B-cell lymphoma. In one embodiment, the hematological cancer is indolent lymphoma including, for example, DLBCL, follicular lymphoma, or marginal zone lymphoma.

The term “prognosis risk,” when used in connection with cancer, refers to the possible outcomes of the cancer, including responsiveness to certain treatments, duration or extent of remission, potential survival rate, probability of relapse, etc. Factors that affect a patient's prognosis risk include, but are not limited to, demographic (e.g., age, race, sex, etc.), disease-specific (e.g., cancer stage), genetic (e.g., risk gene), co-morbid (e.g., other conditions accompanying the cancer), etc. A good “prognosis risk” means that the patient is likely to be responsive to certain treatments, is likely to survive, and/or is unlikely to relapse, etc. A poor “prognosis risk” means that the patient is unlikely to be responsive to certain treatments, is unlikely to survive, and/or is likely to relapse, etc.

As used herein, and unless otherwise specified, the terms “treat,” “treating,” and “treatment” refer to an action that occurs while a patient is suffering from the specified cancer, which reduces the severity of the cancer or retards or slows the progression of the cancer.

The term “sensitivity” or “sensitive” when made in reference to treatment with compound is a relative term which refers to the degree of effectiveness of the compound in lessening or decreasing the progress of a tumor or the disease being treated. For example, the term “increased sensitivity” when used in reference to treatment of a cell or tumor in connection with a compound refers to an increase of, at least about 5%, or more, in the effectiveness of the tumor treatment.

As used herein, the terms “compound” and “treatment compound” are used interchangeably and include the non-limiting examples of compounds disclosed in Section 5.5 below.

As used herein, and unless otherwise specified, the term “therapeutically effective amount” of a compound is an amount sufficient to provide a therapeutic benefit in the treatment or management of a cancer, or to delay or minimize one or more symptoms associated with the presence of the cancer. A therapeutically effective amount of a compound means an amount of therapeutic agent, alone or in combination with other therapies, which provides a therapeutic benefit in the treatment or management of the cancer. The term “therapeutically effective amount” can encompass an amount that improves overall therapy, reduces or avoids symptoms or causes of cancer, or enhances the therapeutic efficacy of another therapeutic agent. The term also refers to the amount of a compound that is sufficient to elicit the biological or medical response of a biological molecule (e.g., a protein, enzyme, RNA, or DNA), cell, tissue, system, animal, or human, which is being sought by a researcher, veterinarian, medical doctor, or clinician.

The term “responsiveness” or “responsive” when used in reference to a treatment refers to the degree of effectiveness of the treatment in lessening or decreasing the symptoms of a disease, e.g., cancer, such as MM or AML, being treated. For example, the term “increased responsiveness” when used in reference to a treatment of a cell or a subject refers to an increase in the effectiveness in lessening or decreasing the symptoms of the disease compared to a reference treatment (e.g., of the same cell or subject, or of a different cell or subject) when measured using any methods known in the art. In certain embodiments, the increase in the effectiveness is at least about 5%, at least about 10%, at least about 20%, at least about 30%, at least about 40%, or at least about 50%.

An improvement in the cancer or cancer-related disease can be characterized as a complete or partial response. “Complete response” refers to an absence of clinically detectable disease with normalization of any previously abnormal radiographic studies, bone marrow, and cerebrospinal fluid (CSF) or abnormal monoclonal protein measurements. “Partial response” refers to at least about 10%, about 20%, about 30%, about 40%, about 50%, about 60%, about 70%, about 80%, or about 90% decrease in all measurable tumor burden (i.e., the number of malignant cells present in the subject, or the measured bulk of tumor masses or the quantity of abnormal monoclonal protein) in the absence of new lesions. The term “treatment” contemplates both a complete and a partial response.

The term “likelihood” generally refers to an increase in the probability of an event. The term “likelihood” when used in reference to the effectiveness of a patient tumor response generally contemplates an increased probability that the rate of tumor progress or tumor cell growth will decrease. The term “likelihood” when used in reference to the effectiveness of a patient tumor response can also generally mean the increase of indicators, such as mRNA or protein expression, that may evidence an increase in the progress in treating the tumor.

The term “predict” generally means to determine or tell in advance. When used to “predict” the effectiveness of a cancer treatment, for example, the term “predict” can mean that the likelihood of the outcome of the cancer treatment can be determined at the outset, before the treatment has begun, or before the treatment period has progressed substantially.

The term “monitor,” as used herein, generally refers to the overseeing, supervision, regulation, watching, tracking, or surveillance of an activity. For example, the term “monitoring the effectiveness of a compound” refers to tracking the effectiveness in treating cancer in a patient or in a tumor cell culture. Similarly, the term “monitoring,” when used in connection with patient compliance, either individually, or in a clinical trial, refers to the tracking or confirming that the patient is actually taking a drug being tested as prescribed. The monitoring can be performed, for example, by following the expression of mRNA or protein biomarkers.

The term “regulate” as used herein refers to controlling the activity of a molecule or biological function, such as enhancing or diminishing the activity or function.

The term “refractory” or “resistant” refers to a circumstance where patients, even after intensive treatment, have residual cancer cells (e.g., leukemia or lymphoma cells) in their lymphatic system, blood, and/or blood forming tissues (e.g., marrow).

A “biological marker” or “biomarker” is a substance whose detection indicates a particular biological state, such as, for example, the presence of cancer. In some embodiments, biomarkers can be determined individually. In other embodiments, several biomarkers can be measured simultaneously. In some embodiments, a “biomarker” indicates a change in the level of mRNA expression that may correlate with the risk or progression of a disease, or with the susceptibility of the disease to a given treatment. In some embodiments, the biomarker is a nucleic acid, such as mRNA or cDNA. In additional embodiments, a “biomarker” indicates a change in the level of polypeptide or protein expression that may correlate with the risk or progression of a disease, or patient's susceptibility to treatment. In some embodiments, the biomarker can be a polypeptide or protein, or a fragment thereof. The relative level of specific proteins can be determined by methods known in the art. For example, antibody-based methods, such as an immunoblot, enzyme-linked immunosorbent assay (ELISA), or other methods can be used.

A “gene set,” as used herein, refers to one or more genes that are chosen by a skilled person in the art. The genes can be grouped based on their relationship with each other, their association with certain cell types, biological functions, phenotypes, or cellular pathways, etc., or solely the discretion of the skilled person in the art. A gene set, as used herein, can comprise as few as only one gene or as many as hundreds, thousands, or hundreds of thousands of genes.

A “signature” or “gene signature,” as used herein, refers to a group of genes. In some embodiments, the group of genes are related to each other because of their association with certain cell types, biological functions, phenotypes, or cellular pathways, etc. A signature can be defined by a skilled person in the art based on different experimental data and/or statistical analysis methods, i.e., a particular signature may contain various numbers of genes or different specific genes depending on the criteria that the skilled person in the art chooses. An example of a gene signature is a LSC signature.

A “LSC17” or “LSC17 signature,” as used herein, refers to a gene signature comprising the following 17 genes: AKR1C3, ARHGAP22, CD34, CDK6, CPXM1, DNMT3B, DPYSL3, EMP1, GPR56, KIAA0125, LAPTM4B, MMRN1, NGFRAP1, NYNRIN, SMIM24, SOCS2, and ZBTB46. A “LSC4” or “LSC4 signature” as used herein, refers to a gene signature comprising the following 4 genes: TNFRSF4, SLC4A1, SLC7A7, and AIM2. A “LSC3” or “LSC3 signature” as used herein, refers to a gene signature comprising the following 3 genes: SLC4A1, SLC7A7, and AIM2. A “LSC17 score” or “LSC17 signature score,” as used herein, refers to the score calculated based on the expression level of the LSC17 signature that comprises the following 17 genes: AKR1C3, ARHGAP22, CD34, CDK6, CPXM1, DNMT3B, DPYSL3, EMP1, GPR56, KIAA0125, LAPTM4B, MMRN1, NGFRAP1, NYNRIN, SMIM24, SOCS2, and ZBTB46. Similarly, a “LSC4 score” or “LSC4 signature score,” as used herein, is a score calculated based on the expression level of the LSC4 signature described above. A “LSC3 score” or “LSC3 signature score,” as used herein, is a score calculated based on the expression level of the LSC3 signature described above.

The terms “polypeptide” and “protein,” as used interchangeably herein, refer to a polymer of three or more amino acids in a serial array, linked through peptide bonds. The term “polypeptide” includes proteins, protein fragments, protein analogues, oligopeptides, and the like. The term “polypeptide” as used herein can also refer to a peptide. The amino acids making up the polypeptide may be naturally derived or may be synthetic. The polypeptide can be purified from a biological sample. The polypeptide, protein, or peptide also encompasses modified polypeptides, proteins, and peptides, e.g., glycopolypeptides, glycoproteins, or glycopeptides; or lipopolypeptides, lipoproteins, or lipopeptides.

The term “expressed” or “expression” as used herein refers to the transcription from a gene to give an RNA nucleic acid molecule at least complementary in part to a region of one of the two nucleic acid strands of the gene. The term “expressed” or “expression” as used herein also refers to the translation from the RNA molecule to give a protein, a polypeptide, or a portion thereof.

The term “expression level” refers to the amount, accumulation, or rate of a biomarker molecule or a gene set. An expression level can be represented, for example, by the amount or the rate of synthesis of a messenger RNA (mRNA) encoded by a gene, the amount or the rate of synthesis of a polypeptide or protein encoded by a gene, or the amount or the rate of synthesis of a biological molecule accumulated in a cell or biological fluid. The term “expression level” refers to an absolute amount of a molecule in a sample or a relative amount of the molecule, determined under steady-state or non-steady-state conditions.

An mRNA that is “upregulated” is generally increased upon a given treatment or condition, or in certain patient groups. An mRNA that is “downregulated” generally refers to a decrease in the level of expression of the mRNA in response to a given treatment or condition, or in certain patient groups. In some situations, the mRNA level can remain unchanged upon a given treatment or condition. An mRNA from a patient sample can be “upregulated” when treated with a drug, as compared to a non-treated control. This upregulation can be, for example, an increase of about 5%, about 10%, about 20%, about 30%, about 40%, about 50%, about 60%, about 70%, about 80%, about 90%, about 100%, about 200%, about 300%, about 500%, about 1,000%, about 5,000%, or more of the comparative control mRNA level. Alternatively, an mRNA can be “downregulated”, or expressed at a lower level, in response to administration of certain compounds or other agents. A downregulated mRNA can be, for example, present at a level of about 99%, about 95%, about 90%, about 80%, about 70%, about 60%, about 50%, about 40%, about 30%, about 20%, about 10%, about 1%, or less of the comparative control mRNA level.

Similarly, the level of a polypeptide or protein biomarker from a patient sample can be increased when treated with a drug, as compared to a non-treated control. This increase can be about 5%, about 10%, about 20%, about 30%, about 40%, about 50%, about 60%, about 70%, about 80%, about 90%, about 100%, about 200%, about 300%, about 500%, about 1,000%, about 5,000%, or more of the comparative control protein level. Alternatively, the level of a protein biomarker can be decreased in response to administration of certain compounds or other agents. This decrease can be, for example, present at a level of about 99%, about 95%, about 90%, about 80%, about 70%, about 60%, about 50%, about 40%, about 30%, about 20%, about 10%, about 1%, or less of the comparative control protein level.

The terms “determining,” “measuring,” “evaluating,” “assessing,” and “assaying” as used herein generally refer to any form of measurement, and include determining whether an element is present or not. These terms include quantitative and/or qualitative determinations. Assessing may be relative or absolute. “Assessing the presence of” can include determining the amount of something present, as well as determining whether it is present or absent.

The terms “nucleic acid” and “polynucleotide” are used interchangeably herein to describe a polymer of any length composed of nucleotides, e.g., deoxyribonucleotides or ribonucleotides, or compounds produced synthetically, which can hybridize with naturally occurring nucleic acids in a sequence specific manner analogous to that of two naturally occurring nucleic acids, e.g., can participate in Watson-Crick base pairing interactions. As used herein in the context of a polynucleotide sequence, the term “bases” (or “base”) is synonymous with “nucleotides” (or “nucleotide”), i.e., the monomer subunit of a polynucleotide. The terms “nucleoside” and “nucleotide” are intended to include those moieties that contain not only the known purine and pyrimidine bases, but also other heterocyclic bases that have been modified. Such modifications include methylated purines or pyrimidines, acylated purines or pyrimidines, alkylated riboses or other heterocycles. In addition, the terms “nucleoside” and “nucleotide” include those moieties that contain not only conventional ribose and deoxyribose sugars, but other sugars as well. Modified nucleosides or nucleotides also include modifications on the sugar moiety, e.g., wherein one or more of the hydroxyl groups are replaced with halogen atoms or aliphatic groups, or are functionalized as ethers, amines, or the like. “Analogues” refer to molecules having structural features that are recognized in the literature as being mimetics, derivatives, having analogous structures, or other like terms, and include, for example, polynucleotides incorporating non-natural nucleotides, nucleotide mimetics such as 2′-modified nucleosides, peptide nucleic acids, oligomeric nucleoside phosphonates, and any polynucleotide that has added substituent groups, such as protecting groups or linking moieties.

The term “complementary” refers to specific binding between polynucleotides based on the sequences of the polynucleotides. As used herein, a first polynucleotide and a second polynucleotide are complementary if they bind to each other in a hybridization assay under stringent conditions, e.g., if they produce a given or detectable level of signal in a hybridization assay. Portions of polynucleotides are complementary to each other if they follow conventional base-pairing rules, e.g., A pairs with T (or U) and G pairs with C, although small regions (e.g., fewer than about 3 bases) of mismatch, insertion, or deleted sequence may be present.

The terms “isolated” and “purified” refer to isolation of a substance (such as mRNA, DNA, or protein) such that the substance comprises a substantial portion of the sample in which it resides, i.e., greater than the portion of the substance that is typically found in its natural or un-isolated state. Typically, a substantial portion of the sample comprises, e.g., greater than 1%, greater than 2%, greater than 5%, greater than 10%, greater than 20%, greater than 50%, or more, usually up to about 90%-100% of the sample. For example, a sample of isolated mRNA can typically comprise at least about 1% total mRNA. Techniques for purifying polynucleotides are well known in the art and include, for example, gel electrophoresis, ion-exchange chromatography, affinity chromatography, flow sorting, and sedimentation according to density.

As used herein, the term “bound” indicates direct or indirect attachment. In the context of chemical structures, “bound” (or “bonded”) may refer to the existence of a chemical bond directly joining two moieties or indirectly joining two moieties (e.g., via a linking group or any other intervening portion of the molecule). The chemical bond may be a covalent bond, an ionic bond, a coordination complex, hydrogen bonding, van der Waals interactions, or hydrophobic stacking, or may exhibit characteristics of multiple types of chemical bonds. In certain instances, “bound” includes embodiments where the attachment is direct and embodiments where the attachment is indirect.

The term “sample” as used herein relates to a material or mixture of materials, typically, although not necessarily, in fluid form, containing one or more components of interest.

“Biological sample” as used herein refers to a sample obtained from a biological subject, including a sample of biological tissue or fluid origin, obtained, reached, or collected in vivo or in situ. A biological sample also includes samples from a region of a biological subject containing precancerous or cancer cells or tissues. Such samples can be, but are not limited to, organs, tissues, and cells isolated from a mammal. Exemplary biological samples include but are not limited to cell lysate, cells, tissues, organs, organelles, a biological fluid, a blood sample, a urine sample, a skin sample, and the like. Preferred biological samples include, but are not limited to, whole blood, partially purified blood, PBMC, tissue biopsies (including tumor biopsies), circulating tumor cells, and the like.

The term “polymerase chain reaction” or “PCR” as used herein generally refers to a procedure wherein small amounts of a nucleic acid, RNA and/or DNA, are amplified as described, for example, in U.S. Pat. No. 4,683,195. Generally, sequence information from the ends or beyond of the region of interest needs to be available, such that oligonucleotide primers can be designed; these primers will be identical or similar in sequence to opposite strands of the template to be amplified. The 5′ terminal nucleotides of the two primers may coincide with the ends of the amplified material. PCR can be used to amplify specific RNA sequences, specific DNA sequences from total genomic DNA, and cDNA transcribed from total cellular RNA, bacteriophage, or plasmid sequences, etc. See generally Mullis et al., Cold Spring Harbor Symp. Quant. Biol. 1987, 51:263-273; PCR Technology (Stockton Press, NY, Erlich, ed., 1989).

“Tautomer” as used herein refers to isomeric forms of a compound that are in equilibrium with each other. The concentrations of the isomeric forms will depend on the environment the compound is found in and may be different depending upon, for example, whether the compound is a solid or is in an organic or aqueous solution. For example, in aqueous solution, pyrazoles may exhibit the following isomeric forms, which are referred to as tautomers of each other:

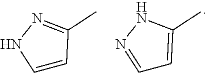

As used herein and unless otherwise indicated, the term “pharmaceutically acceptable salt” encompasses non-toxic acid and base addition salts of the compound to which the term refers. Acceptable non-toxic acid addition salts include those derived from organic and inorganic acids know in the art, which include, for example, hydrochloric acid, hydrobromic acid, phosphoric acid, sulfuric acid, methanesulphonic acid, acetic acid, tartaric acid, lactic acid, succinic acid, citric acid, malic acid, maleic acid, sorbic acid, aconitic acid, salicylic acid, phthalic acid, embolic acid, enanthic acid, and the like. Compounds that are acidic in nature are capable of forming salts with various pharmaceutically acceptable bases. The bases that can be used to prepare pharmaceutically acceptable base addition salts of such acidic compounds are those that form non-toxic base addition salts, i.e., salts containing pharmacologically acceptable cations such as, but not limited to, alkali metal or alkaline earth metal salts (calcium, magnesium, sodium, or potassium salts in particular). Suitable organic bases include, but are not limited to, N,N-dibenzylethylenediamine, chloroprocaine, choline, diethanolamine, ethylenediamine, meglumaine (N-methylglucamine), lysine, and procaine.

As used herein and unless otherwise indicated, the term “solvate” means a compound provided herein or a salt thereof that further includes a stoichiometric or non-stoichiometric amount of solvent bound by non-covalent intermolecular forces. Where the solvent is water, the solvate is a hydrate.

As used herein and unless otherwise indicated, the term “co-crystal” means a crystalline form that contains more than one compound in a crystal lattice. Co-crystals include crystalline molecular complexes of two or more non-volatile compounds bound together in a crystal lattice through non-ionic interactions. As used herein, co-crystals include pharmaceutical co-crystals wherein the crystalline molecular complexes containing a therapeutic compound and one or more additional non-volatile compound(s) (referred to herein as counter-molecule(s)). A counter-molecule in a pharmaceutical co-crystal is typically a non-toxic pharmaceutically acceptable molecule, such as, for example, food additives, preservatives, pharmaceutical excipients, or other active pharmaceutical ingredients (API). In some embodiments, pharmaceutical co-crystals enhance certain physicochemical properties of drug products (e.g., solubility, dissolution rate, bioavailability, and/or stability) without compromising the chemical structural integrity of the API. See, e.g., Jones et al., MRS Bulletin 2006, 31, 875-879; Trask, Mol. Pharmaceutics 2007, 4(3):301-309; Schultheiss & Newman, Crystal Growth & Design 2009, 9(6):2950-2967; Shan & Zaworotko, Drug Discovery Today 2008, 13(9/10):440-446; and Vishweshwar et al., J. Pharm. Sci. 2006, 95(3):499-516.

As used herein, and unless otherwise specified, the term “stereoisomer” encompasses all enantiomerically/stereoisomerically pure and enantiomerically/stereoisomerically enriched compounds of this invention.

As used herein and unless otherwise indicated, the term “stereoisomerically pure” means a composition that comprises one stereoisomer of a compound and is substantially free of other stereoisomers of that compound. For example, a stereoisomerically pure composition of a compound having one chiral center will be substantially free of the opposite enantiomer of the compound. A stereoisomerically pure composition of a compound having two chiral centers will be substantially free of other diastereomers of the compound. A typical stereoisomerically pure compound comprises greater than about 80% by weight of one stereoisomer of the compound and less than about 20% by weight of other stereoisomers of the compound, more preferably greater than about 90% by weight of one stereoisomer of the compound and less than about 10% by weight of the other stereoisomers of the compound, even more preferably greater than about 95% by weight of one stereoisomer of the compound and less than about 5% by weight of the other stereoisomers of the compound, and most preferably greater than about 97% by weight of one stereoisomer of the compound and less than about 3% by weight of the other stereoisomers of the compound.

As used herein and unless otherwise indicated, the term “stereoisomerically enriched” means a composition that comprises greater than about 60% by weight of one stereoisomer of a compound, preferably greater than about 70% by weight, more preferably greater than about 80% by weight of one stereoisomer of a compound. As used herein and unless otherwise indicated, the term “enantiomerically pure” means a stereomerically pure composition of a compound having one chiral center. Similarly, the term “stereoisomerically enriched” means a stereoisomerically enriched composition of a compound having one chiral center.

As used herein and unless otherwise indicated, the term “prodrug” means a derivative of a compound that can hydrolyze, oxidize, or otherwise react under biological conditions (in-vitro or in-vivo) to provide the compound. Examples of prodrugs include, but are not limited to, derivatives of compounds described herein (e.g., Compound 1) that include biohydrolyzable moieties such as biohydrolyzable amides, biohydrolyzable esters, biohydrolyzable carbamates, biohydrolyzable carbonates, biohydrolyzable ureides, and biohydrolyzable phosphate analogues.

It should also be noted compounds can contain unnatural proportions of atomic isotopes at one or more of the atoms. For example, the compounds may be radiolabeled with radioactive isotopes, such as for example tritium (³H), iodine-125 (¹²⁵I), sulfur-35 (³⁵S), or carbon-14 (¹⁴C), or may be isotopically enriched, such as with deuterium (²H), carbon-13 (¹³C), or nitrogen-15 (¹⁵N). As used herein, an “isotopologue” is an isotopically enriched compound. The term “isotopically enriched” refers to an atom having an isotopic composition other than the natural isotopic composition of that atom. “Isotopically enriched” may also refer to a compound containing at least one atom having an isotopic composition other than the natural isotopic composition of that atom. The term “isotopic composition” refers to the amount of each isotope present for a given atom. Radiolabeled and isotopically enriched compounds are useful as therapeutic agents, e.g., cancer and inflammation therapeutic agents, research reagents, e.g., binding assay reagents, and diagnostic agents, e.g., in vivo imaging agents. All isotopic variations of the compounds as described herein, whether radioactive or not, are intended to be encompassed within the scope of the embodiments provided herein. In some embodiments, there are provided isotopologues of the compounds, for example, the isotopologues are deuterium, carbon-13, or nitrogen-15 enriched compounds. In some embodiments, isotopologues provided herein are deuterium enriched compounds. In some embodiments, isotopologues provided herein are deuterium enriched compounds, where the deuteration occurs on the chiral center. In some embodiments, provided herein are isotopologues of the compounds provided herein, where deuteration occurs on the chiral center. In some embodiments, provided herein are isotopologues of Compound D, where deuteration occurs on the chiral center.

The term “about” or “approximately” means an acceptable error for a particular value as determined by one of ordinary skill in the art, which depends in part on how the value is measured or determined. In certain embodiments, the term “about” or “approximately” means within 1, 2, 3, or 4 standard deviations. In certain embodiments, the term “about” or “approximately” means within 50%, 20%, 15%, 10%, 9%, 8%, 7%, 6%, 5%, 4%, 3%, 2%, 1%, 0.5%, or 0.05% of a given value or range.

It should be noted that if there is a discrepancy between a depicted structure and a name given to that structure, the depicted structure is to be accorded more weight. In addition, if the stereochemistry of a structure or a portion of a structure is not indicated with, for example, bold or dashed lines, the structure or portion of the structure is to be interpreted as encompassing all stereoisomers of it.

The practice of the embodiments provided herein will employ, unless otherwise indicated, conventional techniques of molecular biology, microbiology, and immunology, which are within the skill of those working in the art. Such techniques are explained fully in the literature. Examples of particularly suitable texts for consultation include the following: Sambrook et al., Molecular Cloning: A Laboratory Manual (4th ed. 2014); Glover, ed., DNA Cloning, Volumes I and II (2^{nd} ed. 1995); Immunochemical Methods in Cell and Molecular Biology (Academic Press, London); Scopes, Protein Purification: Principles and Practice (Springer Verlag, N.Y., 3rd ed. 1993); and Weir & Blackwell, eds., Handbook of Experimental Immunology, Volumes I-IV (5^{th} ed. 1996).

### 5.2. Gene Sets, Biomarkers and Methods of Use Thereof

5.2.1 Gene Sets

The methods provided herein are based, in part, on the finding that detectable increase in expression level of certain gene sets (or gene signatures) is observed in subjects with cancer (e.g., a hematological cancer such as lymphoma, MM, or leukemia) who are responsive to a given treatment, e.g., a compound, such as Compound D, or a stereoisomer or a mixture of stereoisomers, tautomer, pharmaceutically acceptable salt, solvate, isotopologue, prodrug, hydrate, co-crystal, clathrate, or a polymorph thereof, and that the expression level of the gene set may be used for predicting the responsiveness of the subjects to the treatment. In some embodiments, the compound is as described herein in Section 5.5. In one embodiment, the compound is Compound D.

In certain embodiments, the gene set is a gene signature that comprises a plurality of genes that are related by their association with certain cell types, biological functions, phenotypes, or cellular pathways, etc. For example, in one specific embodiment, the genes within the gene signature are related by their association with stem cells or a subgroup of stem cells (e.g., LSC).

In some embodiments, the gene signature comprises at least one gene selected from the group of genes that are related by their association with certain cell types, biological functions, or cellular pathways, etc. In other embodiments, the signature comprises two, three, four, five, six, seven, eight, nine, ten, twenty, thirty, forty, fifty, or all genes selected from the group of genes that are related.

In one aspect, provided herein is a method of identifying a subject having cancer who is likely to be responsive to a treatment comprising a compound provided herein or predicting the responsiveness of a subject having or suspected of having cancer to a treatment comprising the compound, comprising: i. providing a sample from the subject; ii. measuring gene expression level of one or more genes in the sample; iii. calculating a leukemic stem cell (LSC) signature score for the sample based on the gene expression level of the one or more genes; and iv. identifying the subject as being likely to be responsive to the treatment comprising the compound if the level of the LSC signature score is higher than a reference level thereof, wherein the treatment compound is Compound D, or a stereoisomer or mixture of stereoisomers, isotopologue, pharmaceutically acceptable salt, tautomer, solvate, hydrate, co-crystal, clathrate, or polymorph thereof.

In some embodiments, provided herein is a method of treating a subject having cancer with a compound, comprising identifying the subject having cancer that may be responsive to the treatment comprising the compound using the methods provided herein (e.g., described above), and administering the subject a therapeutically effective amount of the compound if the subject is identified as being likely to be responsive to the treatment comprising the compound.

In certain embodiments, the cancer is a hematological cancer. In one embodiment, the hematological cancer is lymphoma. In another embodiment, the hematological cancer is leukemia. In yet another embodiment, the hematological cancer is MM. In a specific embodiment, the leukemia is ALL. In another specific embodiment, the leukemia is AML. In yet another specific embodiment, the leukemia is CLL. In still another embodiment, the leukemia is CML.

In some embodiments, the AML is relapsed. In certain embodiments, the AML is refractory. In other embodiments, the AML is resistant to conventional therapy.

In a specific embodiment, provided herein is a method of identifying a subject having AML who is likely to be responsive to a treatment comprising a compound provided herein or predicting the responsiveness of a subject having or suspected of having AML to a treatment comprising the compound, comprising: i. providing a sample from the subject; ii. measuring gene expression level of one or more genes in the sample; iii. calculating a leukemic stem cell (LSC) signature score for the sample based on the gene expression level of the one or more genes; and iv. identifying the subject as being likely to be responsive to the treatment comprising the compound if the level of the LSC signature score is higher than a reference level thereof, wherein the treatment compound is Compound D, or a stereoisomer or mixture of stereoisomers, isotopologue, pharmaceutically acceptable salt, tautomer, solvate, hydrate, co-crystal, clathrate, or polymorph thereof.

In certain embodiments, the method provided herein is a method of identifying a subject having cancer who is likely to be responsive to a treatment compound. In some embodiments, the method provided herein is a method of predicting the responsiveness of a subject having or suspected of having cancer to a treatment compound. In other embodiments, the method provided herein is a method of treating cancer with a treatment compound. In yet other embodiments, the cancer is characterized by an increased level of a LSC signature (or higher LSC signature score). In still other embodiments, the LSC signature is a LSC signature described herein. In one embodiment, provided herein is a method of treating cancer characterized by an increased level of a LSC signature (or higher LSC signature score) described herein with a treatment compound. In another embodiment, provided herein is a method of treating leukemia characterized by an increased level of a LSC signature (or higher LSC signature score) described herein with a treatment compound. In yet another embodiment, provided herein is a method of treating AML characterized by an increased level of a LSC signature (or higher LSC signature score) described herein with a treatment compound.

In certain embodiments of the methods provided herein, the reference level (reference level of the LSC signature score) is the level of the LSC signature (or LSC signature score) in a control. In some embodiments, the control is obtained from a healthy subject not having cancer. In other embodiments, the control is obtained from a subject having cancer but with good prognosis risk. In yet other embodiments, the control is obtained from a subject having cancer and the cancer has been ameliorated or cured by a treatment other than administering to the subject the treatment compounds described herein. In other embodiments, the control is obtained from a subject having cancer but not responsive to the treatment compound. In yet other embodiments, the control is from the same tissue or cell source (e.g., blood or certain blood cells) as the sample. In still other embodiments, the control is a cell line (e.g., an AML cell line). In one embodiment, the reference level is the level of the LSC signature in a control that is obtained from a healthy subject not having cancer, and the control is from the same tissue or cell source (e.g., blood or certain blood cells) as the sample. In another embodiment, the reference level is the level of the LSC signature in a control that is obtained from a subject having cancer but with good prognosis risk, and the control is from the same tissue or cell source (e.g., blood or certain blood cells) as the sample. In yet another embodiment, the reference level is the level of the LSC signature in a control that is obtained from a subject having cancer and the cancer has been ameliorated or cured by a treatment other than administering to the subject the treatment compounds described herein, and the control is from the same tissue or cell source (e.g., blood or certain blood cells) as the sample. In still another embodiment, the reference level is the level of the LSC signature in a control that is obtained from a subject having cancer but not responsive to the treatment compound, and the control is from the same tissue or cell source (e.g., blood or certain blood cells) as the sample. In yet another embodiment, the reference level is the level of the LSC signature in a control that is a cell line. In still another embodiment, the reference level is the level of the LSC signature in a control cell line that is derived from the same cell source (e.g., white blood cells, blast cells, etc.) as cancer. In one embodiment, the reference level is the level of the LSC signature in a control that is a cancer cell line. In another embodiment, the reference level is the level of the LSC signature in a control that is an AML cell line. In yet another embodiment, the reference level (or the reference score of LSC signature) is determined based on the LSC signature scores obtained from a population. In some embodiments, the reference score of LSC signature is pre-determined.

In some embodiments, the subject has received a prior treatment before the methods provided herein. In certain embodiments, the prior treatment is a treatment other than administering to the subject the same treatment compound as the methods provided herein. In other embodiments, the prior treatment is administering to the subject the same treatment compound as the methods provided herein. In one embodiment, the prior treatment comprises the same treatment compound with the same dosage regime as the methods provided herein. In another embodiment, the prior treatment comprises the same treatment compound with a different dosage regime (e.g., a different amount and/or administration frequency of the treatment compound) compared to the methods provided herein. In some embodiments where the subject has received a prior treatment before the methods provided herein, the control is obtained from the same subject before the prior treatment. In specific embodiments, the control is from the same tissue or cell source (e.g., blood or certain blood cells) before the prior treatment as the sample. In some embodiments, the prior treatment is one or more agents selected from the group consisting of daunorubicin, cytarabine (ara-C), and gemtuzumab ozogamicin, or resistant to chemotherapies.

In certain embodiments, the gene set comprises a gene signature that is related to certain cell types (e.g., stem cells). In some embodiments, the gene set comprises a gene signature that is related to certain biological functions (e.g., protein metabolism). In other embodiments, the gene set comprises a gene signature that is related to certain cellular pathways (e.g., a UPR pathway). In one embodiment, the gene set comprises a gene signature related to leukemic stem cells (LSC). In another embodiment, the gene set comprises a LSC gene signature.

In some embodiments, the LSC gene signature comprises one or more genes selected from a group consisting of CD34, SPINK2, LAPTM48, HOXA5, GUCY1A3, SHANK3, ANGPT1, ARHGAP22, LOC284422, MYCN, MAMDC2, PRSSL1, KIAA0125, GPSM1, HOXA9, MMRN1, FSCN1, DNMT38, HOXA6, AIF1L, SOCS2, CDK6, FAM69B, NGFRAP1, C3orf54, CPXM1, TNFRSF4, ZBTB46, DPYSL3, NYNRIN, COL24A1, FAM30A, C10orf140, SPNS2, GPR56, AKR1C3, FLT3, TFPI, KCNK17, EPDR1, C1orf150, BIVM, H2AFY2, VWF, EMP1, RAGE, ATP8B4, GATA2, SLC25A37, SGK, LOC652694, ITPR3, LOC654103, CXCR4, FCRL3, RBM38, LILRA5, IL18RAP, CCDC109B, ISG20, MTSS1, CECR1, ADAM19, FCGR2A, AIM2, NPL, IL10RA, CTSL1, GNLY, CKAP4, ADM, KLRB1, SLC15A3, FGR, FCRLA, IL2RB, CXCL16, SLC4A1, GZMH, FLJ22662, LOC647506, GIMAP4, JAZF1, CTSH, GZMA, CHST15, AQP9, CD247, BCL6, SLC7A7, E2F2, LOC647450, GZMB, LOC652493, HBM, CD14, ALAS2, HBB, LOC642113, AHSP, FCN1, CD48, HBA2, and HBA1.

In other embodiments, the LSC gene signature comprises one or more genes selected from a group consisting of CD34, SPINK2, LAPTM48, HOXA5, GUCY1A3, SHANK3, ANGPT1, ARHGAP22, LOC284422, MYCN, MAMDC2, PRSSL1, KIAA0125, GPSM1, HOXA9, MMRN1, FSCN1, DNMT38, HOXA6, AIF1L, SOCS2, CDK6, FAM69B, NGFRAP1, C3orf54, CPXM1, TNFRSF4, ZBTB46, DPYSL3, NYNRIN, COL24A1, FAM30A, C10orf140, SPNS2, GPR56, AKR1C3, FLT3, TFPI, KCNK17, EPDR1, C1orf150, BIVM, H2AFY2, VWF, EMP1, RAGE, ATP8B4, and GATA2.

In certain embodiments, the LSC signature comprises at least one gene selected from Table 1.

TABLE 1
LSC17 Signature
AKR1C3
ARHGAP22
CD34
CDK6
CPXM1
DNMT3B
DPYSL3
EMP1
GPR56
KIAA0125
LAPTM4B
MMRN1
NGFRAP1
NYNRIN
SMIM24
SOCS2
ZBTB46

In certain embodiments, the LSC signature comprises at least one gene selected from the group consisting of AKR1C3, ARHGAP22, CD34, CDK6, CPXM1, DNMT3B, DPYSL3, EMP1, GPR56, KIAA0125, LAPTM4B, MMRN1, NGFRAP1, NYNRIN, SMIM24, SOCS2, and ZBTB46. In one embodiment, the LSC signature comprises AKR1C3. In one embodiment, the LSC signature comprises ARHGAP22. In another embodiment, the LSC signature comprises CD34. In yet another embodiment, the LSC signature comprises CDK6. In still another embodiment, the LSC signature comprises CPXM1. In one embodiment, the LSC signature comprises DNMT3B. In another embodiment, the LSC signature comprises DPYSL3. In yet another embodiment, the LSC signature comprises EMP1. In still another embodiment, the LSC signature comprises GPR56. In one embodiment, the LSC signature comprises KIAA0125. In another embodiment, the LSC signature comprises LAPTM4B. In yet another embodiment, the LSC signature comprises MMRN1. In still another embodiment, the LSC signature comprises NGFRAP1. In one embodiment, the LSC signature comprises NYNRIN. In another embodiment, the LSC signature comprises SMIM24. In yet another embodiment, the LSC signature comprises SOCS2. In still another embodiment, the LSC signature comprises ZBTB46.

In certain embodiments, the LSC signature comprises two genes selected from Table 1. In some embodiments, the LSC signature comprises three genes selected from Table 1. In other embodiments, the LSC signature comprises four genes selected from Table 1. In yet other embodiments, the LSC signature comprises five genes selected from Table 1. In still other embodiments, the LSC signature comprises six genes selected from Table 1. In certain embodiments, the LSC signature comprises seven genes selected from Table 1. In some embodiments, the LSC signature comprises eight genes selected from Table 1. In other embodiments, the LSC signature comprises nine genes selected from Table 1. In yet other embodiments, the LSC signature comprises ten genes selected from Table 1. In still other embodiments, the LSC signature comprises twelve genes selected from Table 1. In certain embodiments, the LSC signature comprises fourteen genes selected from Table 1. In some embodiments, the LSC signature comprises sixteen genes selected from Table 1. In other embodiments, the LSC signature comprises all seventeen genes selected from Table 1, which is referred to as “LSC17” or “LSC17 signature.”

In some embodiments, the LSC signature score (LSC17 score) is calculated as follows: (expression level of DNMT3B×weight of DNMTT3B)+(expression level of ZBTB46×weight of ZBTB46)+(expression level of NYNRIN×weight of NYNRIN)+(expression level of ARHGAP22×weight of ARHGAP22)+(expression level of LAPTM4B×weight of LAPTM4B)+(expression level of MMRN1×weight of MMRN1)+(expression level of DPYSL3×weight of DPYSL3)+(expression level of KIAA0125×weight of KIAA0125)+(expression level of CDK6×weight of CDK6)+(expression level of CPXM1×weight of CPXM1)+(expression level of SOCS2×weight of SOCS2)+(expression level of SMIM24×weight of SMIM24)+(expression level of EMP1×weight of EMP1)+(expression level of NGFRAP1×weight of NGFRAP1)+(expression level of CD34×weight of CD34)+(expression level of AKR1C3×weight of AKR1C3)+(expression level of GPR56×weight of GPR56); and the weight of DNMTT3B is in a range from 0.06 to 0.1, the weight of ZBTB46 is in a range from −0.05 to −0.01, the weight of NYNRIN is in a range from −0.01 to 0.03, the weight of ARHGAP22 is in a range from −0.03 to 0.01, the weight of LAPTM4B is in a range from −0.015 to 0.025, the weight of MMRN1 is in a range from 0.005 to 0.045, the weight of DPYSL3 is in a range from 0.01 to 0.05, the weight of KIAA0125 is in a range from 0.009 to 0.039, the weight of CDK6 is in a range from −0.09 to −0.05, the weight of CPXM1 is in a range from −0.045 to −0.005, the weight of SOCS2 is in a range from 0.007 to 0.047, the weight of SMIM24 is in a range from −0.043 to −0.003, the weight of EMP1 is in a range from 0.01 to 0.035, the weight of NGFRAP1 is in a range from 0.025 to 0.065, the weight of CD34 is in a range from 0.01 to 0.05, the weight of AKR1C3 is in a range from −0.06 to −0.02, and the weight of GPR56 is in a range from 0.03 to 0.07.

In some embodiments, the weight of DNMTT3B is in a range from 0.08 to 0.09, the weight of ZBTB46 is in a range from −0.03 to −0.04, the weight of NYNRIN is in a range from −0.008 to 0.009, the weight of ARHGAP22 is in a range from −0.015 to 0.01, the weight of LAPTM4B is in a range from −0.006 to 0.005, the weight of MMRN1 is in a range from 0.02 to 0.03, the weight of DPYSL3 is in a range from 0.02 to 0.03, the weight of KIAA0125 is in a range from 0.01 to 0.02, the weight of CDK6 is in a range from −0.08 to −0.07, the weight of CPXM1 is in a range from −0.02 to −0.03, the weight of SOCS2 is in a range from 0.02 to 0.03, the weight of SMIM24 is in a range from −0.02 to −0.03, the weight of EMP1 is in a range from 0.014 to 0.02, the weight of NGFRAP1 is in a range from 0.04 to 0.05, the weight of CD34 is in a range from 0.03 to 0.04, the weight of AKR1C3 is in a range from −0.04 to −0.05, and the weight of GPR56 is in a range from 0.04 to 0.055.

In some embodiments, the weight for DNMT3B is about 0.0874, the weight of ZBTB46 is about −0.0347, the weight of NYNRIN is about 0.00865, the weight of ARHGAP22 is about −0.0138, the weight of LAPTM4B is about 0.00582, the weight of MMRN1 is about 0.0258, the weight of DPYSL3 is about 0.0284, the weight of KIAA0125 is about 0.0196, the weight of CDK6 is about −0.0704, the weight of CPXM1 is about −0.0258, the weight of SOCS2 is about 0.0271, the weight of SMIM24 is about −0.0226, the weight of EMP1 is about 0.0146, the weight of NGFRAP1 is about 0.0465, the weight of CD34 is about 0.0338, the weight of AKR1C3 is about −0.0402, and the weight of GPR56 is about 0.0501.

In a specific embodiment, the LSC signature score (LSC17 score) is calculated as follows: (expression level of DNMT3B×0.0874)+(expression level of ZBTB46×−0.0347)+(expression level of NYNRIN×0.00865)+(expression level of ARHGAP22×−0.0138)+(expression level of LAPTM4B×0.00582)+(expression level of MMRN1×0.0258)+(expression level of DPYSL3×0.0284)+(expression level of KIAA0125×0.0196)+(expression level of CDK6×−0.0704)+(expression level of CPXM1×−0.0258)+(expression level of SOCS2×0.0271)+(expression level of SMIM24×−0.0226)+(expression level of EMP1×0.0146)+(expression level of NGFRAP1×0.0465)+(expression level of CD34×0.0338)+(expression level of AKR1C3×−0.0402)+(expression level of GPR56×0.0501).

In some embodiments, the reference level is 0.1, 0.2, 0.3, 0.4, 0.5, 0.6, 0.7, 0.8. 0.9, 1.0, 1.1, 1.2, 1.3, 1.4, 1.5, 1.6, 1.7, 1.8, 1.9, or 2.

In certain embodiments, the LSC signature comprises at least one gene selected from TNFRSF4, SLC4A1, SLC7A7, and AIM2. In one embodiment, the LSC signature comprises TNFRSF4. In one embodiment, the LSC signature comprises SLC4A1. In another embodiment, the LSC signature comprises SLC7A7. In yet another embodiment, the LSC signature comprises AIM2.

In certain embodiments, the LSC signature comprises two genes selected from TNFRSF4, SLC4A1, SLC7A7, and AIM2. In some embodiments, the LSC signature comprises three genes selected from TNFRSF4, SLC4A1, SLC7A7, and AIM2. In some embodiments, the LSC signature consists of TNFRSF4, SLC4A1, SLC7A7, and AIM2, which is referred to as LSC4 or LSC4 signature.

In some embodiments, the LSC signature score (LSC4 signature score) is calculated as follows: (expression level of TNFRSF4×weight of TNFRSF4)+(expression level of SLC4A1×weight of SLC4A1)+(expression level of SLC7A7×weight of SLC7A7)+(expression level of AIM2×weight of AIM2); and wherein the weight of TNFRSF4 is in a range from −2 to −1, the weight of SLC4A1 is in a range from 11 to 15, the weight of SLC7A7 is in a range from −5.5 to −1.5, the weight of AIM2 is in a range from −5 to −1.

In some embodiments, the weight of TNFRSF4 is in a range from −1.5 to −1, the weight of SLC4A1 is in a range from 13 to 14, the weight of SLC7A7 is in a range from −4 to −3, the weight of AIM2 is in a range from −3 to −4.

In some embodiments, the weight of TNFRSF4 is about −1.13, the weight of SLC4A1 is about 13.59, the weight of SLC7A7 is about −3.57, and the weight of AIM2 is about −3.04.

In a specific embodiment, LSC signature score is calculated as follows: (expression level of TNFRSF4×−1.13)+(expression level of SLC4A1×13.59)+(expression level of SLC7A7×−3.57)+(expression level of AIM2×−3.04).

In certain embodiments, the LSC signature comprises at least one gene selected from SLC4A1, SLC7A7, and AIM2. In certain embodiments, the LSC signature comprises two genes selected from SLC4A1, SLC7A7, and AIM2. In some embodiments, the LSC signature consists of SLC4A1, SLC7A7, and AIM2, which is referred to as LSC3 or LSC3 signature.

In some embodiments, the LSC signature score (LSC3 signature score) is calculated as follows: (expression level of SLC4A1×weight of SLC4A1)+(expression level of SLC7A7×weight of SLC7A7)+(expression level of AIM2×weight of AIM2); and wherein the weight of SLC4A1 is in a range from 11 to 15, the weight of SLC7A7 is in a range from −5.5 to −1.5, the weight of AIM2 is in a range from −5 to −1.

In some embodiments, the weight of SLC4A1 is in a range from 13 to 14, the weight of SLC7A7 is in a range from −4 to −3, the weight of AIM2 is in a range from −3 to −4.

In some embodiments, the weight of SLC4A1 is about 13.59, the weight of SLC7A7 is about −3.57, and the weight of AIM2 is about −3.04.

In a specific embodiment, LSC signature score is calculated as follows: (expression level of SLC4A1×13.59)+(expression level of SLC7A7×−3.57)+(expression level of AIM2×−3.04).

In some embodiments, the method provided herein comprises determining that the patient is likely to be responsive to the treatment comprising the compound provided herein if the LSC signature score in the sample is about 5%, about 10%, about 20%, about 30%, about 40%, about 50%, about 60%, about 70%, about 80%, about 90%, about 100%, about 2 times, about 5 times, about 10 times, about 20 times, about 50 times, or about 100 times higher than the reference score of the LSC signature. In some embodiments, the LSC signature score is LSC17 signature score. In some embodiments, the LSC signature score is LSC4 signature score. In other embodiments, the LSC signature score is LSC3 signature score.

5.2.2 Cell Surface Markers

As shown in Section 6 below, treatment with the present compounds (e.g., Compound D) induces reduction or increase of certain type of cells with certain cell markers. For example, the proportion of primitive cells and/or the proportion differentiated leukemia cells changes upon treatment with Compound D. Therefore, in another aspect, provided herein is a method of predicting responsiveness to a treatment compound (e.g., Compound D, or a stereoisomer or a mixture of stereoisomers, tautomer, pharmaceutically acceptable salt, solvate, isotopologue, prodrug, hydrate, co-crystal, clathrate, or a polymorph thereof) based on the reduction or increase of certain types of cells or associated cell surface markers.

In some embodiments, provided herein is a method of identifying a subject having cancer who is likely to be responsive to a treatment comprising a compound or predicting the responsiveness of a subject having or suspected of having cancer to a treatment comprising the compound, comprising: i. providing a sample from the subject; ii. administering the compound to the sample; iii. measuring the proportion of one or more types of cells; iv. identifying the subject as being likely to be responsive to the treatment comprising the compound if the proportion of the one or more types of cells differentiates from a reference proportion of the cells, wherein the treatment compound is 2-(4-chlorophenyl)-N-((2-(2,6-dioxopiperidin-3-yl)-1-oxoisoindolin-5-yl)methyl)-2,2-difluoroacetamide (Compound D), which has the following structure:

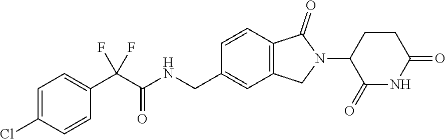 [SMILES: O=C1CCC(N2Cc3cc(CNC(=O)C(F)(F)c4ccc(Cl)cc4)ccc3C2=O)C(=O)N1]

or a stereoisomer or mixture of stereoisomers, isotopologue, pharmaceutically acceptable salt, tautomer, solvate, hydrate, co-crystal, clathrate, or polymorph thereof.

In some embodiments, the method further comprises administering to the subject a therapeutically effective amount of the compound if the subject is identified as being likely to be responsive to the treatment comprising the compound.

In some embodiments, the reference proportion of a type of cells is the proportion of the type of cells in the sample prior to administering the compound. In other embodiments, the reference proportion of a type of cells is a pre-determined proportion. In yet other embodiments, the reference proportion of a type of cells is the proportion of the type of cells in sample obtained from a subject that is not responsive to the treatment with the compound.

In some embodiments, the method comprises measuring the proportion of primitive cells and/or the proportion differentiated leukemia cells. In some embodiments, a reduction of the proportion of primitive cells as compared to the proportion prior to administering the compound indicates that the subject is likely to be responsive to the treatment comprising the compound. In other embodiments, an increase of the proportion of differentiated leukemia cells as compared to the proportion prior to administering the compound indicates that the subject is likely to be responsive to the treatment comprising the compound.

In some embodiments, the method comprises measuring and comparing the proportion of CD34+, CD15+ cells, CD14+ cells, and/or CD11b+ cells prior to and after administering a compound to a sample.

In some embodiments, the method comprises measuring the proportion of CD34+ cells, and wherein a reduction of the proportion of CD34+ cells as compared to the proportion of CD34+ cells prior to administering the compound indicates the subject is likely to be responsive to the treatment comprising the compound.

In other embodiments, the method comprises measuring the proportion of CD15+ cells and/or CD14+ cells, and wherein an increase of the proportion of CD15+ cells and/or CD14+ cells as compared to the proportion of CD15+ cells and/or CD14+ cells prior to administering the compound indicates the subject is likely to be responsive to the treatment comprising the compound.

In other embodiments, provided herein is a method of identifying a subject having cancer who is likely to be responsive to a treatment comprising a compound or predicting the responsiveness of a subject having or suspected of having cancer to a treatment comprising the compound, comprising: i. providing a sample from the subject; ii. administering the compound to the sample; iii. measuring the level of one or more cell surface markers; iv. identifying the subject as being likely to be responsive to the treatment comprising the compound if the level of the one or more cell surface markers differentiates from a reference level, wherein the treatment compound is Compound D or a stereoisomer or mixture of stereoisomers, isotopologue, pharmaceutically acceptable salt, tautomer, solvate, hydrate, co-crystal, clathrate, or polymorph thereof.

In some embodiments, the one or more cell surface marker is selected from the group consisting of CD34, CD15, CD14, and CD11b.

In some embodiments, the method comprises measuring the level of CD34 prior to and after administration of the compound (e.g., Compound D), and a reduction of the level of CD34 after administering the compound indicates the subject is likely to be responsive to the treatment comprising the compound.

In other embodiments, the method comprises measuring the level of CD15 and/or CD14 prior to and after administration of the compound (e.g., Compound D), and an increase of the level of CD15 and/or CD14 after administration of the compound indicates the subject is likely to be responsive to the treatment comprising the compound.

Methods of determining a proportion of a cell type with certain cell surface markers and methods of determining the level of a cell surface marker in a sample are known in the art. Exemplary methods are illustrated in Section 6 below.

In some embodiments, an increase means an increase of at least 5%, 10%, 15%, 20%, 30%, 40%, 50%, 60%, 70%, 80%, 90%, or more as compared with a reference. In some embodiments, a reduction means a decrease of at least 5%, 10%, 15%, 20%, 30%, 40%, 50%, 60%, 70%, 80%, 90%, or more as compared with a reference.

In certain embodiments, the cancer is blood cancer. In one embodiment, the blood cancer is lymphoma. In another embodiment, the blood cancer is leukemia. In yet another embodiment, the blood cancer is MM. In a specific embodiment, the leukemia is ALL. In another specific embodiment, the leukemia is AML. In yet another specific embodiment, the leukemia is CLL. In still another embodiment, the leukemia is CML.

In some embodiments, the AML is relapsed. In certain embodiments, the AML is refractory. In other embodiments, the AML is resistant to conventional therapy.

In a specific embodiment, provided herein is a method of identifying a subject having AML who is likely to be responsive to a treatment comprising a compound provided herein or predicting the responsiveness of a subject having or suspected of having AML to a treatment comprising the compound using the methods described above.

5.2.3 Selective Treatments

In some embodiments of various methods provided herein (including those described above), a compound provided herein is administered to a patient that has been determined likely to be responsive to the compound. So, in one aspect, provided herein is a selective treatment method comprising administering a compound to a patient that has been determined likely to be responsive to the compound based on the methods described here (including those described above).

In another particular embodiment, the compound is Compound D or a stereoisomer or mixture of stereoisomers, isotopologue, pharmaceutically acceptable salt, tautomer, solvate, hydrate, co-crystal, clathrate, or polymorph thereof.

In some embodiments of the various methods provided herein, a treatment compound is administered to a patient likely to be responsive to the treatment compound. Also provided herein are methods of treating patients who have been previously treated for cancer but are non-responsive to standard therapies, as well as those who have not previously been treated. The invention also encompasses methods of treating patients regardless of patient's age, although some diseases or disorders are more common in certain age groups. The invention further encompasses methods of treating patients who have undergone surgery in an attempt to treat the disease or condition at issue, as well as those who have not. Because patients with cancer have heterogeneous clinical manifestations and varying clinical outcomes, the treatment given to a patient may vary, depending on his/her prognosis. The skilled clinician will be able to readily determine without undue experimentation specific secondary agents, types of surgery, and types of non-drug based standard therapy that can be effectively used to treat an individual patient with cancer.

Dosing and Administration

In certain embodiments, a therapeutically or prophylactically effective amount of the compound provided herein. In certain embodiments, a therapeutically or prophylactically effective amount of Compound D is from about 0.005 to about 20 mg per day, from about 0.05 to 20 mg per day, from about 0.01 to about 10 mg per day, from about 0.01 to about 7 mg per day, from about 0.01 to about 5 mg per day, from about 0.01 to about 3 mg per day, from about 0.05 to about 10 mg per day, from about 0.05 to about 7 mg per day, from about 0.05 to about 5 mg per day, from about 0.05 to about 3 mg per day, from about 0.1 to about 15 mg per day, from about 0.1 to about 10 mg per day, from about 0.1 to about 7 mg per day, from about 0.1 to about 5 mg per day, from about 0.1 to about 3 mg per day, from about 0.5 to about 10 mg per day, from about 0.05 to about 5 mg per day, from about 0.5 to about 3 mg per day, from about 0.5 to about 2 mg per day, from about 0.3 to about 10 mg per day, from about 0.3 to about 8.5 mg per day, from about 0.3 to about 8.1 mg per day, from about 0.6 to about 10 mg per day or from about 0.6 to about 5 mg per day. In one embodiment, a therapeutically or prophylactically effective amount of Compound D is from about 0.005 to about 20 mg per day. In one embodiment, a therapeutically or prophylactically effective amount of Compound D is, from about 0.05 to 20 mg per day. In one embodiment, a therapeutically or prophylactically effective amount of Compound D is from about 0.01 to about 10 mg per day. In one embodiment, a therapeutically or prophylactically effective amount of Compound D is from about 0.01 to about 7 mg per day. In one embodiment, a therapeutically or prophylactically effective amount of Compound D is from about 0.01 to about 5 mg per day. In one embodiment, a therapeutically or prophylactically effective amount of Compound D is from about 0.01 to about 3 mg per day. In one embodiment, a therapeutically or prophylactically effective amount of Compound D is from about 0.05 to about 10 mg per day. In one embodiment, a therapeutically or prophylactically effective amount of Compound D is from about 0.05 to about 7 mg per day. In one embodiment, a therapeutically or prophylactically effective amount of Compound D is from about 0.05 to about 5 mg per day. In one embodiment, a therapeutically or prophylactically effective amount of Compound D is from about 0.05 to about 3 mg per day. In one embodiment, a therapeutically or prophylactically effective amount of Compound D is from about 0.1 to about 15 mg per day. In one embodiment, a therapeutically or prophylactically effective amount of Compound D is from about 0.1 to about 10 mg per day. In one embodiment, a therapeutically or prophylactically effective amount of Compound D is from about 0.1 to about 7 mg per day. In one embodiment, a therapeutically or prophylactically effective amount of Compound D is from about 0.1 to about 5 mg per day. In one embodiment, a therapeutically or prophylactically effective amount of Compound D is from about 0.1 to about 3 mg per day. In one embodiment, a therapeutically or prophylactically effective amount of Compound D is from about 0.5 to about 10 mg per day. In one embodiment, a therapeutically or prophylactically effective amount of Compound D is from about 0.5 to about 5 mg per day. In one embodiment, a therapeutically or prophylactically effective amount of Compound D is from about 0.5 to about 3 mg per day. In one embodiment, a therapeutically or prophylactically effective amount of Compound D is from about 0.5 to about 2 mg per day. In one embodiment, a therapeutically or prophylactically effective amount of Compound D is from about 0.3 to about 10 mg per day. In one embodiment, a therapeutically or prophylactically effective amount of Compound D is from about 0.3 to about 8.5 mg per day. In one embodiment, a therapeutically or prophylactically effective amount of Compound D is from about 0.3 to about 8.1 mg per day. In one embodiment, a therapeutically or prophylactically effective amount of Compound D is from about 0.6 to about 10 mg per day or from about 0.6 to about 5 mg per day.

In certain embodiments, the therapeutically or prophylactically effective amount is about 0.1, about 0.2, about 0.5, about 1, about 2, about 3, about 4, about 5, about 6, about 7, about 8, about 9, or about 10 mg per day. In some such embodiments, the therapeutically or prophylactically effective amount is about 0.5, about 0.6, about 0.75, about 1, about 2, about 3, about 4, about 5, about 6 or about 7 mg per day. In some such embodiments, the therapeutically or prophylactically effective amount is about 0.6, about 1.2, about 1.8, about 2.4, or about 3.6 mg per day. In certain embodiments, the therapeutically or prophylactically effective amount is about 0.1 mg per day. In certain embodiments, the therapeutically or prophylactically effective amount is about 0.2 mg per day. In certain embodiments, the therapeutically or prophylactically effective amount is about 0.5 mg per day. In certain embodiments, the therapeutically or prophylactically effective amount is about 1 mg per day. In certain embodiments, the therapeutically or prophylactically effective amount is about 2 mg per day. In certain embodiments, the therapeutically or prophylactically effective amount is about 3 mg per day. In certain embodiments, the therapeutically or prophylactically effective amount is about 4 mg per day. In certain embodiments, the therapeutically or prophylactically effective amount is about 5 mg per day. In certain embodiments, the therapeutically or prophylactically effective amount is about 6 mg per day. In certain embodiments, the therapeutically or prophylactically effective amount is about 7 mg per day. In certain embodiments, the therapeutically or prophylactically effective amount is about 8 mg per day. In certain embodiments, the therapeutically or prophylactically effective amount is about 9 mg per day. In certain embodiments, the therapeutically or prophylactically effective amount is about 10 mg per day.

In one embodiment, the recommended daily dose range of Compound D, for the conditions described herein lie within the range of from about 0.01 mg to about 20 mg per day, preferably given as a single once-a-day dose, or in divided doses throughout a day. In one embodiment, the recommended daily dose range of Compound D, for the conditions described herein lie within the range of from about 0.01 mg to about 15 mg per day, preferably given as a single once-a-day dose, or in divided doses throughout a day. In one embodiment, the recommended daily dose range of Compound D, for the conditions described herein lie within the range of from about 0.01 mg to about 12 mg per day, preferably given as a single once-a-day dose, or in divided doses throughout a day. In some embodiments, the dosage ranges from about 0.1 mg to about 10 mg per day. In other embodiments, the dosage ranges from about 0.5 to about 5 mg per day. Specific doses per day include 0.1, 0.2, 0.5, 0.6, 1, 1.2, 1.5, 1.8, 2, 2.4, 2.5, 3, 3.5, 3.6, 4, 4.5, 5, 5.5, 6, 6.5, 7, 7.2, 7.5, 8, 8.5, 9, 9.5, 10, 10.5, 11, 11.5, 12, 12.5, 13, 13.5, 14, 14.4, 14.5 or 15 mg per day. In other embodiments, the dosage ranges from about 0.5 to about 5 mg per day. Specific doses per day include 0.1, 0.2, 0.5, 0.6, 1, 1.2, 1.5, 1.8, 2, 2.4, 2.5, 3, 3.5, 3.6, 4, 4.5, 5, 5.5, 6, 6.5, 7, 7.5, 8, 8.5, 9, 9.5 or 10 mg per day. In one embodiment, the dose per day is 0.1 mg per day. In one embodiment, the dose per day is 0.2 mg per day. In one embodiment, the dose per day is 0.5 mg per day. In one embodiment, the dose per day is 0.6 mg per day. In one embodiment, the dose per day is 1 mg per day. In one embodiment, the dose per day is 1.2 mg per day. In one embodiment, the dose per day is 1.5 mg per day. In one embodiment, the dose per day is 1.8 mg per day. In one embodiment, the dose per day is 2 mg per day. In one embodiment, the dose per day is 2.4 mg per day. In one embodiment, the dose per day is 2.5 mg per day. In one embodiment, the dose per day is 3 mg per day. In one embodiment, the dose per day is 3.5 mg per day. In one embodiment, the dose per day is 3.6 mg per day. In one embodiment, the dose per day is 4 mg per day. In one embodiment, the dose per day is 4.5 mg per day. In one embodiment, the dose per day is 5 mg per day. In one embodiment, the dose per day is 5.5 mg per day. In one embodiment, the dose per day is 6 mg per day. In one embodiment, the dose per day is 6.5 mg per day. In one embodiment, the dose per day is 7 mg per day. In one embodiment, the dose per day is 7.2 mg per day. In one embodiment, the dose per day is 7.5 mg per day. In one embodiment, the dose per day is 8 mg per day. In one embodiment, the dose per day is 8.5 mg per day. In one embodiment, the dose per day is 9 mg per day. In one embodiment, the dose per day is 9.5 mg per day. In one embodiment, the dose per day is 10 mg per day. In one embodiment, the dose per day is 12 mg per day. In one embodiment, the dose per day is 10 mg per day. In one embodiment, the dose per day is 12 mg per day. In one embodiment, the dose per day is 14.4 mg per day. In one embodiment, the dose per day is 15 mg per day.

In a specific embodiment, the recommended starting dosage may be 0.1, 0.5, 0.6, 0.7, 1, 1.2, 1.5, 1.8, 2, 2.4, 2.5, 3, 3.5, 3.6, 4, 4.5, 5, 5.5, 6, 6.5 or 7 mg per day. In another embodiment, the recommended starting dosage may be 0.1, 0.5, 0.6, 1, 1.2, 1.8, 2, 2.4, 3, 3.6, 4, or 5 mg per day. In one embodiment, the dose may be escalated to 7, 8, 9 10, 12, or 15 mg/day. In one embodiment, the dose may be escalated to 7, 8, 9 or 10 mg/day.

In a specific embodiment, Compound D can be administered in an amount of about 0.1 mg/day to patients with leukemia, including AML. In a particular embodiment, Compound D can be administered in an amount of about 1 mg/day to patients with leukemia, including AML. In a particular embodiment, Compound D can be administered in an amount of about 3 mg/day to patients with leukemia, including AML. In a particular embodiment, Compound D can be administered in an amount of about 4 mg/day to patients with leukemia, including AML. In a particular embodiment, Compound D provided herein can be administered in an amount of about 5 mg/day to patients with leukemia, including AML. In a particular embodiment, Compound D provided herein can be administered in an amount of about 6 mg/day to patients with leukemia, including AML. In a particular embodiment, Compound D provided herein can be administered in an amount of about 7 mg/day to patients with leukemia, including AML. In a particular embodiment, Compound D provided herein can be administered in an amount of about 10 mg/day to patients with leukemia, including AML. In a particular embodiment, Compound D provided herein can be administered in an amount of about 12 mg/day to patients with leukemia, including AML. In a particular embodiment, Compound D provided herein can be administered in an amount of about 15 mg/day to patients with leukemia, including AML.

In a specific embodiment, Compound D can be administered in an amount of about 0.1 mg/day to patients with MDS. In a particular embodiment, Compound D can be administered in an amount of about 1 mg/day to patients with MDS. In a particular embodiment, Compound D can be administered in an amount of about 3 mg/day to patients with MDS. In a particular embodiment, Compound D can be administered in an amount of about 4 mg/day to patients with MDS. In a particular embodiment, Compound D provided herein can be administered in an amount of about 5 mg/day to patients with MDS. In a particular embodiment, Compound D provided herein can be administered in an amount of about 6 mg/day to patients with MDS. In a particular embodiment, Compound D provided herein can be administered in an amount of about 7 mg/day to patients with MDS. In a particular embodiment, Compound D provided herein can be administered in an amount of about 10 mg/day to patients with MDS. In a particular embodiment, Compound D provided herein can be administered in an amount of about 12 mg/day to patients with MDS. In a particular embodiment, Compound D provided herein can be administered in an amount of about 15 mg/day to patients with MDS.

In certain embodiments, the therapeutically or prophylactically effective amount is from about 0.001 to about 20 mg/kg/day, from about 0.01 to about 15 mg/kg/day, from about 0.01 to about 10 mg/kg/day, from about 0.01 to about 9 mg/kg/day, 0.01 to about 8 mg/kg/day, from about 0.01 to about 7 mg/kg/day, from about 0.01 to about 6 mg/kg/day, from about 0.01 to about 5 mg/kg/day, from about 0.01 to about 4 mg/kg/day, from about 0.01 to about 3 mg/kg/day, from about 0.01 to about 2 mg/kg/day, from about 0.01 to about 1 mg/kg/day, or from about 0.01 to about 0.05 mg/kg/day. In certain embodiments, the therapeutically or prophylactically effective amount is from about 0.001 to about 20 mg/kg/day. In certain embodiments, the therapeutically or prophylactically effective amount is from about 0.01 to about 15 mg/kg/day. In certain embodiments, the therapeutically or prophylactically effective amount is from about 0.01 to about 10 mg/kg/day. In certain embodiments, the therapeutically or prophylactically effective amount is from about 0.01 to about 9 mg/kg/day. In certain embodiments, the therapeutically or prophylactically effective amount is 0.01 to about 8 mg/kg/day. In certain embodiments, the therapeutically or prophylactically effective amount is from about 0.01 to about 7 mg/kg/day. In certain embodiments, the therapeutically or prophylactically effective amount is from about 0.01 to about 6 mg/kg/day. In certain embodiments, the therapeutically or prophylactically effective amount is from about 0.01 to about 5 mg/kg/day. In certain embodiments, the therapeutically or prophylactically effective amount is from about 0.01 to about 4 mg/kg/day. In certain embodiments, the therapeutically or prophylactically effective amount is from about 0.01 to about 3 mg/kg/day. In certain embodiments, the therapeutically or prophylactically effective amount is from about 0.01 to about 2 mg/kg/day. In certain embodiments, the therapeutically or prophylactically effective amount is from about 0.01 to about 1 mg/kg/day. In certain embodiments, the therapeutically or prophylactically effective amount is from about 0.01 to about 0.05 mg/kg/day.

The administered dose can also be expressed in units other than mg/kg/day. For example, doses for parenteral administration can be expressed as mg/m²/day. One of ordinary skill in the art would readily know how to convert doses from mg/kg/day to mg/m²/day to given either the height or weight of a subject or both (see, www.fda.gov/cder/cancer/animalframe.htm). For example, a dose of 1 mg/kg/day for a 65 kg human is approximately equal to 38 mg/m²/day.

In certain embodiments, the amount of Compound D administered is sufficient to provide a plasma concentration of the compound at steady state, ranging from about 0.001 to about 500 μM, about 0.002 to about 200 μM, about 0.005 to about 100 μM, about 0.01 to about 50 μM, from about 1 to about 50 μM, about 0.02 to about 25 μM, from about 0.05 to about 20 μM, from about 0.1 to about 20 μM, from about 0.5 to about 20 μM, or from about 1 to about 20 μM. In certain embodiments, the amount of Compound D administered is sufficient to provide a plasma concentration of the compound at steady state, ranging from about 0.001 to about 500 μM, about 0.002 to about 200 μM, about 0.005 to about 100 μM, about 0.01 to about 50 μM, from about 1 to about 50 μM, about 0.02 to about 25 μM, from about 0.05 to about 20 μM, from about 0.1 to about 20 μM, from about 0.5 to about 20 μM, or from about 1 to about 20 μM.

In other embodiments, the amount of a formulation of Compound D administered is sufficient to provide a plasma concentration of the compound at steady state, ranging from about 5 to about 100 nM, about 5 to about 50 nM, about 10 to about 100 nM, about 10 to about 50 nM or from about 50 to about 100 nM. In other embodiments, the amount of a formulation of Compound D administered is sufficient to provide a plasma concentration of the compound at steady state, ranging from about 5 to about 100 nM. In other embodiments, the amount of a formulation of Compound D administered is sufficient to provide a plasma concentration of the compound at steady state, ranging from about 5 to about 50 nM. In other embodiments, the amount of a formulation of Compound D administered is sufficient to provide a plasma concentration of the compound at steady state, ranging from about 10 to about 100 nM. In other embodiments, the amount of a formulation of Compound D administered is sufficient to provide a plasma concentration of the compound at steady state, ranging from about 10 to about 50 nM. In other embodiments, the amount of a formulation of Compound D administered is sufficient to provide a plasma concentration of the compound at steady state, ranging from about 50 to about 100 nM.

As used herein, the term “plasma concentration at steady state” is the concentration reached after a period of administration of a formulation provided herein. Once steady state is reached, there are minor peaks and troughs on the time dependent curve of the plasma concentration of the solid form.

In certain embodiments, the amount of a formulation of Compound D administered is sufficient to provide a maximum plasma concentration (peak concentration) of the compound, ranging from about 0.001 to about 500 μM, about 0.002 to about 200 μM, about 0.005 to about 100 μM, about 0.01 to about 50 μM, from about 1 to about 50 μM, about 0.02 to about 25 μM, from about 0.05 to about 20 μM, from about 0.1 to about 20 μM, from about 0.5 to about 20 μM, or from about 1 to about 20 μM. In certain embodiments, the amount of a formulation of Compound D administered is sufficient to provide a maximum plasma concentration (peak concentration) of the compound, ranging from about 0.001 to about 500 μM. In certain embodiments, the amount of a formulation of Compound D administered is sufficient to provide a maximum plasma concentration (peak concentration) of the compound, ranging from about 0.002 to about 200 μM. In certain embodiments, the amount of a formulation of Compound D administered is sufficient to provide a maximum plasma concentration (peak concentration) of the compound, ranging from about 0.005 to about 100 μM. In certain embodiments, the amount of a formulation of Compound D administered is sufficient to provide a maximum plasma concentration (peak concentration) of the compound, ranging from about 0.01 to about 50 μM. In certain embodiments, the amount of a formulation of Compound D administered is sufficient to provide a maximum plasma concentration (peak concentration) of the compound, ranging from about 1 to about 50 μM. In certain embodiments, the amount of a formulation of Compound D administered is sufficient to provide a maximum plasma concentration (peak concentration) of the compound, ranging from about 0.02 to about 25 μM. In certain embodiments, the amount of a formulation of Compound D administered is sufficient to provide a maximum plasma concentration (peak concentration) of the compound, ranging from about 0.05 to about 20 μM. In certain embodiments, the amount of a formulation of Compound D administered is sufficient to provide a maximum plasma concentration (peak concentration) of the compound, ranging from about 0.1 to about 20 μM. In certain embodiments, the amount of a formulation of Compound D administered is sufficient to provide a maximum plasma concentration (peak concentration) of the compound, ranging from about 0.5 to about 20 μM. In certain embodiments, the amount of a formulation of Compound D administered is sufficient to provide a maximum plasma concentration (peak concentration) of the compound, ranging from about 1 to about 20 μM.

In certain embodiments, the amount of a formulation of Compound D administered is sufficient to provide a minimum plasma concentration (trough concentration) of the compound, ranging from about 0.001 to about 500 μM, about 0.002 to about 200 μM, about 0.005 to about 100 μM, about 0.01 to about 50 μM, from about 1 to about 50 μM, about 0.01 to about 25 μM, from about 0.01 to about 20 μM, from about 0.02 to about 20 μM, from about 0.02 to about 20 μM, or from about 0.01 to about 20 μM. In certain embodiments, the amount of a formulation of Compound D administered is sufficient to provide a minimum plasma concentration (trough concentration) of the compound, ranging from about 0.001 to about 500 μM. In certain embodiments, the amount of a formulation of Compound D administered is sufficient to provide a minimum plasma concentration (trough concentration) of the compound, ranging from about 0.002 to about 200 μM. In certain embodiments, the amount of a formulation of Compound D administered is sufficient to provide a minimum plasma concentration (trough concentration) of the compound, ranging from about 0.005 to about 100 μM. In certain embodiments, the amount of a formulation of Compound D administered is sufficient to provide a minimum plasma concentration (trough concentration) of the compound, ranging from about 0.01 to about 50 μM. In certain embodiments, the amount of a formulation of Compound D administered is sufficient to provide a minimum plasma concentration (trough concentration) of the compound, ranging from about 1 to about 50 μM, about 0.01 to about 25 μM. In certain embodiments, the amount of a formulation of Compound D administered is sufficient to provide a minimum plasma concentration (trough concentration) of the compound, ranging from about 0.01 to about 20 μM. In certain embodiments, the amount of a formulation of Compound D administered is sufficient to provide a minimum plasma concentration (trough concentration) of the compound, ranging from about 0.02 to about 20 μM. In certain embodiments, the amount of a formulation of Compound D administered is sufficient to provide a minimum plasma concentration (trough concentration) of the compound, ranging from about 0.02 to about 20 μM. In certain embodiments, the amount of a formulation of Compound D administered is sufficient to provide a minimum plasma concentration (trough concentration) of the compound, ranging from about 0.01 to about 20 μM.

In certain embodiments, the amount of a formulation of Compound D administered is sufficient to provide an area under the curve (AUC) of the compound, ranging from about 100 to about 100,000 ng*hr/mL, from about 1,000 to about 50,000 ng*hr/mL, from about 5,000 to about 25,000 ng*hr/mL, or from about 5,000 to about 10,000 ng*hr/mL. In certain embodiments, the amount of a formulation of Compound D administered is sufficient to provide an area under the curve (AUC) of the compound, ranging from about 100 to about 100,000 ng*hr/mL. In certain embodiments, the amount of a formulation of Compound D administered is sufficient to provide an area under the curve (AUC) of the compound, ranging from about 1,000 to about 50,000 ng*hr/mL. In certain embodiments, the amount of a formulation of Compound D administered is sufficient to provide an area under the curve (AUC) of the compound, ranging from about 5,000 to about 25,000 ng*hr/mL. In certain embodiments, the amount of a formulation of Compound D administered is sufficient to provide an area under the curve (AUC) of the compound, ranging from about 5,000 to about 10,000 ng*hr/mL.

In certain embodiments, the patient to be treated with one of the methods provided herein has not been treated with anti-cancer therapy prior to the administration of a formulation of Compound D provided herein. In certain embodiments, the patient to be treated with one of the methods provided herein has been treated with anti-cancer therapy prior to the administration of a formulation of Compound D provided herein. In certain embodiments, the patient to be treated with one of the methods provided herein has developed drug resistance to the anti-cancer therapy.

The methods provided herein encompass treating a patient regardless of patient's age, although some diseases or disorders are more common in certain age groups.

The formulation of Compound D provided herein can be delivered as a single dose such as, e.g., a single bolus injection, or over time, such as, e.g., continuous infusion over time or divided bolus doses over time. The formulation of Compound D can be administered repeatedly if necessary, for example, until the patient experiences stable disease or regression, or until the patient experiences disease progression or unacceptable toxicity. For example, stable disease for solid tumors generally means that the perpendicular diameter of measurable lesions has not increased by 25% or more from the last measurement. Response Evaluation Criteria in Solid Tumors (RECIST) Guidelines, Journal of the National Cancer Institute 92(3): 205-216 (2000). Stable disease or lack thereof is determined by methods known in the art such as evaluation of patient symptoms, physical examination, visualization of the tumor that has been imaged using X-ray, CAT, PET, or MRI scan and other commonly accepted evaluation modalities.

The formulation of Compound D provided herein can be administered once daily (QD) or divided into multiple daily doses such as twice daily (BID), three times daily (TID), and four times daily (QID). In addition, the administration can be continuous (i.e., daily for consecutive days or every day), intermittent, e.g., in cycles (i.e., including days, weeks, or months of rest without drug). As used herein, the term “daily” is intended to mean that a therapeutic compound is administered once or more than once each day, for example, for a period of time. The term “continuous” is intended to mean that a therapeutic compound is administered daily for an uninterrupted period of at least 10 days to 52 weeks. The term “intermittent” or “intermittently” as used herein is intended to mean stopping and starting at either regular or irregular intervals. For example, intermittent administration of the formulation of Compound D is administration for one to six days per week, administration in cycles (e.g., daily administration for one to ten consecutive days of a 28 day cycle, then a rest period with no administration for rest of the 28 day cycle; or daily administration for two to eight consecutive weeks, then a rest period with no administration for up to one week), or administration on alternate days. Cycling therapy with Compound D is discussed elsewhere herein.

In some embodiments, the frequency of administration is in the range of about a daily dose to about a monthly dose. In certain embodiments, administration is once a day, twice a day, three times a day, four times a day, once every other day, twice a week, once every week, once every two weeks, once every three weeks, or once every four weeks. In one embodiment, Compound D is administered once a day. In another embodiment, Compound D is administered twice a day. In yet another embodiment, Compound D provided herein is administered three times a day. In still another embodiment, Compound D provided herein is administered four times a day. In still another embodiment, Compound D provided herein is administered once every other day. In still another embodiment, Compound D provided herein is administered twice a week. In still another embodiment, Compound D provided herein is administered once every week. In still another embodiment, Compound D provided herein is administered once every two weeks. In still another embodiment, Compound D provided herein is administered once every three weeks. In still another embodiment, Compound D provided herein is administered once every four weeks.

In certain embodiments, a formulation of Compound D provided herein is administered once per day from one day to six months, from one week to three months, from one week to four weeks, from one week to three weeks, or from one week to two weeks. In certain embodiments, a formulation of Compound D provided herein is administered once per day for one week, two weeks, three weeks, or four weeks. In one embodiment, a formulation of Compound D provided herein is administered once per day for 1 day. In one embodiment, a formulation of Compound D provided herein is administered once per day for 2 days. In one embodiment, a formulation of Compound D provided herein is administered once per day for 3 days. In one embodiment, a formulation of Compound D provided herein is administered once per day for 4 days. In one embodiment, a formulation of Compound D provided herein is administered once per day for 5 days. In one embodiment, a formulation of Compound D provided herein is administered once per day for 6 days. In one embodiment, a formulation of Compound D provided herein is administered once per day for one week. In one embodiment, a formulation of Compound D provided herein is administered once per day for up to 10 days. In another embodiment, a formulation of Compound D provided herein is administered once per day for two weeks. In yet another embodiment, a formulation of Compound D provided herein is administered once per day for three weeks. In still another embodiment, a formulation of Compound D provided herein is administered once per day for four weeks.

### Combination Therapy

In one embodiment, provided herein is a method of treating, preventing, and/or managing cancer, comprising administering to a patient Compound D in combination with one or more second agents selected from JAK inhibitors, FLT3 inhibitors, mTOR inhibitors, spliceosome inhibitors, BET inhibitors, SMG1 inhibitors, ERK inhibitors, LSD1 inhibitors, BH3 mimetics, topoisomerase inhibitors, and RTK inhibitors, and optionally in combination with radiation therapy, blood transfusions, or surgery. Examples of second active agents are disclosed herein.

In one embodiment, provided herein is a method of treating, preventing, and/or managing cancer, comprising administering to a patient a formulation of Compound D provided herein in combination with one or more second active agents, and optionally in combination with radiation therapy, blood transfusions, or surgery. Examples of second active agents are disclosed herein.

As used herein, the term “in combination” includes the use of more than one therapy (e.g., one or more prophylactic and/or therapeutic agents). However, the use of the term “in combination” does not restrict the order in which therapies (e.g., prophylactic and/or therapeutic agents) are administered to a patient with a disease or disorder. E.g., “in combination” may include administration as a mixture, simultaneous administration using separate formulations, and consecutive administration in any order. “Consecutive” means that a specific time has passed between the administration of the active agents. For example, “consecutive” may be that more than 10 minutes have passed between the administration of the separate active agents. The time period can then be more than 10 min, more than 30 minutes, more than 1 hour, more than 3 hours, more than 6 hours or more than 12 hours. E.g., a first therapy (e.g., a prophylactic or therapeutic agent such as a formulation of Compound D provided herein) can be administered prior to (e.g., 5 minutes, 15 minutes, 30 minutes, 45 minutes, 1 hour, 2 hours, 4 hours, 6 hours, 12 hours, 24 hours, 48 hours, 72 hours, 96 hours, 1 week, 2 weeks, 3 weeks, 4 weeks, 5 weeks, 6 weeks, 8 weeks, or 12 weeks before), concomitantly with, or subsequent to (e.g., 5 minutes, 15 minutes, 30 minutes, 45 minutes, 1 hour, 2 hours, 4 hours, 6 hours, 12 hours, 24 hours, 48 hours, 72 hours, 96 hours, 1 week, 2 weeks, 3 weeks, 4 weeks, 5 weeks, 6 weeks, 8 weeks, or 12 weeks after) the administration of a second therapy (e.g., a prophylactic or therapeutic agent) to the subject. Triple therapy is also contemplated herein.

In one embodiment, administration of Compound D, including a formulation of Compound D provided herein, and one or more second active agents to a patient can occur simultaneously or sequentially by the same or different routes of administration. In one embodiment, administration of Compound D, including a formulation of Compound D provided herein, and one or more second active agents to a patient can occur simultaneously or sequentially by the same or different routes of administration. The suitability of a particular route of administration employed for a particular active agent will depend on the active agent itself (e.g., whether it can be administered orally without decomposing prior to entering the blood stream) and the cancer being treated.

The route of administration of Compound D, including a formulation of Compound D provided herein, is independent of the route of administration of a second therapy. Thus, in one embodiment, Compound D, including a formulation of Compound D provided herein, is administered intravenously, and the second therapy can be administered orally, parenterally, intraperitoneally, intravenously, intraarterially, transdermally, sublingually, intramuscularly, rectally, transbuccally, intranasally, liposomally, via inhalation, vaginally, intraocularly, via local delivery by catheter or stent, subcutaneously, intraadiposally, intraarticularly, intrathecally, or in a slow release dosage form. In one embodiment, Compound D, including a formulation of Compound D provided herein, and a second therapy are administered by the same mode of administration, by IV. In another embodiment, Compound D, including a formulation of Compound D provided herein, is administered by one mode of administration, e.g., by IV, whereas the second agent (an anti-cancer agent) is administered by another mode of administration, e.g., orally.

In one embodiment, the second active agent is administered intravenously or subcutaneously and once or twice daily in an amount of from about 1 to about 1000 mg, from about 5 to about 500 mg, from about 10 to about 350 mg, or from about 50 to about 200 mg. The specific amount of the second active agent will depend on the specific agent used, the type of disease being treated and/or managed, the severity and stage of disease, and the amount of Compound D and any optional additional active agents concurrently administered to the patient.

One or more second active ingredients or agents can be used together with Compound D in the methods and compositions provided herein. Second active agents can be large molecules (e.g., proteins) or small molecules (e.g., synthetic inorganic, organometallic, or organic molecules).

Examples of large molecule active agents include, but are not limited to, hematopoietic growth factors, cytokines, and monoclonal and polyclonal antibodies, particularly, therapeutic antibodies to cancer antigens. Typical large molecule active agents are biological molecules, such as naturally occurring or synthetic or recombinant proteins. Proteins that are particularly useful in the methods and compositions provided herein include proteins that stimulate the survival and/or proliferation of hematopoietic precursor cells and immunologically active poietic cells in vitro or in vivo. Other useful proteins stimulate the division and differentiation of committed erythroid progenitors in cells in vitro or in vivo. Particular proteins include, but are not limited to: interleukins, such as IL-2 (including recombinant IL-II (“rIL2”) and canarypox IL-2), IL-10, IL-12, and IL-18; interferons, such as interferon alfa-2a, interferon alfa-2b, interferon alfa-n1, interferon alfa-n3, interferon beta-I a, and interferon gamma-I b; GM-CF and GM-CSF; and EPO.

In certain embodiments, GM-CSF, G-CSF, SCF or EPO is administered subcutaneously during about five days in a four- or six-week cycle in an amount ranging from about 1 to about 750 mg/m²/day, from about 25 to about 500 mg/m²/day, from about 50 to about 250 mg/m²/day, or from about 50 to about 200 mg/m²/day. In certain embodiments, GM-CSF may be administered in an amount of from about 60 to about 500 mcg/m² intravenously over 2 hours or from about 5 to about 12 mcg/m²/day subcutaneously. In certain embodiments, G-CSF may be administered subcutaneously in an amount of about 1 mcg/kg/day initially and can be adjusted depending on rise of total granulocyte counts. The maintenance dose of G-CSF may be administered in an amount of about 300 (in smaller patients) or 480 mcg subcutaneously. In certain embodiments, EPO may be administered subcutaneously in an amount of 10,000 Unit 3 times per week.

Particular proteins that can be used in the methods and compositions include, but are not limited to: filgrastim, which is sold in the United States under the trade name Neupogen® (Amgen, Thousand Oaks, Calif.); sargramostim, which is sold in the United States under the trade name Leukine® (Immunex, Seattle, Wash.); and recombinant EPO, which is sold in the United States under the trade name Epogen® (Amgen, Thousand Oaks, Calif.).

Recombinant and mutated forms of GM-CSF can be prepared as described in U.S. Pat. Nos. 5,391,485; 5,393,870; and 5,229,496; all of which are incorporated herein by reference. Recombinant and mutated forms of G-CSF can be prepared as described in U.S. Pat. Nos. 4,810,643; 4,999,291; 5,528,823; and 5,580,755; the entireties of which are incorporated herein by reference.

Also provided for use in combination with Compound D, including a formulation of Compound D, are native, naturally occurring, and recombinant proteins. Further encompassed are mutants and derivatives (e.g., modified forms) of naturally occurring proteins that exhibit, in vivo, at least some of the pharmacological activity of the proteins upon which they are based. Examples of mutants include, but are not limited to, proteins that have one or more amino acid residues that differ from the corresponding residues in the naturally occurring forms of the proteins. Also encompassed by the term “mutants” are proteins that lack carbohydrate moieties normally present in their naturally occurring forms (e.g., nonglycosylated forms). Examples of derivatives include, but are not limited to, pegylated derivatives and fusion proteins, such as proteins formed by fusing IgG1 or IgG3 to the protein or active portion of the protein of interest. See, e.g., Penichet, M. L. and Morrison, S. L., J. Immunol. Methods 248:91-101 (2001).

Antibodies that can be used in combination with Compound D, including a formulation of Compound D provided herein, include monoclonal and polyclonal antibodies. Examples of antibodies include, but are not limited to, trastuzumab (Herceptin®), rituximab (Rituxan®), bevacizumab (Avastin™), pertuzumab (Omnitarg™), tositumomab (Bexxar), edrecolomab (Panorex®), and G250. The formulation of Compound D can also be combined with, or used in combination with, anti-TNF-α antibodies, and/or anti-EGFR antibodies, such as, for example, Erbitux® or panitumumab.

Large molecule active agents may be administered in the form of anti-cancer vaccines. For example, vaccines that secrete, or cause the secretion of, cytokines such as IL-2, G-CSF, and GM-CSF can be used in the methods and pharmaceutical compositions provided. See, e.g., Emens, L. A., et al., Curr. Opinion Mol. Ther. 3(1):77-84 (2001).

Second active agents that are small molecules can also be used to alleviate adverse effects associated with the administration of a formulation of Compound D provided herein. However, like some large molecules, many are believed to be capable of providing a synergistic effect when administered with (e.g., before, after, or simultaneously) Compound D, including a formulation of Compound D provided herein. Examples of small molecule second active agents include, but are not limited to, anti-cancer agents, antibiotics, immunosuppressive agents, and steroids.

In certain embodiments, the second agent is an HSP inhibitor, a proteasome inhibitor, a FLT3 inhibitor or an mTOR inhibitor. In some embodiments, the mTOR inhibitor is a mTOR kinase inhibitor.

Examples of anti-cancer agents to be used within the methods or compositions described herein include, but are not limited to: acivicin; aclarubicin; acodazole hydrochloride; acronine; adozelesin; aldesleukin; altretamine; ambomycin; ametantrone acetate; amsacrine; anastrozole; anthramycin; asparaginase; asperlin; azacitidine; azetepa; azotomycin; batimastat; benzodepa; bicalutamide; bisantrene hydrochloride; bisnafide dimesylate; bizelesin; bleomycin sulfate; brequinar sodium; bropirimine; busulfan; cactinomycin; calusterone; caracemide; carbetimer; carboplatin; carmustine; carubicin hydrochloride; carzelesin; cedefingol; celecoxib (COX-2 inhibitor); chlorambucil; cirolemycin; cisplatin; cladribine; clofarabine; crisnatol mesylate; cyclophosphamide; Ara-C; dacarbazine; dactinomycin; daunorubicin hydrochloride; decitabine; dexormaplatin; dezaguanine; dezaguanine mesylate; diaziquone; docetaxel; doxorubicin; doxorubicin hydrochloride; droloxifene; droloxifene citrate; dromostanolone propionate; duazomycin; edatrexate; eflornithine hydrochloride; elsamitrucin; enloplatin; enpromate; epipropidine; epirubicin hydrochloride; erbulozole; esorubicin hydrochloride; estramustine; estramustine phosphate sodium; etanidazole; etoposide; etoposide phosphate; etoprine; fadrozole hydrochloride; fazarabine; fenretinide; floxuridine; fludarabine phosphate; fluorouracil; flurocitabine; fosquidone; fostriecin sodium; gemcitabine; gemcitabine hydrochloride; hydroxyurea; idarubicin hydrochloride; ifosfamide; ilmofosine; iproplatin; irinotecan; irinotecan hydrochloride; lanreotide acetate; letrozole; leuprolide acetate; liarozole hydrochloride; lometrexol sodium; lomustine; losoxantrone hydrochloride; masoprocol; maytansine; mechlorethamine hydrochloride; megestrol acetate; melengestrol acetate; melphalan; menogaril; mercaptopurine; methotrexate; methotrexate sodium; metoprine; meturedepa; mitindomide; mitocarcin; mitocromin; mitogillin; mitomalcin; mitomycin; mitosper; mitotane; mitoxantrone hydrochloride; mycophenolic acid; nocodazole; nogalamycin; omacetaxine; ormaplatin; oxisuran; paclitaxel; pegaspargase; peliomycin; pentamustine; peplomycin sulfate; perfosfamide; pipobroman; piposulfan; piroxantrone hydrochloride; plicamycin; plomestane; porfimer sodium; porfiromycin; prednimustine; procarbazine hydrochloride; puromycin; puromycin hydrochloride; pyrazofurin; riboprine; safingol; safingol hydrochloride; semustine; simtrazene; sorafenib; sparfosate sodium; sparsomycin; spirogermanium hydrochloride; spiromustine; spiroplatin; streptonigrin; streptozocin; sulofenur; talisomycin; tecogalan sodium; taxotere; tegafur; teloxantrone hydrochloride; temoporfin; teniposide; teroxirone; testolactone; thiamiprine; thioguanine; thiotepa; tiazofurin; tirapazamine; toremifene citrate; trestolone acetate; triciribine phosphate; trimetrexate; trimetrexate glucuronate; triptorelin; tubulozole hydrochloride; uracil mustard; uredepa; vapreotide; verteporfin; vinblastine sulfate; vincristine sulfate; vindesine; vindesine sulfate; vinepidine sulfate; vinglycinate sulfate; vinleurosine sulfate; vinorelbine tartrate; vinrosidine sulfate; vinzolidine sulfate; vorozole; zeniplatin; zinostatin; and zorubicin hydrochloride.

Other anti-cancer drugs to be included within the methods herein include, but are not limited to: 20-epi-1,25 dihydroxyvitamin D3; 5-ethynyluracil; abiraterone; aclarubicin; acylfulvene; adecypenol; adozelesin; aldesleukin; ALL-TK antagonists; altretamine; ambamustine; amidox; amifostine; aminolevulinic acid; amrubicin; amsacrine; anagrelide; anastrozole; andrographolide; angiogenesis inhibitors; antagonist D; antagonist G; antarelix; anti-dorsalizing morphogenetic protein-1; antiandrogen, prostatic carcinoma; antiestrogen; antineoplaston; antisense oligonucleotides; aphidicolin glycinate; apoptosis gene modulators; apoptosis regulators; apurinic acid; ara-CDP-DL-PTBA; arginine deaminase; asulacrine; atamestane; atrimustine; axinastatin 1; axinastatin 2; axinastatin 3; azasetron; azatoxin; azatyrosine; baccatin III derivatives; balanol; batimastat; BCR/ABL antagonists; benzochlorins; benzoylstaurosporine; beta lactam derivatives; beta-alethine; betaclamycin B; betulinic acid; bFGF inhibitor; bicalutamide; bisantrene; bisaziridinylspermine; bisnafide; bistratene A; bizelesin; breflate; bropirimine; budotitane; buthionine sulfoximine; calcipotriol; calphostin C; camptothecin derivatives; capecitabine; carboxamide-amino-triazole; carboxyamidotriazole; CaRest M3; CARN 700; cartilage derived inhibitor; carzelesin; casein kinase inhibitors (ICOS); castanospermine; cecropin B; cetrorelix; chlorins; chloroquinoxaline sulfonamide; cicaprost; cis-porphyrin; cladribine; clomifene analogues; clotrimazole; collismycin A; collismycin B; combretastatin A4; combretastatin analogue; conagenin; crambescidin 816; crisnatol; cryptophycin 8; cryptophycin A derivatives; curacin A; cyclopentanthraquinones; cycloplatam; cypemycin; Ara-C ocfosfate; cytolytic factor; cytostatin; dacliximab; decitabine; dehydrodidemnin B; deslorelin; dexamethasone; dexifosfamide; dexrazoxane; dexverapamil; diaziquone; didemnin B; didox; diethylnorspermine; dihydro-5-azacytidine; dihydrotaxol, 9-; dioxamycin; diphenyl spiromustine; docetaxel; docosanol; dolasetron; doxifluridine; doxorubicin; droloxifene; dronabinol; duocarmycin SA; ebselen; ecomustine; edelfosine; edrecolomab; eflornithine; elemene; emitefur; epirubicin; epristeride; estramustine analogue; estrogen agonists; estrogen antagonists; etanidazole; etoposide phosphate; exemestane; fadrozole; fazarabine; fenretinide; filgrastim; finasteride; flavopiridol; flezelastine; fluasterone; fludarabine; fluorodaunorunicin hydrochloride; forfenimex; formestane; fostriecin; fotemustine; gadolinium texaphyrin; gallium nitrate; galocitabine; ganirelix; gelatinase inhibitors; gemcitabine; glutathione inhibitors; hepsulfam; heregulin; hexamethylene bisacetamide; hypericin; ibandronic acid; idarubicin; idoxifene; idramantone; ilmofosine; ilomastat; imatinib (e.g., Gleevec®); imiquimod; immunostimulant peptides; insulin-like growth factor-1 receptor inhibitor; interferon agonists; interferons; interleukins; iobenguane; iododoxorubicin; ipomeanol, 4-; iroplact; irsogladine; isobengazole; isohomohalicondrin B; itasetron; jasplakinolide; kahalalide F; lamellarin-N triacetate; lanreotide; leinamycin; lenograstim; lentinan sulfate; leptolstatin; letrozole; leukemia inhibiting factor; leukocyte alpha interferon; leuprolide+estrogen+progesterone; leuprorelin; levamisole; liarozole; linear polyamine analogue; lipophilic disaccharide peptide; lipophilic platinum compounds; lissoclinamide 7; lobaplatin; lombricine; lometrexol; lonidamine; losoxantrone; loxoribine; lurtotecan; lutetium texaphyrin; lysofylline; lytic peptides; maitansine; mannostatin A; marimastat; masoprocol; maspin; matrilysin inhibitors; matrix metalloproteinase inhibitors; menogaril; merbarone; meterelin; methioninase; metoclopramide; MIF inhibitor; mifepristone; miltefosine; mirimostim; mitoguazone; mitolactol; mitomycin analogues; mitonafide; mitotoxin fibroblast growth factor-saporin; mitoxantrone; mofarotene; molgramostim; Erbitux, human chorionic gonadotrophin; monophosphoryl lipid A+myobacterium cell wall sk; mopidamol; mustard anti-cancer agent; mycaperoxide B; mycobacterial cell wall extract; myriaporone; N-acetyldinaline; N-substituted benzamides; nafarelin; nagrestip; naloxone+pentazocine; napavin; naphterpin; nartograstim; nedaplatin; nemorubicin; neridronic acid; nilutamide; nisamycin; nitric oxide modulators; nitroxide antioxidant; nitrullyn; oblimersen (Genasense); O⁶-benzylguanine; octreotide; okicenone; oligonucleotides; onapristone; ondansetron; ondansetron; oracin; oral cytokine inducer; ormaplatin; osaterone; oxaliplatin; oxaunomycin; paclitaxel; paclitaxel analogues; paclitaxel derivatives; palauamine; palmitoylrhizoxin; pamidronic acid; panaxytriol; panomifene; parabactin; pazelliptine; pegaspargase; peldesine; pentosane polysulfate sodium; pentostatin; pentrozole; perflubron; perfosfamide; perillyl alcohol; phenazinomycin; phenylacetate; phosphatase inhibitors; picibanil; pilocarpine hydrochloride; pirarubicin; piritrexim; placetin A; placetin B; plasminogen activator inhibitor; platinum complex; platinum compounds; platinum-triamine complex; porfimer sodium; porfiromycin; prednisone; propyl bis-acridone; prostaglandin J2; proteasome inhibitors; protein A-based immune modulator; protein kinase C inhibitor; protein kinase C inhibitors, microalgal; protein tyrosine phosphatase inhibitors; purine nucleoside phosphorylase inhibitors; purpurins; pyrazoloacridine; pyridoxylated hemoglobin polyoxyethylene conjugate; raf antagonists; raltitrexed; ramosetron; ras farnesyl protein transferase inhibitors; ras inhibitors; ras-GAP inhibitor; retelliptine demethylated; rhenium Re 186 etidronate; rhizoxin; ribozymes; RII retinamide; rohitukine; romurtide; roquinimex; rubiginone B1; ruboxyl; safingol; saintopin; SarCNU; sarcophytol A; sargramostim; Sdi 1 mimetics; semustine; senescence derived inhibitor 1; sense oligonucleotides; signal transduction inhibitors; sizofiran; sobuzoxane; sodium borocaptate; sodium phenylacetate; solverol; somatomedin binding protein; sonermin; sparfosic acid; spicamycin D; spiromustine; splenopentin; spongistatin 1; squalamine; stipiamide; stromelysin inhibitors; sulfinosine; superactive vasoactive intestinal peptide antagonist; suradista; suramin; swainsonine; tallimustine; tamoxifen methiodide; tauromustine; tazarotene; tecogalan sodium; tegafur; tellurapyrylium; telomerase inhibitors; temoporfin; teniposide; tetrachlorodecaoxide; tetrazomine; thaliblastine; thiocoraline; thrombopoietin; thrombopoietin mimetic; thymalfasin; thymopoietin receptor agonist; thymotrinan; thyroid stimulating hormone; tin ethyl etiopurpurin; tirapazamine; titanocene bichloride; topsentin; toremifene; translation inhibitors; tretinoin; triacetyluridine; triciribine; trimetrexate; triptorelin; tropisetron; turosteride; tyrosine kinase inhibitors; tyrphostins; UBC inhibitors; ubenimex; urogenital sinus-derived growth inhibitory factor; urokinase receptor antagonists; vapreotide; variolin B; velaresol; veramine; verdins; verteporfin; vinorelbine; vinxaltine; vitaxin; vorozole; zanoterone; zeniplatin; zilascorb; and zinostatin stimalamer.

In certain embodiments, the second agent is selected from one or more checkpoint inhibitors. In one embodiment, one checkpoint inhibitor is used in combination with Compound D or a formulation of Compound D in the methods provided herein. In another embodiment, two checkpoint inhibitors are used in combination with Compound D or a formulation of Compound D in connection with the methods provided herein. In yet another embodiment, three or more checkpoint inhibitors are used in combination with Compound D or a formulation of Compound D in connection with the methods provided herein.

As used herein, the term “immune checkpoint inhibitor” or “checkpoint inhibitor” refers to molecules that totally or partially reduce, inhibit, interfere with or modulate one or more checkpoint proteins. Without being limited by a particular theory, checkpoint proteins regulate T-cell activation or function. Numerous checkpoint proteins are known, such as CTLA-4 and its ligands CD80 and CD86; and PD-1 with its ligands PD-L1 and PD-L2 (Pardoll, Nature Reviews Cancer, 2012, 12, 252-264). These proteins appear responsible for co-stimulatory or inhibitory interactions of T-cell responses. Immune checkpoint proteins appear to regulate and maintain self-tolerance and the duration and amplitude of physiological immune responses. Immune checkpoint inhibitors include antibodies or are derived from antibodies.

In one embodiment, the checkpoint inhibitor is a CTLA-4 inhibitor. In one embodiment, the CTLA-4 inhibitor is an anti-CTLA-4 antibody. Examples of anti-CTLA-4 antibodies include, but are not limited to, those described in U.S. Pat. Nos. 5,811,097; 5,811,097; 5,855,887; 6,051,227; 6,207,157; 6,682,736; 6,984,720; and 7,605,238, all of which are incorporated herein in their entireties. In one embodiment, the anti-CTLA-4 antibody is tremelimumab (also known as ticilimumab or CP-675,206). In another embodiment, the anti-CTLA-4 antibody is ipilimumab (also known as MDX-010 or MDX-101). Ipilimumab is a fully human monoclonal IgG antibody that binds to CTLA-4. Ipilimumab is marketed under the trade name Yervoy™.

In one embodiment, the checkpoint inhibitor is a PD-1/PD-L1 inhibitor. Examples of PD-1/PD-L1 inhibitors include, but are not limited to, those described in U.S. Pat. Nos. 7,488,802; 7,943,743; 8,008,449; 8,168,757; 8,217,149, and PCT Patent Application Publication Nos. WO2003042402, WO2008156712, WO2010089411, WO2010036959, WO2011066342, WO2011159877, WO2011082400, and WO2011161699, all of which are incorporated herein in their entireties.

In one embodiment, the checkpoint inhibitor is a PD-1 inhibitor. In one embodiment, the PD-1 inhibitor is an anti-PD-1 antibody. In one embodiment, the anti-PD-1 antibody is BGB-A317, nivolumab (also known as ONO-4538, BMS-936558, or MDX1106) or pembrolizumab (also known as MK-3475, SCH 900475, or lambrolizumab). In one embodiment, the anti-PD-1 antibody is nivolumab. Nivolumab is a human IgG4 anti-PD-1 monoclonal antibody, and is marketed under the trade name Opdivo™. In another embodiment, the anti-PD-1 antibody is pembrolizumab. Pembrolizumab is a humanized monoclonal IgG4 antibody and is marketed under the trade name Keytruda™. In yet another embodiment, the anti-PD-1 antibody is CT-011, a humanized antibody. CT-011 administered alone has failed to show response in treating acute myeloid leukemia (AML) at relapse. In yet another embodiment, the anti-PD-1 antibody is AMP-224, a fusion protein. In another embodiment, the PD-1 antibody is BGB-A317. BGB-A317 is a monoclonal antibody in which the ability to bind Fc gamma receptor I is specifically engineered out, and which has a unique binding signature to PD-1 with high affinity and superior target specificity.

In one embodiment, the checkpoint inhibitor is a PD-L1 inhibitor. In one embodiment, the PD-L1 inhibitor is an anti-PD-L1 antibody. In one embodiment, the anti-PD-L1 antibody is MEDI4736 (durvalumab). In another embodiment, the anti-PD-L1 antibody is BMS-936559 (also known as MDX-1105-01). In yet another embodiment, the PD-L1 inhibitor is atezolizumab (also known as MPDL3280A, and Tecentriq®).

In one embodiment, the checkpoint inhibitor is a PD-L2 inhibitor. In one embodiment, the PD-L2 inhibitor is an anti-PD-L2 antibody. In one embodiment, the anti-PD-L2 antibody is rHIgM12B7A.

In one embodiment, the checkpoint inhibitor is a lymphocyte activation gene-3 (LAG-3) inhibitor. In one embodiment, the LAG-3 inhibitor is IMP321, a soluble Ig fusion protein (Brignone et al., J. Immunol., 2007, 179, 4202-4211). In another embodiment, the LAG-3 inhibitor is BMS-986016.

In one embodiment, the checkpoint inhibitor is a B7 inhibitor. In one embodiment, the B7 inhibitor is a B7-H3 inhibitor or a B7-H4 inhibitor. In one embodiment, the B7-H3 inhibitor is MGA271, an anti-B7-H3 antibody (Loo et al., Clin. Cancer Res., 2012, 3834).

In one embodiment, the checkpoint inhibitor is a TIM3 (T-cell immunoglobulin domain and mucin domain 3) inhibitor (Fourcade et al., J. Exp. Med., 2010, 207, 2175-86; Sakuishi et al., J. Exp. Med., 2010, 207, 2187-94).

In one embodiment, the checkpoint inhibitor is an OX40 (CD134) agonist. In one embodiment, the checkpoint inhibitor is an anti-OX40 antibody. In one embodiment, the anti-OX40 antibody is anti-OX-40. In another embodiment, the anti-OX40 antibody is MEDI6469.

In one embodiment, the checkpoint inhibitor is a GITR agonist. In one embodiment, the checkpoint inhibitor is an anti-GITR antibody. In one embodiment, the anti-GITR antibody is TRX518.

In one embodiment, the checkpoint inhibitor is a CD137 agonist. In one embodiment, the checkpoint inhibitor is an anti-CD137 antibody. In one embodiment, the anti-CD137 antibody is urelumab. In another embodiment, the anti-CD137 antibody is PF-05082566.

In one embodiment, the checkpoint inhibitor is a CD40 agonist. In one embodiment, the checkpoint inhibitor is an anti-CD40 antibody. In one embodiment, the anti-CD40 antibody is CF-870,893.

In one embodiment, the checkpoint inhibitor is recombinant human interleukin-15 (rhIL-15).

In one embodiment, the checkpoint inhibitor is an IDO inhibitor. In one embodiment, the IDO inhibitor is INCB024360. In another embodiment, the IDO inhibitor is indoximod.

In certain embodiments, the combination therapies provided herein include two or more of the checkpoint inhibitors described herein (including checkpoint inhibitors of the same or different class). Moreover, the combination therapies described herein can be used in combination with second active agents as described herein where appropriate for treating diseases described herein and understood in the art.

In certain embodiments, Compound D can be used in combination with one or more immune cells expressing one or more chimeric antigen receptors (CARs) on their surface (e.g., a modified immune cell). Generally, CARs comprise an extracellular domain from a first protein e.g., an antigen-binding protein), a transmembrane domain, and an intracellular signaling domain. In certain embodiments, once the extracellular domain binds to a target protein such as a tumor-associated antigen (TAA) or tumor-specific antigen (TSA), a signal is generated via the intracellular signaling domain that activates the immune cell, e.g., to target and kill a cell expressing the target protein.

Extracellular domains: The extracellular domains of the CARs bind to an antigen of interest. In certain embodiments, the extracellular domain of the CAR comprises a receptor, or a portion of a receptor, that binds to said antigen. In certain embodiments, the extracellular domain comprises, or is, an antibody or an antigen-binding portion thereof. In specific embodiments, the extracellular domain comprises, or is, a single chain Fv (scFv) domain. The single-chain Fv domain can comprise, for example, a VL linked to VH by a flexible linker, wherein said VL and VH are from an antibody that binds said antigen.

In certain embodiments, the antigen recognized by the extracellular domain of a polypeptide described herein is a tumor-associated antigen (TAA) or a tumor-specific antigen (TSA). In various specific embodiments, the tumor-associated antigen or tumor-specific antigen is, without limitation, Her2, prostate stem cell antigen (PSCA), alpha-fetoprotein (AFP), carcinoembryonic antigen (CEA), cancer antigen-125 (CA-125), CA19-9, calretinin, MUC-1, B cell maturation antigen (BCMA), epithelial membrane protein (EMA), epithelial tumor antigen (ETA), tyrosinase, melanoma-24 associated antigen (MAGE), CD19, CD22, CD27, CD30, CD34, CD45, CD70, CD99, CD117, EGFRvIII (epidermal growth factor variant III), mesothelin, PAP (prostatic acid phosphatase), prostein, TARP (T cell receptor gamma alternate reading frame protein), Trp-p8, STEAPI (six-transmembrane epithelial antigen of the prostate 1), chromogranin, cytokeratin, desmin, glial fibrillary acidic protein (GFAP), gross cystic disease fluid protein (GCDFP-15), HMB-45 antigen, protein melan-A (melanoma antigen recognized by T lymphocytes; MART-I), myo-D1, muscle-specific actin (MSA), neurofilament, neuron-specific enolase (NSE), placental alkaline phosphatase, synaptophysis, thyroglobulin, thyroid transcription factor-1, the dimeric form of the pyruvate kinase isoenzyme type M2 (tumor M2-PK), an abnormal ras protein, or an abnormal p53 protein. In certain other embodiments, the TAA or TSA recognized by the extracellular domain of a CAR is integrin αvβ3 (CD61), galactin, or Ral-B.

In certain embodiments, the TAA or TSA recognized by the extracellular domain of a CAR is a cancer/testis (CT) antigen, e.g., BAGE, CAGE, CTAGE, FATE, GAGE, HCA661, HOM-TES-85, MAGEA, MAGEB, MAGEC, NA88, NY-ESO-1, NY-SAR-35, OY-TES-1, SPANXBI, SPA17, SSX, SYCPI, or TPTE.

In certain other embodiments, the TAA or TSA recognized by the extracellular domain of a CAR is a carbohydrate or ganglioside, e.g., fuc-GMI, GM2 (oncofetal antigen-immunogenic-1; OFA-I-1); GD2 (OFA-I-2), GM3, GD3, and the like.

In certain other embodiments, the TAA or TSA recognized by the extracellular domain of a CAR is alpha-actinin-4, Bage-1, BCR-ABL, Bcr-Abl fusion protein, beta-catenin, CA 125, CA 15-3 (CA 27.29\BCAA), CA 195, CA 242, CA-50, CAM43, Casp-8, cdc27, cdk4, cdkn2a, CEA, coa-1, dek-can fusion protein, EBNA, EF2, Epstein Barr virus antigens, ETV6-AML1 fusion protein, HLA-A2, HLA-A11, hsp70-2, KIAA0205, Mart2, Mum-1, 2, and 3, neo-PAP, myosin class I, OS-9, pml-RARα fusion protein, PTPRK, K-ras, N-ras, triosephosphate isomerase, Gage 3,4,5,6,7, GnTV, Herv-K-mel, Lage-1, NA-88, NY-Eso-1/Lage-2, SP17, SSX-2, TRP2-Int2, gp100 (Pme117), tyrosinase, TRP-1, TRP-2, MAGE-1, MAGE-3, RAGE, GAGE-1, GAGE-2, p15(58), RAGE, SCP-1, Hom/Me1-40, PRAIVIE, p53, HRas, HER-2/neu, E2A-PRL, H4-RET, IGH-IGK, MYL-RAR, human papillomavirus (HPV) antigens E6 and E7, TSP-180, MAGE-4, MAGE-5, MAGE-6, p185erbB2, p180erbB-3, c-met, nm-23H1, PSA, TAG-72-4, CA 19-9, CA 72-4, CAM 17.1, NuMa, K-ras, 13-Catenin, Mum-1, p16, TAGE, PSMA, CT7, telomerase, 43-9F, 5T4, 791Tgp72, 13HCG, BCA225, BTAA, CD68\KP1, C0-029, FGF-5, G250, Ga733 (EpCAM), HTgp-175, M344, MA-50, MG7-Ag, MOV18, NB\70K, NY-C0-1, RCAS1, SDCCAG16, TA-90, TAAL6, TAG72, TLP, or TPS.

In various specific embodiments, the tumor-associated antigen or tumor-specific antigen is an AML-related tumor antigen, as described in S. Anguille et al, Leukemia (2012), 26, 2186-2196.

Other tumor-associated and tumor-specific antigens are known to those in the art.

Receptors, antibodies, and scFvs that bind to TSAs and TAAs, useful in constructing chimeric antigen receptors, are known in the art, as are nucleotide sequences that encode them.

In certain specific embodiments, the antigen recognized by the extracellular domain of a chimeric antigen receptor is an antigen not generally considered to be a TSA or a TAA, but which is nevertheless associated with tumor cells, or damage caused by a tumor. In certain embodiments, for example, the antigen is, e.g., a growth factor, cytokine or interleukin, e.g., a growth factor, cytokine, or interleukin associated with angiogenesis or vasculogenesis. Such growth factors, cytokines, or interleukins can include, e.g., vascular endothelial growth factor (VEGF), basic fibroblast growth factor (bFGF), platelet-derived growth factor (PDGF), hepatocyte growth factor (HGF), insulin-like growth factor (IGF), or interleukin-8 (IL-8). Tumors can also create a hypoxic environment local to the tumor. As such, in other specific embodiments, the antigen is a hypoxia-associated factor, e.g., HIF-1α, HIF-1β, HIF-2α, HIF-2β, HIF-3α, or HIF-3β. Tumors can also cause localized damage to normal tissue, causing the release of molecules known as damage associated molecular pattern molecules (DAMPs; also known as alarmins). In certain other specific embodiments, therefore, the antigen is a DAMP, e.g., a heat shock protein, chromatin-associated protein high mobility group box 1 (HMGB 1), S100A8 (MRP8, calgranulin A), S100A9 (MRP14, calgranulin B), serum amyloid A (SAA), or can be a deoxyribonucleic acid, adenosine triphosphate, uric acid, or heparin sulfate.

Transmembrane domain: In certain embodiments, the extracellular domain of the CAR is joined to the transmembrane domain of the polypeptide by a linker, spacer or hinge polypeptide sequence, e.g., a sequence from CD28 or a sequence from CTLA4. The transmembrane domain can be obtained or derived from the transmembrane domain of any transmembrane protein, and can include all or a portion of such transmembrane domain. In specific embodiments, the transmembrane domain can be obtained or derived from, e.g., CD8, CD16, a cytokine receptor, and interleukin receptor, or a growth factor receptor, or the like.

Intracellular signaling domains: In certain embodiments, the intracellular domain of a CAR is or comprises an intracellular domain or motif of a protein that is expressed on the surface of T cells and triggers activation and/or proliferation of said T cells. Such a domain or motif is able to transmit a primary antigen-binding signal that is necessary for the activation of a T lymphocyte in response to the antigen's binding to the CAR's extracellular portion. Typically, this domain or motif comprises, or is, an ITAM (immunoreceptor tyrosine-based activation motif). ITAM-containing polypeptides suitable for CARs include, for example, the zeta CD3 chain (CD3ζ) or ITAM-containing portions thereof. In a specific embodiment, the intracellular domain is a CD3ζ intracellular signaling domain. In other specific embodiments, the intracellular domain is from a lymphocyte receptor chain, a TCR/CD3 complex protein, an Fe receptor subunit or an IL-2 receptor subunit. In certain embodiments, the CAR additionally comprises one or more co-stimulatory domains or motifs, e.g., as part of the intracellular domain of the polypeptide. The one or more co-stimulatory domains or motifs can be, or can comprise, one or more of a co-stimulatory CD27 polypeptide sequence, a co-stimulatory CD28 polypeptide sequence, a co-stimulatory OX40 (CD134) polypeptide sequence, a co-stimulatory 4-1BB (CD137) polypeptide sequence, or a co-stimulatory inducible T-cell costimulatory (ICOS) polypeptide sequence, or other costimulatory domain or motif, or any combination thereof.

The CAR may also comprise a T cell survival motif. The T cell survival motif can be any polypeptide sequence or motif that facilitates the survival of the T lymphocyte after stimulation by an antigen. In certain embodiments, the T cell survival motif is, or is derived from, CD3, CD28, an intracellular signaling domain of IL-7 receptor (IL-7R), an intracellular signaling domain of IL-12 receptor, an intracellular signaling domain of IL-15 receptor, an intracellular signaling domain of IL-21 receptor, or an intracellular signaling domain of transforming growth factor β (TGFβ) receptor.

The modified immune cells expressing the CARs can be, e.g., T lymphocytes (T cells, e.g., CD4+ T cells or CD8+ T cells), cytotoxic lymphocytes (CTLs) or natural killer (NK) cells. T lymphocytes used in the compositions and methods provided herein may be naive T lymphocytes or MHC-restricted T lymphocytes. In certain embodiments, the T lymphocytes are tumor infiltrating lymphocytes (TILs). In certain embodiments, the T lymphocytes have been isolated from a tumor biopsy, or have been expanded from T lymphocytes isolated from a tumor biopsy. In certain other embodiments, the T cells have been isolated from, or are expanded from T lymphocytes isolated from, peripheral blood, cord blood, or lymph. Immune cells to be used to generate modified immune cells expressing a CAR can be isolated using art-accepted, routine methods, e.g., blood collection followed by apheresis and optionally antibody-mediated cell isolation or sorting.

The modified immune cells are preferably autologous to an individual to whom the modified immune cells are to be administered. In certain other embodiments, the modified immune cells are allogeneic to an individual to whom the modified immune cells are to be administered. Where allogeneic T lymphocytes or NK cells are used to prepare modified T lymphocytes, it is preferable to select T lymphocytes or NK cells that will reduce the possibility of graft-versus-host disease (GVHD) in the individual. For example, in certain embodiments, virus-specific T lymphocytes are selected for preparation of modified T lymphocytes; such lymphocytes will be expected to have a greatly reduced native capacity to bind to, and thus become activated by, any recipient antigens. In certain embodiments, recipient-mediated rejection of allogeneic T lymphocytes can be reduced by co-administration to the host of one or more immunosuppressive agents, e.g., cyclosporine, tacrolimus, sirolimus, cyclophosphamide, or the like.

T lymphocytes, e.g., unmodified T lymphocytes, or T lymphocytes expressing CD3 and CD28, or comprising a polypeptide comprising a CD3ζ signaling domain and a CD28 co-stimulatory domain, can be expanded using antibodies to CD3 and CD28, e.g., antibodies attached to beads; see, e.g., U.S. Pat. Nos. 5,948,893; 6,534,055; 6,352,694; 6,692,964; 6,887,466; and 6,905,681.

The modified immune cells, e.g., modified T lymphocytes, can optionally comprise a “suicide gene” or “safety switch” that enables killing of substantially all of the modified immune cells when desired. For example, the modified T lymphocytes, in certain embodiments, can comprise an HSV thymidine kinase gene (HSV-TK), which causes death of the modified T lymphocytes upon contact with gancyclovir. In another embodiment, the modified T lymphocytes comprise an inducible caspase, e.g., an inducible caspase 9 (icaspase9), e.g., a fusion protein between caspase 9 and human FK506 binding protein allowing for dimerization using a specific small molecule pharmaceutical. See Straathof et al., Blood 105(11):4247-4254 (2005).

Specific second active agents useful in the methods or compositions include, but are not limited to, rituximab, oblimersen (Genasense®), remicade, docetaxel, celecoxib, melphalan, dexamethasone (Decadron®), steroids, gemcitabine, cisplatinum, temozolomide, etoposide, cyclophosphamide, temodar, carboplatin, procarbazine, gliadel, tamoxifen, topotecan, methotrexate, Arisa®, Taxol, taxotere, fluorouracil, leucovorin, irinotecan, xeloda, interferon alpha, pegylated interferon alpha (e.g., PEG INTRON-A), capecitabine, cisplatin, thiotepa, fludarabine, carboplatin, liposomal daunorubicin, Ara-C, doxetaxol, pacilitaxel, vinblastine, IL-2, GM-CSF, dacarbazine, vinorelbine, zoledronic acid, palmitronate, biaxin, busulphan, prednisone, bisphosphonate, arsenic trioxide, vincristine, doxorubicin (Doxil®), paclitaxel, ganciclovir, adriamycin, estramustine sodium phosphate (Emcyt®), sulindac, and etoposide.

In certain embodiments of the methods provided herein, use of a second active agent in combination with Compound D, including a formulation of Compound D provided herein, may be modified or delayed during or shortly following administration of Compound D, including a formulation of Compound D provided herein, as deemed appropriate by the practitioner of skill in the art. In certain embodiments, subjects being administered Compound D, including a formulation of Compound D provided herein, alone or in combination with other therapies may receive supportive care including antiemetics, myeloid growth factors, and transfusions of platelets, when appropriate. In some embodiments, subjects being administered Compound D, including a formulation of Compound D provided herein, may be administered a growth factor as a second active agent according to the judgment of the practitioner of skill in the art. In some embodiments, provided is administration of Compound D, including a formulation of Compound D provided herein, in combination with erythropoietin or darbepoetin (Aranesp).

In one aspect, provided herein is a method of treating, preventing, managing, and/or ameliorating locally advanced or metastatic transitional cell bladder cancer comprising administering a formulation of Compound D with gemcitabine, cisplatinum, 5-fluorouracil, mitomycin, methotrexate, vinblastine, doxorubicin, carboplatin, thiotepa, paclitaxel, docetaxel, atezolizumab, avelumab, durvalumab, Keytruda (pembrolizumab) and/or nivolumab.

In one aspect, methods of treating, preventing, managing, and/or ameliorating a cancer provided herein comprise administering a formulation of Compound D in combination with a second active ingredient as follows: temozolomide to pediatric patients with relapsed or progressive brain tumors or recurrent neuroblastoma; celecoxib, etoposide and cyclophosphamide for relapsed or progressive CNS cancer; temodar to patients with recurrent or progressive meningioma, malignant meningioma, hemangiopericytoma, multiple brain metastases, relapsed brain tumors, or newly diagnosed glioblastoma multiforms; irinotecan to patients with recurrent glioblastoma; carboplatin to pediatric patients with brain stem glioma; procarbazine to pediatric patients with progressive malignant gliomas; cyclophosphamide to patients with poor prognosis malignant brain tumors, newly diagnosed or recurrent glioblastoma multiforms; Gliadel® for high grade recurrent malignant gliomas; temozolomide and tamoxifen for anaplastic astrocytoma; or topotecan for gliomas, glioblastoma, anaplastic astrocytoma or anaplastic oligodendroglioma.

In one aspect, methods of treating, preventing, managing, and/or ameliorating a metastatic breast cancer provided herein comprise administering a formulation of Compound D with methotrexate, cyclophosphamide, capecitabine, 5-fluorouracil, taxane, temsirolimus, ABRAXANE® (paclitaxel protein-bound particles for injectable suspension) (albumin-bound), lapatinib, herceptin, pamidronate disodium, eribulin mesylate, everolimus, gemcitabine, palbociclib, ixabepilone, kadcyla, pertuzumab, theotepa, anastrozole, docetaxel, doxorubicin hydrochloride, epirubicin hydrochloride, toremifene, fulvestrant, goserelin acetate, ribociclib, megestrol acetate, vinblastin, aromatase inhibitors, such as letrozole, exemestane, selective estrogen modulators, estrogen receptor antagonists, anthracyclines, emtansine, and/or pexidartinib to patients with metastatic breast cancer.

In one aspect, methods of treating, preventing, managing, and/or ameliorating neuroendocrine tumors provided herein comprise administering a formulation of Compound D with at least one of everolimus, avelumab, sunitinib, nexavar, leucovorin, oxaliplatin, temozolomide, capecitabine, bevacizumab, doxorubicin (Adriamycin), fluorouracil (Adrucil, 5-fluorouracil), streptozocin (Zanosar), dacarbazine, sandostatin, lanreotide, and/or pasireotide to patients with neuroendocrine tumors.

In one aspect, methods of treating, preventing, managing, and/or ameliorating a metastatic breast cancer provided herein comprise administering a formulation of Compound D with methotrexate, gemcitabine, cisplatin, cetuximab, 5-fluorouracil, bleomycin, docetaxel, carboplatin, hydroxyurea, pembrolizumab and/or nivolumab to patients with recurrent or metastatic head or neck cancer.

In one aspect, methods of treating, preventing, managing, and/or ameliorating a pancreatic cancer provided herein comprise administering a formulation of Compound D with gemcitabine, ABRAXANE®, 5-fluorouracil, afinitor, irinotecan, mitomycin C, sunitinib, sunitinibmalate, and/or tarceva to patients with pancreatic cancer.

In one aspect, methods of treating, preventing, managing, and/or ameliorating a colon or rectal cancer provided herein comprise administering a formulation of Compound D with ARISA®, avastatin, oxaliplatin, 5-fluorouracil, irinotecan, capecitabine, cetuximab, ramucirumab, panitumumab, bevacizumab, leucovorin calcium, lonsurf, regorafenib, ziv-aflibercept, Taxol, and/or taxotere.

In one aspect, methods of treating, preventing, managing, and/or ameliorating a refractory colorectal cancer provided herein comprise administering a formulation of Compound D with capecitabine and/or vemurafenib to patients with refractory colorectal cancer, or patients who fail first line therapy or have poor performance in colon or rectal adenocarcinoma.

In one aspect, methods of treating, preventing, managing, and/or ameliorating a colorectal cancer provided herein comprise administering a formulation of Compound D with fluorouracil, leucovorin, and/or irinotecan to patients with colorectal cancer, including stage 3 and stage 4, or to patients who have been previously treated for metastatic colorectal cancer.

In certain embodiments, a formulation of Compound D provided herein is administered to patients with refractory colorectal cancer in combination with capecitabine, xeloda, and/or irinotecan.

In certain embodiments, a formulation of Compound D provided herein is administered with capecitabine and irinotecan to patients with refractory colorectal cancer or to patients with unresectable or metastatic colorectal carcinoma.

In one aspect, the methods provided herein comprise administering a formulation of Compound D with interferon alpha or capecitabine to patients with unresectable or metastatic hepatocellular carcinoma; or with cisplatin and thiotepa, or with sorafenib tosylate to patients with primary or metastatic liver cancer.

In one aspect, the methods provided herein comprise administering a formulation of Compound D with doxorubicin, paclitaxel, vinblastine, pegylated interferon alpha and/or recombinant interferon alpha-2b to patients with Kaposi's sarcoma.

In one aspect, the methods provided herein comprise administering a formulation of Compound D with at least one of enasidenib, arsenic trioxide, fludarabine, carboplatin, daunorubicin, cyclophosphamide, cytarabine, doxorubicin, idarubicin, mitoxantrone hydrochloride, thioguanine, vincristine, midostaurin and/or topotecan to patients with acute myeloid leukemia, including refractory or relapsed or high-risk acute myeloid leukemia.

In one aspect, the methods provided herein comprise administering a formulation of Compound D with at least one of enasidenib, liposomal daunorubicin, topotecan and/or cytarabine to patients with unfavorable karyotype acute myeloblastic leukemia.

In one aspect, the methods provided herein comprise administering Compound D with an IDH2 inhibitor to a patient having leukemia, wherein the leukemia is characterized by the presence of a mutant allele of IDH2. Exemplary IDH2 inhibitors are disclosed in U.S. Pat. Nos. 9,732,062; 9,724,350; 9,738,625; and 9,579,324; and US Publication Nos. 2016-0159771 and US 2016-0158230 A1. In one aspect, the methods provided herein comprise administering Compound D with enasidenib to a patient having leukemia, wherein the leukemia is characterized by the presence of a mutant allele of IDH2. In certain embodiments, the combination of Compound D and an IDH2 inhibitor increases differentiated cells (CD34-/CD38) and erythroblasts in a patient having acute myeloid leukemia, wherein the acute myeloid leukemia is characterized by the presence of IDH2 R140Q. In certain embodiments, the combination of Compound D and an IDH2 inhibitor reduces progenitor cells (CD34+/CD38+) and HSC in a patient having acute myeloid leukemia, wherein the acute myeloid leukemia is characterized by the presence of IDH2 R140Q.

In one aspect, the methods provided herein comprise administering Compound D with enasidenib to a patient having acute myeloid leukemia, wherein the acute myeloid leukemia is characterized by the presence of a mutant allele of IDH2. In one embodiment, the mutant allele of IDH2 is IDH2 R140Q or R172K.

In one aspect, the methods provided herein comprise administering a formulation of Compound D with enasidenib to a patient having leukemia, wherein the leukemia is characterized by the presence of a mutant allele of IDH2. In one aspect, the methods provided herein comprise administering a formulation of Compound D with enasidenib to a patient having acute myeloid leukemia, wherein the acute myeloid leukemia is characterized by the presence of a mutant allele of IDH2. In one embodiment, the mutant allele of IDH2 is IDH2 R140Q or R172K.

In one aspect, the methods provided herein comprise administering Compound D with 6-(6-(trifluoromethyl)pyridin-2-yl)-N2-(2-(trifluoromethyl)pyridin-4-yl)-1,3,5-triazine-2,4-diamine (Compound 2) to a patient having leukemia, wherein the leukemia is characterized by the presence of a mutant allele of IDH2. In one aspect, the methods provided herein comprise administering Compound D with Compound 2 to a patient having acute myeloid leukemia, wherein the acute myeloid leukemia is characterized by the presence of a mutant allele of IDH2. In one embodiment, the mutant allele of IDH2 is IDH2 R140Q or R172K.

In one aspect, the methods provided herein comprise administering a formulation of Compound D with Compound 2 to a patient having leukemia, wherein the leukemia is characterized by the presence of a mutant allele of IDH2. In one aspect, the methods provided herein comprise administering a formulation of Compound D with Compound 2 to a patient having acute myeloid leukemia, wherein the acute myeloid leukemia is characterized by the presence of a mutant allele of IDH2. In one embodiment, the mutant allele of IDH2 is IDH2 R140Q or R172K.

In one aspect, the methods provided herein comprise administering a formulation of Compound D with methotrexate, mechlorethamine hydrochloride, afatinib dimaleate, pemetrexed, bevacizumab, carboplatin, cisplatin, ceritinib, crizotinib, ramucirumab, pembrolizumab, docetaxel, vinorelbine tartrate, gemcitabine, ABRAXANE®, erlotinib, geftinib, irinotecan, everolimus, alectinib, brigatinib, nivolumab, osimertinib, atezolizumab, necitumumab and/or to patients with non-small cell lung cancer.

In one aspect, the methods provided herein comprise administering a formulation of Compound D with carboplatin and irinotecan to patients with non-small cell lung cancer.

In one aspect, the methods provided herein comprise administering a formulation of Compound D with doxetaxol to patients with non-small cell lung cancer who have been previously treated with carbo/etoposide and radiotherapy.

In one aspect, the methods provided herein comprise administering a formulation of Compound D with carboplatin and/or taxotere, or in combination with carboplatin, pacilitaxel and/or thoracic radiotherapy to patients with non-small cell lung cancer.

In one aspect, the methods provided herein comprise administering a formulation of Compound D with taxotere to patients with stage IIIB or IV non-small cell lung cancer.

In one aspect, the methods provided herein comprise administering a formulation of Compound D with oblimersen (Genasense®), methotrexate, mechlorethamine hydrochloride, etoposide, topotecan and/or doxorubicin to patients with small cell lung cancer.

In one aspect, the methods provided herein comprise administering a formulation of Compound D with Venetoclax, ABT-737 (Abbott Laboratories) and/or obatoclax (GX15-070) to patients with lymphoma and other blood cancers.

In one aspect, the methods provided herein comprise administering a formulation of Compound D with a second active ingredient such as vinblastine or fludarabine adcetris, ambochlorin, becenum, bleomycin, brentuximab vedotin, carmustinem chlorambucil, cyclophosphamide, dacarbazine, doxorubicin, lomustine, matulane, mechlorethamine hydrochloride, prednisone, procarbazine hydrochloride, vincristine, methotrexate, nelarabin, belinostat, bendamustine HCl, tositumomab, and iodine 131 tositumomab, denileukin diftitox, dexamethasone, pralatrexate, prelixafor, obinutuzumab, ibritumomab, tiuxefan, ibritinib, idelasib, intron A, romidepsin, lenalidomide, rituximab, and/or vorinostat to patients with various types of lymphoma, including, but not limited to, Hodgkin's lymphoma, non-Hodgkin's lymphoma, cutaneous T-Cell lymphoma, cutaneous B-Cell lymphoma, diffuse large B-Cell lymphoma or relapsed or refractory low grade follicular lymphoma.

In one aspect, the methods provided herein comprise administering a formulation of Compound D with taxotere, dabrafenib, imlygic, ipilimumab, pembrolizumab, nivolumab, trametinib, vemurafenib, talimogene laherparepvec, IL-2, IFN, GM-CSF, and/or dacarbazine, aldesleukin, cobimetinib, Intron A®, peginterferon Alfa-2b, and/or trametinib to patients with various types or stages of melanoma.

In one aspect, the methods provided herein comprise administering a formulation of Compound D with vinorelbine or pemetrexed disodium to patients with malignant mesothelioma, or stage IIIB non-small cell lung cancer with pleural implants or malignant pleural effusion mesothelioma syndrome.

In one aspect, the methods of treating patients with various types or stages of multiple myeloma provided herein comprise administering a formulation of Compound D with dexamethasone, zoledronic acid, palmitronate, GM-CSF, biaxin, vinblastine, melphalan, busulphan, cyclophosphamide, IFN, prednisone, bisphosphonate, celecoxib, arsenic trioxide, PEG INTRON-A, vincristine, becenum, bortezomib, carfilzomib, doxorubicin, panobinostat, lenalidomide, pomalidomide, thalidomide, mozobil, carmustine, daratumumab, elotuzumab, ixazomib citrate, plerixafor or a combination thereof.

In certain embodiments, a formulation of Compound D provided herein is administered to patients with various types or stages of multiple myeloma in combination with chimeric antigen receptor (CAR) T-cells. In certain embodiments the CAR T cell in the combination targets B cell maturation antigen (BCMA), and in more specific embodiments, the CAR T cell is bb2121 or bb21217. In some embodiments, the CAR T cell is JCARH125.

In certain embodiments, a formulation of Compound D provided herein is administered to patients with relapsed or refractory multiple myeloma in combination with doxorubicin (Doxil®), vincristine and/or dexamethasone (Decadron®).

In certain embodiments, the methods provided herein comprise administering a formulation of Compound D to patients with various types or stages of ovarian cancer such as peritoneal carcinoma, papillary serous carcinoma, refractory ovarian cancer or recurrent ovarian cancer, in combination with Taxol, carboplatin, doxorubicin, gemcitabine, cisplatin, xeloda, paclitaxel, dexamethasone, avastin, cyclophosphamide, topotecan, olaparib, thiotepa, melphalan, niraparib tosylate monohydrate, rubraca or a combination thereof.

In certain embodiments, the methods provided herein comprise administering a formulation of Compound D to patients with various types or stages of prostate cancer, in combination with xeloda, 5 FU/LV, gemcitabine, irinotecan plus gemcitabine, cyclophosphamide, vincristine, dexamethasone, GM-CSF, celecoxib, taxotere, ganciclovir, paclitaxel, adriamycin, docetaxel, estramustine, Emcyt, denderon, zytiga, bicalutamide, cabazitaxel, degarelix, enzalutamide, zoladex, leuprolide acetate, mitoxantrone hydrochloride, prednisone, sipuleucel-T, radium 223 dichloride, or a combination thereof.

In certain embodiments, the methods provided herein comprise administering a formulation of Compound D to patients with various types or stages of renal cell cancer, in combination with capecitabine, IFN, tamoxifen, IL-2, GM-CSF, Celebrex®, flutamide, goserelin acetate, nilutamide or a combination thereof.

In certain embodiments, the methods provided herein comprise administering a formulation of Compound D to patients with various types or stages of gynecologic, uterus or soft tissue sarcoma cancer in combination with IFN, dactinomycin, doxorubicin, imatinib mesylate, pazopanib, hydrochloride, trabectedin, eribulin mesylate, olaratumab, a COX-2 inhibitor such as celecoxib, and/or sulindac.

In one aspect, the methods provided herein comprise administering a formulation of Compound D to patients with various types or stages of solid tumors in combination with celecoxib, etoposide, cyclophosphamide, docetaxel, apecitabine, IFN, tamoxifen, IL-2, GM-CSF, or a combination thereof.

In one aspect, the methods provided herein comprise administering a formulation of Compound D to patients with scleroderma or cutaneous vasculitis in combination with Celebrex, etoposide, cyclophosphamide, docetaxel, apecitabine, IFN, tamoxifen, IL-2, GM-CSF, or a combination thereof.

In one aspect, the methods provided herein comprise administering a formulation of Compound D to patients with MDS in combination with azacitidine, cytarabine, daunorubicin, decitabine, idarubicin, lenalidomide, enasidenib, or a combination thereof.

In one aspect, the methods provided herein comprise administering Compound D to patients with hematological cancer in combination with one or more second agents selected from JAK inhibitors, FLT3 inhibitors, mTOR inhibitors, spliceosome inhibitors, BET inhibitors, SMG1 inhibitors, ERK inhibitors, LSD1 inhibitors, BH3 mimetics, topoisomerase inhibitors, and RTK inhibitors. In one aspect, the methods provided herein comprise administering a formulation of Compound D to patients with a hematological cancer in combination with one or more second agents selected from JAK inhibitors, FLT3 inhibitors, mTOR inhibitors, spliceosome inhibitors, BET inhibitors, SMG1 inhibitors, ERK inhibitors, LSD1 inhibitors, BH3 mimetics, topoisomerase inhibitors, and RTK inhibitors.

In one aspect, the methods provided herein comprise administering Compound D to patients with leukemia in combination with one or more second agents selected from JAK inhibitors, FLT3 inhibitors, mTOR inhibitors, spliceosome inhibitors, BET inhibitors, SMG1 inhibitors, ERK inhibitors, LSD1 inhibitors, BH3 mimetics, topoisomerase inhibitors, and RTK inhibitors. In certain embodiments, a formulation of Compound D provided herein is administered to patients with leukemia in combination with one or more second agents selected from JAK inhibitors, FLT3 inhibitors, mTOR inhibitors, spliceosome inhibitors, BET inhibitors, SMG1 inhibitors, ERK inhibitors, LSD1 inhibitors, BH3 mimetics, topoisomerase inhibitors, and RTK inhibitors.

In one aspect, the methods provided herein comprise administering Compound D to patients with AML in combination with one or more second agents selected from JAK inhibitors, FLT3 inhibitors, mTOR inhibitors, spliceosome inhibitors, BET inhibitors, SMG1 inhibitors, ERK inhibitors, LSD1 inhibitors, BH3 mimetics, topoisomerase inhibitors, and RTK inhibitors. In certain embodiments, a formulation of Compound D provided herein is administered to patients with AML in combination with one or more second agents selected from JAK inhibitors, FLT3 inhibitors, mTOR inhibitors, spliceosome inhibitors, BET inhibitors, SMG1 inhibitors, ERK inhibitors, LSD1 inhibitors, BH3 mimetics, topoisomerase inhibitors, and RTK inhibitors.

In one aspect, the methods provided herein comprise administering Compound D to patients with leukemia in combination with an mTOR inhibitor. In certain embodiments, a formulation of Compound D provided herein is administered to patients with leukemia in combination with an mTOR inhibitor. In certain embodiments, the mTOR inhibitor is selected from everolimus, MLN-0128 and AZD8055. In some embodiments, the mTOR inhibitor is an mTOR kinase inhibitor. In certain embodiments, the mTOR kinase inhibitor is selected from 7-(6-(2-hydroxypropan-2-yl)pyridin-3-yl)-1-((trans)-4-methoxycyclohexyl)-3,4-dihydropyrazino[2,3-b]pyrazin-2(1H)-one (CC-223) and 1-ethyl-7-(2-methyl-6-(1H-1,2,4-triazol-3-yl)pyridin-3-yl)-3,4-dihydropyrazino[2,3-b]pyrazin-2(1H)-one (CC-115). In certain embodiments, Compound D is administered to patients with leukemia in combination with 7-(6-(2-hydroxypropan-2-yl)pyridin-3-yl)-1-((trans)-4-methoxycyclohexyl)-3,4-dihydropyrazino[2,3-b]pyrazin-2(1H)-one (CC-223). In certain embodiments, Compound D is administered to patients with leukemia in combination with 1-ethyl-7-(2-methyl-6-(1H-1,2,4-triazol-3-yl)pyridin-3-yl)-3,4-dihydropyrazino[2,3-b]pyrazin-2(1H)-one (CC-115). In certain embodiments, Compound D is administered to patients with leukemia in combination with everolimus. In certain embodiments, Compound D is administered to patients with leukemia in combination with MLN-0128. In certain embodiments, Compound D is administered to patients with leukemia in combination with AZD8055.

In one aspect, the methods provided herein comprise administering Compound D to patients with AML in combination with an mTOR inhibitor. In certain embodiments, a formulation of Compound D provided herein is administered to patients with AML in combination with an mTOR inhibitor. In certain embodiments, the mTOR inhibitor is selected from everolimus, MLN-0128 and AZD8055. In some embodiments, the mTOR inhibitor is an mTOR kinase inhibitor. In certain embodiments, the mTOR kinase inhibitor is selected from 7-(6-(2-hydroxypropan-2-yl)pyridin-3-yl)-1-((trans)-4-methoxycyclohexyl)-3,4-dihydropyrazino[2,3-b]pyrazin-2(1H)-one (CC-223) and 1-ethyl-7-(2-methyl-6-(1H-1,2,4-triazol-3-yl)pyridin-3-yl)-3,4-dihydropyrazino[2,3-b]pyrazin-2(1H)-one (CC-115). In certain embodiments, Compound D is administered to patients with AML in combination with 1-ethyl-7-(2-methyl-6-(1H-1,2,4-triazol-3-yl)pyridin-3-yl)-3,4-dihydropyrazino[2,3-b]pyrazin-2(1H)-one. In certain embodiments, Compound D is administered to patients with AML in combination with everolimus. In certain embodiments, everolimus is administered to patients with AML prior to administration of Compound D. In certain embodiments, Compound D is administered to patients with AML in combination with MLN-0128. In certain embodiments, Compound D is administered to patients with AML in combination with AZD8055.

In one aspect, the methods provided herein comprise administering Compound D to patients with MPN in combination with a JAK inhibitor. In certain embodiments, a formulation of Compound D provided herein is administered to patients with MPN in combination with a JAK inhibitor. In one aspect the JAK inhibitor is selected from a JAK1 inhibitor, a JAK2 inhibitor and a JAK3 inhibitor. In certain embodiments, the JAK inhibitor is selected from tofacitinib, momelotinib, filgotinib, decernotinib, barcitinib, ruxolitinib, fedratinib, NS-018 and pacritinib. In certain embodiments, the JAK inhibitor is selected from tofacitinib, momelotinib, ruxolitinib, fedratinib, NS-018 and pacritinib. In certain embodiments, Compound D is administered to patients with MPN in combination with tofacitinib. In certain embodiments, Compound D is administered to patients with MPN in combination with momelotinib. In certain embodiments, Compound D is administered to patients with MPN in combination with filgotinib. In certain embodiments, Compound D is administered to patients with MPN in combination with decernotinib. In certain embodiments, Compound D is administered to patients with MPN in combination with barcitinib. In certain embodiments, Compound D is administered to patients with MPN in combination with ruxolitinib. In certain embodiments, Compound D is administered to patients with MPN in combination with fedratinib. In certain embodiments, Compound D is administered to patients with MPN in combination with NS-018. In certain embodiments, Compound D is administered to patients with MPN in combination with pacritinib. In certain embodiments, the MPN is IL-3 independent. In certain embodiments, the MPN is characterized by a JAK 2 mutation, for example, a JAK2V617F mutation.

In one aspect, the methods provided herein comprise administering Compound D to patients with myelofibrosis in combination with a JAK inhibitor. In certain embodiments, a formulation of Compound D provided herein is administered to patients with myelofibrosis in combination with a JAK inhibitor. In one aspect the JAK inhibitor is selected from a JAK1 inhibitor, a JAK2 inhibitor and a JAK3 inhibitor. In certain embodiments, the JAK inhibitor is selected from tofacitinib, momelotinib, ruxolitinib, fedratinib, NS-018 and pacritinib. In certain embodiments, Compound D is administered to patients with myelofibrosis in combination with tofacitinib. In certain embodiments, Compound D is administered to patients with myelofibrosis in combination with momelotinib. In certain embodiments, Compound D is administered to patients with myelofibrosis in combination with ruxolitinib. In certain embodiments, Compound D is administered to patients with myelofibrosis in combination with fedratinib. In certain embodiments, Compound D is administered to patients with myelofibrosis in combination with NS-018. In certain embodiments, Compound D is administered to patients with myelofibrosis in combination with pacritinib. In certain embodiments, the myeolofibrosis is characterized by a JAK 2 mutation, for example, a JAK2V617F mutation. In some embodiments, the myelofibrosis is primary myelofibrosis. In other embodiments, the myelofibrosis is secondary myelofibrosis. In some such embodiments, the secondary myelofibrosis is post polycythemia vera myelofibrosis. In other embodiments, the secondary myelofibrosis is post essential thrombocythemia myelofibrosis.

In one aspect, the methods provided herein comprise administering Compound D to patients with leukemia in combination with a JAK inhibitor. In certain embodiments, a formulation of Compound D provided herein is administered to patients with leukemia in combination with a JAK inhibitor. In one aspect the JAK inhibitor is selected from a JAK1 inhibitor, a JAK2 inhibitor and a JAK3 inhibitor. In certain embodiments, the JAK inhibitor is selected from tofacitinib, momelotinib, filgotinib, decernotinib, barcitinib, ruxolitinib, fedratinib, NS-018 and pacritinib. In certain embodiments, the JAK inhibitor is selected from momelotinib, ruxolitinib, fedratinib, NS-018 and pacritinib. In certain embodiments, Compound D is administered to patients with leukemia in combination with tofacitinib. In certain embodiments, Compound D is administered to patients with leukemia in combination with momelotinib. In certain embodiments, Compound D is administered to patients with leukemia in combination with filgotinib. In certain embodiments, Compound D is administered to patients with leukemia in combination with decernotinib. In certain embodiments, Compound D is administered to patients with leukemia in combination with barcitinib. In certain embodiments, Compound D is administered to patients with leukemia in combination with ruxolitinib. In certain embodiments, Compound D is administered to patients with leukemia in combination with fedratinib. In certain embodiments, Compound D is administered to patients with leukemia in combination with NS-018. In certain embodiments, Compound D is administered to patients with leukemia in combination with pacritinib. In certain embodiments, the MPN is characterized by a JAK 2 mutation, for example, a JAK2V617F mutation.

In one aspect, the methods provided herein comprise administering Compound D to patients with AML in combination with a JAK inhibitor. In certain embodiments, a formulation of Compound D provided herein is administered to patients with AML in combination with a JAK inhibitor. In one aspect the JAK inhibitor is selected from a JAK1 inhibitor, a JAK2 inhibitor and a JAK3 inhibitor. In certain embodiments, the JAK inhibitor is selected from tofacitinib, momelotinib, filgotinib, decernotinib, barcitinib, ruxolitinib, fedratinib, NS-018 and pacritinib. In certain embodiments, the JAK inhibitor is selected from momelotinib, ruxolitinib, fedratinib, NS-018 and pacritinib. In certain embodiments, Compound D is administered to patients with AML in combination with tofacitinib. In certain embodiments, Compound D is administered to patients with AML in combination with momelotinib. In certain embodiments, Compound D is administered to patients with AML in combination with filgotinib. In certain embodiments, Compound D is administered to patients with AML in combination with decernotinib. In certain embodiments, Compound D is administered to patients with AML in combination with barcitinib. In certain embodiments, Compound D is administered to patients with AML, in combination with ruxolitinib. In certain embodiments, Compound D is administered to patients with AML in combination with fedratinib. In certain embodiments, Compound D is administered to patients with AML in combination with NS-018. In certain embodiments, Compound D is administered to patients with AML in combination with pacritinib. In certain embodiments, the MPN is characterized by a JAK 2 mutation, for example, a JAK2V617F mutation.

In one aspect, the methods provided herein comprise administering Compound D to patients with leukemia in combination with a FLT3 kinase inhibitor. In certain embodiments, a formulation of Compound D provided herein is administered to patients with leukemia in combination with a FLT3 kinase inhibitor. In certain embodiments, the FLT3 kinase inhibitor is selected from quizartinib, sunitinib, sunitinib malate, midostaurin, pexidartinib, lestaurtinib, tandutinib, and crenolanib. In certain embodiments, Compound D is administered to patients with leukemia in combination with quizartinib. In certain embodiments, Compound D is administered to patients with leukemia in combination with sunitinib. In certain embodiments, Compound D is administered to patients with leukemia in combination with midostaurin. In certain embodiments, Compound D is administered to patients with leukemia in combination with pexidartinib. In certain embodiments, Compound D is administered to patients with leukemia in combination with lestaurtinib. In certain embodiments, Compound D is administered to patients with leukemia in combination with tandutinib. In certain embodiments, Compound D is administered to patients with leukemia in combination with crenolanib. In certain embodiments, the patient carries a FLT3-ITD mutation.

In one aspect, the methods provided herein comprise administering Compound D to patients with AML in combination with a FLT3 kinase inhibitor. In certain embodiments, a formulation of Compound D provided herein is administered to patients with AML in combination with a FLT3 kinase inhibitor. In certain embodiments, the FLT3 kinase inhibitor is selected from quizartinib, sunitinib, sunitinib malate, midostaurin, pexidartinib, lestaurtinib, tandutinib, quizartinib and crenolanib. In certain embodiments, Compound D is administered to patients with AML in combination with quizartinib. In certain embodiments, Compound D is administered to patients with AML in combination with sunitinib. In certain embodiments, Compound D is administered to patients with AML in combination with midostaurin. In certain embodiments, Compound D is administered to patients with AML in combination with pexidartinib. In certain embodiments, Compound D is administered to patients with AML in combination with lestaurtinib. In certain embodiments, Compound D is administered to patients with AML in combination with tandutinib. In certain embodiments, Compound D is administered to patients with AML in combination with crenolanib. In certain embodiments, the patient carries a FLT3-ITD mutation.

In certain embodiments, Compound D is administered to patients with leukemia in combination with a spliceosome inhibitor. In certain embodiments, Compound D is administered to patients with AML in combination with a spliceosome inhibitor. In certain embodiments, the spliceosome inhibitor is pladienolide B, 6-deoxypladienolide D, or H3B-8800.

In one aspect, the methods provided herein comprise administering Compound D to patients with leukemia in combination with an SMG1 kinase inhibitor. In certain embodiments, a formulation of Compound D provided herein is administered to patients with leukemia in combination with an SMG1 kinase inhibitor. In one aspect, the methods provided herein comprise administering Compound D to patients with AML in combination with an SMG1 kinase inhibitor. In certain embodiments, a formulation of Compound D provided herein is administered to patients with AML in combination with an SMG1 kinase inhibitor. In certain embodiments, the SMG1 inhibitor is 1-ethyl-7-(2-methyl-6-(1H-1,2,4-triazol-3-yl)pyridin-3-yl)-3,4-dihydropyrazino[2,3-b]pyrazin-2(1H)-one, chloro-N,N-diethyl-5-((4-(2-(4-(3-methylureido)phenyl)pyridin-4-yl)pyrimidin-2-yl)amino)benzenesulfonamide (compound Ii), or a compound disclosed in A. Gopalsamy et al, Bioorg. Med Chem Lett. 2012, 22:6636-66412 (for example, chloro-N,N-diethyl-5-((4-(2-(4-(3-methylureido)phenyl)pyridin-4-yl)pyrimidin-2-yl)amino)benzenesulfonamide.

In one aspect, the methods provided herein comprise administering Compound D to patients with leukemia in combination with a BCL2 inhibitor. In certain embodiments, a formulation of Compound D provided herein is administered to patients with leukemia in combination with a BCL2 inhibitor. In certain embodiments, Compound D is administered to patients with AML in combination with a BCL2 inhibitor. In certain embodiments, a formulation of Compound D provided herein is administered to patients with AML in combination with a BCL2 inhibitor, for example, venetoclax or navitoclax. In certain embodiments, the BCL2 inhibitor is venetoclax.

In one embodiment, provided herein is a method for treating of AML that is resistant to treatment with a BCL2 inhibitor, comprising administering Compound D. In one embodiment, provided herein is a method for treating of AML that has acquired resistance to venetoclax treatment, comprising administering Compound D. In one embodiment, provided herein is a method for treating of AML that has acquired resistance to venetoclax treatment, comprising administering a combination of Compound D and a BCL2 inhibitor. In one embodiment, provided herein is a method for treating of AML that has acquired resistance to venetoclax treatment, comprising administering a combination of Compound D and venetoclax.

In one aspect, the methods provided herein comprise administering Compound D to patients with leukemia in combination with a topoisomerase inhibitor. In certain embodiments, a formulation of Compound D provided herein is administered to patients with leukemia in combination with a topoisomerase inhibitor. In certain embodiments, Compound D is administered to patients with AML in combination with a topoisomerase inhibitor. In certain embodiments, a formulation of Compound D provided herein is administered to patients with AML in combination with a topoisomerase inhibitor, for example, irinotecan, topotecan, camptothecin, lamellarin D, etoposide, teniposide, doxorubicin, daunorubicin, mitoxantrone, amsacrine, ellipticines, aurintricarboxylic acid, or HU-331. In certain embodiments, the topoisomerase inhibitor is topotecan.

In certain embodiments, Compound D is administered to patients with leukemia in combination with a BET inhibitor. In certain embodiments, Compound D is administered to patients with AML in combination with a BET inhibitor. In certain embodiments, the BET inhibitor is selected from GSK525762A, OTX015, BMS-986158, TEN-010, CPI-0610, INCB54329, BAY1238097, FT-1101, C90010, ABBV-075, BI 894999, GS-5829, GSK1210151A (I-BET-151), CPI-203, RVX 208, XD46, MS436, PFI-1, RVX2135, ZEN3365, XD14, ARV-771, MZ-1, PLX5117, 4-[2-(cyclopropylmethoxy)-5-(methanesulfonyl)phenyl]-2-methylisoquinolin-1(2H)-one (Compound A), EP11313 and EP11336.

In certain embodiments, Compound D is administered to patients with leukemia in combination with an LSD1 inhibitor. In certain embodiments, Compound D is administered to patients with AML in combination with an LSD1 inhibitor. In certain embodiments, the LSD1 inhibitor is selected from ORY-1001, ORY-2001, INCB-59872, IMG-7289, TAK 418, GSK-2879552, and 4-[2-(4-amino-piperidin-1-yl)-5-(3-fluoro-4-methoxy-phenyl)-1-methyl-6-oxo-1,6-dihydropyrimidin-4-yl]-2-fluoro-benzonitrile or a salt thereof (e.g. besylate salt, Compound B).

In one aspect, the methods provided herein comprise administering Compound D to patients with leukemia in combination with triptolide, retaspimycin, alvespimycin, 7-(6-(2-hydroxypropan-2-yl)pyridin-3-yl)-1-((trans)-4-methoxycyclohexyl)-3,4-dihydropyrazino[2,3-b]pyrazin-2(1H)-one (CC-223), 1-ethyl-7-(2-methyl-6-(1H-1,2,4-triazol-3-yl)pyridin-3-yl)-3,4-dihydropyrazino[2,3-b]pyrazin-2(1H)-one (CC-115), rapamycin, MLN-0128, everolimus, AZD8055, pladienolide B, topotecan, thioguanine, mitoxantrone, etoposide, decitabine, daunorubicin, clofarabine, cladribine, 6-mercaptopurine, chloro-N,N-diethyl-5-((4-(2-(4-(3-methylureido)phenyl)pyridin-4-yl)pyrimidin-2-yl)amino)benzenesulfonamide (compound Ii), fedratinib, sunitinib, pexidartinib, midostaurin, lestaurtinib, momelotinib, quizartinib, and crenolanib.

In one aspect, the methods provided herein comprise administering Compound D to patients with AML, in combination with triptolide, retaspimycin, alvespimycin, 7-(6-(2-hydroxypropan-2-yl)pyridin-3-yl)-1-((trans)-4-methoxycyclohexyl)-3,4-dihydropyrazino[2,3-b]pyrazin-2(1H)-one (CC-223), 1-ethyl-7-(2-methyl-6-(1H-1,2,4-triazol-3-yl)pyridin-3-yl)-3,4-dihydropyrazino[2,3-b]pyrazin-2(1H)-one (CC-115), rapamycin, MLN-0128, everolimus, AZD8055, pladienolide B, topotecan, thioguanine, mitoxantrone, etoposide, decitabine, daunorubicin, clofarabine, cladribine, 6-mercaptopurine, chloro-N,N-diethyl-5-((4-(2-(4-(3-methylureido)phenyl)pyridin-4-yl)pyrimidin-2-yl)amino)benzenesulfonamide (compound Ii), fedratinib, sunitinib, pexidartinib, midostaurin, lestaurtinib, momelotinib, quizartinib, and crenolanib.

In one aspect, the methods provided herein comprise administering Compound D to patients with cancer in combination with an mTOR inhibitor, wherein the cancer is selected from breast cancer, kidney cancer, pancreatic cancer, gastrointestinal cancer, lung cancer, neuroendocrine tumor (NET), and renal cell carcinoma (RCC). In certain embodiments, a formulation of Compound D provided herein is administered to patients with cancer in combination with a topoisomerase inhibitor. In certain embodiments, a formulation of Compound D provided herein is administered to cancer patients in combination with an mTOR inhibitor, wherein the cancer is selected from breast cancer, kidney cancer, pancreatic cancer, gastrointestinal cancer, lung cancer, neuroendocrine tumor (NET), and renal cell carcinoma. In certain embodiments, the mTOR inhibitor is selected from everolimus, MLN-0128 and AZD8055. In some embodiments, the mTOR inhibitor is an mTOR kinase inhibitor. In certain embodiments, the mTOR kinase inhibitor is selected from 7-(6-(2-hydroxypropan-2-yl)pyridin-3-yl)-1-((trans)-4-methoxycyclohexyl)-3,4-dihydropyrazino[2,3-b]pyrazin-2(1H)-one (CC-223) and 1-ethyl-7-(2-methyl-6-(1H-1,2,4-triazol-3-yl)pyridin-3-yl)-3,4-dihydropyrazino[2,3-b]pyrazin-2(1H)-one (CC-115). In one embodiment, the mTOR kinase inhibitor is 7-(6-(2-hydroxypropan-2-yl)pyridin-3-yl)-1-((trans)-4-methoxycyclohexyl)-3,4-dihydropyrazino[2,3-b]pyrazin-2(1H)-one (CC-223). In one embodiment, the mTOR kinase inhibitor is 1-ethyl-7-(2-methyl-6-(1H-1,2,4-triazol-3-yl)pyridin-3-yl)-3,4-dihydropyrazino[2,3-b]pyrazin-2(1H)-one (CC-115). In one embodiment, the mTOR inhibitor is everolimus. In one embodiment, the mTOR inhibitor is temsirolimus. In one embodiment, the mTOR inhibitor is MLN-0128. In one embodiment, the mTOR inhibitor is AZD8055.

In certain embodiments, Compound D is administered to breast cancer patients in combination with everolimus. In certain embodiments, a formulation of Compound D provided herein is administered to breast cancer patients in combination with everolimus.

In certain embodiments, Compound D is administered to kidney cancer patients in combination with everolimus. In certain embodiments, a formulation of Compound D provided herein is administered to kidney cancer patients in combination with everolimus.

In certain embodiments, Compound D is administered to pancreatic cancer patients in combination with everolimus. In certain embodiments, a formulation of Compound D provided herein is administered to pancreatic cancer patients in combination with everolimus.

In certain embodiments, Compound D is administered to gastrointestinal cancer patients in combination with everolimus. In certain embodiments, a formulation of Compound D provided herein is administered to gastrointestinal cancer patients in combination with everolimus.

In certain embodiments, Compound D is administered to lung cancer patients in combination with everolimus. In certain embodiments, a formulation of Compound D provided herein is administered to lung cancer patients in combination with everolimus.

In certain embodiments, Compound D is administered to neuroendocrine tumor patients in combination with everolimus. In certain embodiments, a formulation of Compound D provided herein is administered to neuroendocrine tumor patients in combination with everolimus.

In certain embodiments, Compound D is administered to renal cell carcinoma patients in combination with everolimus. In certain embodiments, a formulation of Compound D provided herein is administered to renal cell carcinoma patients in combination with everolimus.

Also encompassed herein is a method of increasing the dosage of an anti-cancer drug or agent that can be safely and effectively administered to a patient, which comprises administering to the patient (e.g., a human) Compound D, for example, a formulation of Compound D provided herein in combination with the second anti-cancer drug. Patients that can benefit by this method are those likely to suffer from an adverse effect associated with anti-cancer drugs for treating a specific cancer of the skin, subcutaneous tissue, lymph nodes, brain, lung, liver, bone, intestine, colon, heart, pancreas, adrenal, kidney, prostate, breast, colorectal, or combinations thereof. The administration of Compound D, for example, a formulation of Compound D provided herein, alleviates or reduces adverse effects which are of such severity that it would otherwise limit the amount of anti-cancer drug.

Also encompassed herein is a method of decreasing the dosage of an anti-cancer drug or agent that can be safely and effectively administered to a patient, which comprises administering to the patient (e.g., a human) Compound D, for example, a formulation of Compound D provided herein in combination with the second anti-cancer drug. Patients that can benefit by this method are those likely to suffer from an adverse effect associated with anti-cancer drugs for treating a specific cancer of the skin, subcutaneous tissue, lymph nodes, brain, lung, liver, bone, intestine, colon, heart, pancreas, adrenal, kidney, prostate, breast, colorectal, or combinations thereof. The administration of Compound D, for example, a formulation of Compound D provided herein, potentiates the activity of the anti-cancer drug, which allows for a reduction in dose of the anti-cancer drug while maintaining efficacy, which in turn can alleviate or reduce the adverse effects which are of such severity that it limited the amount of anti-cancer drug.

In one embodiment, Compound D is administered daily in an amount ranging from about 0.1 to about 20 mg, from about 1 to about 15 mg, from about 1 to about 10 mg, or from about 1 to about 15 mg prior to, during, or after the occurrence of the adverse effect associated with the administration of an anti-cancer drug to a patient. In certain embodiments, Compound D is administered in combination with specific agents such as heparin, aspirin, coumadin, or G-CSF to avoid adverse effects that are associated with anti-cancer drugs such as but not limited to neutropenia or thrombocytopenia.

In one embodiment, Compound D, for example, a formulation of Compound D provided herein, is administered to patients with diseases and disorders associated with or characterized by, undesired angiogenesis in combination with additional active ingredients, including, but not limited to, anti-cancer drugs, anti-inflammatories, antihistamines, antibiotics, and steroids.

In another embodiment, encompassed herein is a method of treating, preventing and/or managing cancer, which comprises administering Compound D, for example, a formulation of Compound D provided herein, in conjunction with (e.g. before, during, or after) at least one anti-cancer therapy including, but not limited to, surgery, immunotherapy, biological therapy, radiation therapy, or other non-drug based therapy presently used to treat, prevent and/or manage cancer. The combined use of the compound provided herein and other anti-cancer therapy may provide a unique treatment regimen that is unexpectedly effective in certain patients. Without being limited by theory, it is believed that Compound D may provide additive or synergistic effects when given concurrently with at least one anti-cancer therapy.

As discussed elsewhere herein, encompassed herein is a method of reducing, treating and/or preventing adverse or undesired effects associated with other anti-cancer therapy including, but not limited to, surgery, chemotherapy, radiation therapy, hormonal therapy, biological therapy and immunotherapy. Compound D, for example, a formulation of Compound D provided herein, and other active ingredient can be administered to a patient prior to, during, or after the occurrence of the adverse effect associated with other anti-cancer therapy.

In certain embodiments, the methods provided herein comprise administration of one or more of calcium, calcitriol, or vitamin D supplementation with Compound D. In certain embodiments, the methods provided herein comprise administration of calcium, calcitriol, and vitamin D supplementation prior to the treatment with Compound D. In certain embodiments, the methods provided herein comprise administration of calcium, calcitriol, and vitamin D supplementation prior to the administration of first dose of Compound D in each cycle. In certain embodiments, the methods provided herein comprise administration of calcium, calcitriol, and vitamin D supplementation at least up to 3 days prior to the treatment with Compound D. In certain embodiments, the methods provided herein comprise administration of calcium, calcitriol, and vitamin D supplementation prior to the administration of first dose of Compound D in each cycle. In certain embodiments, the methods provided herein comprise administration of calcium, calcitriol, and vitamin D supplementation at least up to 3 days prior to the administration of first dose of Compound D in each cycle. In certain embodiments, the methods provided herein comprise administration of calcium, calcitriol, and vitamin D supplementation prior to administration of first dose of Compound D in each cycle and continues after administration of the last dose of Compound D in each cycle. In certain embodiments, the methods provided herein comprise administration of calcium, calcitriol, and vitamin D supplementation at least up to 3 days prior to administration of first dose of Compound D in each cycle and continues until at least up to 3 days after administration of the last dose of Compound D in each cycle (e.g., at least up to day 8 when Compound D is administered on Days 1-5). In one embodiment, the methods provided herein comprise administration of calcium, calcitriol, and vitamin D supplementation at least up to 3 days prior to administration of day 1 of each cycle and continue until 3 days after the last dose of Compound D in each cycle (eg, Day 8 when Compound D is administered on Days 1-5, Day 13 when Compound D is administered on Days 1-3 and Days 8-10).

In certain embodiments, calcium supplementation is administered to deliver at least 1200 mg of elemental calcium per day given in divided doses. In certain embodiments, calcium supplementation is administered as calcium carbonate in a dose of 500 mg administered three times a day per orally (PO).

In certain embodiments, calcitriol supplementation is administered to deliver 0.25 calcitriol (PO) once daily.

In certain embodiments, vitamin D supplementation is administered to deliver about 500 IU to about 50,000 IU vitamin D once daily. In certain embodiments, vitamin D supplementation is administered to deliver about 1000 IU vitamin D once daily. In certain embodiments, vitamin D supplementation is administered to deliver about 50,000 IU vitamin D weekly. In certain embodiments, vitamin D supplementation is administered to deliver about 1000 IU vitamin D2 or D3 once daily. In certain embodiments, vitamin D supplementation is administered to deliver about 500 IU vitamin D once daily. In certain embodiments, vitamin D supplementation is administered to deliver about 50,000 IU vitamin D weekly. In certain embodiments, vitamin D supplementation is administered to deliver about 20,000 IU vitamin D weekly. In certain embodiments, vitamin D supplementation is administered to deliver about 1000 IU vitamin D2 or D3 once daily. In certain embodiments, vitamin D supplementation is administered to deliver about 50,000 IU vitamin D2 or D3 weekly. In certain embodiments, vitamin D supplementation is administered to deliver about 20,000 IU vitamin D2 or D3 weekly.

In certain embodiments, a formulation of Compound D provided herein and doxetaxol are administered to patients with non-small cell lung cancer who were previously treated with carbo/VP 16 and radiotherapy.

Use with Transplantation Therapy

Compound D, for example, a formulation of Compound D provided herein, can be used to reduce the risk of Graft Versus Host Disease (GVHD). Therefore, encompassed herein is a method of treating, preventing and/or managing cancer, which comprises administering Compound D, for example, a formulation of Compound D provided herein, in conjunction with transplantation therapy.

As those of ordinary skill in the art are aware, the treatment of cancer is often based on the stages and mechanism of the disease. For example, as inevitable leukemic transformation develops in certain stages of cancer, transplantation of peripheral blood stem cells, hematopoietic stem cell preparation or bone marrow may be necessary. The combined use of Compound D, for example, a formulation of Compound D provided herein, and transplantation therapy provides a unique and unexpected synergism. In particular, a formulation of Compound D provided herein exhibits immunomodulatory activity that may provide additive or synergistic effects when given concurrently with transplantation therapy in patients with cancer.

Compound D, for example, a formulation of Compound D provided herein, can work in combination with transplantation therapy reducing complications associated with the invasive procedure of transplantation and risk of GVHD. Encompassed herein is a method of treating, preventing and/or managing cancer which comprises administering to a patient (e.g., a human) formulation of Compound D provided herein before, during, or after the transplantation of umbilical cord blood, placental blood, peripheral blood stem cell, hematopoietic stem cell preparation, or bone marrow. Some examples of stem cells suitable for use in the methods provided herein are disclosed in U.S. Pat. No. 7,498,171, the disclosure of which is incorporated herein by reference in its entirety.

In one embodiment, Compound D, for example, a formulation of Compound D provided herein, is administered to patients with acute myeloid leukemia before, during, or after transplantation.

In one embodiment, Compound D, for example, a formulation of Compound D provided herein, is administered to patients with multiple myeloma before, during, or after the transplantation of autologous peripheral blood progenitor cells.

In one embodiment, Compound D, for example, a formulation of Compound D provided herein, is administered to patients with NHL (e.g., DLBCL) before, during, or after the transplantation of autologous peripheral blood progenitor cells.

Cycling Therapy

In certain embodiments, Compound D, for example, a formulation of Compound D provided herein, are cyclically administered to a patient independent of the cancer treated. Cycling therapy involves the administration of an active agent for a period of time, followed by a rest for a period of time, and repeating this sequential administration. Cycling therapy can reduce the development of resistance to one or more of the therapies, avoid or reduce the side effects of one of the therapies, and/or improve the efficacy of the treatment.

In certain embodiments, Compound D, for example, a formulation of Compound D provided herein, is administered daily in a single or divided dose in a four- to six-week cycle with a rest period of about a week or two weeks. In certain embodiments, Compound D, for example, a formulation of Compound D provided herein, is administered daily in single or divided doses for one to ten consecutive days of a 28-day cycle, then a rest period with no administration for rest of the 28-day cycle. The cycling method further allows the frequency, number, and length of dosing cycles to be increased. Thus, encompassed herein in certain embodiments is the administration of Compound D, for example, a formulation of Compound D provided herein, for more cycles than are typical when it is administered alone. In certain embodiments, Compound D, for example, a formulation of Compound D provided herein, is administered for a greater number of cycles that would typically cause dose-limiting toxicity in a patient to whom a second active ingredient is not also being administered.

In one embodiment, Compound D, for example, a formulation of Compound D provided herein, is administered daily and continuously for three or four weeks to administer a dose of Compound D from about 0.1 to about 20 mg/d followed by a break of one or two weeks.

In another embodiment, Compound D, for example, a formulation of Compound D provided herein, is administered intravenously and a second active ingredient is administered orally, with administration of Compound D, for example, a formulation of Compound D provided herein, occurring 30 to 60 minutes prior to a second active ingredient, during a cycle of four to six weeks. In certain embodiments, the combination of Compound D, for example, a formulation of Compound D provided herein, and a second active ingredient is administered by intravenous infusion over about 90 minutes every cycle. In certain embodiments, one cycle comprises the administration from about 0.1 to about 150 mg/day of Compound D, for example, a formulation of Compound D provided herein, and from about 50 to about 200 mg/m2/day of a second active ingredient daily for three to four weeks and then one or two weeks of rest. In certain embodiments, the number of cycles during which the combinatorial treatment is administered to a patient is ranging from about one to about 24 cycles, from about two to about 16 cycles, or from about four to about three cycles.

In one embodiment, a cycling therapy provided herein comprises administering Compound D, for example, a formulation of Compound D provided herein, in a treatment cycle which includes an administration period of up to 5 days followed by a rest period. In one embodiment, the treatment cycle includes an administration period of 5 days followed by a rest period. In one embodiment, the treatment cycle includes an administration period of up to 10 days followed by a rest period. In one embodiment, the rest period is from about 10 days up to about 40 days. In one embodiment, the treatment cycle includes an administration period of up to 10 days followed by a rest period from about 10 days up to about 40 days. In one embodiment, the treatment cycle includes an administration period of up to 10 days followed by a rest period from about 23 days up to about 37 days. In one embodiment, the rest period is from about 23 days up to about 37 days. In one embodiment, the rest period is 23 days. In one embodiment, the treatment cycle includes an administration period of up to 10 days followed by a rest period of 23 days. In one embodiment, the rest period is 37 days. In one embodiment, the treatment cycle includes an administration period of up to 10 days followed by a rest period of 37 days.

In one embodiment, the treatment cycle includes an administration of Compound D, for example, a formulation of Compound D provided herein, on days 1 to 5 of a 28-day cycle. In another embodiment, the treatment cycle includes an administration of Compound D, for example, a formulation of Compound D provided herein, on days 1 to 10 of a 28-day cycle. In one embodiment, the treatment cycle includes an administration on days 1 to 5 of a 42-day cycle. In another embodiment, the treatment cycle includes an administration on days 1 to 10 of a 42-day cycle. In another embodiment, the treatment cycle includes an administration on days 1 to 5 and 15 to 19 of a 28-day cycle. In another embodiment, the treatment cycle includes an administration on days 1 to 3 and 8 to 10 of a 28-day cycle.

In one embodiment, the treatment cycle includes an administration of Compound D, for example, a formulation of Compound D provided herein, on days 1 to 21 of a 28-day cycle. In another embodiment, the treatment cycle includes an administration on days 1 to 5 of a 7-day cycle. In another embodiment, the treatment cycle includes an administration on days 1 to 7 of a 7-day cycle.

Any treatment cycle described herein can be repeated for at least 2, 3, 4, 5, 6, 7, 8, or more cycles. In certain instances, the treatment cycle as described herein includes from 1 to about 24 cycles, from about 2 to about 16 cycles, or from about 2 to about 4 cycles. In certain instances, a treatment cycle as described herein includes from 1 to about 4 cycles. In certain embodiments, cycle 1 to 4 are all 28-day cycles. In certain embodiments, cycle 1 is a 42-day cycle and cycles 2 to 4 are 28-day cycles. In some embodiments, Compound D, for example, a formulation of Compound D provided herein, is administered for 1 to 13 cycles of 28 days (e.g. about 1 year). In certain instances, the cycling therapy is not limited to the number of cycles, and the therapy is continued until disease progression. Cycles, can in certain instances, include varying the duration of administration periods and/or rest periods described herein.

In one embodiment the treatment cycle includes administering Compound D at a dosage amount of about 0.3 mg/day, 0.6 mg/day, 1.2 mg/day, 1.8 mg/day, 2.4 mg/day, 3.6 mg/day, 5.4 mg/day, 7.2 mg/day, 8.1 mg/day, 9.0 mg/day, 10.0 mg/day, 10.8 mg/day, or 12.2 mg/day administered once per day. In one embodiment the treatment cycle includes administering Compound D at a dosage amount of about 0.3 mg/day, 0.6 mg/day, 1.2 mg/day, 1.8 mg/day, 2.4 mg/day, 3.6 mg/day, 5.4 mg/day, 7.2 mg/day, 8.1 mg/day, 9.0 mg/day, 10.0 mg/day, 10.8 mg/day, 12.2 mg/day, or 20 mg/day administered once per day. In one embodiment the treatment cycle includes administering Compound D at a dosage amount of about 0.6 mg/day, 1.2 mg/day, 1.8 mg/day, 2.4 mg/day, or 3.6 mg/day, administered once per day. In some such embodiments, the treatment cycle includes administering Compound D at a dosage amount of about 0.6 mg, 1.2 mg, 1.8 mg, 2.4 mg, or 3.6 mg on days 1 to 3 of a 28-day cycle. In other embodiments, the treatment cycle includes administering Compound D at a dosage amount of about 0.6 mg, 1.2 mg, 1.8 mg, 2.4 mg, or 3.6 mg on days 1 to 5 and 15 to 19 of a 28-day cycle. In other embodiments, the treatment cycle includes administering Compound D at a dosage amount of about 0.6 mg, 1.2 mg, 1.8 mg, 2.4 mg, 3.6 mg, 5.4 mg/day, 7.2 mg/day, 8.1 mg/day, 9.0 mg/day, or 10.0 mg/day, on days 1 to 5 and 15 to 19 of a 28-day cycle.

Compound D, for example, a formulation of Compound D provided herein, can be administered at the same amount for all administration periods in a treatment cycle. Alternatively, in one embodiment, the compound is administered at different doses in the administration periods.

In one embodiment, a formulation of Compound D provided herein is administered to a subject in a cycle, wherein the cycle comprises administering the formulation for at least 5 days in a 28-day cycle. In one embodiment, a formulation of Compound D provided herein is administered to a subject in a cycle, wherein the cycle comprises administering the formulation on days 1 to 5 of a 28-day cycle. In one embodiment, the formulation is administered to deliver Compound D in a dose of about 0.1 mg to about 20 mg on days 1 to 5 of a 28-day cycle. In one embodiment, the formulation is administered to deliver Compound D in a dose of about 0.5 mg to about 5 mg on days 1 to 5 of a 28-day cycle. In one embodiment, the formulation is administered to deliver Compound D in a dose of about 0.5 mg to about 10 mg on days 1 to 5 of a 28-day cycle. In one embodiment, a formulation of Compound D provided herein is administered to a subject in a cycle, wherein the cycle comprises administering the formulation on days 1 to 5 and 15 to 19 of a 28-day cycle. In one embodiment, the formulation is administered to deliver Compound D in a dose of about 0.1 mg to about 20 mg on days 1 to 5 and 15 to 19 of a 28-day cycle. In one embodiment, the formulation is administered to deliver Compound D in a dose of about 0.5 mg to about 5 mg on days 1 to 5 and 15 to 19 of a 28-day cycle. In one embodiment, the formulation is administered to deliver Compound D in a dose of about 0.5 mg to about 10 mg on days 1 to 5 and 15 to 19 of a 28-day cycle.

In one embodiment, provided herein is a method of treating of AML by administering to a subject a formulation of Compound D provided herein in a cycle, wherein the cycle comprises administering the formulation to deliver Compound D in a dose of about 0.1 mg to about 20 mg for at least 5 days in a 28-day cycle. In one embodiment, provided herein is a method of treating of AML by administering to a subject a formulation of Compound D provided herein in a cycle, wherein the cycle comprises administering the formulation to deliver Compound D in a dose of about 0.1 mg to about 20 mg on days 1 to 5 of a 28-day cycle. In one embodiment, provided herein is a method of treating of AML by administering to a subject a formulation of Compound D provided herein in a cycle, wherein the cycle comprises administering the formulation to deliver Compound D in a dose of about 0.1 mg to about 5 mg on days 1 to 5 of a 28-day cycle. In one embodiment, provided herein is a method of treating of AML by administering to a subject a formulation of Compound D provided herein in a cycle, wherein the cycle comprises administering the formulation to deliver Compound D in a dose of about 0.5 mg to about 5 mg on days 1 to 5 of a 28-day cycle. In another embodiment, provided herein is a method of treating of AML by administering to a subject a formulation of Compound D provided herein in a cycle, wherein the cycle comprises administering the formulation to deliver Compound D in a dose of about 0.1 mg to about 20 mg on days 1 to 5 and 15 to 19 of a 28-day cycle. In one embodiment, provided herein is a method of treating of AML by administering to a subject a formulation of Compound D provided herein in a cycle, wherein the cycle comprises administering the formulation to deliver Compound D in a dose of about 0.1 mg to about 5 mg on days 1 to 5 and 15 to 19 of a 28-day cycle. In one embodiment, provided herein is a method of treating of AML by administering to a subject a formulation of Compound D provided herein in a cycle, wherein the cycle comprises administering the formulation to deliver Compound D in a dose of about 0.5 mg to about 5 mg on days 1 to 5 and 15 to 19 of a 28-day cycle.

In one embodiment, provided herein is a method of treating of MDS by administering to a subject a formulation of Compound D provided herein in a cycle, wherein the cycle comprises administering the formulation to deliver Compound D in a dose of about 0.1 mg to about 20 mg for at least 5 days in a 28-day cycle. In one embodiment, provided herein is a method of treating of MDS by administering to a subject a formulation of Compound D provided herein in a cycle, wherein the cycle comprises administering the formulation to deliver Compound D in a dose of about 0.1 mg to about 20 mg on days 1 to 5 of a 28-day cycle. In one embodiment, provided herein is a method of treating of MDS by administering to a subject a formulation of Compound D provided herein in a cycle, wherein the cycle comprises administering the formulation to deliver Compound D in a dose of about 0.1 mg to about 5 mg on days 1 to 5 of a 28-day cycle. In one embodiment, provided herein is a method of treating of MDS by administering to a subject a formulation of Compound D provided herein in a cycle, wherein the cycle comprises administering the formulation to deliver Compound D in a dose of about 0.5 mg to about 5 mg on days 1 to 5 of a 28-day cycle. In another embodiment, provided herein is a method of treating of MDS by administering to a subject a formulation of Compound D provided herein in a cycle, wherein the cycle comprises administering the formulation to deliver Compound D in a dose of about 0.1 mg to about 20 mg on days 1 to 5 and 15 to 19 of a 28-day cycle. In one embodiment, provided herein is a method of treating of MDS by administering to a subject a formulation of Compound D provided herein in a cycle, wherein the cycle comprises administering the formulation to deliver Compound D in a dose of about 0.1 mg to about 5 mg on days 1 to 5 and 15 to 19 of a 28-day cycle. In one embodiment, provided herein is a method of treating of MDS by administering to a subject a formulation of Compound D provided herein in a cycle, wherein the cycle comprises administering the formulation to deliver Compound D in a dose of about 0.5 mg to about 5 mg on days 1 to 5 and 15 to 19 of a 28-day cycle.

### 5.3. Methods of Detecting and Quantifying Gene Sets or Biomarkers

In certain embodiments, provided herein are methods of detecting and quantifying the RNA (e.g., mRNA) level of a gene set, such as a gene signature or a biomarker provided herein, from a biological sample. The methods of detecting and quantifying the mRNA level of a gene set include any methods known in the art that can detect or quantify mRNA, such as transcriptomic profiling, quantitative RT-PCR (qRT-PCR), ribonuclease protection assays, Northern blots, etc.

Any suitable assay platform can be used to determine the presence of mRNA in a sample. For example, an assay may be in the form of a dipstick, a membrane, a chip, a disk, a test strip, a filter, a microsphere, a slide, a multi-well plate, or an optical fiber. An assay system may have a solid support on which a nucleic acid corresponding to the mRNA is attached. The solid support may comprise, for example, a plastic, silicon, a metal, a resin, glass, a membrane, a particle, a precipitate, a gel, a polymer, a sheet, a sphere, a polysaccharide, a capillary, a film, a plate, or a slide. The assay components can be prepared and packaged together as a kit for detecting an mRNA.

The nucleic acid can be labeled, if desired, to make a population of labeled mRNAs. In general, a sample can be labeled using methods that are well known in the art (e.g., using DNA ligase, terminal transferase, or by labeling the RNA backbone, etc.). See, e.g., Ausubel et al., Short Protocols in Molecular Biology (Wiley & Sons, 3rd ed. 1995); Sambrook et al., Molecular Cloning: A Laboratory Manual (Cold Spring Harbor, N.Y., 3rd ed. 2001). In some embodiments, the sample is labeled with fluorescent label. Exemplary fluorescent dyes include, but are not limited to, xanthene dyes, fluorescein dyes (e.g., fluorescein isothiocyanate (FITC), 6-carboxyfluorescein (FAM), 6 carboxy-2′,4′,7′,4,7-hexachlorofluorescein (HEX), 6-carboxy-4′,5′-dichloro-2′,7′-dimethoxyfluorescein (JOE)), rhodamine dyes (e.g., rhodamine 110 (R110), N,N,N′,N′-tetramethyl-6-carboxyrhodamine (TAMRA), 6-carboxy-X-rhodamine (ROX), 5-carboxyrhodamine 6G (R6G5 or G5), 6-carboxyrhodamine 6G (R6G6 or G6)), cyanine dyes (e.g., Cy3, Cy5 and Cy7), Alexa dyes (e.g., Alexa-fluor-555), coumarin, Diethylaminocoumarin, umbelliferone, benzimide dyes (e.g., Hoechst 33258), phenanthridine dyes (e.g., Texas Red), ethidium dyes, acridine dyes, carbazole dyes, phenoxazine dyes, porphyrin dyes, polymethine dyes, BODIPY dyes, quinoline dyes, Pyrene, Fluorescein Chlorotriazinyl, eosin dyes, Tetramethylrhodamine, Lissamine, Napthofluorescein, and the like.

Examples of PCR methods can be found in U.S. Pat. No. 6,927,024, which is incorporated by reference herein in its entirety. Examples of RT-PCR methods can be found in U.S. Pat. No. 7,122,799, which is incorporated by reference herein in its entirety. A method of fluorescent in situ PCR is described in U.S. Pat. No. 7,186,507, which is incorporated by reference herein in its entirety.

In some embodiments, qRT-PCR can be used for both the detection and quantification of RNA targets (Bustin et al., Clin. Sci. 2005, 109:365-379). Quantitative results obtained by qRT-PCR are generally more informative than qualitative data. Thus, in some embodiments, qRT-PCR-based assays can be useful to measure mRNA levels during cell-based assays. The qRT-PCR method is also useful to monitor patient therapy. Examples of qRT-PCR-based methods can be found, for example, in U.S. Pat. No. 7,101,663, which is incorporated by reference herein in its entirety. Instruments for qRT-PCR, such as the Applied Biosystems 7500, are available commercially, so are the reagents, such as TaqMan® Sequence Detection Chemistry. For example, TaqMan® Gene Expression Assays can be used, following the manufacturer's instructions. These kits are pre-formulated gene expression assays for rapid, reliable detection and quantification of human, mouse, and rat mRNA transcripts. An exemplary qRT-PCR program, for example, is 50° C. for 2 minutes, 95° C. for 10 minutes, 40 cycles of 95° C. for 15 seconds, then 60° C. for 1 minute.

To determine the cycle number at which the fluorescence signal associated with a particular amplicon accumulation crosses the threshold (referred to as the CT), the data can be analyzed, for example, using 7500 Real-Time PCR System Sequence Detection software vs. using the comparative CT relative quantification calculation method. Using this method, the output is expressed as a fold-change of expression levels. In some embodiments, the threshold level can be selected to be automatically determined by the software. In some embodiments, the threshold level is set to be above the baseline but sufficiently low to be within the exponential growth region of an amplification curve.

In some embodiments, provided herein are methods of detecting and quantifying the cDNA level of a gene set, such as a gene signature or a biomarker provided herein, from a biological sample. In certain embodiments, the methods further comprises generating cDNA from the mRNA obtained from the sample. Any known methods of generating cDNA from mRNA in the art can be used herein. The methods of detecting and quantifying the cDNA level of a gene set include any methods known in the art that can detect or quantify cDNA, such as DNA microarrays, high throughput sequencing, Southern blots, etc.

In some embodiments, provided herein are methods of detecting and quantifying the protein level of a gene set, such as a gene signature or a biomarker provided herein, from a biological sample. The methods of detecting and quantifying the protein level of a gene set include any methods known in the art that can detect or quantify proteins, such as mass spectrometry, immunohistochemistry, flow cytometry, cytometry bead array, ELISA, Western blots, etc. Several types of ELISA are commonly used, including direct ELISA, indirect ELISA, and sandwich ELISA.

### 5.4. Subjects and Samples

In certain embodiments, the various methods provided herein use samples (e.g., biological samples) from subjects or individuals (e.g., patients). The subject can be a patient, such as, a patient with a cancer (e.g., lymphoma, MM, or leukemia). The subject can be a mammal, for example, a human. The subject can be male or female, and can be an adult, a child, or an infant. Samples can be analyzed at a time during an active phase of a cancer (e.g., lymphoma, MM, or leukemia), or when the cancer (e.g., lymphoma, MM, or leukemia) is inactive. In certain embodiments, more than one sample from a subject can be obtained.

In certain embodiments, the sample used in the methods provided herein comprises body fluids from a subject. Non-limiting examples of body fluids include blood (e.g., whole blood), blood plasma, amniotic fluid, aqueous humor, bile, cerumen, cowper's fluid, pre-ejaculatory fluid, chyle, chyme, female ejaculate, interstitial fluid, lymph, menses, breast milk, mucus, pleural fluid, pus, saliva, sebum, semen, serum, sweat, tears, urine, vaginal lubrication, vomit, water, feces, internal body fluids (including cerebrospinal fluid surrounding the brain and the spinal cord), synovial fluid, intracellular fluid (the fluid inside cells), and vitreous humor (the fluid in the eyeball). In some embodiments, the sample is a blood sample. The blood sample can be obtained using conventional techniques as described in, e.g., Innis et al, eds., PCR Protocols (Academic Press, 1990). White blood cells can be separated from blood samples using conventional techniques or commercially available kits, e.g., RosetteSep kit (Stein Cell Technologies, Vancouver, Canada). Sub-populations of white blood cells, e.g., mononuclear cells, B cells, T cells, monocytes, granulocytes, or lymphocytes, can be further isolated using conventional techniques, e.g., magnetically activated cell sorting (MACS) (Miltenyi Biotec, Auburn, Calif.) or fluorescently activated cell sorting (FACS) (Becton Dickinson, San Jose, Calif.).

In one embodiment, the blood sample is from about 0.1 mL to about 10.0 mL, from about 0.2 mL to about 7 mL, from about 0.3 mL to about 5 mL, from about 0.4 mL to about 3.5 mL, or from about 0.5 mL to about 3 mL. In another embodiment, the blood sample is about 0.3, about 0.4, about 0.5, about 0.6, about 0.7, about 0.8, about 0.9, about 1.0, about 1.5, about 2.0, about 2.5, about 3.0, about 3.5, about 4.0, about 4.5, about 5.0, about 6.0, about 7.0, about 8.0, about 9.0, or about 10.0 mL.

In some embodiments, the sample used in the present methods comprises a biopsy (e.g., a tumor biopsy). The biopsy can be from any organ or tissue, for example, skin, liver, lung, heart, colon, kidney, bone marrow, teeth, lymph node, hair, spleen, brain, breast, or other organs. Any biopsy technique known by those skilled in the art can be used for isolating a sample from a subject, for instance, open biopsy, close biopsy, core biopsy, incisional biopsy, excisional biopsy, or fine needle aspiration biopsy.

In one embodiment, the sample used in the methods provided herein is obtained from the subject prior to the subject receiving a treatment for the disease or disorder. In another embodiment, the sample is obtained from the subject during the subject receiving a treatment for the disease or disorder. In another embodiment, the sample is obtained from the subject after the subject receiving a treatment for the disease or disorder. In various embodiments, the treatment comprises administering a compound (e.g., a compound provided in Section 5.5 below) to the subject.

5.1. Types of Cells

In certain embodiments, the sample used in the methods provided herein comprises a plurality of cells, such as cancer (e.g., lymphoma, MM, or leukemia) cells. Such cells can include any type of cells, e.g., stem cells, blood cells (e.g., peripheral blood mononuclear cells (PBMC)), lymphocytes, B cells, T cells, monocytes, granulocytes, immune cells, or cancer cells.

B cells (B lymphocytes) include, for example, plasma B cells, memory B cells, B1 cells, B2 cells, marginal-zone B cells, and follicular B cells. B cells can express immunoglobulins (antibodies) and B cell receptor.

Specific cell populations can be obtained using a combination of commercially available antibodies (e.g., antibodies from Quest Diagnostic (San Juan Capistrano, Calif.) or Dako (Denmark)).

In certain embodiments, the cells in the methods provided herein are PBMC. In certain embodiments, the sample used in the methods provided herein is from a disease tissue, e.g., from an individual having cancer (e.g., lymphoma, MM, or leukemia).

In certain embodiments, cell lines are used as disease models for evaluating effects of compounds, studying mechanisms of action, or establishing reference levels of biomarkers, etc. In some embodiments, the cells used in the methods provided herein are from a cancer (e.g., AML) cell line. In certain embodiments, the cells are from a lymphoma cell line. In other embodiments, the cells are from an MM cell line. In other embodiments, the cells are from a leukemia cell line. In some embodiments, the leukemia cell line is a CLL cell line. In other embodiments, the leukemia cell line is an ALL cell line. In yet other embodiments, the leukemia cell line is a CML cell line. In yet other embodiments, the leukemia cell line is an AML cell line. In one embodiment, the AML cell line is KG-1 cell line. In another embodiment, the AML cell line is KG-la cell line. In yet another embodiment, the AML cell line is KASUMI-1 cell line. In still another embodiment, the AML cell line is NB4 cell line. In one embodiment, the AML cell line is MV-4-11 cell line. In another embodiment, the AML cell line is MOLM-13 cell line. In yet another embodiment, the AML cell line is HL-60 cell line. In still another embodiment, the AML cell line is U-937 cell line. In one embodiment, the AML cell line is OCI-AML2 cell line. In another embodiment, the AML cell line is OCI-AML3 cell line. In yet another embodiment, the AML cell line is HNT-34 cell line. In still another embodiment, the AML cell line is ML-2 cell line. In one embodiment, the AML cell line is AML-193 cell line. In another embodiment, the AML cell line is F36-P cell line. In yet another embodiment, the AML cell line is KASUMI-3 cell line. In still another embodiment, the AML cell line is MUTZ-8 cell line. In one embodiment, the AML cell line is GDM-1 cell line. In another embodiment, the AML cell line is SIG-M5 cell line. In yet another embodiment, the AML cell line is TF-1 cell line. In still another embodiment, the AML cell line is Nomo-1 cell line. In one embodiment, the AML cell line is UT-7 cell line. In another embodiment, the AML cell line is THP-1 cell line.

In certain embodiments, the methods provided herein are useful for detecting gene rearrangement in cells from a healthy individual. In certain embodiments, the number of cells used in the methods provided herein can range from a single cell to about 10⁹ cells. In some embodiments, the number of cells used in the methods provided herein is about 1×10⁴, about 5×10⁴, about 1×10⁵, about 5×10⁵, about 1×10⁶, about 5×10⁶, about 1×10⁷, about 5×10⁷, about 1×10⁸, about 5×10⁸, or about 1×10⁹.

The number and type of cells collected from a subject can be monitored, for example, by measuring changes in cell surface markers using standard cell detection techniques such as flow cytometry, cell sorting, immunocytochemistry (e.g., staining with tissue specific or cell-marker specific antibodies), fluorescence activated cell sorting (FACS), magnetic activated cell sorting (MACS), by examining the morphology of cells using light or confocal microscopy, and/or by measuring changes in gene expression using techniques well known in the art, such as PCR and gene expression profiling. These techniques can be used, too, to identify cells that are positive for one or more particular markers.

In certain embodiments, subsets of cells are used in the methods provided herein. Methods of sorting and isolating specific populations of cells are well-known in the art and can be based on cell size, morphology, or intracellular or extracellular markers. Such methods include, but are not limited to, flow cytometry, flow sorting, FACS, bead-based separation such as magnetic cell sorting, size-based separation (e.g., a sieve, an array of obstacles, or a filter), sorting in a microfluidics device, antibody-based separation, sedimentation, affinity adsorption, affinity extraction, density gradient centrifugation, laser capture microdissection, etc. FACS is a well-known method for separating particles, including cells, based on the fluorescent properties of the particles (Kamarch, Methods Enzymol. 1987, 151:150-165). Laser excitation of fluorescent moieties in the individual particles results in a small electrical charge allowing electromagnetic separation of positive and negative particles from a mixture. In one embodiment, cell surface marker-specific antibodies or ligands are labeled with distinct fluorescent labels. Cells are processed through the cell sorter, allowing separation of cells based on their ability to bind to the antibodies used. FACS sorted particles may be directly deposited into individual wells of 96-well or 384-well plates to facilitate separation and cloning.

In one embodiment, RNA (e.g., mRNA) or protein is purified from a tumor, and the level of a gene set is measured by mRNA or protein expression analysis. In certain embodiments, the level of a gene set is measured by transcriptomic profiling, qRT-PCR, microarray, high throughput sequencing, or other similar methods known in the art. In other embodiments, the level of a gene set is measured by ELISA, flow cytometry, immunofluorescence, or other similar methods known in the art.

### 5.5. Compounds

The compound suitable for use in the methods and formulations provided herein is Compound D: 2-(4-chlorophenyl)-N-((2-(2,6-dioxopiperidin-3-yl)-1-oxoisoindolin-5-yl)methyl)-2,2-difluoroacetamide having the structure:

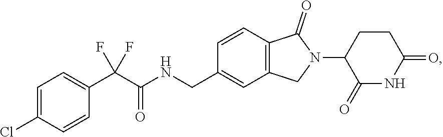 [SMILES: O=C1CCC(N2Cc3cc(CNC(=O)C(F)(F)c4ccc(Cl)cc4)ccc3C2=O)C(=O)N1]

or its stereoisomers or mixture of stereoisomers, isotopologues, pharmaceutically acceptable salts, tautomers, solvates, hydrates, co-crystals, clathrates, or polymorphs thereof. In certain embodiments, Compound D refers to 2-(4-chlorophenyl)-N-((2-(2,6-dioxopiperidin-3-yl)-1-oxoisoindolin-5-yl)methyl)-2,2-difluoroacetamide.

Compound D can be prepared according to the methods described in the Examples provided herein or as described in U.S. Pat. No. 9,499,514, the disclosure of which is incorporated herein by reference in its entirety. The compound can also be synthesized according to other methods apparent to those of skill in the art based upon the teaching herein.

In certain embodiments, Compound D is a solid. In certain embodiments, Compound D is a hydrate. In certain embodiments, Compound D is solvated. In certain embodiments, Compound D is anhydrous.

In certain embodiments, Compound D is amorphous. In certain embodiments, Compound D is crystalline. In certain embodiments, Compound D is in a crystalline form described in U.S. Publication No. 2017-0197934 filed on Jan. 6, 2017, which is incorporated herein by reference in its entirety.

The solid forms of Compound D can be prepared according to the methods described in the disclosure of U.S. Publication No. 2017-0197934 filed on Jan. 6, 2017. The solid forms can also be prepared according to other methods apparent to those of skill in the art.

In one embodiment, Compound D is polymorph Form A, Form B, Form C, Form D, Form E or an amorphous form of 2-(4-chlorophenyl)-N-((2-(2,6-dioxopiperidin-3-yl)-1-oxoisoindolin-5-yl)methyl)-2,2-difluoroacetamide. Polymorphs of 2-(4-chlorophenyl)-N-((2-(2,6-dioxopiperidin-3-yl)-1-oxoisoindolin-5-yl)methyl)-2,2-difluoroacetamide are briefly described herein. In certain embodiments, Compound D has a polymorph form as described in US Publication No. 2019/0030018, the disclosure of which is incorporated herein by reference in its entirety, and portion of which is described in more detail below.

### Form A of 2-(4-chlorophenyl)-N-((2-(2,6-dioxopiperidin-3-yl)-1-oxoisoindolin-5-yl)methyl)-2,2-difluoroacetamide

In certain embodiments, the formulations provided herein are prepared from Form A of Compound D.

In one embodiment, Form A is an anhydrous form of Compound D. In another embodiment, Form A of Compound D is crystalline.

In certain embodiments, Form A is obtained by crystallization from certain solvent systems, for example, solvent systems comprising one or more of the following solvents: acetone and the solvent mixture of isopropanol and water at room temperature. In certain embodiments, Form A is obtained as an intermediate solid form from slurries at elevated temperature, for example about 50° C., in ethanol/water (1:1), acetone or acetonitrile.

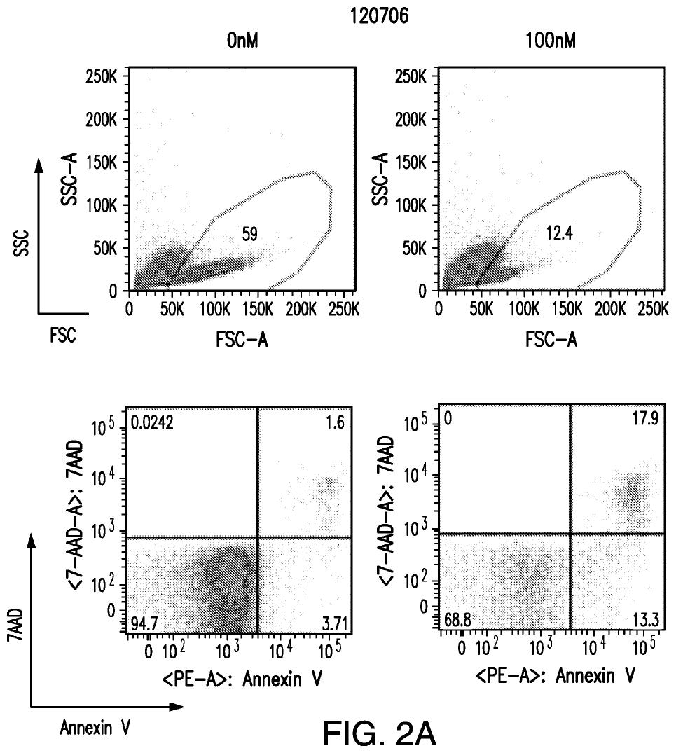

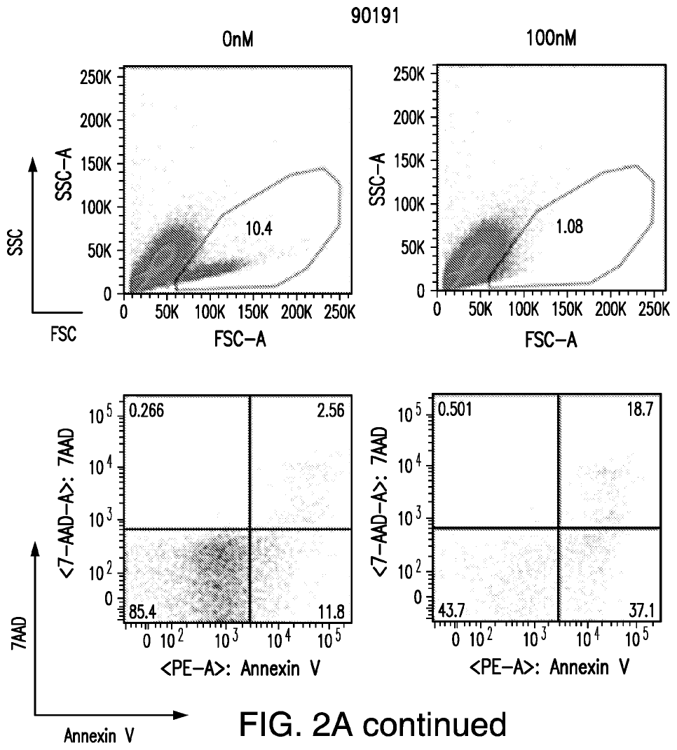

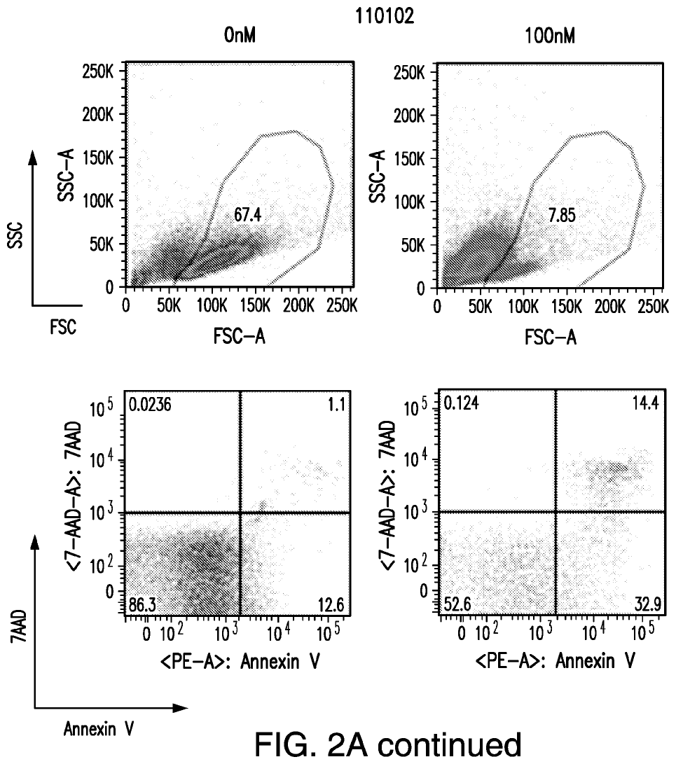

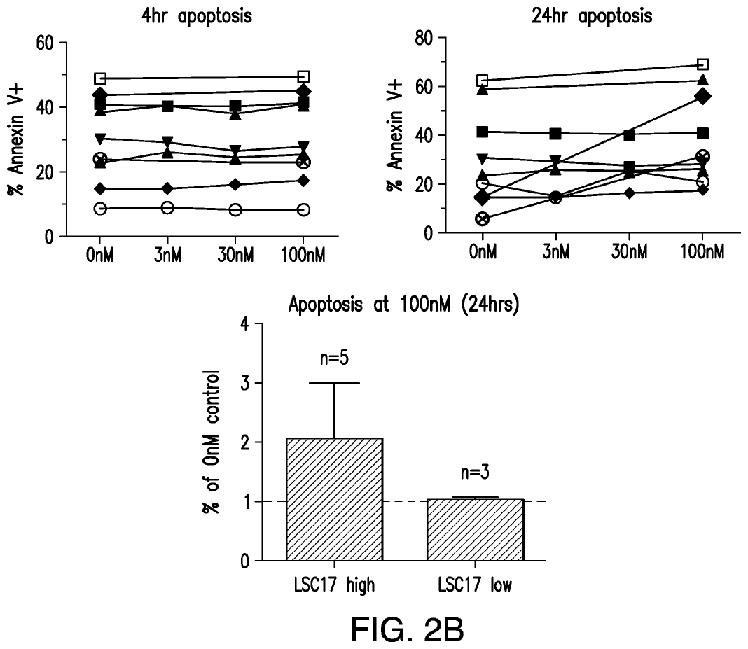

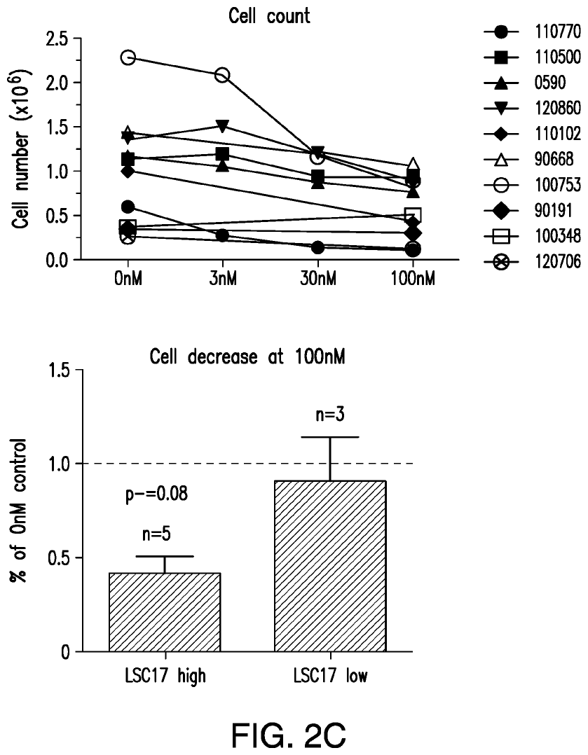

In certain embodiments, Form A is substantially crystalline, as indicated by, e.g., X-ray powder diffraction measurements. In one embodiment, Form A of Compound D has an X-ray powder diffraction pattern substantially as shown in FIG. 2 of US Publication No. 2019/0030018.

In one embodiment, Form A of Compound D has one or more characteristic X-ray powder diffraction peaks at a two-theta angle of approximately 11.5, 15.6, 16.6, 17.2, 18.1, 19.0, 19.6, 21.1, 23.2 or 24.8 degrees 2θ as depicted in FIG. 2 of US Publication No. 2019/0030018. In another embodiment, Form A of Compound D has one, two, three or four characteristic X-ray powder diffraction peaks at a two-theta angle of approximately 15.6, 16.6, 17.2 or 24.8 degrees 2θ. In another embodiment, Form A of Compound D has one, two, three, four, five, six or seven characteristic X-ray powder diffraction peaks as set forth in Table A. In another embodiment, Form A of Compound D has one, two, or three characteristic X-ray powder diffraction peaks as set forth in Table A.

TABLE A

| | Pos. | d-spacing | Rel. Int. |
|---|---|---|---|
| No. | [°2 Th.] | [Å] | [%] |
| 1 | 7.23 | 12.2187 | 17.6 |
| 2 | 11.52 | 7.6789 | 29.7 |
| 3 | 15.22 | 5.8209 | 7.5 |
| 4 | 15.62 | 5.6720 | 31.2 |
| 5 | 16.58 | 5.3466 | 40.3 |
| 6 | 17.19 | 5.1576 | 100.0 |
| 7 | 18.08 | 4.9056 | 22.3 |
| 8 | 19.00 | 4.6702 | 19.6 |
| 9 | 19.60 | 4.5302 | 22.1 |
| 10 | 21.05 | 4.2197 | 29.2 |
| 11 | 21.74 | 4.0884 | 8.3 |
| 12 | 22.01 | 4.0388 | 7.1 |
| 13 | 22.47 | 3.9576 | 6.0 |
| 14 | 23.22 | 3.8312 | 28.6 |
| 15 | 24.17 | 3.6825 | 5.6 |
| 16 | 24.77 | 3.5945 | 57.2 |
| 17 | 25.59 | 3.4813 | 14.6 |
| 18 | 25.94 | 3.4356 | 10.5 |
| 19 | 26.63 | 3.3470 | 17.4 |
| 20 | 27.73 | 3.2172 | 10.0 |
| 21 | 28.51 | 3.1307 | 7.1 |
| 22 | 29.88 | 2.9906 | 19.3 |
| 23 | 30.76 | 2.9065 | 7.1 |
| 24 | 31.59 | 2.8327 | 11.1 |
| 25 | 34.82 | 2.5766 | 4.8 |
| 26 | 36.05 | 2.4913 | 4.3 |

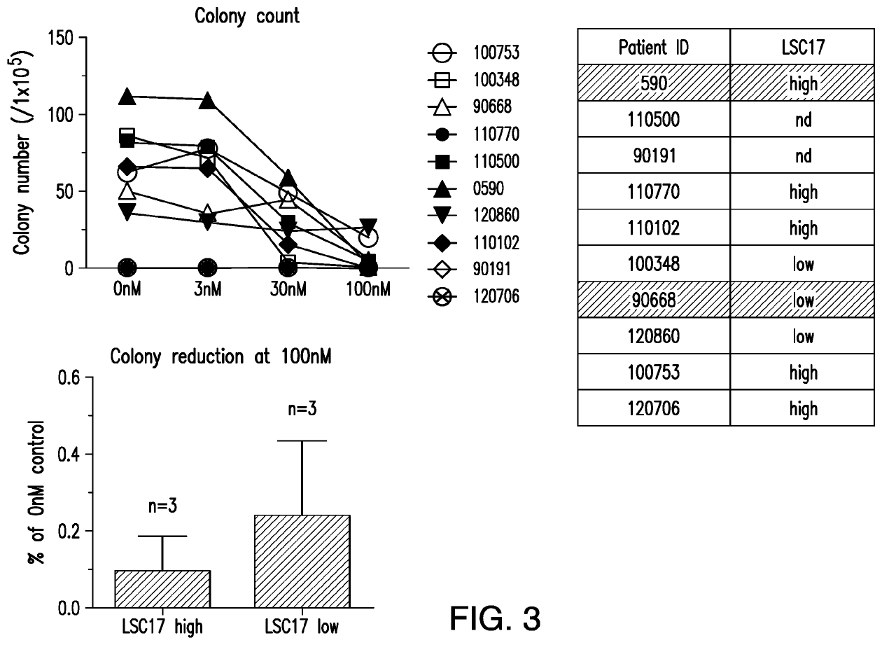

In one embodiment, Form A of Compound D has the SEM picture as shown in FIG. 3 of US Publication No. 2019/0030018.

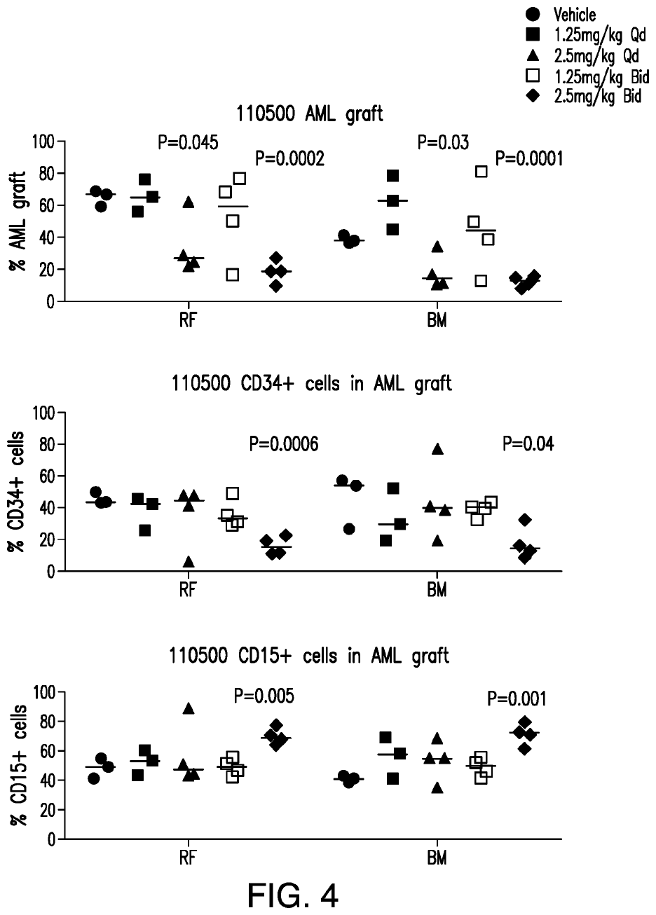

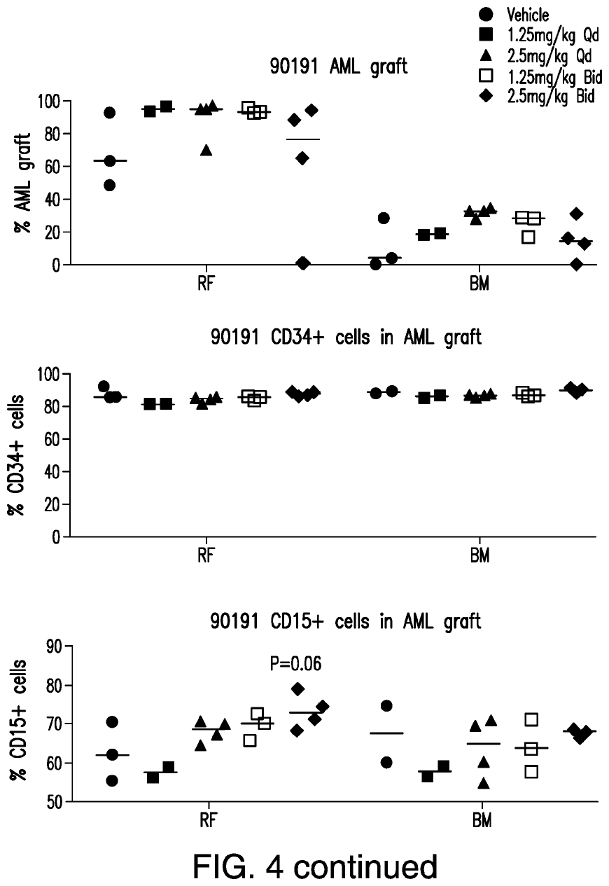

In one embodiment, the crystalline form of Compound D has a thermogravimetric (TGA) thermograph corresponding substantially to the representative TGA thermogram as depicted in FIG. 4 of US Publication No. 2019/0030018. In certain embodiments, no TGA weight loss is observed for Form A.

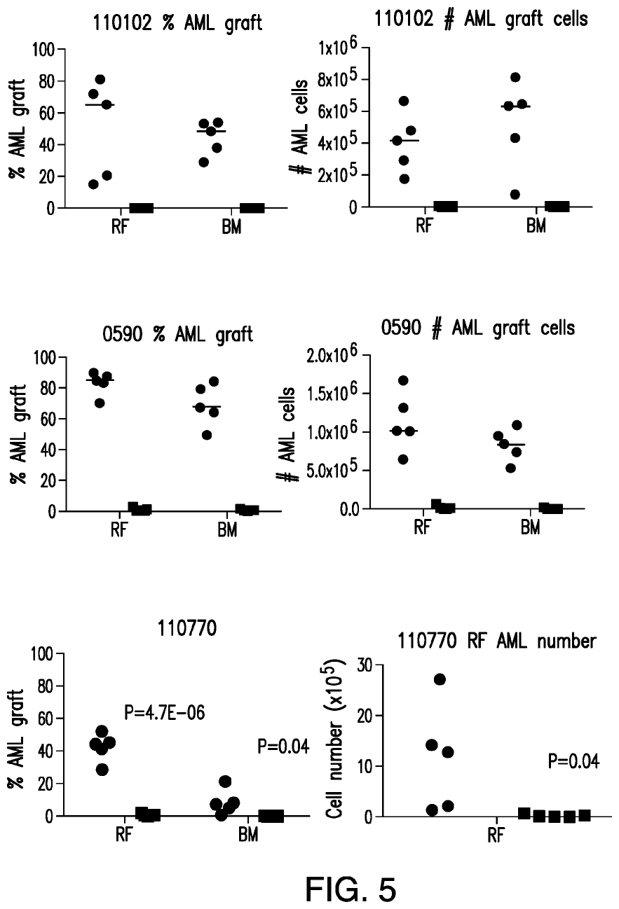

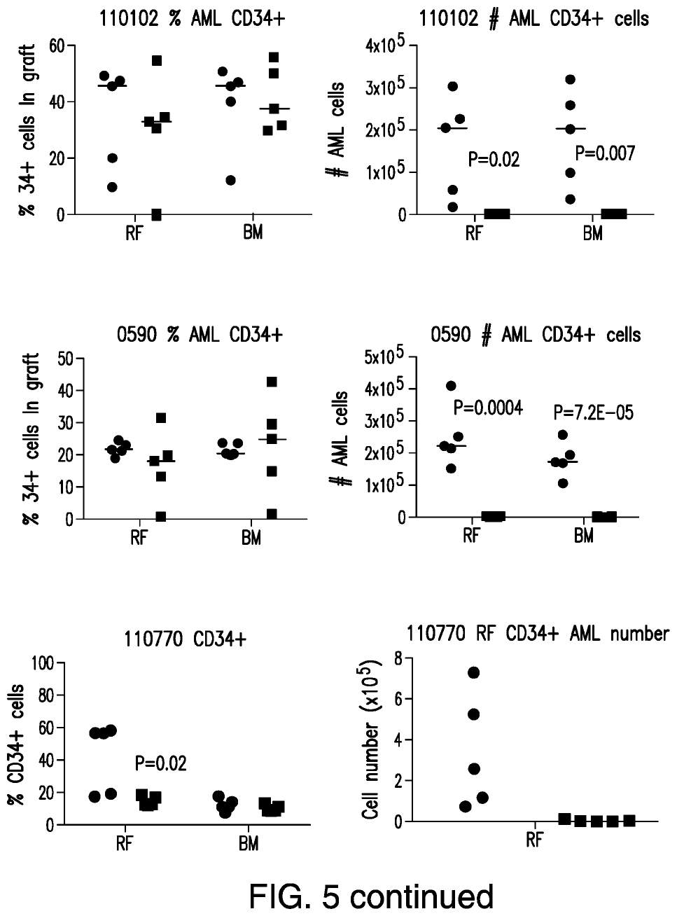

In one embodiment, crystalline form A of Compound D has a DSC thermogram corresponding substantially as depicted in FIG. 5 of US Publication No. 2019/0030018. In certain embodiments, Form A is characterized by a DSC plot comprising a melting event with an onset temperature of 229° C. and heat of fusion of 118 J/g.

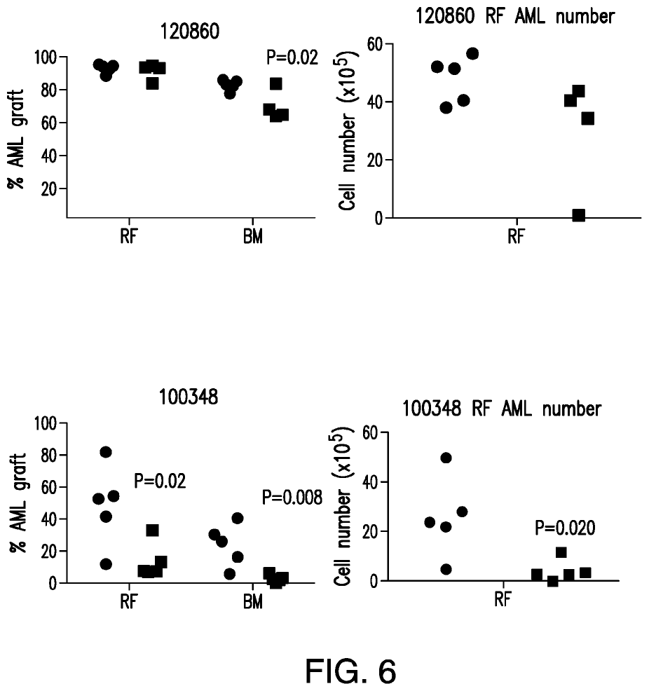

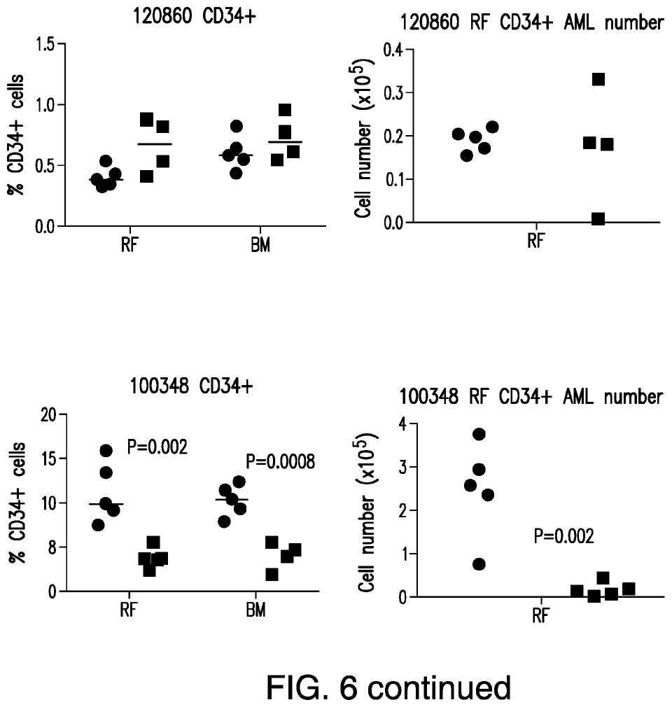

In certain embodiments, Form A is characterized by dynamic vapor sorption analysis. A representative dynamic vapor sorption (DVS) isotherm plot is shown in FIG. 6 of US Publication No. 2019/0030018. In certain embodiments, when the relative humidity (“RH”) is increased from about 0% to about 90% RH, Form A exhibits less than 1.5%, less than 1.2% or about 1.2% w/w water uptake. In certain embodiments, Form A comprises less than 0.1% water as determined in a coulometric Karl Fischer (KF) titrator equipped with an oven sample processor set at 225° C.

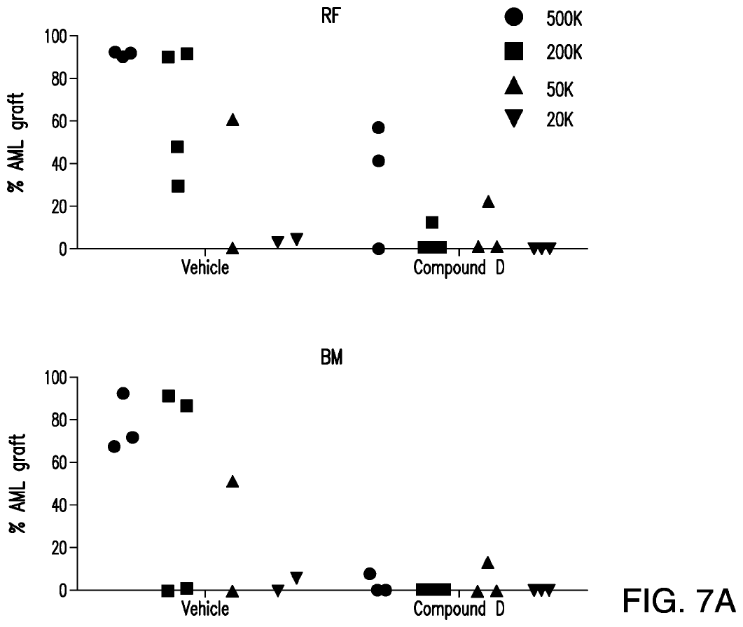

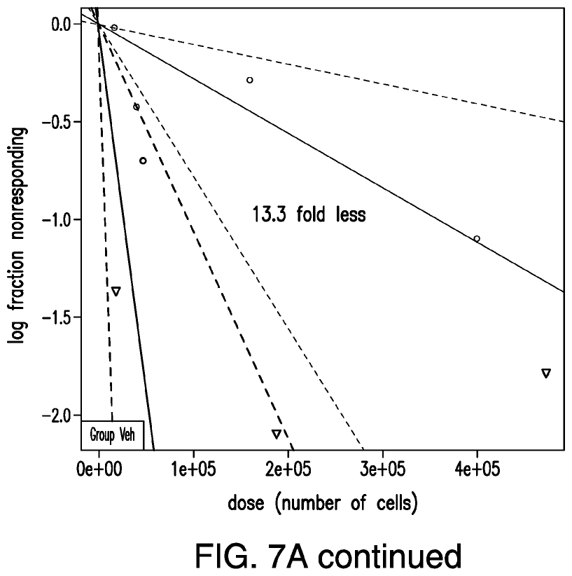

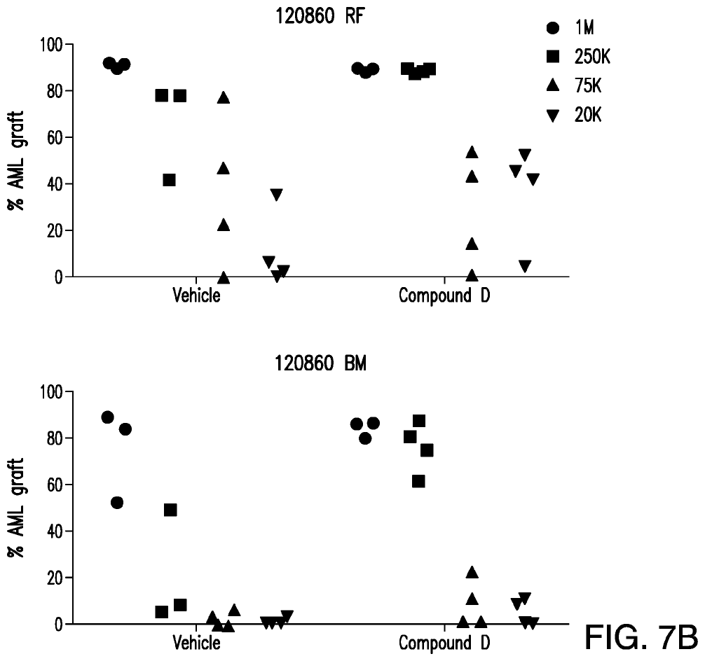

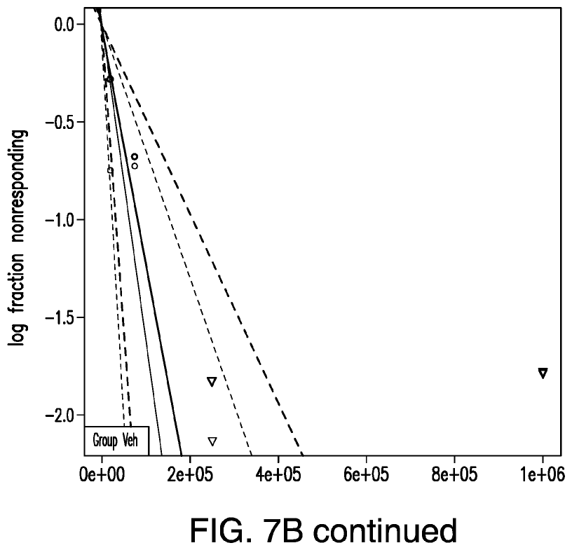

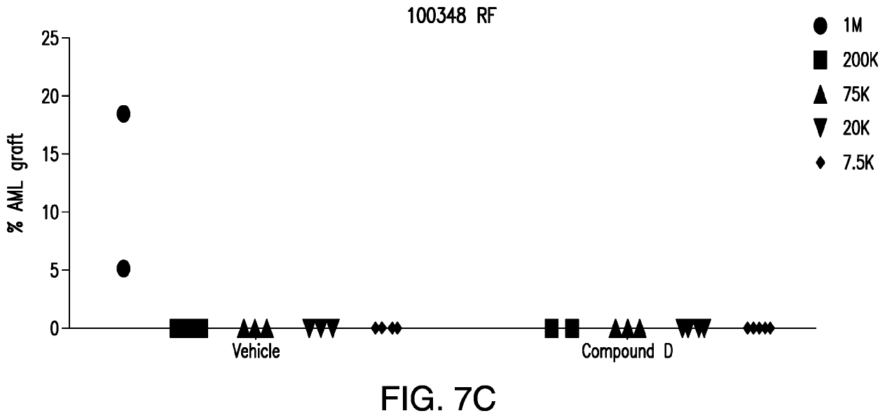

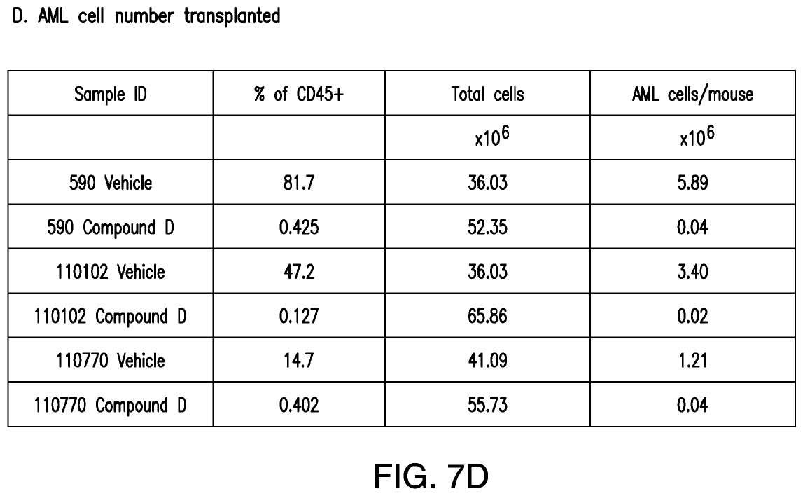

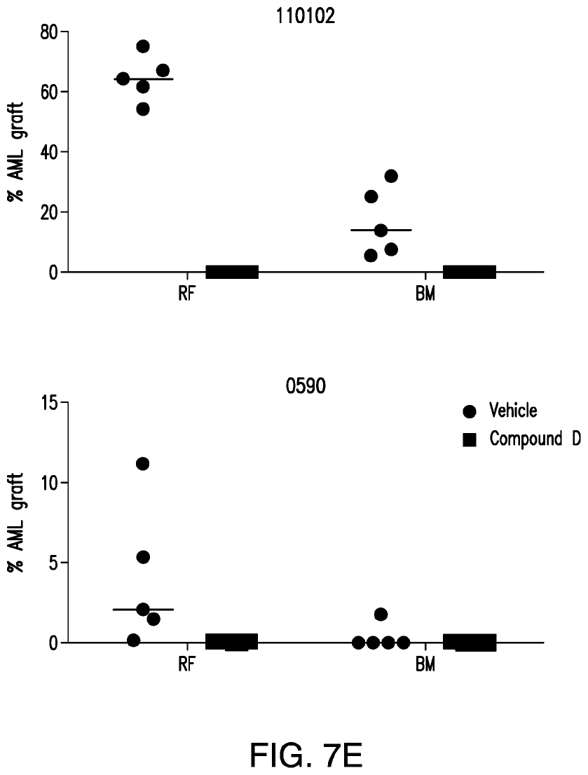

In certain embodiments, no significant degradation or residual solvent for Form A is observed by ¹H NMR (see FIG. 7 of US Publication No. 2019/0030018).

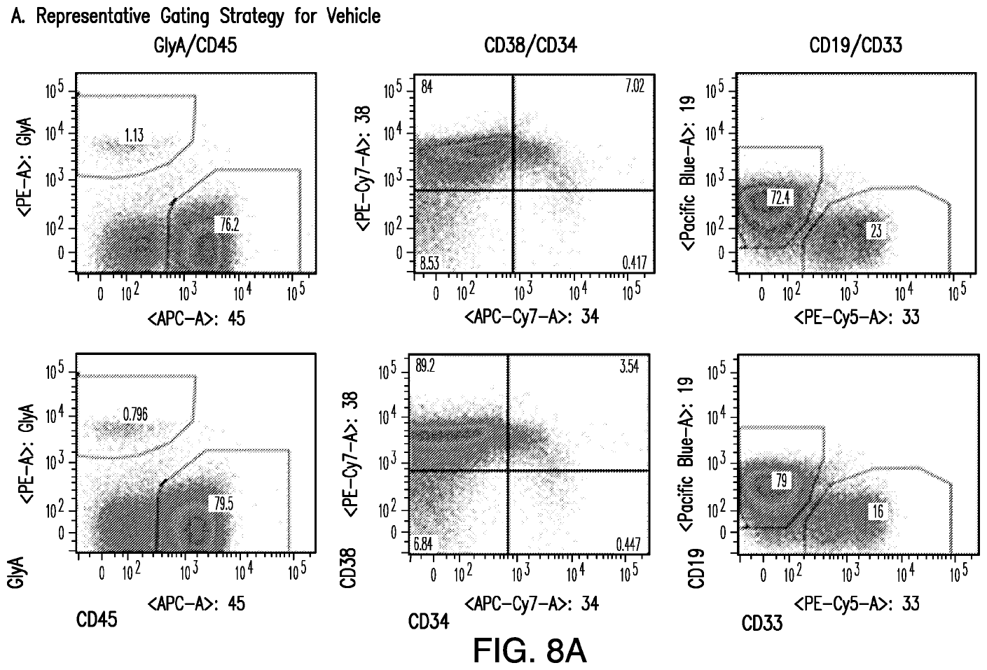

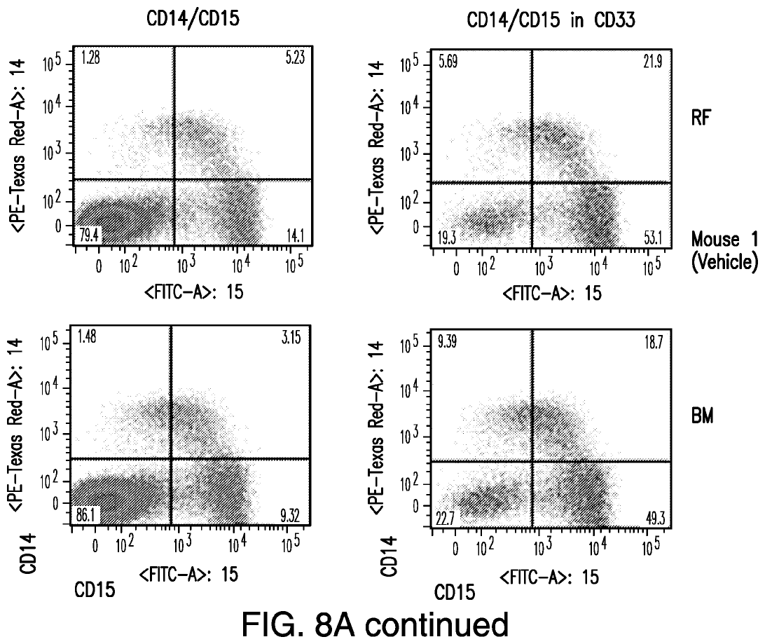

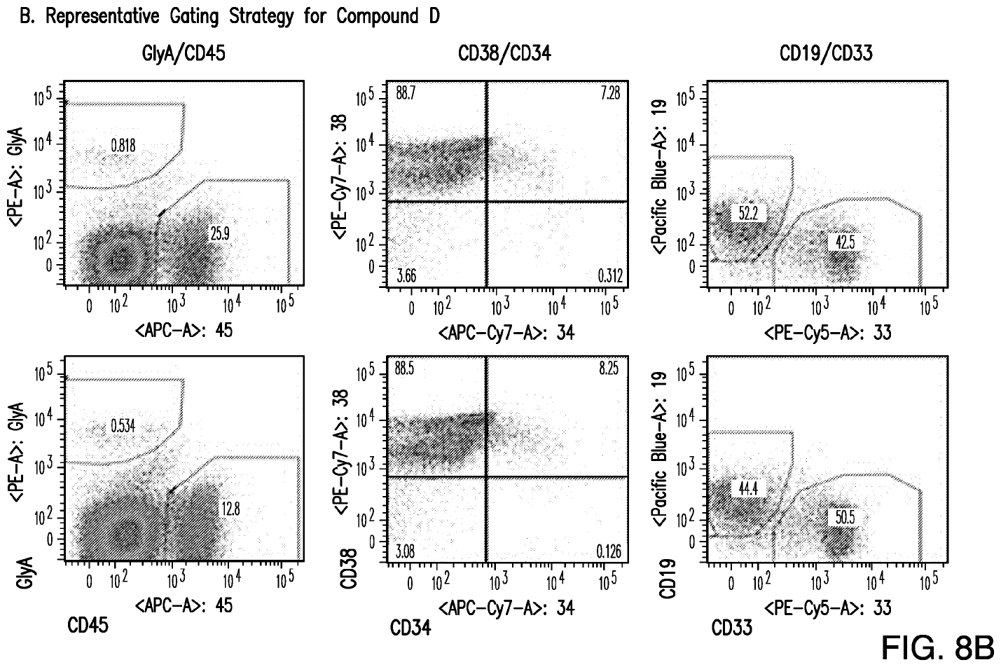

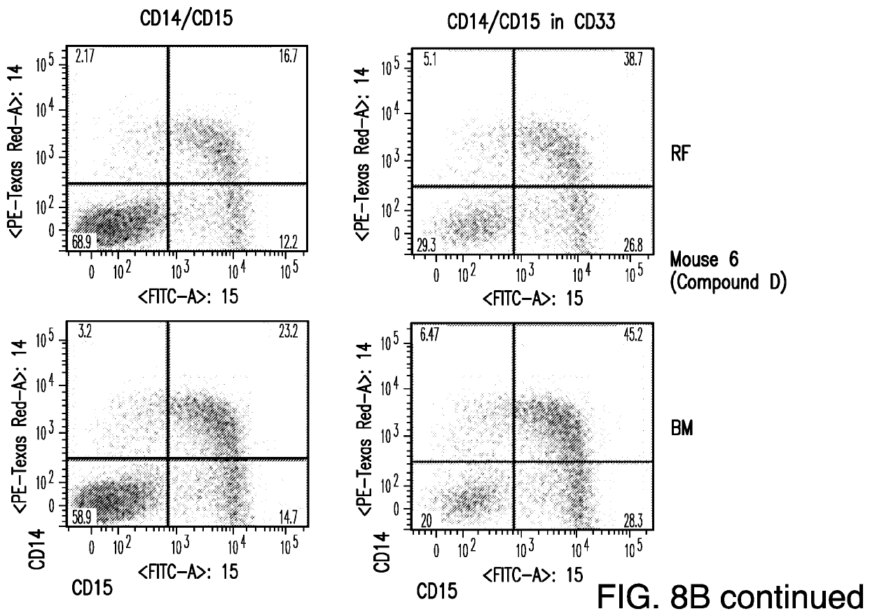

In certain embodiments, Form A of Compound D is characterized by its stability profile upon compression. In certain embodiments, Form A is stable, e.g., its XRPD pattern remains substantially unchanged with broader diffraction peaks, upon application of 2000-psi pressure for about 1 minute (see FIG. 8 of US Publication No. 2019/0030018).

In still another embodiment, Form A of Compound D is substantially pure. In certain embodiments, the substantially pure Form A of Compound D is substantially free of other solid forms, e.g., amorphous form. In certain embodiments, the purity of the substantially pure Form A of Compound D is no less than about 95% pure, no less than about 96% pure, no less than about 97% pure, no less than about 98% pure, no less than about 98.5% pure, no less than about 99% pure, no less than about 99.5% pure, or no less than about 99.8% pure.

Certain embodiments Form A of Compound D is substantially pure. In certain embodiments herein Form A of Compound D is substantially free of other solid forms comprising Compound D including, e.g., Forms B, C, D, E and/or an amorphous solid form comprising Compound D. In certain embodiments, Form A is a mixture of solid forms comprising Compound D, including, e.g., a mixture comprising one or more of the following: Forms B, C, D, E and an amorphous solid form comprising Compound D.

### Form B of 2-(4-chlorophenyl)-N-((2-(2,6-dioxopiperidin-3-yl)-1-oxoisoindolin-5-yl)methyl)-2,2-difluoroacetamide

In certain embodiments, the formulations provided herein are prepared from anhydrous Form B of Compound D.

In certain embodiments, Form B is obtained by anti-solvent recrystallization from certain solvent systems, for example, solvent systems comprising one or more of the following solvents: methanol/water, DMSO/isopropanol, DMSO/toluene, and DMSO/water. In certain embodiments, Form B is obtained by cooling recrystallization from THF/water (1:1).

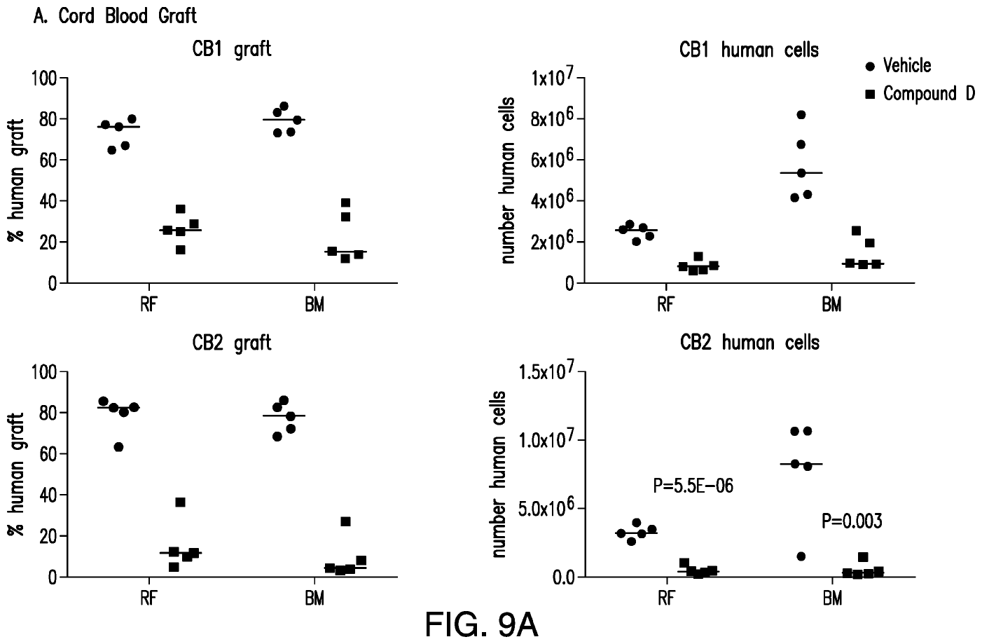

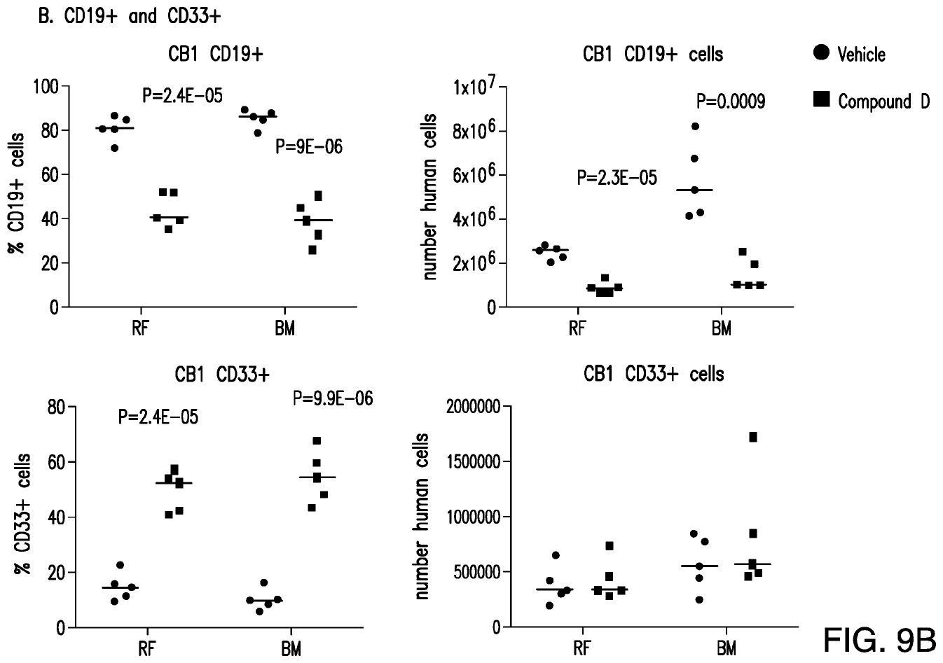

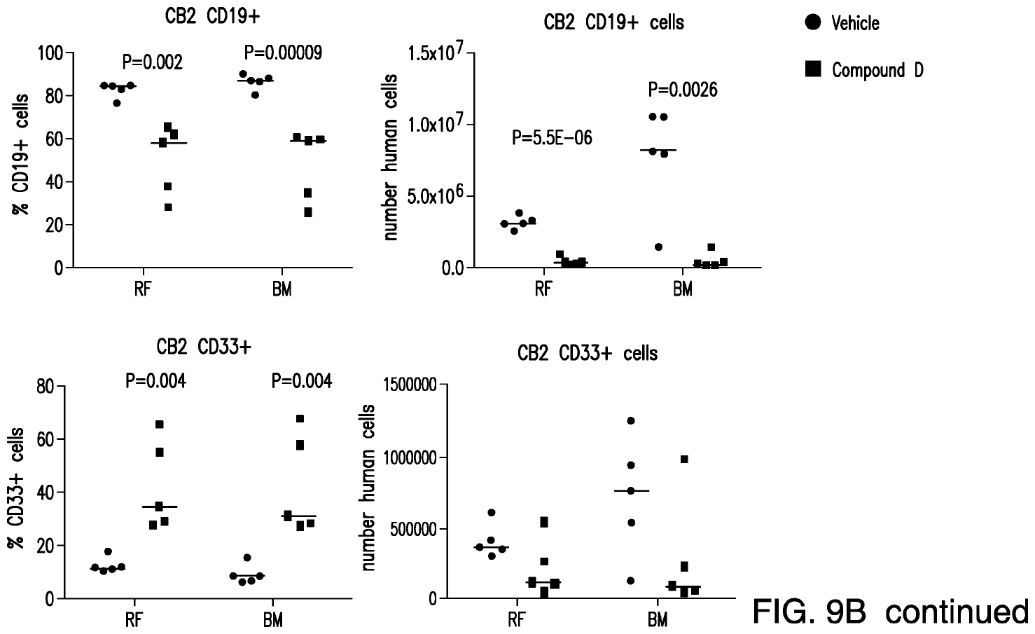

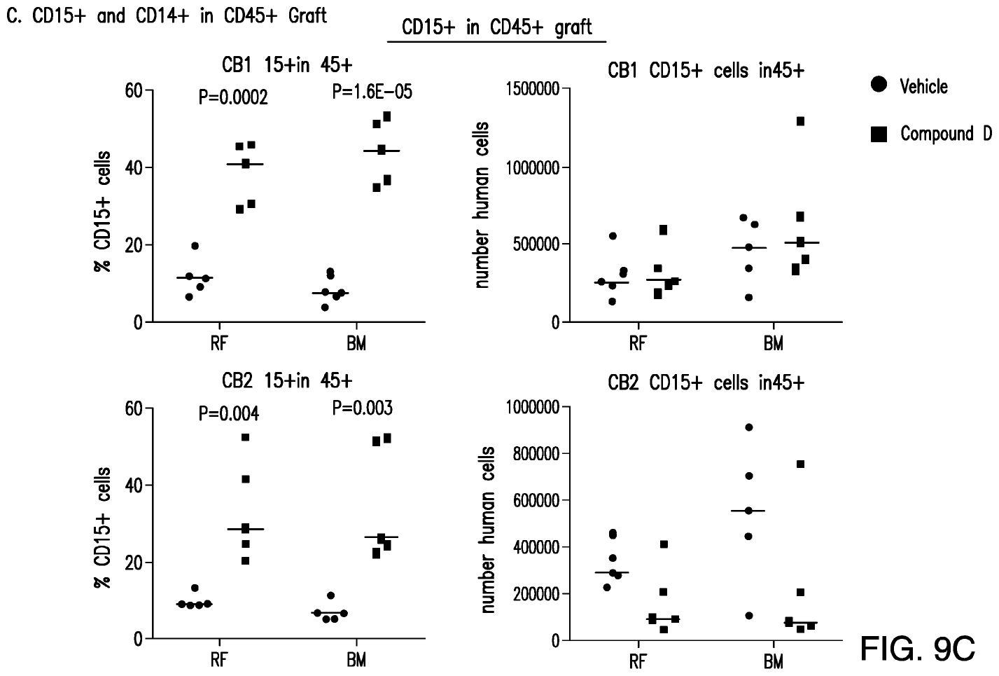

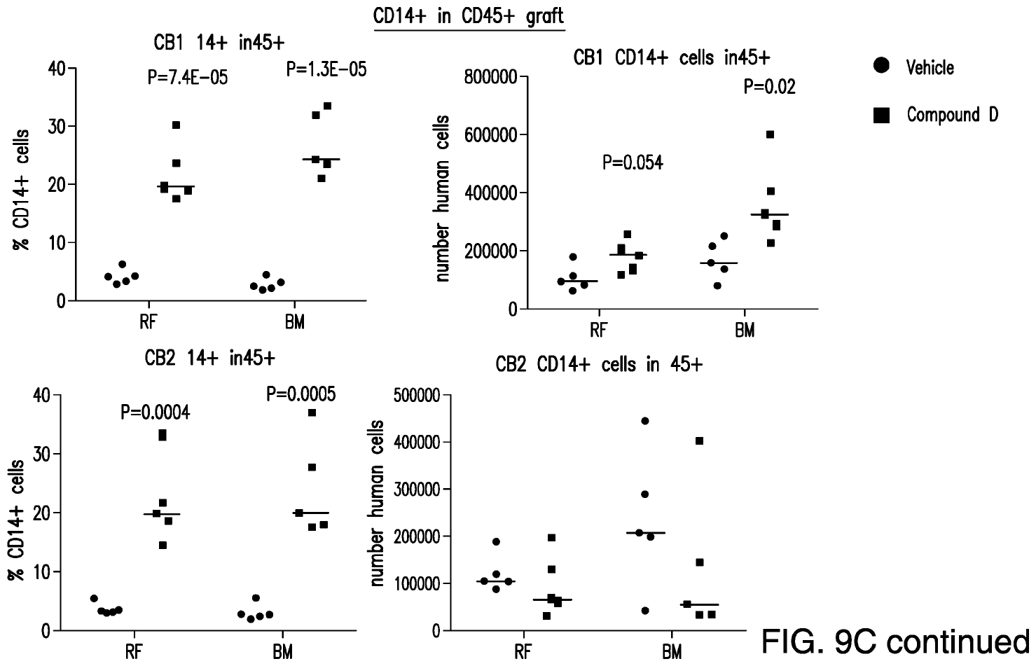

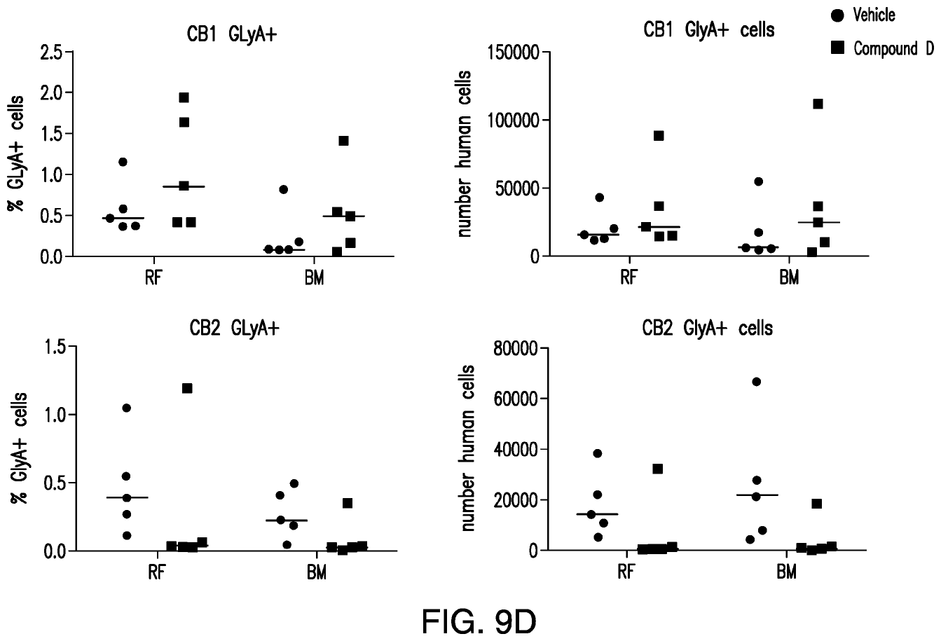

In certain embodiments, Form B is crystalline, as indicated by, e.g., X-ray powder diffraction measurements. In one embodiment, Form B of Compound D has an X-ray powder diffraction pattern substantially as shown in FIG. 9 of US Publication No. 2019/0030018.

In one embodiment, Form B of Compound D has one or more characteristic X-ray powder diffraction peaks at a two-theta angle of approximately 15.4, 16.3, 16.7, 17.7, 20.4, 25.6 or 27.5, degrees 2θ as depicted in FIG. 9 of US Publication No. 2019/0030018. In another embodiment, Form B of Compound D has one, two, three or four characteristic X-ray powder diffraction peaks at a two-theta angle of approximately 16.7, 25.6, 15.4 or 16.3 degrees 2θ. In another embodiment, Form B of Compound D has one, two, three, four, five, six or seven characteristic X-ray powder diffraction peaks as set forth in Table B. In another embodiment, Form B of Compound D has one, two, or three characteristic X-ray powder diffraction peaks as set forth in Table B.

TABLE B

| No. | Pos. [°2 Th.] | d-spacing [Å] | Rel. Int. [%] |
|---|---|---|---|
| 1 | 7.01 | 12.6035 | 9.3 |
| 2 | 11.58 | 7.6444 | 8.3 |
| 3 | 11.80 | 7.5027 | 6.8 |
| 4 | 12.73 | 6.9551 | 18.4 |
| 5 | 15.38 | 5.7601 | 34.8 |
| 6 | 16.32 | 5.4330 | 31.4 |
| 7 | 16.72 | 5.3012 | 100.0 |
| 8 | 17.72 | 5.0046 | 26.6 |
| 9 | 18.13 | 4.8930 | 19.8 |
| 10 | 18.77 | 4.7271 | 7.5 |
| 11 | 20.41 | 4.3516 | 22.0 |
| 12 | 21.02 | 4.2258 | 15.9 |
| 13 | 21.21 | 4.1881 | 13.5 |
| 14 | 21.93 | 4.0529 | 3.4 |
| 15 | 23.68 | 3.7581 | 14.2 |
| 16 | 25.01 | 3.5601 | 10.4 |
| 17 | 25.63 | 3.4755 | 37.3 |
| 18 | 26.19 | 3.4030 | 9.8 |
| 19 | 26.73 | 3.3349 | 8.5 |
| 20 | 27.45 | 3.2499 | 20.9 |
| 21 | 27.71 | 3.2193 | 9.4 |
| 22 | 28.22 | 3.1623 | 11.8 |
| 23 | 29.48 | 3.0296 | 4.7 |
| 24 | 30.10 | 2.9692 | 15.0 |
| 25 | 31.08 | 2.8775 | 18.3 |
| 26 | 31.65 | 2.8272 | 6.2 |
| 27 | 34.29 | 2.6150 | 3.4 |

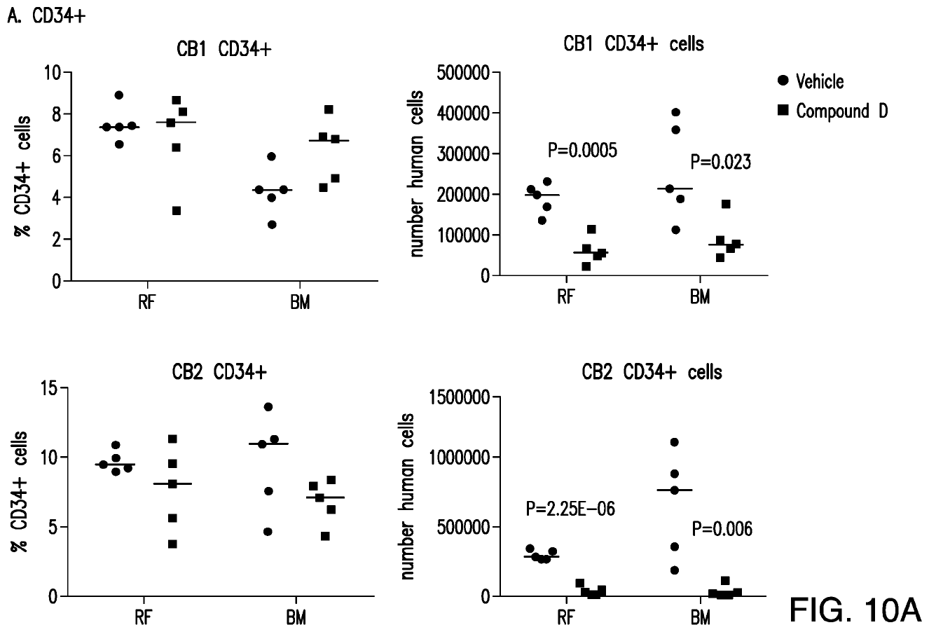

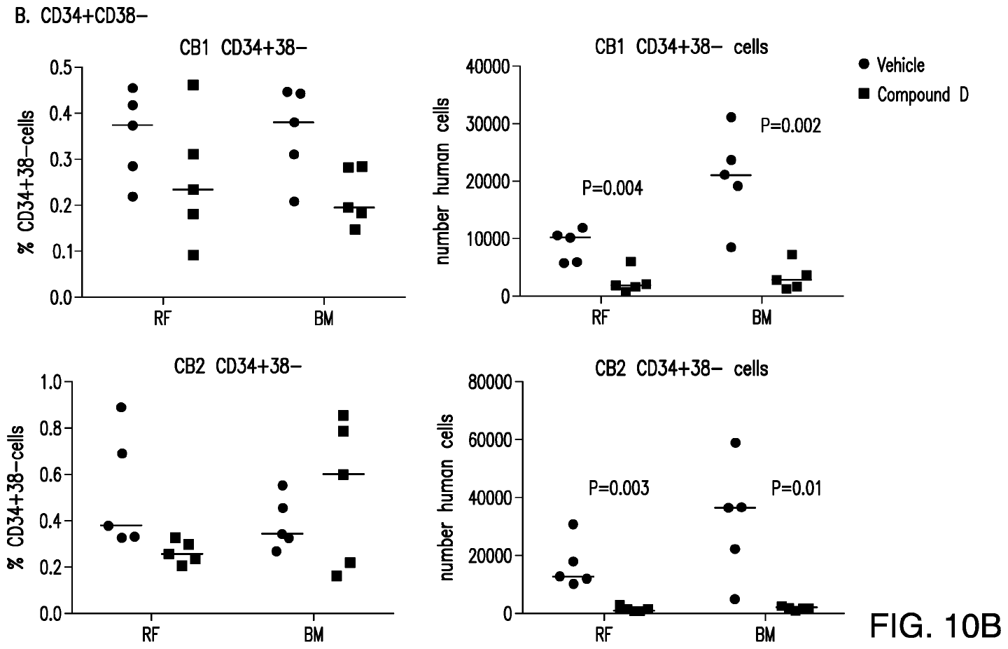

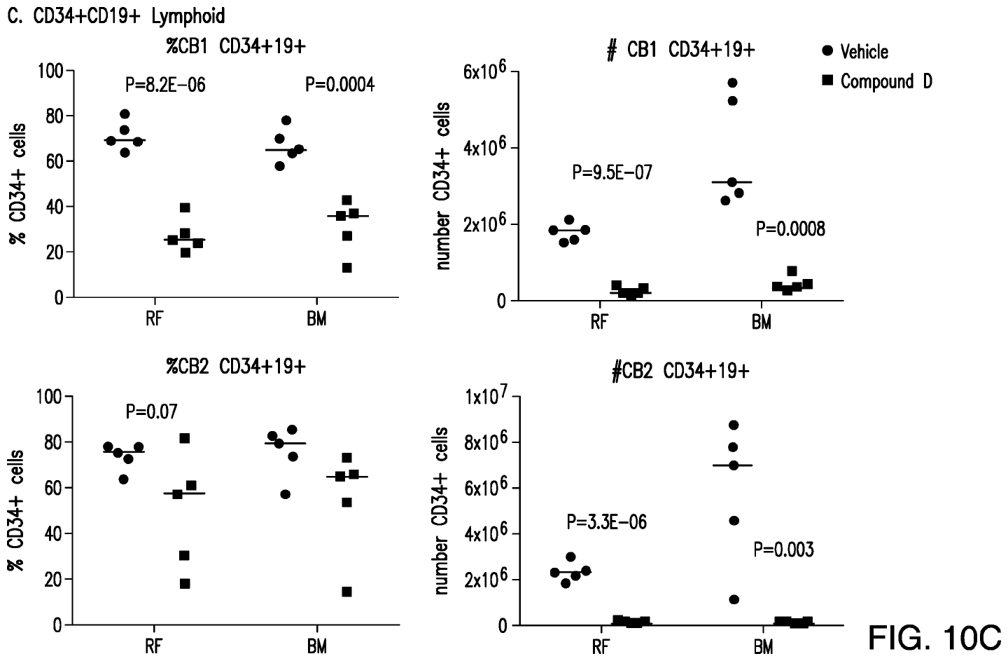

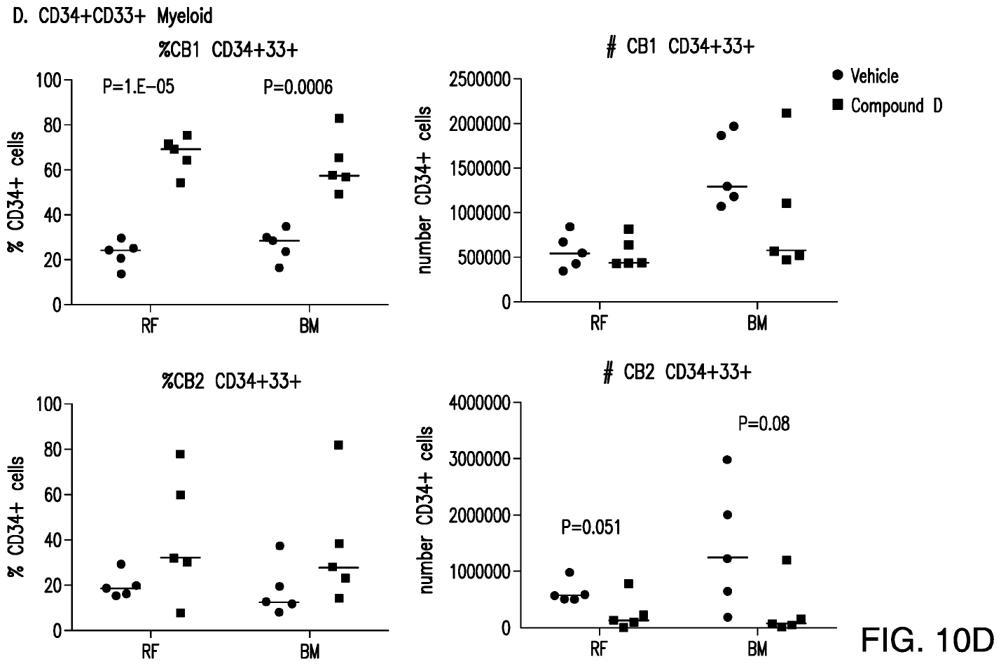

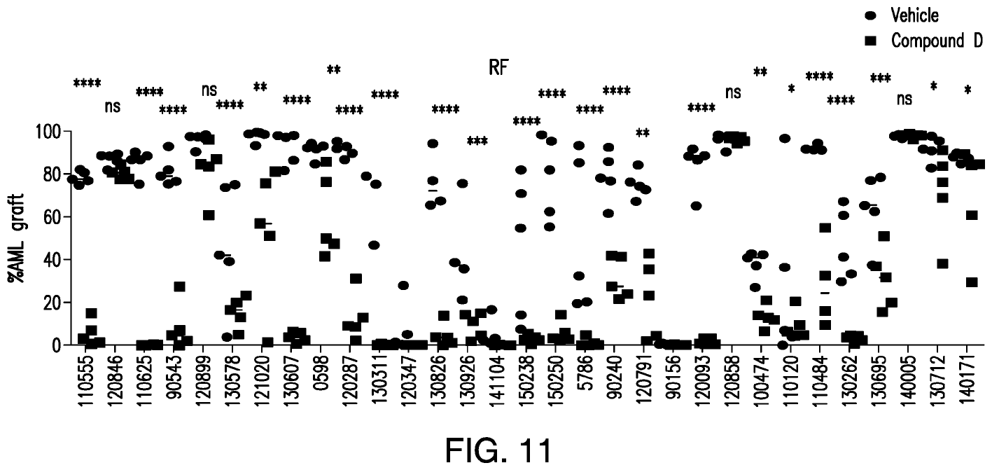

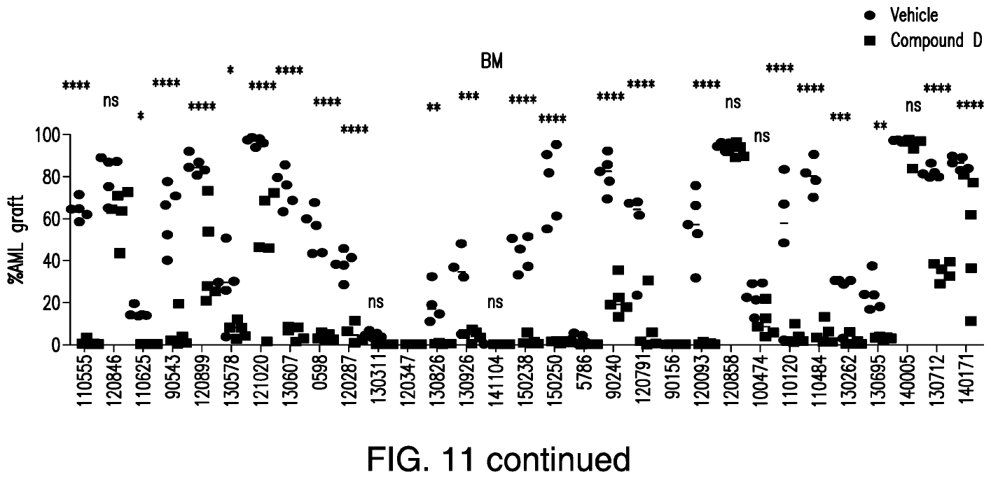

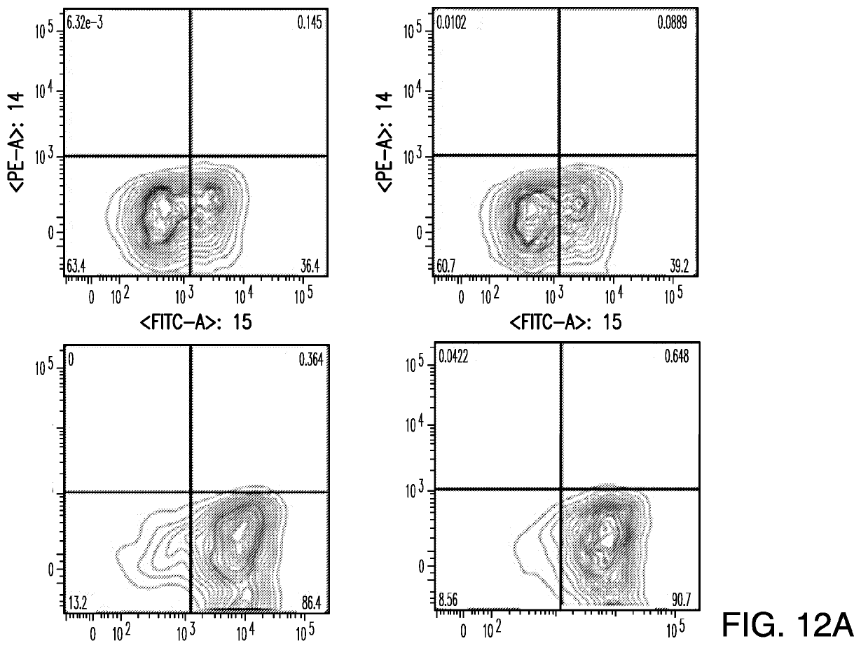

In one embodiment, Form B of Compound D has the SEM picture as shown in FIG. 10 of US Publication No. 2019/0030018. In one embodiment, a crystalline form of Compound D has a thermogravimetric (TGA) thermograph corresponding substantially to the representative TGA thermogram as depicted in FIG. 11 of US Publication No. 2019/0030018. In certain embodiments, Form B shows no TGA weight loss below 170° C. In certain embodiments, Form B shows a TGA weight loss of 0.4% between 170˜230° C.

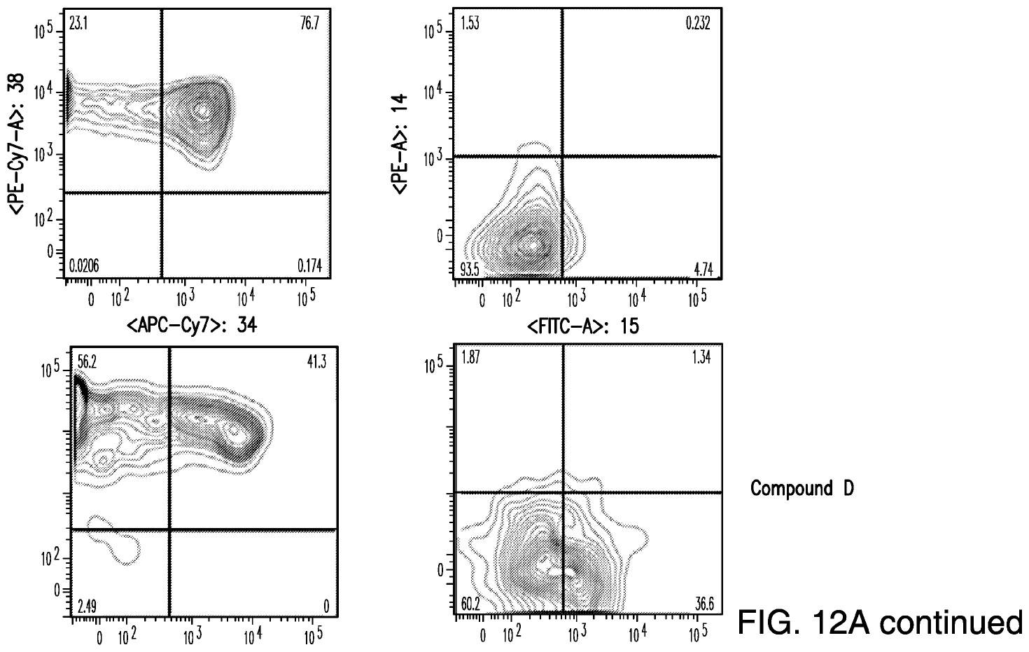

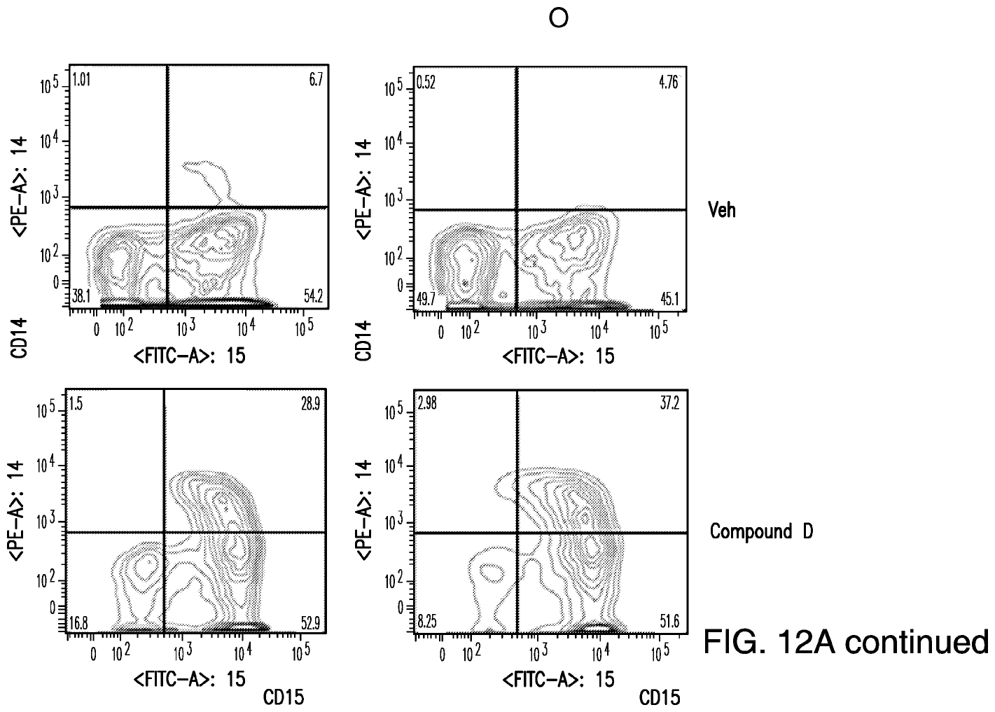

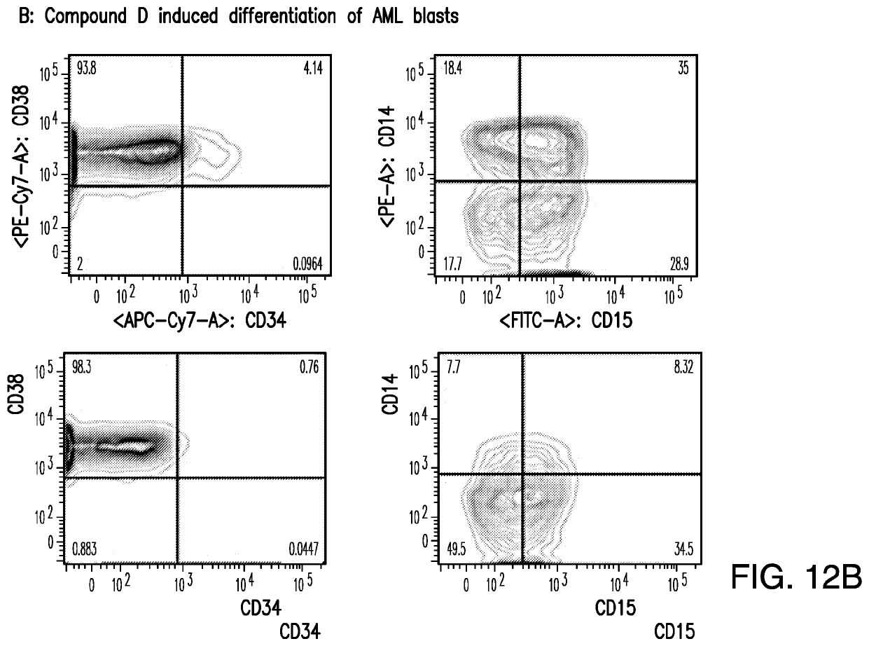

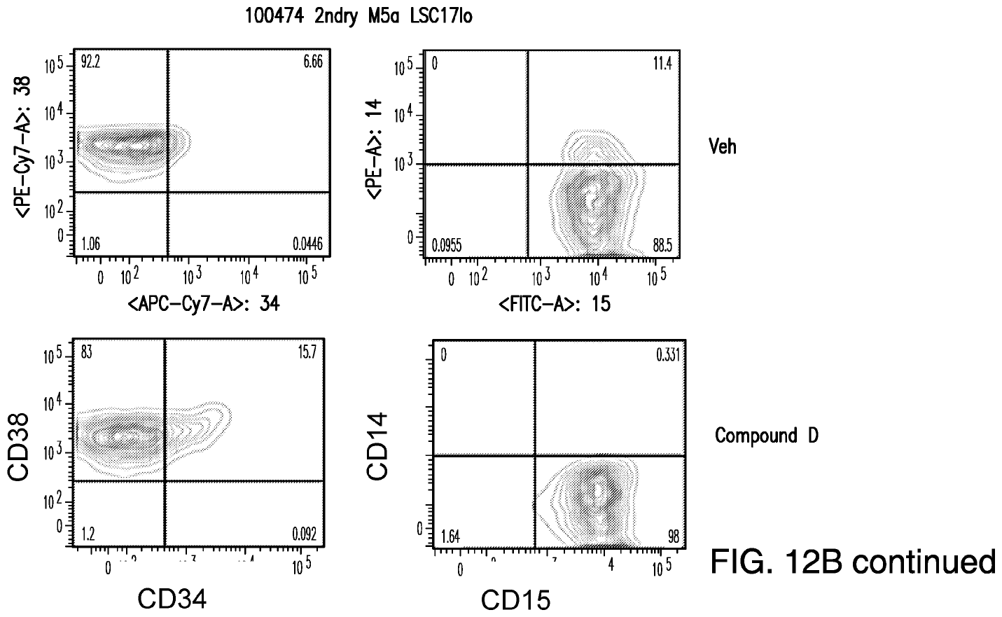

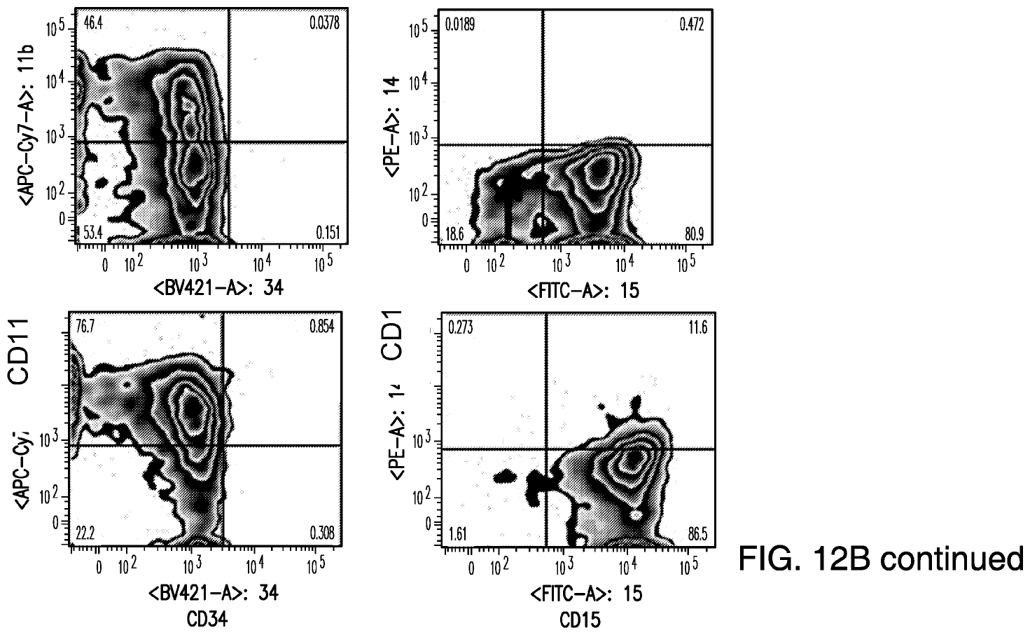

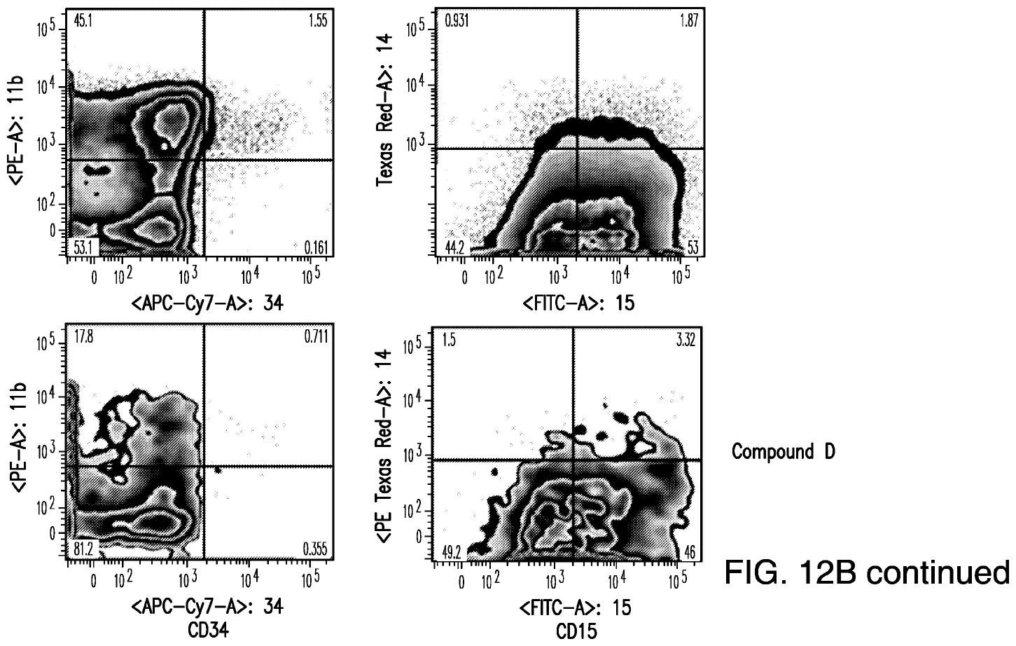

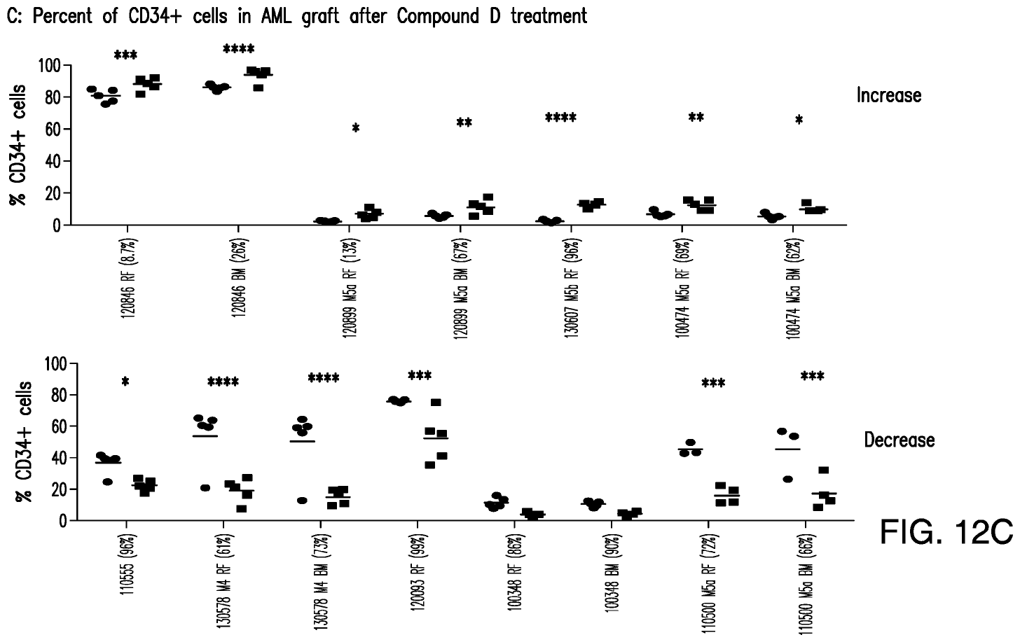

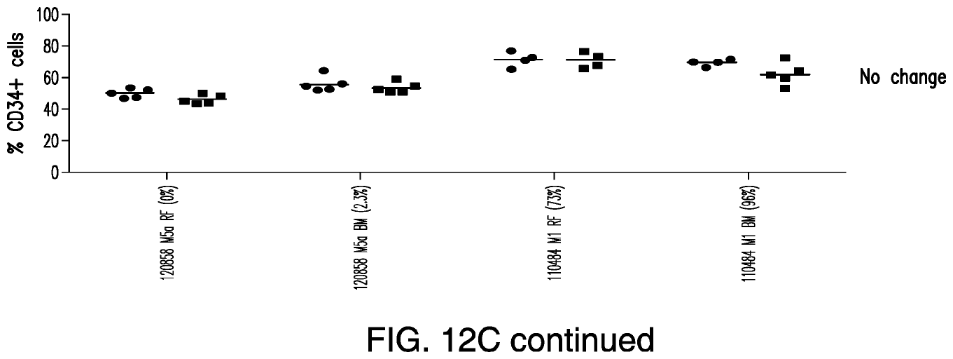

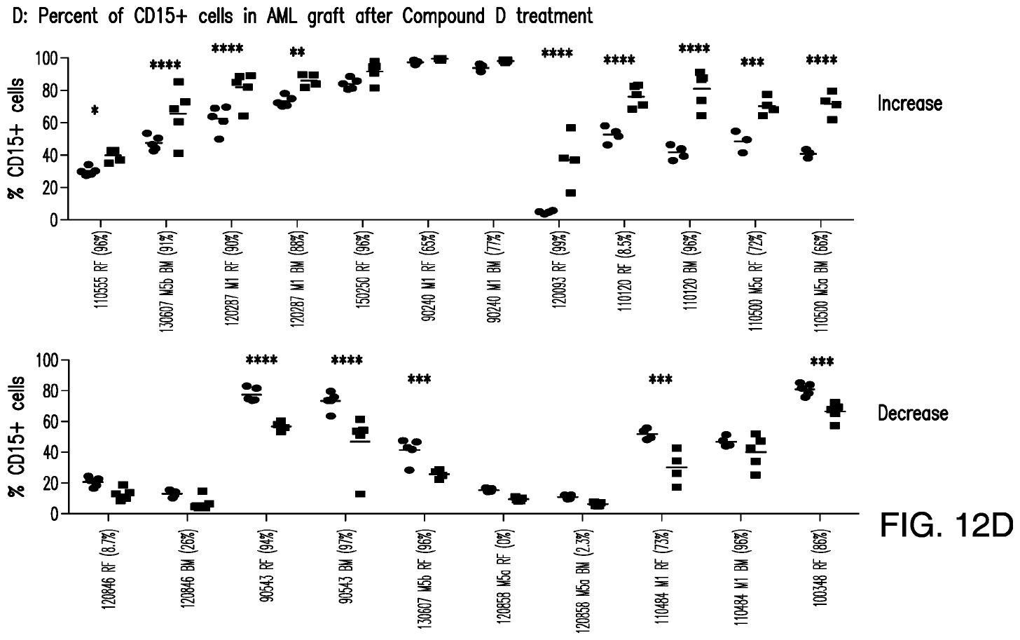

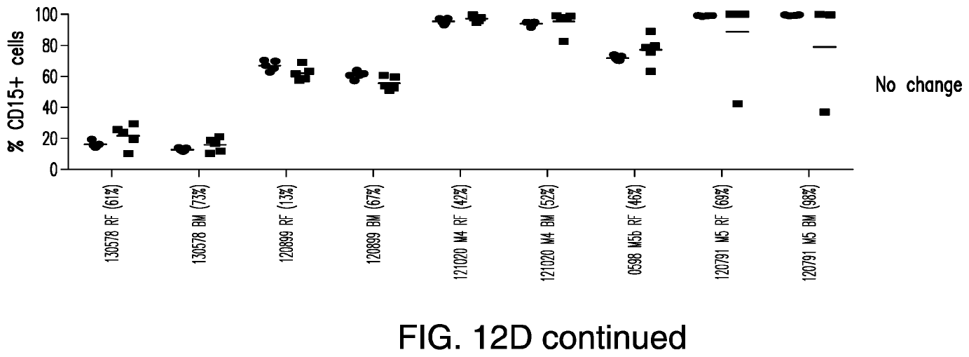

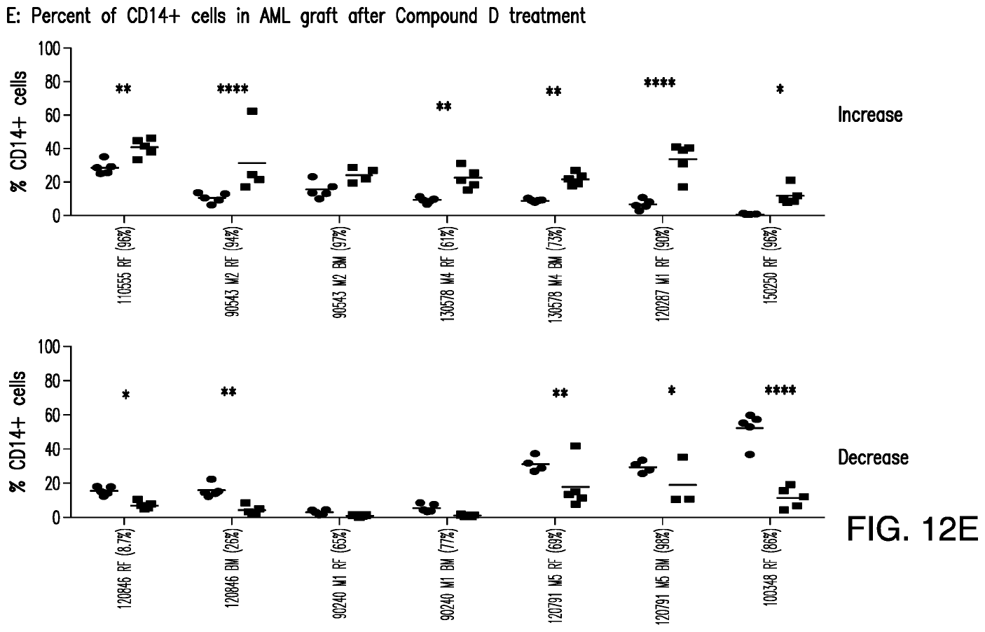

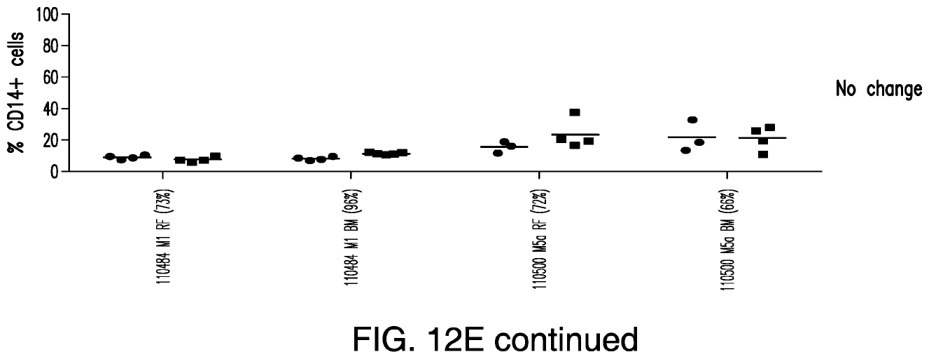

In one embodiment, crystalline Form B of Compound D has a DSC thermogram corresponding substantially as depicted in FIG. 12 of US Publication No. 2019/0030018. In certain embodiments, Form B is characterized by a DSC plot comprising a melt/recrystallization event at 219˜224° C. and a major melting event with a peak temperature of 231° C.

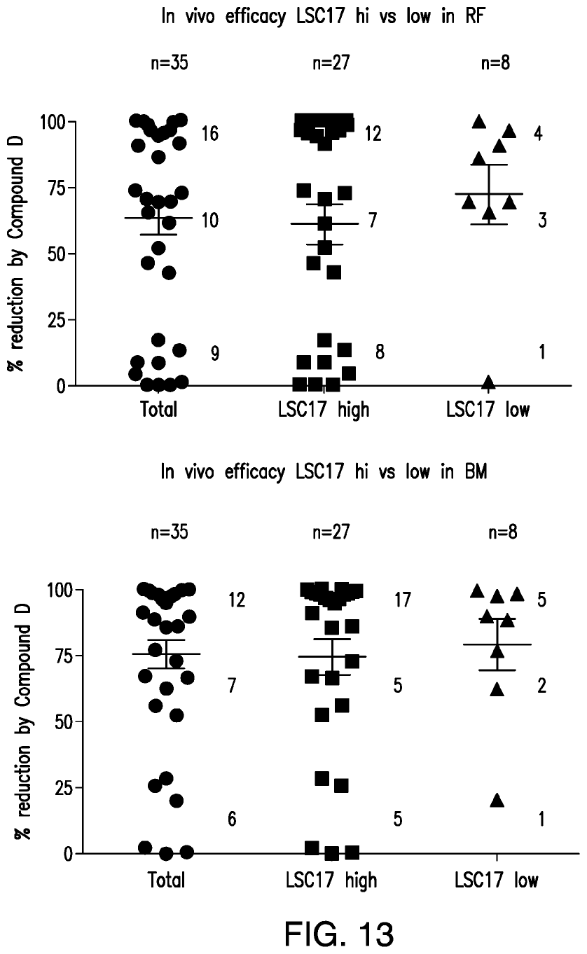

In certain embodiments, Form B is characterized by dynamic vapor sorption analysis. A representative dynamic vapor sorption (DVS) isotherm plot is shown in FIG. 13 of US Publication No. 2019/0030018. In certain embodiments, when the relative humidity (“RH”) is increased from about 0% to about 90% RH, Form B exhibits about 1.4% w/w water uptake. In certain embodiments, Form B comprises less than 0.1% water as determined in a coulometric Karl Fischer (KF) titrator equipped with an oven sample processor set at 225° C.

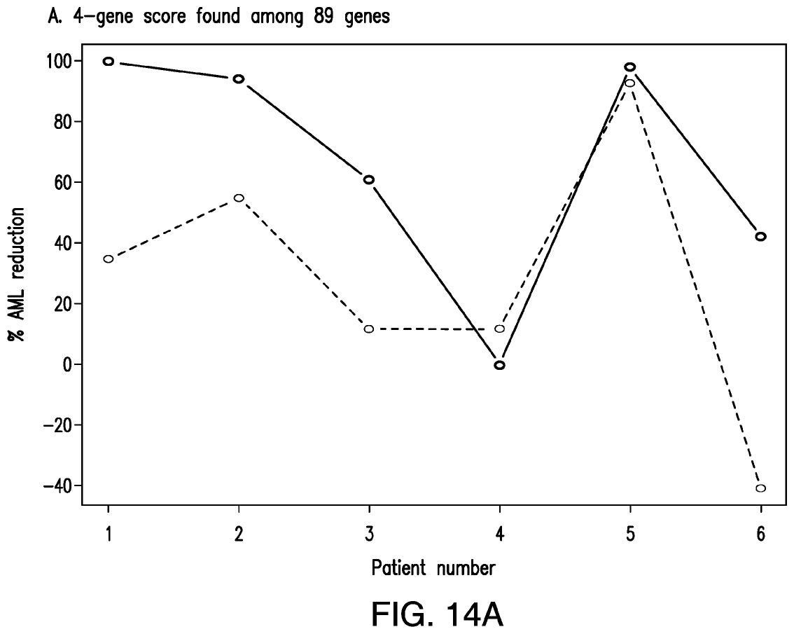

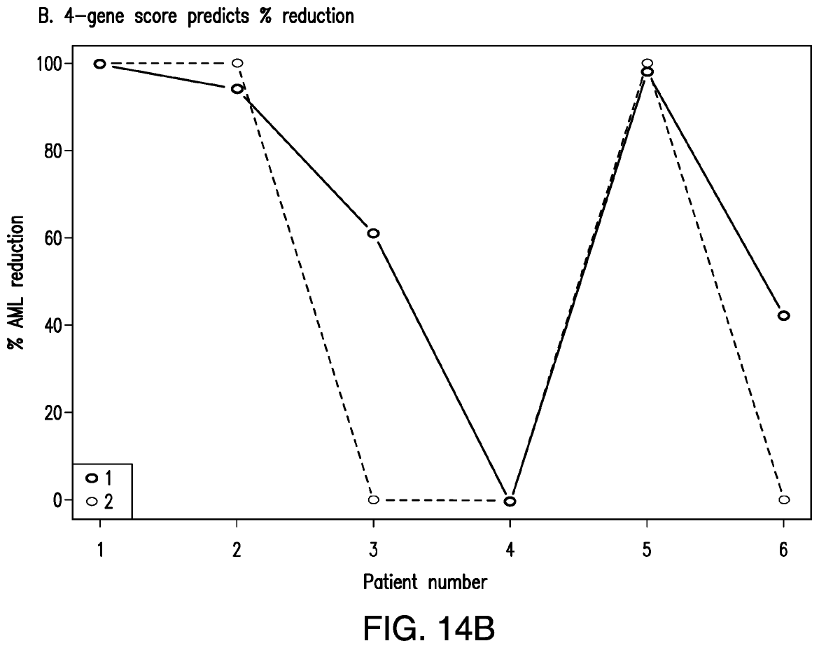

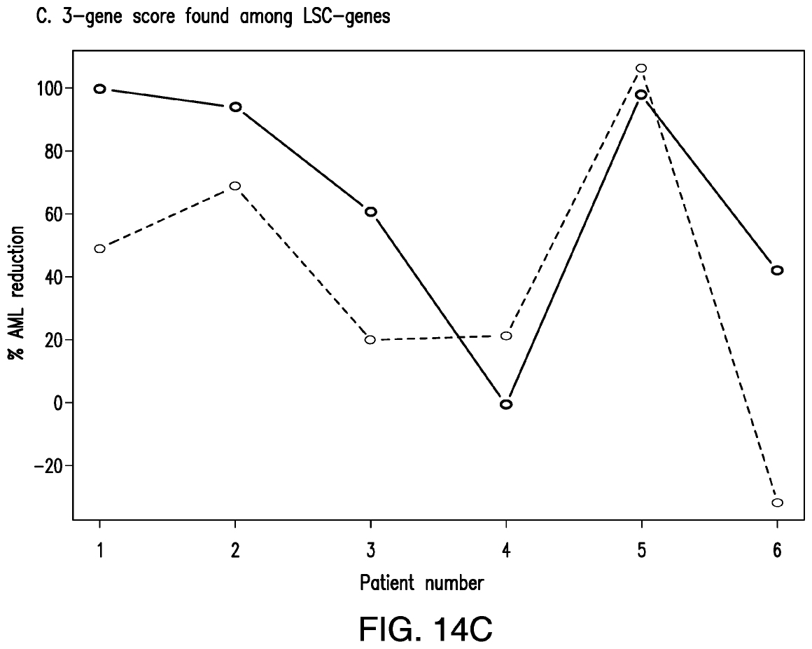

In certain embodiments, Form B shows no significant degradation or residual solvent by ¹H NMR (see FIG. 14 of US Publication No. 2019/0030018).

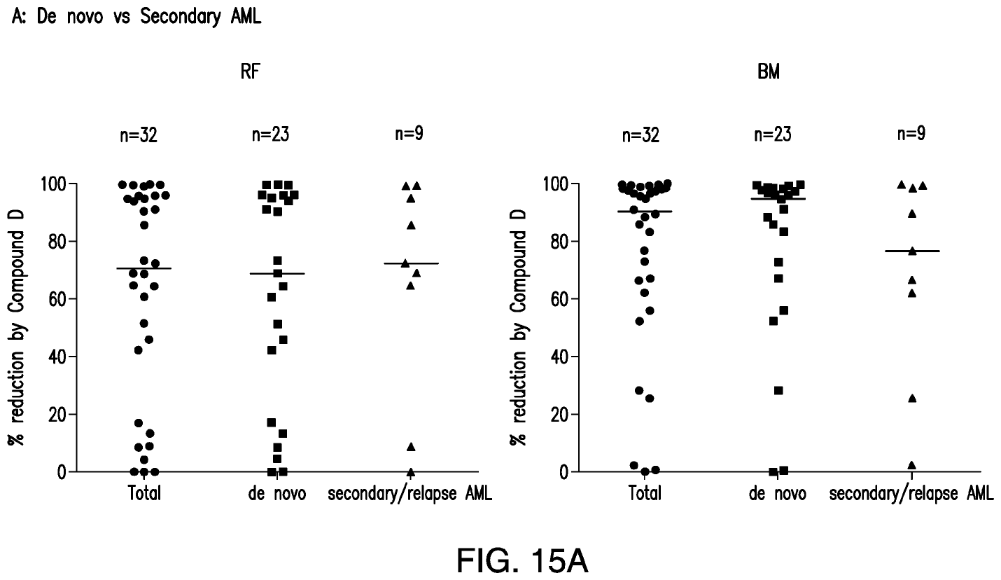

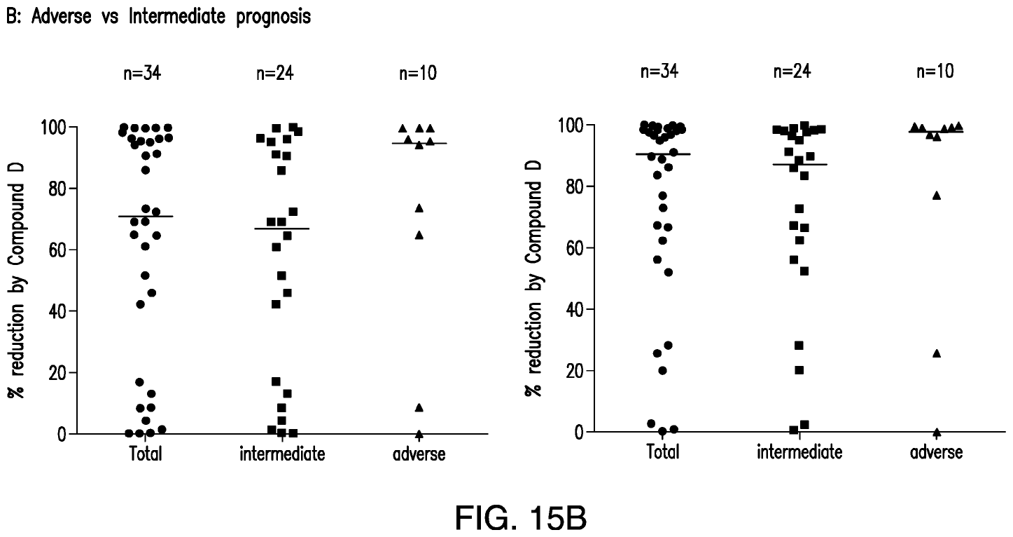

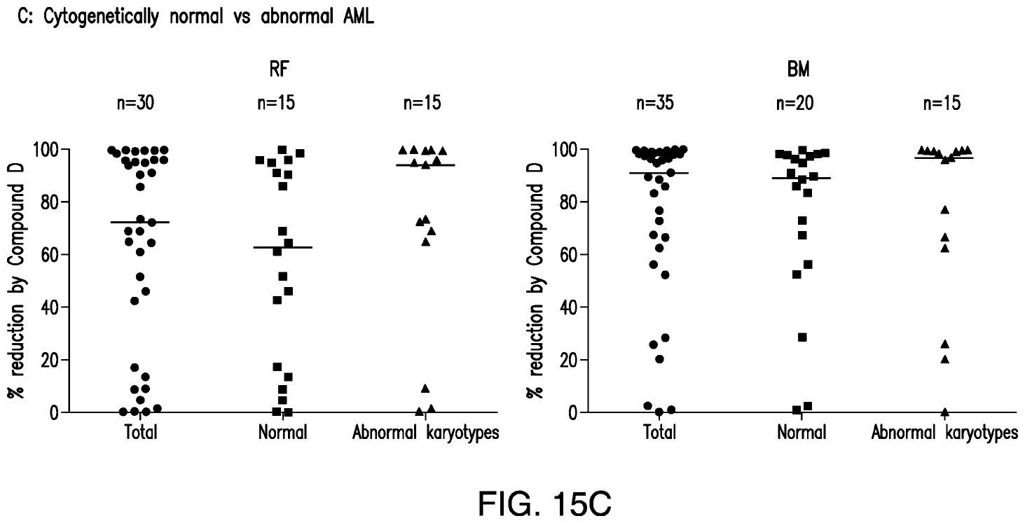

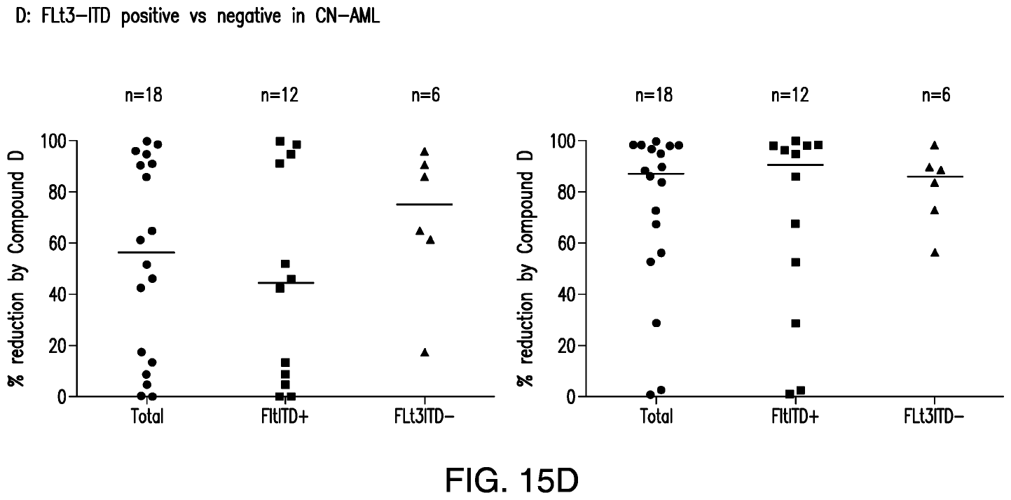

In certain embodiments, Form B of Compound D is characterized by its stability profile upon compression. In certain embodiments, Form B is stable, e.g., its XRPD pattern remains substantially unchanged with broader diffraction peaks, upon application of 2000-psi pressure for about 1 minute (see FIG. 15 of US Publication No. 2019/0030018).

In still another embodiment, Form B of Compound D is substantially pure. In certain embodiments, the substantially pure Form B of Compound D is substantially free of other solid forms, e.g., amorphous form. In certain embodiments, the purity of the substantially pure Form B of Compound D is no less than about 95% pure, no less than about 96% pure, no less than about 97% pure, no less than about 98% pure, no less than about 98.5% pure, no less than about 99% pure, no less than about 99.5% pure, or no less than about 99.8% pure.

Certain embodiments, Form B of Compound D is substantially pure. In certain embodiments, Form B of Compound D is substantially free of other solid forms comprising Compound D including, e.g., Forms A, C, D, E, and/or an amorphous solid form comprising Compound D. In certain embodiments, Form B is a mixture of solid forms comprising Compound D, including, e.g., a mixture comprising one or more of the following: Forms A, C, D, E, and an amorphous solid form comprising Compound D.

### Form C of 2-(4-chlorophenyl)-N-((2-(2,6-dioxopiperidin-3-yl)-1-oxoisoindolin-5-yl)methyl)-2,2-difluoroacetamide

In certain embodiments, the formulations provided herein are prepared from anhydrous Form C of Compound D. In certain embodiments, Form C is the most thermodynamically stable anhydrate among the crystal forms of Compound D.

In certain embodiments, Form C is obtained by slurrying Compound D in certain solvent systems, for example, solvent systems comprising one or more of the following solvents: acetonitrile/water, acetone, or ethanol/water for extended period of time.

In certain aspects, Form C is obtained by slurrying Form B (1×wt) in acetone (30×vol) at an elevated temperature, for example, from 60-80° C. or 70-75° C. for at least 24 hours, and cooling the mixture to room temperature. In one aspect, the slurrying is conducted at a temperature of 70-75° C. under nitrogen pressure of 50-55-psi. In one aspect, the mixture is cooled to room temperature over at least 6 hours.

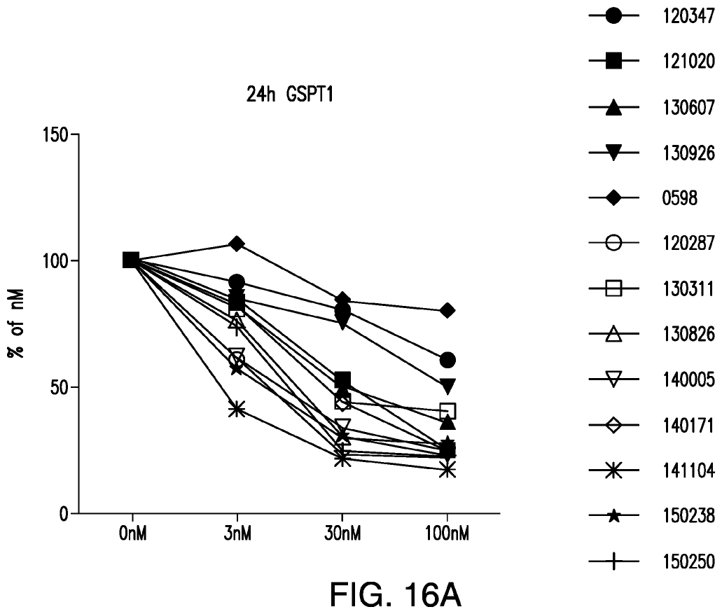

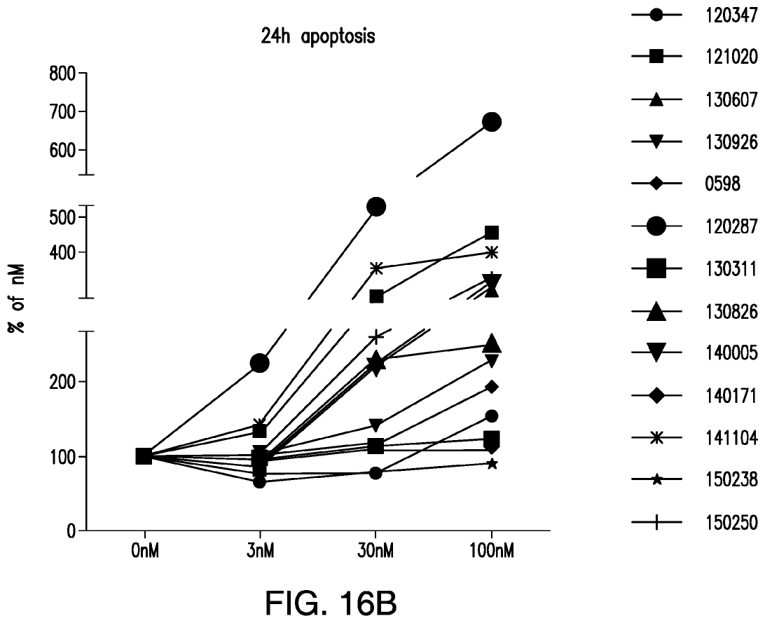

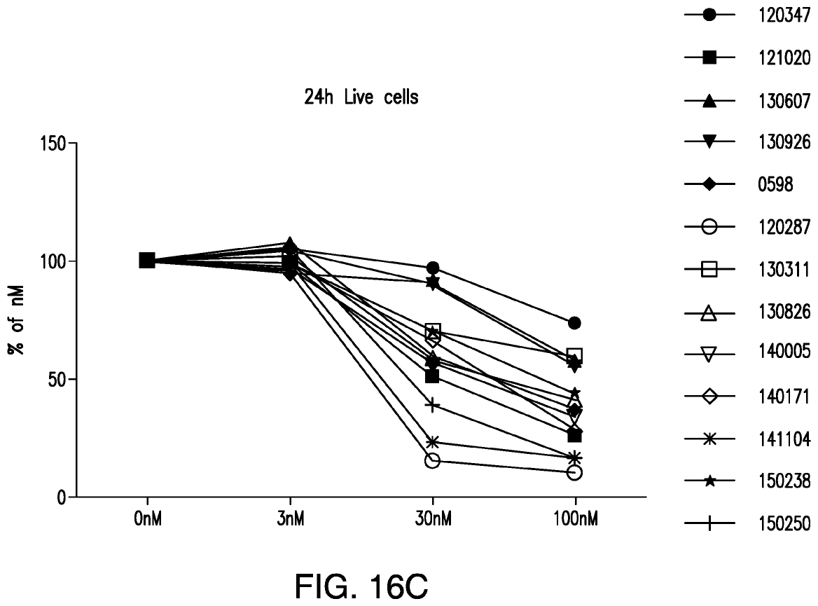

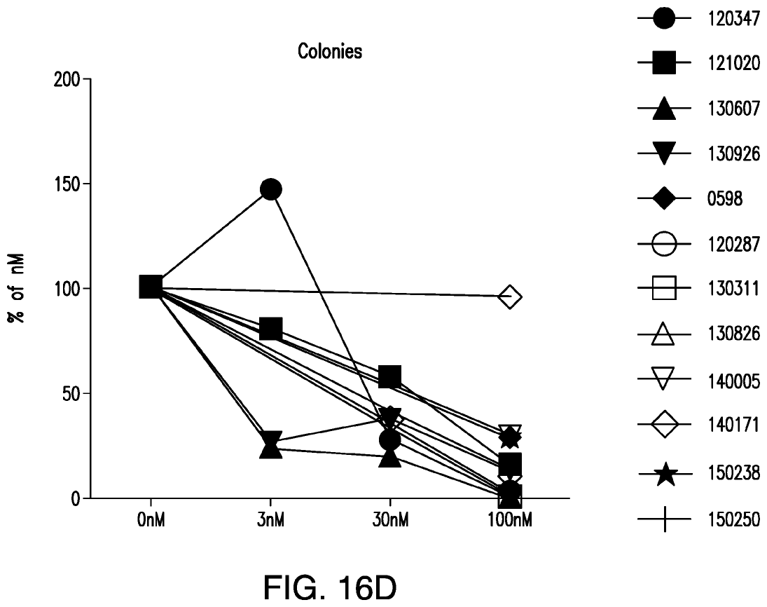

In certain embodiments, Form C is crystalline, as indicated by, e.g., X-ray powder diffraction measurements. In one embodiment, Form C of Compound D has an X-ray powder diffraction pattern substantially as shown in FIG. 16 of US Publication No. 2019/0030018.

In one embodiment, Form C of Compound D has one or more characteristic X-ray powder diffraction peaks at a two-theta angle of approximately 7.4, 11.5, 15.8, 16.7, 16.9, 17.7, 18.4, 19.2, 19.5, 21.1, 23.4, 24.7, or 29.9, degrees 2θ as depicted in FIG. 16 of US Publication No. 2019/0030018. In another embodiment, Form C of Compound D has one, two, three or four characteristic X-ray powder diffraction peaks at a two-theta angle of approximately 16.7, 16.9, 17.7 or 24.7 degrees 2θ. In another embodiment, Form C of Compound D has one, two, three, four, five, six or seven characteristic X-ray powder diffraction peaks as set forth in Table C. In another embodiment, Form C of Compound D has one, two, or three characteristic X-ray powder diffraction peaks as set forth in Table C.

TABLE C

| No. | Pos. [°2 Th.] | d-spacing [Å] | Rel. Int. [%] |
|---|---|---|---|
| 1 | 7.36 | 12.0091 | 32.0 |
| 2 | 9.14 | 9.6750 | 8.3 |
| 3 | 11.51 | 7.6855 | 44.7 |
| 4 | 12.22 | 7.2420 | 4.9 |
| 5 | 15.17 | 5.8398 | 8.4 |
| 6 | 15.82 | 5.6011 | 31.8 |
| 7 | 16.68 | 5.3140 | 57.1 |
| 8 | 16.92 | 5.2392 | 86.8 |
| 9 | 17.72 | 5.0057 | 100.0 |
| 10 | 18.39 | 4.8242 | 21.9 |
| 11 | 19.18 | 4.6268 | 36.4 |
| 12 | 19.45 | 4.5649 | 27.1 |
| 13 | 21.11 | 4.2077 | 40.4 |
| 14 | 21.82 | 4.0724 | 12.4 |
| 15 | 22.28 | 3.9902 | 12.0 |
| 16 | 22.57 | 3.9398 | 17.6 |
| 17 | 23.36 | 3.8082 | 24.7 |
| 18 | 24.26 | 3.6695 | 7.1 |
| 19 | 24.71 | 3.6026 | 72.5 |
| 20 | 25.74 | 3.4615 | 16.9 |
| 21 | 26.03 | 3.4231 | 9.7 |
| 22 | 26.51 | 3.3627 | 17.7 |
| 23 | 27.88 | 3.1998 | 18.0 |
| 24 | 28.70 | 3.1104 | 6.9 |
| 25 | 29.91 | 2.9871 | 30.5 |
| 26 | 30.43 | 2.9375 | 10.7 |
| 27 | 30.83 | 2.9006 | 5.8 |
| 28 | 32.01 | 2.7960 | 16.6 |
| 29 | 37.94 | 2.3718 | 5.5 |

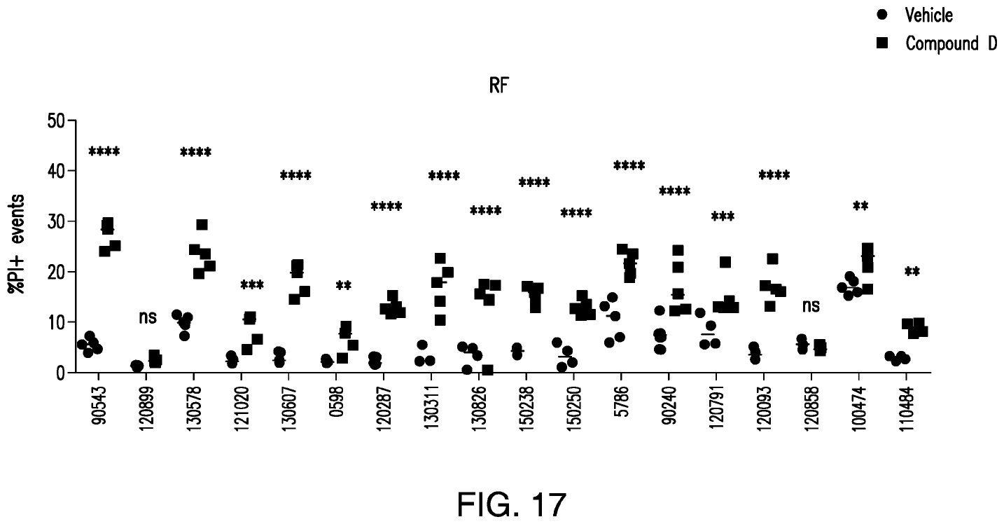

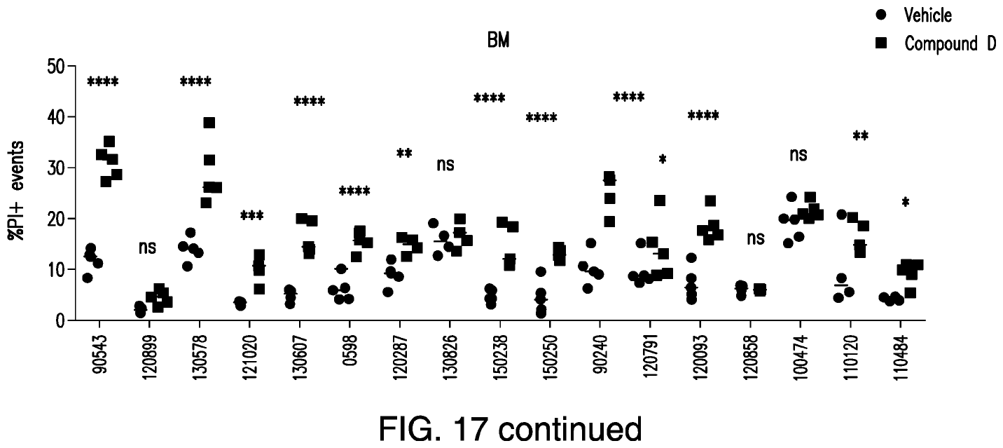

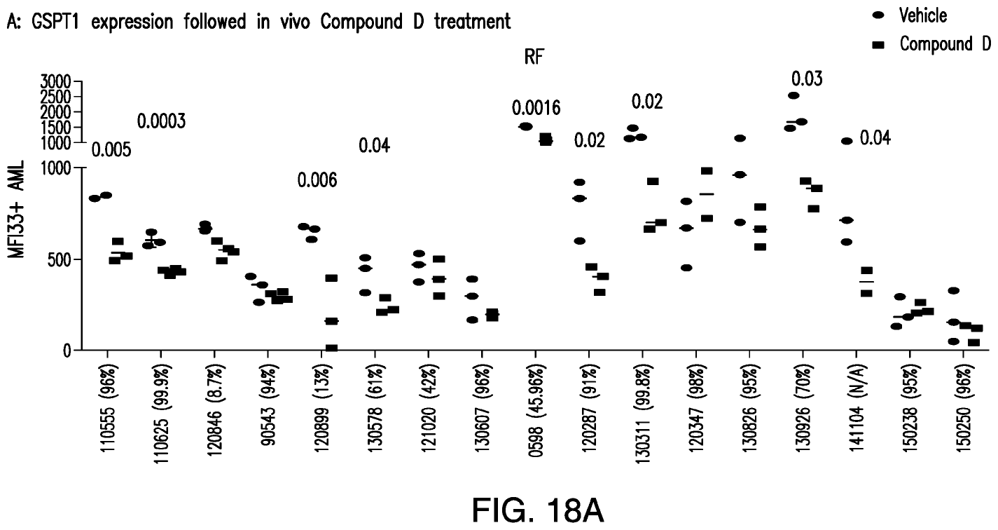

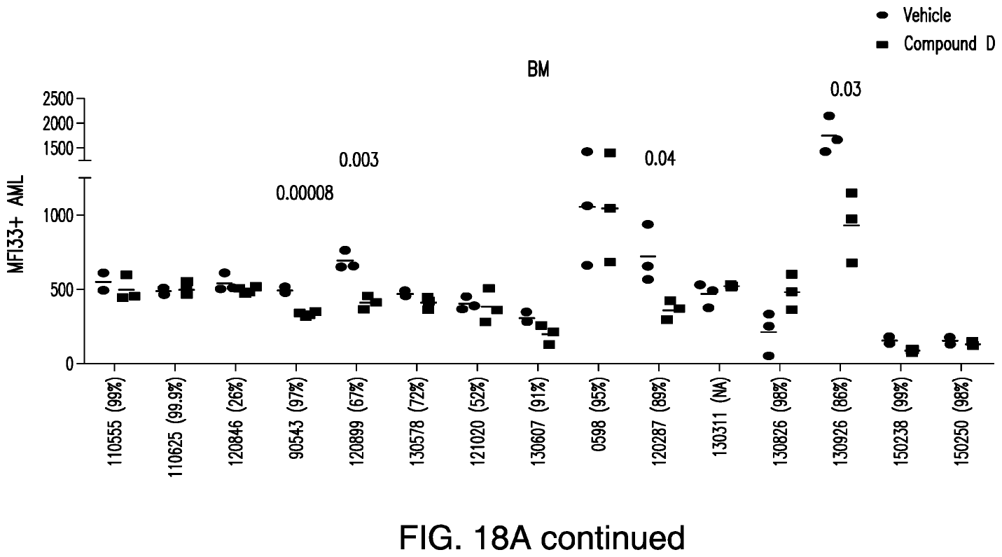

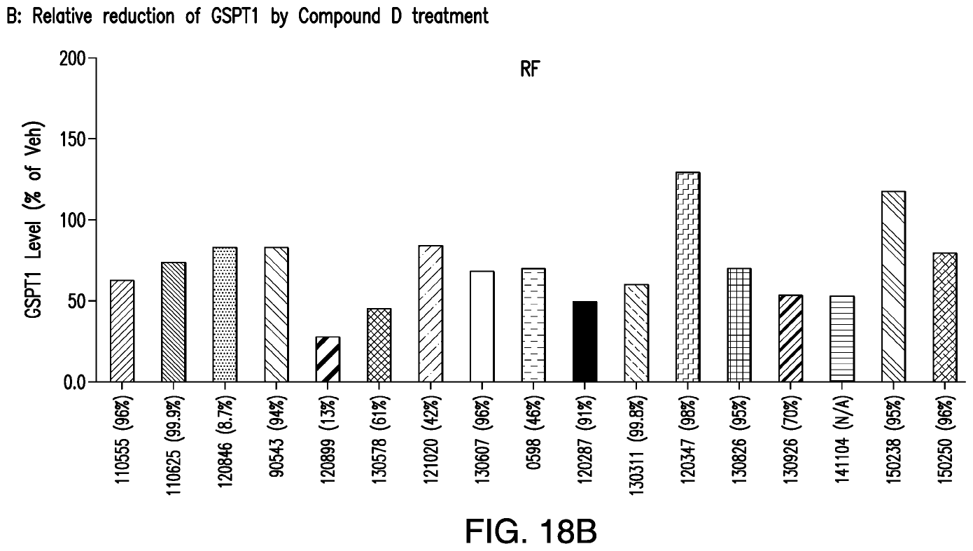

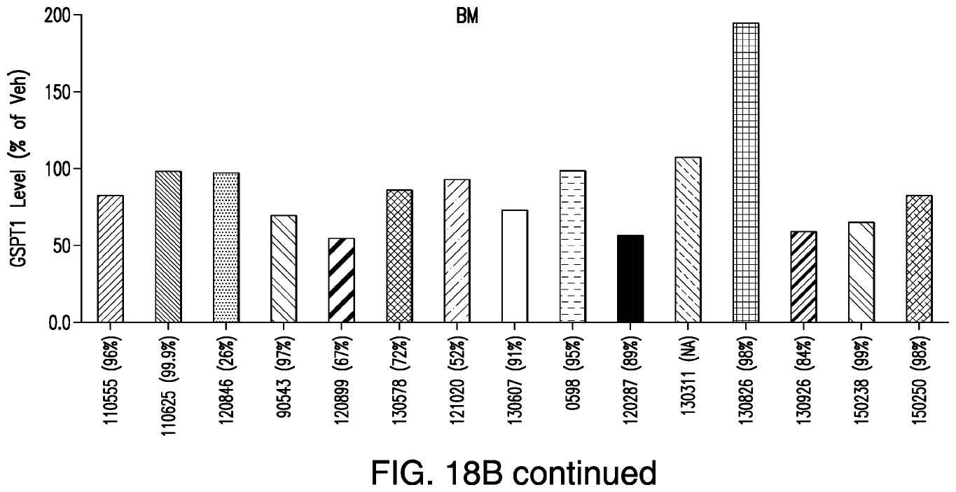

In one embodiment, Form C of Compound D has the SEM picture as shown in FIG. 17 of US Publication No. 2019/0030018. In one embodiment, a crystalline form of Compound D has a thermogravimetric (TGA) thermograph corresponding substantially to the representative TGA thermogram as depicted in FIG. 18 of US Publication No. 2019/0030018. In certain embodiments, Form C shows no TGA weight loss.

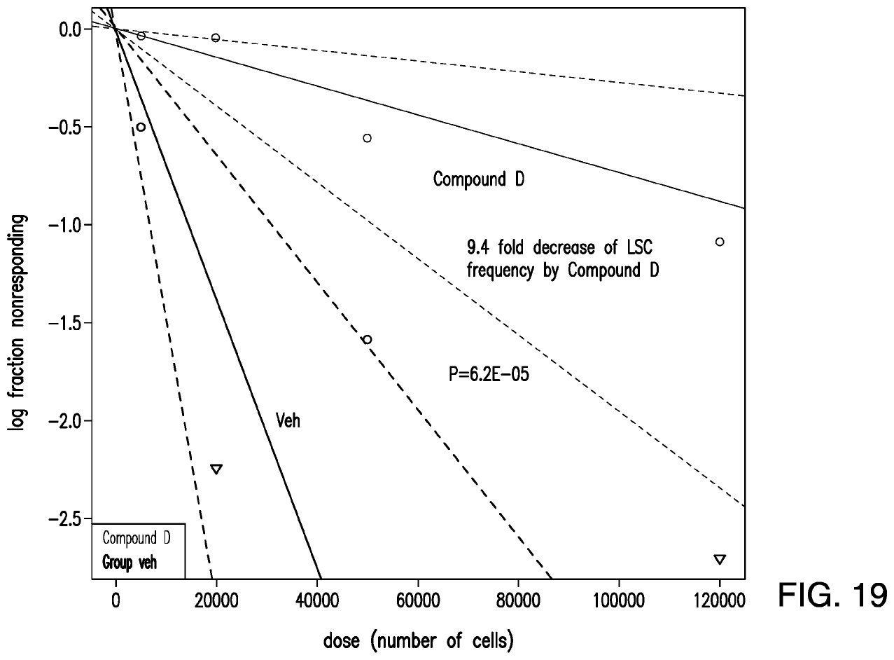

In one embodiment, crystalline Form C of Compound D has a DSC thermogram corresponding substantially as depicted in FIG. 19 of US Publication No. 2019/0030018. In certain embodiments, Form C is characterized by a DSC plot comprising melting event with an onset temperature of 232° C. and heat of fusion of 126 J/g.

In certain embodiments, Form C is characterized by dynamic vapor sorption analysis. A representative dynamic vapor sorption (DVS) isotherm plot is shown in FIG. 20 of US Publication No. 2019/0030018. In certain embodiments, when the relative humidity (“RH”) is increased from about 0% to about 90% RH, Form C exhibits about 0.6% w/w water uptake. In certain embodiments, Form C comprises less than 0.1% water as determined in a coulometric Karl Fischer (KF) titrator equipped with an oven sample processor set at 225° C.

In certain embodiments, Form C shows no significant degradation or residual solvent by ¹H NMR (see FIG. 21 of US Publication No. 2019/0030018).

In certain embodiments, Form C of Compound D is characterized by its stability profile upon compression. In certain embodiments, Form C is stable, e.g., its XRPD pattern remains substantially unchanged with broader diffraction peaks, upon application of 2000-psi pressure for about 1 minute (see FIG. 22 of US Publication No. 2019/0030018).

In still another embodiment, Form C of Compound D is substantially pure. In certain embodiments, the substantially pure Form C of Compound D is substantially free of other solid forms, e.g., amorphous form. In certain embodiments, the purity of the substantially pure Form C of Compound D is no less than about 95% pure, no less than about 96% pure, no less than about 97% pure, no less than about 98% pure, no less than about 98.5% pure, no less than about 99% pure, no less than about 99.5% pure, or no less than about 99.8% pure.

In certain embodiments, Form C of Compound D is substantially pure. In certain embodiments, Form C of Compound D is substantially free of other solid forms comprising Compound D including, e.g., Forms A, B, D, E, and/or an amorphous solid form comprising Compound D. In certain embodiments, Form C is a mixture of solid forms comprising Compound D, including, e.g., a mixture comprising one or more of the following: Forms A, B, D, E, and an amorphous solid form comprising Compound D.

### Form D of 2-(4-chlorophenyl)-N-((2-(2,6-dioxopiperidin-3-yl)-1-oxoisoindolin-5-yl)methyl)-2,2-difluoroacetamide

In certain embodiments, the formulations provided herein are prepared from Form D of Compound D. In certain embodiments, Form D of Compound D is a DMSO solvate.

In certain embodiments, Form D is obtained by heating Form B in DMSO/methyl isobutyl ketone and cooling the solution.

In certain embodiments, Form D is crystalline, as indicated by, e.g., X-ray powder diffraction measurements. In one embodiment, Form D of Compound D has an X-ray powder diffraction pattern substantially as shown in FIG. 23 of US Publication No. 2019/0030018.

In one embodiment, Form D of Compound D has one or more characteristic X-ray powder diffraction peaks at a two-theta angle of approximately 14.1, 14.3, 18.8, 19.1, 23.6 or 24.0 degrees 2θ as depicted in FIG. 23 of US Publication No. 2019/0030018. In another embodiment, Form D of Compound D has one, two, three or four characteristic X-ray powder diffraction peaks at a two-theta angle of approximately 14.1, 14.3, 18.8 or 19.1 degrees 2θ. In another embodiment, Form D of Compound D has one, two, three, four, five, six or seven characteristic X-ray powder diffraction peaks as set forth in Table D. In another embodiment, Form D of Compound D has one, two, or three characteristic X-ray powder diffraction peaks as set forth in Table D.

TABLE D

| | | d-spacing | |
|---|---|---|---|
| No. | Pos. [°2 Th.] | [Å] | Rel. Int. [%] |
| 1 | 4.77 | 18.5435 | 3.0 |
| 2 | 9.57 | 9.2399 | 7.0 |
| 3 | 10.55 | 8.3876 | 3.1 |
| 4 | 11.95 | 7.4070 | 3.7 |
| 5 | 12.50 | 7.0808 | 3.5 |
| 6 | 14.06 | 6.2990 | 100.0 |
| 7 | 14.30 | 6.1927 | 92.9 |
| 8 | 16.13 | 5.4943 | 3.8 |
| 9 | 17.02 | 5.2097 | 8.4 |
| 10 | 17.50 | 5.0676 | 19.8 |
| 11 | 17.78 | 4.9881 | 8.0 |
| 12 | 18.09 | 4.9049 | 7.7 |
| 13 | 18.27 | 4.8561 | 9.0 |
| 14 | 18.75 | 4.7326 | 58.5 |
| 15 | 19.09 | 4.6482 | 63.5 |
| 16 | 21.04 | 4.2228 | 7.3 |
| 17 | 22.77 | 3.9053 | 10.9 |
| 18 | 23.58 | 3.7738 | 53.6 |
| 19 | 24.02 | 3.7045 | 24.6 |
| 20 | 24.90 | 3.5756 | 8.4 |
| 21 | 25.22 | 3.5310 | 10.0 |
| 22 | 26.37 | 3.3796 | 9.4 |
| 23 | 26.63 | 3.3470 | 7.9 |
| 24 | 28.21 | 3.1640 | 5.8 |
| 25 | 29.82 | 2.9958 | 3.0 |
| 26 | 30.16 | 2.9629 | 5.0 |
| 27 | 30.45 | 2.9361 | 6.7 |
| 28 | 32.48 | 2.7566 | 3.3 |
| 29 | 33.03 | 2.7120 | 8.1 |
| 30 | 33.69 | 2.6604 | 3.4 |
| 31 | 35.32 | 2.5413 | 3.0 |
| 32 | 37.96 | 2.3702 | 3.2 |
| 33 | 38.70 | 2.3269 | 3.0 |

In one embodiment, provided herein is a crystalline form of Compound D having a thermogravimetric (TGA) thermograph corresponding substantially to the representative TGA thermogram as depicted in FIG. 24 of US Publication No. 2019/0030018. In certain embodiments, Form D shows TGA weight loss of about 14.1% up to 140° C.

In certain embodiments, Form D comprises DMSO in about 14.3 wt % as measured by gas chromatography.

In still another embodiment, Form D of Compound D is substantially pure. In certain embodiments, the substantially pure Form D of Compound D is substantially free of other solid forms, e.g., amorphous form. In certain embodiments, the purity of the substantially pure Form D of Compound D is no less than about 95% pure, no less than about 96% pure, no less than about 97% pure, no less than about 98% pure, no less than about 98.5% pure, no less than about 99% pure, no less than about 99.5% pure, or no less than about 99.8% pure.

In certain embodiments Form D of Compound D is substantially pure. In certain embodiments, Form D of Compound D is substantially free of other solid forms comprising Compound D including, e.g., Forms A, B, C, E, and/or an amorphous solid form comprising Compound D as provided herein. In certain embodiments, Form D is a mixture of solid forms comprising Compound D, including, e.g., a mixture comprising one or more of the following: Forms A, B, C, E, and an amorphous solid form comprising Compound D.

### Form E of 2-(4-chlorophenyl)-N-((2-(2,6-dioxopiperidin-3-yl)-1-oxoisoindolin-5-yl)methyl)-2,2-difluoroacetamide

In certain embodiments, the formulations provided herein are prepared from Form E of Compound D. In certain embodiments, Form E of Compound D is a DMSO solvate.

In certain embodiments, Form E is obtained from Form C in DMSO/MIBK or DMSO/IPA or DMSO/anisole at room temperature.

In certain embodiments, Form E is crystalline, as indicated by, e.g., X-ray powder diffraction measurements. In one embodiment, Form E of Compound D has an X-ray powder diffraction pattern substantially as shown in FIG. 25 of US Publication No. 2019/0030018.

In one embodiment, Form E of Compound D has one or more characteristic X-ray powder diffraction peaks at a two-theta angle of approximately 10.5, 12.5, 16.1, 17.0, 18.5, 21.2, 21.7, 22.6, 22.9, 23.4, 23.8, 24.1, 25.1 or 26.7, degrees 2θ as depicted in FIG. 25 of US Publication No. 2019/0030018. In another embodiment, Form E of Compound D has one, two, three or four characteristic X-ray powder diffraction peaks at a two-theta angle of approximately 16.1, 17.0, 21.2 or 22.9 degrees 2θ. In another embodiment, Form E of Compound D has one, two, three, four, five, six or seven characteristic X-ray powder diffraction peaks as set forth in Table E. In another embodiment, Form E of Compound D has one, two, or three characteristic X-ray powder diffraction peaks as set forth in Table E.

TABLE E

| | | d-spacing | |
|---|---|---|---|
| No. | Pos. [°2 Th.] | [Å] | Rel. Int. [%] |
| 1 | 4.20 | 21.0329 | 9.6 |
| 2 | 10.48 | 8.4394 | 32.0 |
| 3 | 12.54 | 7.0591 | 28.4 |
| 4 | 14.52 | 6.1023 | 9.9 |
| 5 | 15.51 | 5.7131 | 17.7 |
| 6 | 16.08 | 5.5121 | 100.0 |
| 7 | 16.97 | 5.2256 | 94.5 |
| 8 | 17.77 | 4.9908 | 17.1 |
| 9 | 18.48 | 4.8001 | 20.5 |
| 10 | 19.54 | 4.5422 | 14.7 |
| 11 | 21.15 | 4.2007 | 62.8 |
| 12 | 21.72 | 4.0924 | 20.8 |
| 13 | 22.64 | 3.9270 | 57.4 |
| 14 | 22.91 | 3.8826 | 59.9 |
| 15 | 23.43 | 3.7977 | 23.6 |
| 16 | 23.83 | 3.7348 | 23.2 |
| 17 | 24.13 | 3.6881 | 29.5 |
| 18 | 25.14 | 3.5421 | 35.2 |
| 19 | 26.72 | 3.3362 | 49.5 |
| 20 | 27.68 | 3.2232 | 14.6 |
| 21 | 27.93 | 3.1949 | 15.3 |
| 22 | 28.86 | 3.0942 | 15.6 |
| 23 | 29.08 | 3.0703 | 18.3 |
| 24 | 30.12 | 2.9671 | 7.1 |
| 25 | 30.92 | 2.8923 | 12.8 |
| 26 | 32.35 | 2.7672 | 5.0 |
| 27 | 33.21 | 2.6979 | 6.9 |

In one embodiment, provided herein is a crystalline form of Compound D having a thermogravimetric (TGA) thermograph corresponding substantially to the representative TGA thermogram as depicted in FIG. 26 of US Publication No. 2019/0030018. In certain embodiments, Form E shows TGA weight loss of about 19.4% up to 120° C. In certain embodiments, Form E shows additional weight loss of 24.9% between 120 and 220° C.

In one embodiment, Form E of Compound D is substantially pure. In certain embodiments, the substantially pure Form E of Compound D is substantially free of other solid forms, e.g., amorphous form. In certain embodiments, the purity of the substantially pure Form E of Compound D is no less than about 95% pure, no less than about 96% pure, no less than about 97% pure, no less than about 98% pure, no less than about 98.5% pure, no less than about 99% pure, no less than about 99.5% pure, or no less than about 99.8% pure.

In certain embodiments, Form E of Compound D is substantially pure. In certain embodiments herein, Form E of Compound D is substantially free of other solid forms comprising Compound D including, e.g., Forms A, B, C, D and/or an amorphous solid form comprising Compound D. In certain embodiments, Form E is a mixture of solid forms comprising Compound D, including, e.g., a mixture comprising one or more of the following: Forms A, B, C, D and an amorphous solid form comprising Compound D.

### Amorphous Form of 2-(4-chlorophenyl)-N-((2-(2,6-dioxopiperidin-3-yl)-1-oxoisoindolin-5-yl)methyl)-2,2-difluoroacetamide

In certain embodiments, the formulations provided herein comprise amorphous Compound D.

In certain embodiments, provided herein are methods for making the amorphous form by heating Compound D in THF and water and cooling the solution.

In one embodiment, provided herein is an amorphous solid form of Compound D having a modulated DSC thermogram as depicted in FIG. 27 of US Publication No. 2019/0030018.

In one embodiment, amorphous Compound D has an X-ray powder diffraction pattern substantially as shown in FIG. 28 of US Publication No. 2019/0030018.

In one embodiment, amorphous Compound D has a ¹H NMR spectrum substantially as shown in FIG. 29 of US Publication No. 2019/0030018.

In still another embodiment, amorphous Compound D is substantially pure. In certain embodiments, the substantially pure amorphous Compound D is substantially free of other solid forms, e.g., Form A, Form B, Form C, Form D or Form E. In certain embodiments, the purity of the substantially pure amorphous Compound D is no less than about 95% pure, no less than about 96% pure, no less than about 97% pure, no less than about 98% pure, no less than about 98.5% pure, no less than about 99% pure, no less than about 99.5% pure, or no less than about 99.8% pure.

### Isotopologues of 2-(4-chlorophenyl)-N-((2-(2,6-dioxopiperidin-3-yl)-1-oxoisoindolin-5-yl)methyl)-2,2-difluoroacetamide

Also provided herein are isotopically enriched analogs of 2-(4-chlorophenyl)-N-((2-(2,6-dioxopiperidin-3-yl)-1-oxoisoindolin-5-yl)methyl)-2,2-difluoroacetamide (“isotopologues”) provided herein. Isotopic enrichment (for example, deuteration) of pharmaceuticals to improve pharmacokinetics (“PK”), pharmacodynamics (“PD”), and toxicity profiles, has been demonstrated previously with some classes of drugs. See, for example, Lijinsky et. al., Food Cosmet. Toxicol., 20: 393 (1982); Lijinsky et. al., J. Nat. Cancer Inst., 69: 1127 (1982); Mangold et. al., Mutation Res. 308: 33 (1994); Gordon et. al., Drug Metab. Dispos., 15: 589 (1987); Zello et. al., Metabolism, 43: 487 (1994); Gately et. al., J. Nucl. Med., 27: 388 (1986); Wade D, Chem. Biol. Interact. 117: 191 (1999).

Without being limited by any particular theory, isotopic enrichment of a drug can be used, for example, to (1) reduce or eliminate unwanted metabolites, (2) increase the half-life of the parent drug, (3) decrease the number of doses needed to achieve a desired effect, (4) decrease the amount of a dose necessary to achieve a desired effect, (5) increase the formation of active metabolites, if any are formed, and/or (6) decrease the production of deleterious metabolites in specific tissues and/or create a more effective drug and/or a safer drug for combination therapy, whether the combination therapy is intentional or not.

Replacement of an atom for one of its isotopes often will result in a change in the reaction rate of a chemical reaction. This phenomenon is known as the Kinetic Isotope Effect (“KIE”). For example, if a C—H bond is broken during a rate-determining step in a chemical reaction (i.e. the step with the highest transition state energy), substitution of a deuterium for that hydrogen will cause a decrease in the reaction rate and the process will slow down. This phenomenon is known as the Deuterium Kinetic Isotope Effect (“DKIE”). (See, e.g, Foster et al., Adv. Drug Res., vol. 14, pp. 1-36 (1985); Kushner et al., Can. J. Physiol. Pharmacol., vol. 77, pp. 79-88 (1999)).

The magnitude of the DKIE can be expressed as the ratio between the rates of a given reaction in which a C—H bond is broken, and the same reaction where deuterium is substituted for hydrogen. The DKIE can range from about 1 (no isotope effect) to very large numbers, such as 50 or more, meaning that the reaction can be fifty, or more, times slower when deuterium is substituted for hydrogen. Without being limited by a particular theory, high DKIE values may be due in part to a phenomenon known as tunneling, which is a consequence of the uncertainty principle. Tunneling is ascribed to the small mass of a hydrogen atom, and occurs because transition states involving a proton can sometimes form in the absence of the required activation energy. Because deuterium has more mass than hydrogen, it statistically has a much lower probability of undergoing this phenomenon.

Tritium (“T”) is a radioactive isotope of hydrogen, used in research, fusion reactors, neutron generators and radiopharmaceuticals. Tritium is a hydrogen atom that has 2 neutrons in the nucleus and has an atomic weight close to 3. It occurs naturally in the environment in very low concentrations, most commonly found as T20. Tritium decays slowly (half-life=12.3 years) and emits a low energy beta particle that cannot penetrate the outer layer of human skin. Internal exposure is the main hazard associated with this isotope, yet it must be ingested in large amounts to pose a significant health risk. As compared with deuterium, a lesser amount of tritium must be consumed before it reaches a hazardous level. Substitution of tritium (“T”) for hydrogen results in yet a stronger bond than deuterium and gives numerically larger isotope effects.

Similarly, substitution of isotopes for other elements, including, but not limited to, ¹³C or ¹⁴C for carbon, ³³S, ³⁴S, or ³⁶S for sulfur, ¹⁵N for nitrogen, and ¹⁷O or ¹⁸O for oxygen, will provide a similar kinetic isotope effect.

In certain embodiments, the compound provided herein is a prodrug of a compound provided herein (e.g., a prodrug of Compound D). Exemplary compounds include those disclosed in US Publication No. 2017/0197933, the disclosure of which is incorporated herein by reference in its entirety.

### 5.6. Pharmaceutical Compositions

In some embodiments, the compound provided herein is formulated in a pharmaceutical composition. In some embodiments, Compound D is provided in stable formulations of Compound D. In one embodiment, the formulations of Compound D comprise a solid form of 2-(4-chlorophenyl)-N-((2-(2,6-dioxopiperidin-3-yl)-1-oxoisoindolin-5-yl)methyl)-2,2-difluoroacetamide. In one embodiment, the formulations of Compound D comprise an amorphous form of 2-(4-chlorophenyl)-N-((2-(2,6-dioxopiperidin-3-yl)-1-oxoisoindolin-5-yl)methyl)-2,2-difluoroacetamide.

In certain embodiments, the formulations are prepared with dimethylsulfoxide as a co-solvent or a processing aid. In certain embodiments, the formulations are prepared with formic acid as co-solvent or a processing aid. In certain embodiments, the formulations are prepared without any co-solvent or processing aid.

In certain embodiments, the formulations comprise dimethylsulfoxide as a co-solvent or a processing aid. In certain embodiments, the formulations comprise formic acid as a co-solvent or a processing aid. In certain embodiments, the formulations do not comprise any co-solvent or processing aid.

In certain embodiments, the formulations provided herein are lyophilized formulations. In certain embodiments, the formulations provided herein are reconstituted formulations obtained in a pharmaceutically acceptable solvent to produce a pharmaceutically acceptable solution.

Formulation Ia

In one embodiment, provided herein are formulations comprising Compound D in an amount of about 0.05-0.2%, a citrate buffer in an amount of about 3%-6%, and hydroxypropyl β-cyclodextrin (HPBCD) in an amount of about 92-98% based on total weight of the formulation.

In one embodiment, provided herein are formulations comprising Compound D in an amount of about 0.05-0.2%, a citrate buffer in an amount of about 3%-6%, and sulfobutyl ether-beta-cyclodextrin in an amount of about 92-98% based on total weight of the formulation.

In one embodiment, provided herein are formulations comprising Compound D in an amount of about 0.05-0.2%, a citrate buffer in an amount of about 3%-6%, HPBCD in an amount of about 92-98%, and no more than about 1% dimethyl sulfoxide based on total weight of the formulation.

In one embodiment, provided herein are formulations comprising Compound D in an amount of about 0.05-0.2%, a citrate buffer in an amount of about 3%-6%, sulfobutyl ether-beta-cyclodextrin in an amount of about 92-98%, and no more than about 1% dimethyl sulfoxide based on total weight of the formulation.

In one embodiment, provided herein are formulations comprising Compound D in an amount of about 0.08-0.15%, a citrate buffer in an amount of about 3%-6%, and HPBCD in an amount of about 94-96%, based on total weight of the formulation.

In one embodiment, provided herein are formulations comprising Compound D in an amount of about 0.08-0.15%, a citrate buffer in an amount of about 3%-6%, and sulfobutyl ether-beta-cyclodextrin in an amount of about 94-96%, and based on total weight of the formulation.

In one embodiment, provided herein are formulations comprising Compound D in an amount of about 0.08-0.15%, a citrate buffer in an amount of about 3%-6%, HPBCD in an amount of about 94-96%, and no more than about 1% dimethyl sulfoxide based on total weight of the formulation.

In one embodiment, provided herein are formulations comprising Compound D in an amount of about 0.08-0.15%, a citrate buffer in an amount of about 3%-6%, sulfobutyl ether-beta-cyclodextrin in an amount of about 94-96%, and no more than about 1% dimethyl sulfoxide based on total weight of the formulation.

In one aspect, the formulation provided herein comprises Compound D in an amount of about 0.08 to about 0.15% based on the total weight of the formulation. In certain embodiments, the amount of Compound D is from about 0.09% to about 0.15%, about 0.1% to about 0.13% or about 0.11% to about 0.12% based on the total weight of the formulation. In certain embodiments, the amount of Compound D is about 0.05%, 0.07%, 0.09%, 0.11%, 0.12%, 0.13%, or 0.15% based on the total weight of the formulation. In one embodiment, the amount of Compound D in the formulation is about 0.12% based on the total weight of the formulation.

In another aspect, provided herein is a formulation that comprises Compound D in an amount of about 0.5 mg to about 2 mg in a 20-cc vial. In still another aspect is a formulation that comprises Compound D in an amount of about 0.5 mg to about 1.5 mg, about 0.75 mg to about 1.25 mg, or about 0.8 mg to about 1.1 mg in a 20-cc vial. In one aspect Compound D is present in an amount of about 0.7, 0.75, 0.76, 0.8, 0.9, 1.0, 1.05 or 1.2 mg in a 20-cc vial. In one aspect Compound D is present in an amount of about 1.05 mg in a 20-cc vial.

In one aspect, the formulations provided herein contain a citrate buffer. In one aspect, the amount of citrate buffer in the formulations provided herein is from about 3% to about 6% based on total weight of the formulation. In one aspect, the amount of citrate buffer in the formulations provided herein is about 3%, 3.5%, 4%, 4.2%, 4.5% or 5% based on total weight of the formulation. In one aspect, the amount of citrate buffer in the formulations provided herein is about 4.2% based on total weight of the formulation. In one aspect, the amount of citrate buffer in the formulations provided herein is about 37 mg in a 20 cc vial.

In one embodiment, the citrate buffer comprises anhydrous citric acid and anhydrous sodium citrate. In certain embodiments, the amount of anhydrous citric acid is from about 1.5% to about 3%, about 1.75% to about 2.75%, or about 2% to about 2.5% based on total weight of the formulation. In certain embodiments, the amount of anhydrous citric acid in the formulation is about 1.5%, 1.75%, 2%, 2.1%, or 2.5% based on total weight of the formulation. In one embodiment, the amount of anhydrous citric acid in the formulation is about 2%, 2.1%, 2.22% or 2.3% based on total weight of the formulation. In one embodiment, the amount of anhydrous citric acid in the formulation is about 2.10% based on total weight of the formulation.

In still another aspect is a formulation that comprises anhydrous citric acid in an amount of about 16 mg to about 20 mg in a 20-cc vial. In one embodiment, the amount of anhydrous citric acid is about 16, 17, 18, 18.2, 18.4, 18.6, 18.8, 19 or 20 mg in a 20-cc vial. In one embodiment, the amount of anhydrous citric acid is about 18.6 mg in a 20-cc vial.

In certain embodiments, the amount of anhydrous sodium citrate is from about 1.5% to about 3%, about 1.75% to about 2.75%, or about 2% to about 2.5% based on total weight of the formulation. In certain embodiments, the amount of anhydrous sodium citrate in the formulation is about 1.5%, 1.75%, 2%, 2.1%, or 2.5% based on total weight of the formulation. In one embodiment, the amount of anhydrous sodium citrate in the formulation is about 2%, 2.05%, 2.08% or 2.1% based on total weight of the formulation. In one embodiment, the amount of anhydrous sodium citrate in the formulation is about 2.08% based on total weight of the formulation.

In still another aspect is a formulation that comprises anhydrous sodium citrate in an amount of about 16 mg to about 20 mg in a 20-cc vial. In one embodiment, the amount of anhydrous sodium citrate is about 16, 17, 18, 18.2, 18.4, 18.6, 18.8, 19 or 20 mg in a 20-cc vial. In one embodiment, the amount of anhydrous sodium citrate is about 18.4 mg in a 20-cc vial.

In certain embodiments, the amount of HPBCD in the formulations provided herein is about 94 to about 97% based on total weight of the formulation. In one embodiment, the amount of HPBCD in the formulations provided herein is about 94.5%, 95%, 95.5%, or 96% based on total weight of the formulation. In one embodiment, the amount of HPBCD in the formulations provided herein is about 95% based on total weight of the formulation.

In certain embodiments, the amount of sulfobutyl ether-beta-cyclodextrin in the formulations provided herein is about 94 to about 97% based on total weight of the formulation. In one embodiment, the amount of sulfobutyl ether-beta-cyclodextrin in the formulations provided herein is about 94.5%, 95%, 95.5%, or 96% based on total weight of the formulation. In one embodiment, the amount of sulfobutyl ether-beta-cyclodextrin in the formulations provided herein is about 95% based on total weight of the formulation.

In another aspect is a formulation that comprises HPBCD in an amount of about 800 to 900 mg in a 20-cc vial. In another aspect is a formulation that comprises HPBCD in an amount of about 810 to 880 mg, 820 to 860 mg or 830 to 850 mg in a 20-cc vial. In another aspect is a formulation that comprises HPBCD in an amount of about 840 mg in a 20-cc vial.

In another aspect is a formulation that comprises sulfobutyl ether-beta-cyclodextrin in an amount of about 800 to 900 mg in a 20-cc vial. In another aspect is a formulation that comprises sulfobutyl ether-beta-cyclodextrin in an amount of about 810 to 880 mg, 820 to 860 mg or 830 to 850 mg in a 20-cc vial. In another aspect is a formulation that comprises sulfobutyl ether-beta-cyclodextrin in an amount of about 840 mg in a 20-cc vial.

In another aspect is a formulation that comprises Kleptose®HPB in an amount of about 840 mg in a 20-cc vial.

In one embodiment, the formulations comprise dimethyl sulfoxide in an amount of no more than about 1.5% based on total weight of the formulation. In one embodiment, the formulations comprise dimethyl sulfoxide in an amount of up to 0.1%, 0.2%, 0.3%, 0.4%, 0.6%, 0.7%, 0.8%, 0.9% or 1% based on total weight of the formulation. In one embodiment, the formulations comprise no more than about 0.1%, 0.2%, 0.3%, 0.4%, 0.6%, 0.7%, 0.8%, 0.9% or 1% dimethyl sulfoxide based on total weight of the formulation. In one embodiment, the formulations comprise dimethyl sulfoxide in an amount of up to about 0.1 to about 1.5% based on total weight of the formulation. In one embodiment, the amount of dimethyl sulfoxide in the formulations provided herein is about 0.1 to about 1.3% based on total weight of the formulation. In one embodiment, the amount of dimethyl sulfoxide in the formulations provided herein is about 0.1%, 0.2%, 0.3%, 0.4%, 0.6%, 0.7%, 0.8%, 0.9% or 1% based on total weight of the formulation. In one embodiment, the formulations provided herein do not contain any dimethyl sulfoxide. In one embodiment, the amount of dimethyl sulfoxide in the formulations provided herein is about 0.4% to 0.8% based on total weight of the formulation.

In another aspect is a formulation that comprises dimethyl sulfoxide in an amount of about 4 to 7 mg in a 20-cc vial. In another aspect is a formulation that comprises dimethyl sulfoxide in an amount of about 4.5 to 6.5 mg, or 5 to 6 mg in a 20-cc vial.

In certain embodiments, the formulation provided herein is lyophilized, and the lyophilized formulation upon reconstitution has a pH of about 4 to 5. In certain embodiments, the formulation upon reconstitution has a pH of about 4.2 to 4.4. In one embodiment, the lyophilized formulation upon reconstitution has a pH of about 4, 4.1, 4.2, 4.3, 4.4, 4.5, 4.6, 4.7, 4.8, 4.9 or 5.

In certain embodiments, the lyophilized formulation upon reconstitution has an osmolality of about 250-290 mOsm/kg. In certain embodiments, the lyophilized formulation upon reconstitution has an osmolality of about 260-280 mOsm/kg.

In certain embodiments, provided herein is a container comprising a formulation provided herein. In one aspect, the container is a glass vial. In one aspect, the container is a 20-cc glass vial.

In one aspect provided herein is a formulation in a 20-cc vial that comprises: Compound D at an amount that provides 1.05 mg 2-(4-chlorophenyl)-N-((2-(2,6-dioxopiperidin-3-yl)-1-oxoisoindolin-5-yl)methyl)-2,2-difluoroacetamide and a pharmaceutically acceptable carrier or excipient that includes a bulking agent as described herein. In one embodiment, the formulation further comprises no more than about 7 mg dimethyl sulfoxide as residual solvent. In one embodiment, the formulation comprises no more than about 6 mg dimethyl sulfoxide as residual solvent. In one embodiment, the formulation comprises no more than about 5 mg dimethyl sulfoxide as residual solvent. In one embodiment, the formulation comprises no more than about 4 mg dimethyl sulfoxide as residual solvent. In one embodiment, the formulation comprises from about 3 mg to about 7 mg, about 4 mg to about 6 mg, about 4 mg to about 5 mg or about 5 mg to about 6 mg dimethyl sulfoxide as residual solvent. In one embodiment, the formulation comprises about 4, 4.5, 5, 5.3, 5.5, 5.7, 6 or 6.5 mg dimethyl sulfoxide as residual solvent.

In one embodiment, provided herein are formulations consisting essentially of Compound D in an amount of about 0.05-0.2%, a citrate buffer in an amount of about 3%-6%, and HPBCD in an amount of about 92-98% based on total weight of the formulation.

In one embodiment, provided herein are formulations consisting essentially of Compound D in an amount of about 0.05-0.2%, a citrate buffer in an amount of about 3%-6%, and sulfobutyl ether-beta-cyclodextrin in an amount of about 92-98% based on total weight of the formulation.

In one embodiment, provided herein are formulations consisting essentially of Compound D in an amount of about 0.05-0.2%, a citrate buffer in an amount of about 3%-6%, HPBCD in an amount of about 92-98%, and no more than about 1% dimethyl sulfoxide based on total weight of the formulation.

In one embodiment, provided herein are formulations consisting essentially of Compound D in an amount of about 0.05-0.2%, a citrate buffer in an amount of about 3%-6%, sulfobutyl ether-beta-cyclodextrin in an amount of about 92-98%, and no more than about 1% dimethyl sulfoxide based on total weight of the formulation.

In one aspect provided herein is a formulation in a 20-cc vial that comprises: Compound D at an amount that provides 1.05 mg 2-(4-chlorophenyl)-N-((2-(2,6-dioxopiperidin-3-yl)-1-oxoisoindolin-5-yl)methyl)-2,2-difluoroacetamide, a pharmaceutically acceptable carrier or excipient that includes a buffer and bulking agent as described herein, and about 5 mg to about 6 mg dimethyl sulfoxide as residual solvent. The buffer and bulking agent can be present at an amount as described herein.

In one aspect provided herein is a formulation in a 20-cc vial that comprises: Compound D at an amount that provides 1.05 mg 2-(4-chlorophenyl)-N-((2-(2,6-dioxopiperidin-3-yl)-1-oxoisoindolin-5-yl)methyl)-2,2-difluoroacetamide, 18.6 mg anhydrous citric acid, 18.4 mg anhydrous sodium citrate, 840 mg HPBCD, and about 5 mg to about 6 mg dimethyl sulfoxide as residual solvent as described herein. In one embodiment, the formulation in a 20-cc vial is reconstituted with 3.8 mL sterile water for injection.

In one aspect provided herein is a formulation in a 20-cc vial that consists essentially of: Compound D at an amount that provides 1.05 mg 2-(4-chlorophenyl)-N-((2-(2,6-dioxopiperidin-3-yl)-1-oxoisoindolin-5-yl)methyl)-2,2-difluoroacetamide, 18.6 mg anhydrous citric acid, 18.4 mg anhydrous sodium citrate, 840 mg HPBCD, and about 5 mg to about 6 mg dimethyl sulfoxide as residual solvent as described herein. In one embodiment, the formulation in a 20-cc vial is reconstituted with 3.8 mL sterile water for injection.

In one aspect provided herein is a formulation in a 20-cc vial that consists of: Compound D at an amount that provides 1.05 mg 2-(4-chlorophenyl)-N-((2-(2,6-dioxopiperidin-3-yl)-1-oxoisoindolin-5-yl)methyl)-2,2-difluoroacetamide, 18.6 mg anhydrous citric acid, 18.4 mg anhydrous sodium citrate, 840 mg HPBCD, and about 5 mg to about 6 mg dimethyl sulfoxide as residual solvent as described herein. In one embodiment, the formulation in a 20-cc vial is reconstituted with 3.8 mL sterile water for injection.

In one embodiment, provided herein is an aqueous formulation comprising Compound D in an amount of about 0.05-0.2% based on total weight of the solids, a citrate buffer in an amount of about 3%-6% based on total weight of the solids, HPBCD in an amount of about 92-98% based on total weight of the solids, and a diluent.

In one embodiment, provided herein is an aqueous formulation consisting essentially of Compound D in an amount of about 0.05-0.2% based on total weight of the solids, a citrate buffer in an amount of about 3%-6% based on total weight of the solids, HPBCD in an amount of about 92-98% based on total weight of the solids, and a diluent.

In one aspect provided herein is an aqueous formulation that comprises: Compound D at an amount that provides 1.05 mg 2-(4-chlorophenyl)-N-((2-(2,6-dioxopiperidin-3-yl)-1-oxoisoindolin-5-yl)methyl)-2,2-difluoroacetamide, 18.6 mg anhydrous citric acid, 18.4 mg anhydrous sodium citrate, 840 mg HPBCD, and about 5 mg to about 6 mg dimethyl sulfoxide as residual solvent and about 3.8 mL diluent.

In one aspect provided herein is an aqueous formulation that consists essentially of: Compound D at an amount that provides 1.05 mg 2-(4-chlorophenyl)-N-((2-(2,6-dioxopiperidin-3-yl)-1-oxoisoindolin-5-yl)methyl)-2,2-difluoroacetamide, 18.6 mg anhydrous citric acid, 18.4 mg anhydrous sodium citrate, 840 mg HPBCD, and about 5 mg to about 6 mg dimethyl sulfoxide as residual solvent and about 3.8 mL diluent.

In one aspect provided herein is an aqueous formulation that consists of: Compound D at an amount that provides 1.05 mg 2-(4-chlorophenyl)-N-((2-(2,6-dioxopiperidin-3-yl)-1-oxoisoindolin-5-yl)methyl)-2,2-difluoroacetamide, 18.6 mg anhydrous citric acid, 18.4 mg anhydrous sodium citrate, 840 mg HPBCD, and about 5 mg to about 6 mg dimethyl sulfoxide as residual solvent and about 3.8 mL diluent.

Formulation Ib

In one embodiment, provided herein are formulations comprising Compound D in an amount of about 0.01-0.15%, hydroxypropyl β-cyclodextrin in an amount of about 99.1-99.99%. In one embodiment, provided herein are formulations comprising Compound D in an amount of about 0.01-0.15%, hydroxypropyl β-cyclodextrin in an amount of about 99.1-99.99%, and no more than about 0.5% formic acid based on total weight of the formulation.

In one embodiment, provided herein are formulations comprising Compound D in an amount of about 0.05-0.25% and HPBCD in an amount of about 99.1-99.9% based on total weight of the formulation.

In one embodiment, provided herein are formulations comprising Compound D in an amount of about 0.05-0.25%, HPBCD in an amount of about 99.1-99.9%, and no more than about 0.5% formic acid based on total weight of the formulation.

In one embodiment, provided herein are formulations comprising Compound D in an amount of about 0.05-0.25% and HPBCD in an amount of about 99.75-99.9% based on total weight of the formulation.

In one embodiment, provided herein are formulations comprising Compound D in an amount of about 0.05-0.25%, HPBCD in an amount of about 99.75-99.9%, and no more than about 0.5% formic acid based on total weight of the formulation.

In one embodiment, provided herein are formulations comprising Compound D in an amount of about 0.05-0.25%, HPBCD in an amount of about 99.75-99.9%, and no more than about 0.2% formic acid based on total weight of the formulation.

In one embodiment, provided herein are formulations comprising Compound D in an amount of about 0.08-0.15% and HPBCD in an amount of about 99.8-99.9% based on total weight of the formulation.

In one embodiment, provided herein are formulations comprising Compound D in an amount of about 0.08-0.15%, HPBCD in an amount of about 99.8-99.9%, and no more than about 0.5% formic acid based on total weight of the formulation.

In one embodiment, provided herein are formulations comprising Compound D in an amount of about 0.08-0.15%, HPBCD in an amount of about 99.8-99.9%, and no more than about 0.12% formic acid based on total weight of the formulation.

In one embodiment, provided herein are formulations comprising Compound D in an amount of about 0.12% and HPBCD in an amount of about 99.88% based on total weight of the formulation.

In one embodiment, provided herein are formulations comprising Compound D in an amount of about 0.05-0.25% and sulfobutyl ether-beta-cyclodextrin in an amount of about 99.1-99.9%, based on total weight of the formulation.

In one embodiment, provided herein are formulations comprising Compound D in an amount of about 0.05-0.25%, sulfobutyl ether-beta-cyclodextrin in an amount of about 99.1-99.9%, and no more than about 0.5% formic acid based on total weight of the formulation.

In one embodiment, provided herein are formulations comprising Compound D in an amount of about 0.05-0.25% and sulfobutyl ether-beta-cyclodextrin in an amount of about 99.75-99.9%, based on total weight of the formulation.

In one embodiment, provided herein are formulations comprising Compound D in an amount of about 0.08-0.15% and sulfobutyl ether-beta-cyclodextrin in an amount of about 99.8-99.9% based on total weight of the formulation.

In one embodiment, provided herein are formulations comprising Compound D in an amount of about 0.08-0.15%, sulfobutyl ether-beta-cyclodextrin in an amount of about 99.8-99.9%, and no more than about 0.5% formic acid based on total weight of the formulation.

In one embodiment, provided herein are formulations comprising Compound D in an amount of about 0.12% and sulfobutyl ether-beta-cyclodextrin in an amount of about 99.88% based on total weight of the formulation.

In one aspect, the formulation provided herein comprises Compound D in an amount of about 0.08 to about 0.15% based on the total weight of the formulation. In certain embodiments, the amount of Compound D is from about 0.09% to about 0.15%, about 0.1% to about 0.13% or about 0.11% to about 0.12% based on the total weight of the formulation. In certain embodiments, the amount of Compound D is about 0.05%, 0.07%, 0.09%, 0.11%, 0.12%, 0.13%, or 0.15% based on the total weight of the formulation. In one embodiment, the amount of Compound D in the formulation is about 0.12% based on the total weight of the formulation.

In another aspect, provided herein is a formulation that comprises Compound D in an amount of about 0.5 mg to about 2 mg in a 20-cc vial. In still another aspect is a formulation that comprises Compound D in an amount of about 0.5 mg to about 1.5 mg, about 0.75 mg to about 1.25 mg, or about 0.8 mg to about 1.1 mg in a 20-cc vial. In one aspect Compound D is present in an amount of about 0.7, 0.75, 0.76, 0.8, 0.9, 1.0, 1.05 or 1.2 mg in a 20-cc vial. In one aspect Compound D is present in an amount of about 1 mg in a 20-cc vial.

In one embodiment, the amount of HPBCD in the formulations provided herein is about 97% to about 99.9% based on total weight of the formulation. In one embodiment, the amount of HPBCD in the formulations provided herein is about 98% to about 99.9% based on total weight of the formulation. In one embodiment, the amount of HPBCD in the formulations provided herein is about 99.1%, 99.3%, 99.5%, 99.7% or 99.9% based on total weight of the formulation. In one embodiment, the amount of HPBCD in the formulations provided herein is about 99.5% based on total weight of the formulation. In another aspect is a formulation that comprises HPBCD in an amount of about 750-850 mg in a 20-cc vial. In another aspect is a formulation that comprises HPBCD in an amount of about 790 to 840 mg, 780 to 830 mg or 790 to 810 mg in a 20-cc vial. In another aspect is a formulation that comprises HPBCD in an amount of about 800 mg in a 20-cc vial.

In another aspect is a formulation that comprises Kleptose HPB in an amount of about 800 mg in a 20-cc vial.

In one embodiment, the amount of sulfobutyl ether-beta-cyclodextrin in the formulations provided herein is about 97 to about 99.9% based on total weight of the formulation. In one embodiment, the amount of sulfobutyl ether-beta-cyclodextrin in the formulations provided herein is about 98 to about 99.9% based on total weight of the formulation. In one embodiment, the amount of sulfobutyl ether-beta-cyclodextrin in the formulations provided herein is about 99.1%, 99.3%, 99.5%, 99.7% or 99.9% based on total weight of the formulation. In one embodiment, the amount of sulfobutyl ether-beta-cyclodextrin in the formulations provided herein is about 99.5% based on total weight of the formulation.

In another aspect is a formulation that comprises sulfobutyl ether-beta-cyclodextrin in an amount of about 750 to 850 mg in a 20-cc vial. In another aspect is a formulation that comprises sulfobutyl ether-beta-cyclodextrin in an amount of about 790 to 840 mg, 780 to 830 mg or 790 to 810 mg in a 20-cc vial. In another aspect is a formulation that comprises sulfobutyl ether-beta-cyclodextrin in an amount of about 800 mg in a 20-cc vial.

In another aspect is a formulation that comprises Kleptose HPB in an amount of about 800 mg in a 20-cc vial.

In one embodiment, the formulations comprise formic acid in no more than about 0.5% based on total weight of the formulation. In one embodiment, the formulations comprise formic acid in an amount of up to about 0.05%, 0.07%, 0.09%, 0.1%, 0.2%, 0.3%, 0.4% or 0.5% based on total weight of the formulation. In one embodiment, the formulations comprise formic acid in no more than about 0.05%, 0.07%, 0.09%, 0.1%, 0.2%, 0.3%, 0.4% or 0.5% based on total weight of the formulation. In one embodiment, the amount of formic acid in the formulations provided herein is about 0.05 to about 0.5% based on total weight of the formulation. In one embodiment, the amount of formic acid in the formulations provided herein is about 0.05 to about 0.1% based on total weight of the formulation. In one embodiment, the amount of formic acid in the formulations provided herein is about 0.05%, 0.07%, 0.09%, 0.1%, 0.2%, 0.3%, 0.4% or 0.5% based on total weight of the formulation. In one embodiment, the formulations provided herein do not contain any formic acid. In one embodiment, the amount of formic acid in the formulations provided herein is about 0.05% to 0.09% based on total weight of the formulation.

In another aspect is a formulation that comprises formic acid in an amount of no more than about 1 mg in a 20-cc vial. In another aspect is a formulation that comprises formic acid in an amount of up to about 0.2, 0 5, 0.7, 0.9 mg or 1 mg in a 20-cc vial. In another aspect is a formulation that comprises formic acid in an amount of about 0.3-0.9 mg, or 0.4 to 0.8 mg in a 20-cc vial.

In another aspect, provided herein is a formulation that comprises Compound D in an amount of about 1 mg and HPBCD in an amount of about 800 mg in a 20-cc vial.

In another aspect, provided herein is a formulation that comprises Compound D in an amount of about 1 mg, HPBCD in an amount of about 800 mg and formic acid in an amount of about 0.9 mg in a 20-cc vial.

Formulation Ic

In one embodiment, provided herein are formulations comprising Compound D in an amount of about 0.01 to 0.08% and HPBCD in an amount of about 99.40- to 99.99% based on total weight of the formulation.

In one embodiment, provided herein are formulations comprising Compound D in an amount of about 0.01 to 0.08%, HPBCD in an amount of about 99.40 to 99.99%, and no more than about 0.5% formic acid based on total weight of the formulation.

In one embodiment, provided herein are formulations comprising Compound D in an amount of about 0.03 to 0.06% and HPBCD in an amount of about 99.60 to 99.99% based on total weight of the formulation.

In one embodiment, provided herein are formulations comprising Compound D from about 0.01 to about 0.08%, hydroxypropyl β-cyclodextrin from about 99.40% to about 99.99%, and formic acid from about 0.1 to about 0.3% based on total weight of the formulation

In one aspect, the formulation provided herein comprises Compound D in an amount of about 0.02 to about 0.06% based on the total weight of the formulation. In certain embodiments, the amount of Compound D is from about 0.03% to about 0.06%, or about 0.04% to about 0.06% based on the total weight of the formulation. In certain embodiments, the amount of Compound D is about 0.03%, 0.04%, 0.05% or 0.06% based on the total weight of the formulation. In one embodiment, the amount of Compound D in the formulation is about 0.05% based on the total weight of the formulation.

In another aspect, provided herein is a formulation that comprises Compound D in an amount of about 0.75 mg to about 1.5 mg in a 20-cc vial. In still another aspect is a formulation that comprises Compound D in an amount of about 0.75 mg to about 1.25 mg in a 20-cc vial. In one aspect Compound D is present in an amount of about 0.75, 0.8, 0.9, 1.0, 1.05 or 1.2 mg in a 20-cc vial. In one aspect Compound D is present in an amount of about 1 mg in a 20-cc vial.

In one embodiment, the amount of HPBCD in the formulations provided herein is about 99.40 to about 99.99% based on total weight of the formulation. In one embodiment, the amount of HPBCD in the formulations provided herein is about 99.5, 99.6, 99.7, 99.8, 99.9, 99.95, or 99.99% based on total weight of the formulation. In another aspect is a formulation that comprises HPBCD in an amount of about 1800-1900 mg in a 20-cc vial. In another aspect is a formulation that comprises HPBCD in an amount of about 1850 to 1900 mg in a 20-cc vial. In another aspect is a formulation that comprises HPBCD in an amount of about 1875 mg in a 20-cc vial.

In one embodiment, the formulations comprise formic acid in no more than about 0.5% based on total weight of the formulation. In one embodiment, the formulations comprise formic acid in an amount of up to about 0.05%, 0.07%, 0.09%, 0.1%, 0.2%, 0.3%, 0.4% or 0.5% based on total weight of the formulation. In one embodiment, the formulations comprise formic acid in no more than about 0.05%, 0.07%, 0.09%, 0.1%, 0.2%, 0.3%, 0.4% or 0.5% based on total weight of the formulation. In one embodiment, the amount of formic acid in the formulations provided herein is about 0.05 to about 0.3% based on total weight of the formulation. In one embodiment, the amount of formic acid in the formulations provided herein is about 0.05 to about 0.25% based on total weight of the formulation. In one embodiment, the amount of formic acid in the formulations provided herein is about 0.05%, 0.07%, 0.09%, 0.1%, 0.2%, or 0.3% based on total weight of the formulation. In one embodiment, the formulations provided herein do not contain any formic acid. In one embodiment, the amount of formic acid in the formulations provided herein is about 0.11% to 0.3% based on total weight of the formulation.

In another aspect is a formulation that comprises formic acid in an amount of no more than about 4 mg in a 20-cc vial. In another aspect is a formulation that comprises formic acid in an amount of up to about 1, 1.8, 2, 2.1, 2.5, 3, 3.5, 3.8, 3.9, 4, 4.5, 4.9 mg or 5 mg in a 20-cc vial. In another aspect is a formulation that comprises formic acid in an amount of about 1 to 1.8 mg, 2.1- to 3.8 mg, or 3.9 to 4.9 mg in a 20-cc vial.

In another aspect, provided herein is a formulation that comprises Compound D in an amount of about 1 mg, and HPBCD in an amount of about 1875 mg in a 20-cc vial.

In another aspect, provided herein is a formulation that comprises Compound D in an amount of about 1 mg, HPBCD in an amount of about 1875 mg and formic acid in an amount of about 2.1 to 3.8 mg in a 20-cc vial.

Formulations without Co-Solvent

In one embodiment, provided herein are formulations comprising Compound D in an amount of about 0.15 to 0.5%, a citrate buffer in an amount of about 15% to about 35%, and HPBCD in an amount of about 92% to about 98%, based on total weight of the formulation. In one embodiment, the citrate buffer comprises anhydrous citric acid and anhydrous sodium citrate.

In one embodiment, provided herein are formulations comprising Compound D in an amount of about 0.25 to 0.30%, a citrate buffer in an amount of about 30 to 32%, and HPBCD in an amount of about 67 to 69%, based on total weight of the formulation.

In one embodiment, provided herein are formulations comprising Compound D in an amount of about 0.30- to 0.33%, a citrate buffer in an amount of about 17 to 18%, and HPBCD in an amount of about 80 to 85%, based on total weight of the formulation.

Exemplary Formulations

In one embodiment, provided herein are formulations consisting essentially of Compound D in an amount of about 0.05 to 0.25% and HPBCD in an amount of about 99.75 to 99.95% based on total weight of the formulation.

In one embodiment, provided herein are formulations consisting essentially of Compound D in an amount of about 0.05 to 0.25% and HPBCD in an amount of about 99.75 to 99.99% based on total weight of the formulation.

In one embodiment, provided herein are formulations consisting essentially of Compound D in an amount of about 0.05 to 0.25% and sulfobutyl ether-beta-cyclodextrin in an amount of about 99.75 to 99.95%, based on total weight of the formulation.

In one aspect provided herein is a formulation in a 20-cc vial that comprises: Compound D at an amount that provides 1 mg 2-(4-chlorophenyl)-N-((2-(2,6-dioxopiperidin-3-yl)-1-oxoisoindolin-5-yl)methyl)-2,2-difluoroacetamide, 800 mg HPBCD, and about 0.6 mg formic acid as described herein. In one embodiment, the formulation in a 20-cc vial is reconstituted with 4.5 mL sterile water for injection.

In one aspect provided herein is a formulation in a 20-cc vial that consists essentially of: Compound D at an amount that provides 1 mg 2-(4-chlorophenyl)-N-((2-(2,6-dioxopiperidin-3-yl)-1-oxoisoindolin-5-yl)methyl)-2,2-difluoroacetamide, 800 mg HPBCD, and about 0.6 mg formic acid as described herein. In one embodiment, the formulation in a 20-cc vial is reconstituted with 4.5 mL sterile water for injection.

In one aspect provided herein is a formulation in a 20-cc vial that consists of: Compound D at an amount that provides 1 mg 2-(4-chlorophenyl)-N-((2-(2,6-dioxopiperidin-3-yl)-1-oxoisoindolin-5-yl)methyl)-2,2-difluoroacetamide, 800 mg HPBCD, and about 0.6 mg formic acid as described herein. In one embodiment, the formulation in a 20-cc vial is reconstituted with 4.5 mL sterile water for injection.

In one aspect provided herein is a formulation in a 20-cc vial that comprises: Compound D at an amount that provides 1 mg 2-(4-chlorophenyl)-N-((2-(2,6-dioxopiperidin-3-yl)-1-oxoisoindolin-5-yl)methyl)-2,2-difluoroacetamide, 800 mg sulfobutyl ether-beta-cyclodextrin, and about 0.6 mg formic acid as described herein. In one embodiment, the formulation in a 20-cc vial is reconstituted with 4.5 mL sterile water for injection.

In one aspect provided herein is a formulation in a 20-cc vial that consists essentially of: Compound D at an amount that provides 1 mg 2-(4-chlorophenyl)-N-((2-(2,6-dioxopiperidin-3-yl)-1-oxoisoindolin-5-yl)methyl)-2,2-difluoroacetamide, 800 mg sulfobutyl ether-beta-cyclodextrin, and about 0.6 mg formic acid as described herein. In one embodiment, the formulation in a 20-cc vial is reconstituted with 4.5 mL sterile water for injection.

In one aspect provided herein is a formulation in a 20-cc vial that consists of: Compound D at an amount that provides 1 mg 2-(4-chlorophenyl)-N-((2-(2,6-dioxopiperidin-3-yl)-1-oxoisoindolin-5-yl)methyl)-2,2-difluoroacetamide, 800 mg sulfobutyl ether-beta-cyclodextrin, and about 0.6 mg formic acid as described herein. In one embodiment, the formulation in a 20-cc vial is reconstituted with 4.5 mL sterile water for injection.

In one aspect provided herein is a formulation in a 20-cc vial that comprises: Compound D at an amount that provides 1 mg 2-(4-chlorophenyl)-N-((2-(2,6-dioxopiperidin-3-yl)-1-oxoisoindolin-5-yl)methyl)-2,2-difluoroacetamide, 1875 mg HPBCD, and about 2.1-3.8 mg formic acid as described herein. In one embodiment, the formulation in a 20-cc vial is reconstituted with 12.5 mlL Normal Saline for injection.

In one aspect provided herein is a formulation in a 20-cc vial that consists essentially of: Compound D at an amount that provides 1 mg 2-(4-chlorophenyl)-N-((2-(2,6-dioxopiperidin-3-yl)-1-oxoisoindolin-5-yl)methyl)-2,2-difluoroacetamide, 1875 mg HPBCD, and about 2.1 to 3.8 mg formic acid as described herein. In one embodiment, the formulation in a 20-cc vial is reconstituted with 12.5 ml Normal Saline for injection.

In one aspect provided herein is a formulation in a 20-cc vial that consists of: Compound D at an amount that provides 1 mg 2-(4-chlorophenyl)-N-((2-(2,6-dioxopiperidin-3-yl)-1-oxoisoindolin-5-yl)methyl)-2,2-difluoroacetamide, 1875 mg HPBCD, and about 2.1 to 3.8 mg formic acid as described herein. In one embodiment, the formulation in a 20-cc vial is reconstituted with 12.5 ml Normal Saline for injection.

In one embodiment, provided herein is an aqueous formulation comprising Compound D in an amount of about 0.05 to 0.25% based on total weight of the solids, and HPBCD in an amount of about 99.1 to 99.9% based on total weight of the solids, and a diluent.

In one embodiment, provided herein is an aqueous formulation comprising Compound D in an amount of about 0.05 to 0.25% based on total weight of the solids, and HPBCD in an amount of about 99.75 to 99.95% based on total weight of the solids, and a diluent.

In one embodiment, provided herein is an aqueous formulation consisting essentially of Compound D in an amount of about 0.05 to 0.25% based on total weight of the solids, and HPBCD in an amount of about 99.75-99.95% based on total weight of the solids, and a diluent.

In one aspect provided herein is an aqueous formulation that comprises: Compound D at an amount that provides 1 mg 2-(4-chlorophenyl)-N-((2-(2,6-dioxopiperidin-3-yl)-1-oxoisoindolin-5-yl)methyl)-2,2-difluoroacetamide, 800 mg HPBCD, about 0.6 mg formic acid and about 4.5 mL diluent.

In one aspect provided herein is an aqueous formulation that consists of: Compound D at an amount that provides 1 mg 2-(4-chlorophenyl)-N-((2-(2,6-dioxopiperidin-3-yl)-1-oxoisoindolin-5-yl)methyl)-2,2-difluoroacetamide, 800 mg HPBCD, about 0.6 mg formic acid and about 4.5 mL diluent.

In one embodiment, provided herein is an aqueous formulation comprising Compound D in an amount of about 0.01- to 0.08% based on total weight of the solids, and HPBCD in an amount of about 99.50 to 99.99% based on total weight of the solids, and a diluent.

In one embodiment, provided herein is an aqueous formulation comprising Compound D in an amount of about 0.01 to 0.08% based on total weight of the solids, and HPBCD in an amount of about 99.50 to 99.99% based on total weight of the solids, and a diluent.

In one embodiment, provided herein is an aqueous formulation consisting essentially of Compound D in an amount of about 0.01 to 0.08% based on total weight of the solids, and HPBCD in an amount of about 99.50 to 99.99% based on total weight of the solids, and a diluent.

In one aspect provided herein is an aqueous formulation that comprises: Compound D at an amount that provides 1 mg 2-(4-chlorophenyl)-N-((2-(2,6-dioxopiperidin-3-yl)-1-oxoisoindolin-5-yl)methyl)-2,2-difluoroacetamide, 800 mg HPB CD, about 0.6 mg formic acid and about 4.5 mL diluent.

In one aspect provided herein is an aqueous formulation that consists of: Compound D at an amount that provides 1 mg 2-(4-chlorophenyl)-N-((2-(2,6-dioxopiperidin-3-yl)-1-oxoisoindolin-5-yl)methyl)-2,2-difluoroacetamide, 800 mg HPB CD, about 0.6 mg formic acid and about 4.5 mL diluent.

In certain embodiments, the formulation provided herein is lyophilized, and the lyophilized formulation upon reconstitution has a pH of about 2.5 to 4. In certain embodiments, the lyophilized formulation upon reconstitution has a pH of about 2.5 to 3.5. In certain embodiments, the lyophilized formulation upon reconstitution has a pH of about 3.0 to 3.6. In one embodiment, the lyophilized formulation upon reconstitution has a pH of about 2.5, 3, 3.2, 3.4, 3.6, 3.8 or 4. In one embodiment, the lyophilized formulation upon reconstitution has a pH of about 2.5, 2.8, 3, 3.2, 3.4, 3.6, 3.8 or 4.

In certain embodiments, the lyophilized formulation upon reconstitution has an osmolality of about 260-290 mOsm/kg. In certain embodiments, the lyophilized formulation upon reconstitution has an osmolality of about 280 mOsm/kg. In certain embodiments, the lyophilized formulation upon reconstitution has an osmolality of about 260 to 370 mOsm/kg. In certain embodiments, the lyophilized formulation upon reconstitution has an osmolality of about 360 mOsm/kg. In certain embodiments, the lyophilized formulation upon reconstitution has an osmolality of about 350 to 450 mOsm/kg. In certain embodiments, the lyophilized formulation upon reconstitution has an osmolality of about 416 mOsm.

In certain embodiments, the lyophilized formulation is reconstituted with half normal saline (0.45% sodium chloride sterile solution for injection) and has an osmolality of about 280 to 320 mOsm/kg upon reconstitution. In certain embodiments, the lyophilized formulation is reconstituted with half normal saline (0.45% sodium chloride sterile solution for injection), and has a pH of 3.0 to 3.2 and an osmolality of about 280 to 320 mOsm/kg upon reconstitution. In certain embodiments, the lyophilized formulation is reconstituted with 4.5 mL of half normal saline (0.45% sodium chloride sterile solution for injection), and has a pH of 3.0 to 3.2 and an osmolality of about 280 to 320 mOsm/kg upon reconstitution. In one embodiment, the reconstituted solution of the required dose is diluted with normal saline (0.9% sodium chloride sterile solution for injection) in an infusion bag to a volume to 50 mL for 30-minute intravenous administration.

In certain embodiments, the lyophilized formulation is reconstituted with normal saline and has an osmolality of about 440 mOsm/kg upon reconstitution. In one embodiment, the reconstituted solution of the required dose is diluted with normal saline to a volume to 50 mL to obtain a dosing solution having an osmolality of about 310 to 380 mOsm/kg. In one embodiment, the reconstituted solution of the required dose is diluted with normal saline to a volume to 50 mL to obtain a dosing solution having an osmolality of about 310 to 355 mOsm/kg. In one embodiment, the reconstituted solution of the required dose is diluted with normal saline to a volume to 50 mL to obtain a dosing solution having an osmolality of about 317 to 371 mOsm/kg. In one embodiment, the reconstituted solution of the required dose is diluted with normal saline to a volume to 50 mL to obtain a dosing solution having an osmolality of about 317 mOsm/kg. In one embodiment, the reconstituted solution of the required dose is diluted with normal saline to a volume to 50 mL to obtain a dosing solution having an osmolality of about 371 mOsm/kg. In one embodiment, the osmolality of the dosing solution is no more than 352 mOsm/kg. In one embodiment, the osmolality of the dosing solution having a dose of 4.8 mg Compound D is 352 mOsm/kg.

In certain embodiments, provided herein is a container comprising a formulation provided herein. In one aspect, the container is a glass vial. In one aspect, the container is a 20-cc glass vial.

In one aspect provided herein is a formulation in a 20-cc vial that comprises: Compound D at an amount that provides 1 mg 2-(4-chlorophenyl)-N-((2-(2,6-dioxopiperidin-3-yl)-1-oxoisoindolin-5-yl)methyl)-2,2-difluoroacetamide, and a bulking agent as described herein. In one embodiment, the formulation further comprises no more than about 5 mg formic acid as residual solvent. In one embodiment, the formulation further comprises no more than about 4 mg formic acid as residual solvent. In one embodiment, the formulation further comprises no more than about 3 mg formic acid as residual solvent. In one embodiment, the formulation further comprises no more than about 2 mg formic acid as residual solvent. In one embodiment, the formulation further comprises no more than about 1.5 mg formic acid as residual solvent. In one embodiment, the formulation further comprises no more than about 1 mg formic acid as residual solvent. In one embodiment, the formulation further comprises no more than about 0.8 mg formic acid as residual solvent. In one embodiment, the formulation comprises from about 0.4 mg to about 1.5 mg, about 0.5 mg to about 1 mg, or about 0.5 mg to about 0.9 mg formic acid as residual solvent. In one embodiment, the formulation comprises about 0.4 mg, about 0.6 mg, about 0.8 mg, about 1 mg or about 1.5 mg formic acid as residual solvent. In one embodiment, the formulation comprises formic acid as residual solvent in an amount from about 1.0 mg/mg of Compound D to about 1.8 mg/mg of Compound D, about 2.1 mg/mg of Compound D to about 3.8 mg/mg of Compound D, or about 3.9 mg/mg of Compound D to about 4.9 mg/mg of Compound D.

The formulations of Compound D provided herein can be administered to a patient in need thereof using standard therapeutic methods for delivering Compound D including, but not limited to, the methods described herein. In one embodiment, the formulations provided herein are reconstituted in a pharmaceutically acceptable solvent to produce a pharmaceutically acceptable solution, wherein the solution is administered (such as by intravenous injection) to the patient.

In one aspect, the formulations provided herein lyophilized, and the lyophilized formulations are suitable for reconstitution with a suitable diluent to the appropriate concentration prior to administration. In one embodiment, the lyophilized formulation is stable at room temperature. In one embodiment, the lyophilized formulation is stable at room temperature for up to about 24 months. In one embodiment, the lyophilized formulation is stable at room temperature for up to about 24 months, up to about 18 months, up to about 12 months, up to about 6 months, up to about 3 months or up to about 1 month. In one embodiment, the lyophilized formulation is stable upon storage under accelerated condition of 40° C./75% RH for up to about 12 months, up to about 6 months or up to about 3 months.

The lyophilized formulation provided herein can be reconstituted for parenteral administration to a patient using any pharmaceutically acceptable diluent. Such diluents include, but are not limited to Sterile Water for Injection (SWFI), Dextrose 5% in Water (D5W), or a cosolvent system. Any quantity of diluent may be used to reconstitute the lyophilized formulation such that a suitable solution for injection is prepared. Accordingly, the quantity of the diluent must be sufficient to dissolve the lyophilized formulation. In one embodiment, 1 to 5 mL or 1 to 4 mL of a diluent are used to reconstitute the lyophilized formulation to yield a final concentration of, about 0.05 to 0.3 mg/mL or about 0.15 to 0.25 mg/mL of Compound D. In certain embodiments, the final concentration of Compound D in the reconstituted solution is about 0.25 mg/mL. In certain embodiments, the final concentration of Compound D in the reconstituted solution is about 0.20 mg/mL. In certain embodiments, the volume of the reconstitution diluent varies between 3 ml and 5 ml to yield a final concentration of 0.15 to 0.3 mg/mL. In certain embodiments, depending on the required dose, multiple vials may be used for reconstitution.

The reconstituted solutions of lyophilized formulation can be stored and used within up to about 24 hours, about 12 hours or about 8 hours. In one embodiment, the reconstituted aqueous solution is stable at room temperature from about 1 to 24, 2 to 20, 2 to 15, 2 to 10 hours upon reconstitution. In one embodiment, the reconstituted aqueous solution is stable at room temperature for up to about 20, 15, 12, 10, 8, 6, 4 or 2 hours upon reconstitution. In some embodiments, the solution is used within 8 hours of preparation. In some embodiments, the solution is used within 5 hours of preparation. In some embodiments, the solution is used within 1 hour of preparation.

Process for Making Formulations

The formulations provided herein can be prepared by any of the methods known in the art and as described herein, but all methods include the step of bringing the active ingredient into association with the pharmaceutically acceptable excipient, which constitutes one or more necessary ingredients (such as bulking agent and/or buffer).

In one aspect, the formulations provided herein are prepared by dissolving Compound D, a bulking agent and a citrate buffer in water and dimethyl sulfoxide (DMSO) to obtain a solution, and optionally lyophilizing the solution.

In one embodiment, the process for preparing the formulation comprises: dissolving HPBCD in a citrate buffer to obtain a buffer solution, dissolving Compound D in DMSO to obtain a premix, adding the premix to the buffer solution to obtain a solution; and optionally lyophilizing the solution to produce the lyophilized formulation.

In one embodiment, the process comprises dissolving Kleptose HPB in a 20 mM, pH 4 to 4.5 citrate buffer to obtain a buffer solution, dissolving Compound D in DMSO to obtain an active premix, adding the premix to the buffer solution to obtain a mixture, adding water to the mixture to obtain a bulk solution, filtering the bulk solution through one or more 0.45 μm and 0.22 μm filters to obtain a filtered solution, filling the filtered solution into a vial, and lyophilizing the solution. In one embodiment, the solution is filtered through one 0.45 μm and two 0.22 μm filters. In one embodiment, the process comprises dissolving Kleptose HPB in a 20 mM, pH 4.3 citrate buffer to obtain a buffer solution, dissolving Compound D in DMSO to obtain an active premix, adding the premix to the buffer solution to obtain a mixture, adding water to the mixture to obtain a bulk solution, filtering the bulk solution through one 0.45 μm filter and two 0.22 μm filters to obtain a filtered solution, filling the filtered solution into a 20-cc glass vial, and optionally lyophilizing the solution. In one embodiment, the vial is sealed under nitrogen after lyophilization.

In one aspect, the formulations provided herein are prepared by dissolving Compound D in formic acid to obtain a premix, dissolving HPBCD in water to obtain a solution, adding the premix to the solution to obtain a drug solution; and optionally lyophilizing the drug solution to produce the lyophilized formulation.

In one aspect, the formulations provided herein are prepared by dissolving Compound D in formic acid to obtain an active premix, dissolving Kleptose HPB in water to obtain a Kleptose solution, adding the premix to the Kleptose solution to obtain a mixture, adding water to the mixture to obtain a bulk solution, filtering the bulk solution through one or more 0.45 μm and 0.22 μm filters to obtain a filtered solution, filling the filtered solution into a vial, and lyophilizing the solution. In one embodiment, the solution is filtered through one 0.45 μm and two 0.22 μm filters. In one embodiment, the process comprises dissolving Compound Din formic acid to obtain an active premix, dissolving Kleptose HPB in water to obtain a Kleptose solution, adding the premix to the Kleptose solution to obtain a mixture, adding water to the mixture to obtain a bulk solution, filtering the bulk solution through one 0.45 μm and two 0.22 μm filters to obtain a filtered solution, filling the filtered solution into a 20-cc glass vial, and lyophilizing the solution. In one embodiment, the vial is sealed under nitrogen after lyophilization.

In one aspect, the lyophilization process contains three stages: freezing, primary drying, and secondary drying. A liquid formulation is transformed to a lyophilized powder form by going through complete solidification through freezing stage, sublimation of ice and solvents through primary drying, and desorption of residual moisture and solvents through secondary drying. The shelf temperature and chamber pressure in the primary drying and secondary drying are controlled to obtain the desired quality of the finished drug product. In one aspect of the process, the cake appearance and structure was characterized by visual inspection.

### 5.7. Kits

In one aspect, provided herein is a kit for identifying a subject having cancer who is likely to be responsive to a treatment compound, comprising a means for determining the level of a gene signature (e.g., a LSC signature) in a sample that has been treated with the treatment compound, wherein the treatment compound is a compound described in Section 5.5 above including Compound D.

In another aspect, provided herein is a kit for treating cancer, comprising a means for determining the level of a gene signature (e.g., a LSC signature) in a sample that has been treated with a treatment compound, wherein the treatment compound is a compound described in Section 5.5 above including Compound D.

In yet another aspect, provided herein is a kit for monitoring the efficacy of a treatment compound in treating cancer in a subject, comprising a means for determining the level of a gene signature (e.g., a LSC signature) in a sample that has been treated with the treatment compound, wherein the treatment compound is a compound described in Section 5.5 above including Compound D.

In certain embodiments of various kits provided herein, the treatment compound is Compound D, or a stereoisomer or a mixture of stereoisomers, tautomer, pharmaceutically acceptable salt, solvate, isotopologue, prodrug, hydrate, co-crystal, clathrate, or a polymorph thereof.

In certain embodiments, the cancer is blood cancer. In one embodiment, the blood cancer is lymphoma. In another embodiment, the blood cancer is leukemia. In yet another embodiment, the blood cancer is MM. In a specific embodiment, the leukemia is ALL. In another specific embodiment, the leukemia is AML. In yet another specific embodiment, the leukemia is CLL. In still another embodiment, the leukemia is CML. In some embodiments, the AML is relapsed. In certain embodiments, the AML is refractory. In other embodiments, the AML is resistant to conventional therapy.

In yet other embodiments, the cancer is characterized by an increased level of a LSC signature. In still other embodiments, the LSC signature is a LSC signature described herein. In one embodiment, provided herein is a kit for treating cancer characterized by an increased level of a LSC signature described herein with a treatment compound. In one embodiment, provided herein is a kit for treating leukemia characterized by an increased level of a LSC signature described herein with a treatment compound. In another embodiment, provided herein is a kit for treating AML characterized by an increased level of a LSC signature described herein with a treatment compound.

In certain embodiments, the LSC signature comprises at least one gene selected from the group consisting of AKR1C3, ARHGAP22, CD34, CDK6, CPXM1, DNMT3B, DPYSL3, EMP1, GPR56, KIAA0125, LAPTM4B, MMRN1, NGFRAP1, NYNRIN, SMIM24, SOCS2, and ZBTB46. In some embodiments, the LSC signature comprises two, three, four, five, six, seven, eight, nine, ten, twelve, fourteen, sixteen, or all genes selected from the group consisting of AKR1C3, ARHGAP22, CD34, CDK6, CPXM1, DNMT3B, DPYSL3, EMP1, GPR56, KIAA0125, LAPTM4B, MMRN1, NGFRAP1, NYNRIN, SMIM24, SOCS2, and ZBTB46. In a specific embodiment, the LSC signature is LSC17 signature, comprising AKR1C3, ARHGAP22, CD34, CDK6, CPXM1, DNMT3B, DPYSL3, EMP1, GPR56, KIAA0125, LAPTM4B, MMRN1, NGFRAP1, NYNRIN, SMIM24, SOCS2, and ZBTB46. In some embodiments, the LSC signature is the LSC4 or LSC4 signature provided herein, i.e., a gene signature comprising the following 4 genes: TNFRSF4, SLC4A1, SLC7A7, and AIM2. In other embodiments, the LSC signature is the LSC3 or LSC3 signature provided herein, i.e., a gene signature comprising the following 3 genes: SLC4A1, SLC7A7, and AIM2.

In certain embodiments, the level of the LSC signature in the sample is about 5%, about 10%, about 20%, about 30%, about 40%, about 50%, about 60%, about 70%, about 80%, about 90%, about 100%, about 2 times, about 5 times, about 10 times, about 20 times, about 50 times, or about 100 times higher than the reference level of the LSC signature.

In certain embodiments of various kits provided herein, the sample is obtained from a tumor biopsy, a node biopsy, or a biopsy from the bone marrow, spleen, liver, brain, or breast.

In certain embodiments, provided herein is a kit for detecting the mRNA level of one or more genes of the gene signatures. In certain embodiments, the kit comprises one or more probes that bind specifically to the mRNAs of the one or more genes of the gene signatures. In certain embodiments, the kit further comprises a washing solution. In certain embodiments, the kit further comprises reagents for performing a hybridization assay, mRNA isolation or purification means, detection means, as well as positive and negative controls. In certain embodiments, the kit further comprises an instruction for using the kit. The kit can be tailored for in-home use, clinical use, or research use.

In certain embodiments, provided herein is a kit for detecting the protein level of one or more genes of the gene signatures. In certain embodiments, the kits comprises a dipstick coated with an antibody that recognizes the protein biomarker, washing solutions, reagents for performing the assay, protein isolation or purification means, detection means, as well as positive and negative controls. In certain embodiments, the kit further comprises an instruction for using the kit. The kit can be tailored for in-home use, clinical use, or research use.

Such a kit can employ, for example, a dipstick, a membrane, a chip, a disk, a test strip, a filter, a microsphere, a slide, a multi-well plate, or an optical fiber. The solid support of the kit can be, for example, a plastic, silicon, a metal, a resin, glass, a membrane, a particle, a precipitate, a gel, a polymer, a sheet, a sphere, a polysaccharide, a capillary, a film, a plate, or a slide. The biological sample can be, for example, a cell culture, a cell line, a tissue, an organ, an organelle, a biological fluid, a blood sample, a urine sample, or a skin sample.

In another embodiment, the kit comprises a solid support, nucleic acids attached to the support, where the nucleic acids are complementary to at least 20, 50, 100, 200, 350, or more bases of mRNA, and a means for detecting the expression of the mRNA in a biological sample.

In a specific embodiment, the pharmaceutical or assay kit comprises, in a container, a compound or a pharmaceutical composition thereof, and further comprises, in one or more containers, components for isolating RNA. In another specific embodiment, the pharmaceutical or assay kit comprises, in a container, a compound or a pharmaceutical composition, and further comprises, in one or more containers, components for conducting RT-PCR, qRT-PCR, deep sequencing, or microarray.

In certain embodiments, the kits provided herein employ means for detecting the expression of a biomarker by qRT-PCR, microarray, flow cytometry, or immunofluorescence. In other embodiments, the expression of the biomarker is measured by ELISA-based methodologies or other similar methods known in the art.

In another specific embodiment, the pharmaceutical or assay kit comprises, in a container, a compound or a pharmaceutical composition thereof, and further comprises, in one or more containers, components for isolating protein. In another specific embodiment, the pharmaceutical or assay kit comprises, in a container, a compound or a pharmaceutical composition, and further comprises, in one or more containers, components for conducting flow cytometry or ELISA.

In another aspect, provided herein are kits for determining level of gene signatures that supply the materials necessary to measure the abundance of one or more gene products of the gene signatures or a subset of the gene signatures (e.g., one, two, three, four, five, or more genes) provided herein. Such kits may comprise materials and reagents required for measuring RNA or protein. In some embodiments, such kits include microarrays, wherein the microarray is comprised of oligonucleotides and/or DNA and/or RNA fragments which hybridize to one or more gene products of the gene signatures or a subset of the gene signatures provided herein, or any combination thereof. In some embodiments, such kits may include primers for PCR of either the RNA product or the cDNA copy of the RNA product of the gene signatures or a subset of the gene signatures, or both. In some embodiments, such kits may include primers for PCR as well as probes for qPCR. In some embodiments, such kits may include multiple primers and multiple probes, wherein some of the probes have different fluorophores so as to permit simultaneously measuring multiple gene products of the gene signatures or a subset of the gene signatures provided herein. In some embodiments, such kits may further include materials and reagents for creating cDNA from RNA. In some embodiments, such kits may include antibodies specific for the protein products of the gene signatures or a subset of the gene signatures provided herein. Such kits may additionally comprise materials and reagents for isolating RNA and/or proteins from a biological sample. In addition, such kits may include materials and reagents for synthesizing cDNA from RNA isolated from a biological sample. In some embodiments, such kits may include a computer program product embedded on computer readable media for predicting whether a patient is clinically sensitive to a compound. In some embodiments, the kits may include a computer program product embedded on a computer readable media along with instructions.

In some embodiments, such kits measure the expression of one or more nucleic acid products of the gene signatures or a subset of the gene signatures provided herein. In accordance with this embodiment, the kits may comprise materials and reagents that are necessary for measuring the expression of particular nucleic acid products of the gene signatures or a subset of the gene signatures provided herein. For example, a microarray or RT-PCR kit may be produced for a specific condition and contain only those reagents and materials necessary for measuring the levels of specific RNA transcript products of the gene signatures or a subset of the gene signatures provided herein, to predict whether a hematological cancer in a patient is clinically sensitive to a compound. Alternatively, in some embodiments, the kits can comprise materials and reagents necessary for measuring the expression of particular nucleic acid products of genes other than the gene signatures provided herein. For example, in certain embodiments, the kits comprise materials and reagents necessary for measuring the expression levels of 1, 2, 3, 4, 5, 6, 7, 8, 9, 10, 11, 12, 13, 14, 15, 16, 17, 20, 25, 30, 35, 40, 45, 50, or more of the genes of the gene signatures provided herein, in addition to reagents and materials necessary for measuring the expression levels of at least 1, at least 2, at least 3, at least 4, at least 5, at least 6, at least 7, at least 8, at least 9, at least 10, at least 15, at least 20, at least 25, at least 30, at least 35, at least 40, at least 45, at least 50, or more genes other than the gene signatures provided herein. In other embodiments, the kits contain reagents and materials necessary for measuring the expression levels of at least 1, at least 2, at least 3, at least 4, at least 5, at least 6, at least 7, at least 8, at least 9, at least 10, at least 15, at least 20, at least 25, at least 30, at least 35, at least 40, at least 45, at least 50, or more of the genes of the gene signatures provided herein, and 1, 2, 3, 4, 5, 10, 15, 20, 25, 30, 35, 40, 45, 50, 55, 60, 65, 70, 75, 80, 85, 90, 95, 100, 125, 150, 175, 200, 225, 250, 300, 350, 400, 450, or more genes that are not in the gene signatures provided herein. In certain embodiments, the kits contain reagents and materials necessary for measuring the expression levels of at least 1, at least 2, at least 3, at least 4, at least 5, at least 6, at least 7, at least 8, at least 9, at least 10, at least 15, at least 20, at least 25, at least 30, at least 35, at least 40, at least 45, at least 50, or more of the genes of the gene signatures provided herein, and 1-10, 1-100, 1-150, 1-200, 1-300, 1-400, 1-500, 1-1000, 25-100, 25-200, 25-300, 25-400, 25-500, 25-1000, 100-150, 100-200, 100-300, 100-400, 100-500, 100-1000 or 500-1000 genes that are not in the gene signatures provided herein.

For nucleic acid microarray kits, the kits generally comprise probes attached to a solid support surface. In one such embodiment, probes can be either oligonucleotides or longer probes including probes ranging from 150 nucleotides to 800 nucleotides in length. The probes may be labeled with a detectable label. In a specific embodiment, the probes are specific for one or more of the gene products of the biomarkers provided herein. The microarray kits may comprise instructions for performing the assay and methods for interpreting and analyzing the data resulting from performing the assay. In a specific embodiment, the kits comprise instructions for predicting whether a hematological cancer in a patient is clinically sensitive to a compound. The kits may also comprise hybridization reagents and/or reagents necessary for detecting a signal produced when a probe hybridizes to a target nucleic acid sequence. Generally, the materials and reagents for the microarray kits are in one or more containers. Each component of the kit is generally in its own suitable container.

In certain embodiments, a nucleic acid microarray kit comprises materials and reagents necessary for measuring the expression levels of 1, 2, 3, 4, 5, 6, 7, 8, 9, 10, 15, 20, 25, 30, 35, 40, 45, 50, or more of the genes of the gene signatures provided herein, or a combination thereof, in addition to reagents and materials necessary for measuring the expression levels of at least 1, at least 2, at least 3, at least 4, at least 5, at least 6, at least 7, at least 8, at least 9, at least 10, at least 15, at least 20, at least 25, at least 30, at least 35, at least 40, at least 45, at least 50, or more genes other than those of the gene signatures provided herein. In other embodiments, a nucleic acid microarray kit contains reagents and materials necessary for measuring the expression levels of at least 1, at least 2, at least 3, at least 4, at least 5, at least 6, at least 7, at least 8, at least 9, at least 10, at least 15, at least 20, at least 25, at least 30, at least 35, at least 40, at least 45, at least 50, or more of the genes of the gene signatures provided herein, or any combination thereof, and 1, 2, 3, 4, 5, 10, 15, 20, 25, 30, 35, 40, 45, 50, 55, 60, 65, 70, 75, 80, 85, 90, 95, 100, 125, 150, 175, 200, 225, 250, 300, 350, 400, 450, or more genes that are not of the gene signatures provided herein. In another embodiment, a nucleic acid microarray kit contains reagents and materials necessary for measuring the expression levels of at least 1, at least 2, at least 3, at least 4, at least 5, at least 6, at least 7, at least 8, at least 9, at least 10, at least 15, at least 20, at least 25, at least 30, at least 35, at least 40, at least 45, at least 50, or more of the genes of the gene signatures provided herein, or any combination thereof, and 1-10, 1-100, 1-150, 1-200, 1-300, 1-400, 1-500, 1-1000, 25-100, 25-200, 25-300, 25-400, 25-500, 25-1000, 100-150, 100-200, 100-300, 100-400, 100-500, 100-1000, or 500-1000 genes that are not of the gene signatures provided herein.

For quantitative PCR, the kits generally comprise pre-selected primers specific for particular nucleic acid sequences. The quantitative PCR kits may also comprise enzymes suitable for amplifying nucleic acids (e.g., polymerases such as Taq polymerase), deoxynucleotides, and buffers needed for amplification reaction. The quantitative PCR kits may also comprise probes specific for the nucleic acid sequences associated with or indicative of a condition. The probes may or may not be labeled with a fluorophore. The probes may or may not be labeled with a quencher molecule. In some embodiments, the quantitative PCR kits also comprise components suitable for reverse-transcribing RNA, including enzymes (e.g., reverse transcriptases such as AMV, MMLV, and the like) and primers for reverse transcription along with deoxynucleotides and buffers needed for reverse transcription reaction. Each component of the quantitative PCR kit is generally in its own suitable container. Thus, these kits generally comprise distinct containers suitable for each individual reagent, enzyme, primer and probe. Further, the quantitative PCR kits may comprise instructions for performing the reaction and methods for interpreting and analyzing the data resulting from performing the reaction. In a specific embodiment, the kits contain instructions for predicting whether a hematological cancer in a patient is clinically sensitive to a compound.

For antibody-based kits, the kit can comprise, for example: (1) a first antibody (which may or may not be attached to a solid support) that binds to a peptide, polypeptide or protein of interest; and, optionally, (2) a second, different antibody that binds to either the first antibody or the peptide, polypeptide, or protein, and is conjugated to a detectable label (e.g., a fluorescent label, radioactive isotope, or enzyme). In a specific embodiment, the peptide, polypeptide, or protein of interest is associated with or indicative of a condition (e.g., a disease). The antibody-based kits may also comprise beads for conducting immunoprecipitation. Each component of the antibody-based kits is generally in its own suitable container. Thus, these kits generally comprise distinct containers suitable for each antibody and reagent. Further, the antibody-based kits may comprise instructions for performing the assay and methods for interpreting and analyzing the data resulting from performing the assay. In a specific embodiment, the kits contain instructions for predicting whether a hematological cancer in a patient is clinically sensitive to a compound.

In one embodiment, a kit provided herein comprises a compound provided herein, or a pharmaceutically acceptable salt, solvate, stereoisomer, isotopologue, prodrug, hydrate, co-crystal, clathrate, or a polymorph thereof. Kits may further comprise additional active agents, including but not limited to those disclosed herein.

Kits provided herein may further comprise devices that are used to administer the active ingredients. Examples of such devices include, but are not limited to, syringes, drip bags, patches, and inhalers.

Kits may further comprise cells or blood for transplantation, as well as pharmaceutically acceptable vehicles that can be used to administer one or more active ingredients. For example, if an active ingredient is provided in a solid form that must be reconstituted for parenteral administration, the kit can comprise a sealed container of a suitable vehicle in which the active ingredient can be dissolved to form a particulate-free sterile solution that is suitable for parenteral administration. Examples of pharmaceutically acceptable vehicles include, but are not limited to, water for injection USP; aqueous vehicles (such as, but not limited to, sodium chloride injection, Ringer's injection, dextrose injection, dextrose and sodium chloride injection, and lactated Ringer's injection); water-miscible vehicles (such as, but not limited to, ethyl alcohol, polyethylene glycol, and polypropylene glycol); and non-aqueous vehicles (such as, but not limited to, corn oil, cottonseed oil, peanut oil, sesame oil, ethyl oleate, isopropyl myristate, and benzyl benzoate).

In certain embodiments of the methods and kits provided herein, solid phase supports are used for purifying proteins, labeling samples, or carrying out the solid phase assays. Examples of solid phases suitable for carrying out the methods disclosed herein include beads, particles, colloids, single surfaces, tubes, multi-well plates, microtiter plates, slides, membranes, gels, and electrodes. When the solid phase is a particulate material (e.g., a bead), it is, in one embodiment, distributed in the wells of multi-well plates to allow for parallel processing of the solid phase supports.

It is noted that any combination of the above-listed embodiments, for example, with respect to one or more reagents, such as, without limitation, nucleic acid primers, solid support, and the like, are also contemplated in relation to any of the various methods and/or kits provided herein.

Certain embodiments of the invention are illustrated by the following non-limiting examples.

### EXAMPLES

The examples below are carried out using standard techniques, which are well known and routine to those of skill in the art, except where otherwise described in detail. The examples are intended to be merely illustrative.

### 6.1. Leukemic Stem Cell Signature Scores

6.1.1. LSC17 Score

A 17-gene score using functional leukemia stem cell populations (LSC17 score) was previously reported by Ng et al. (Ng S W et al. Nature. 2016; 540(7633): 433-37), which showed that LSC17 score was highly prognostic for rapid determination of risk and outcome in AML. More specifically, high LSC17 score was associated with initial therapy resistance. Patients with high LSC17 scores had poor outcomes with current treatments including allogeneic stem cell transplantation. Thus, LSC17 score provided clinicians with a tool to identify AML patients who do not benefit from standard therapy according to Ng et al. As described in the following examples, the present disclosure is based, in part, on a surprising finding of a specific correlation between this LSC17 score and responsiveness to Compound D treatment.

The generation and description of the LSC 17 score are provided in more detail in the following paragraphs.

83 cell samples obtained from 78 AML patients were sorted into fractions based on expression of CD34 and CD38. LSC activity in each fraction were assessed by xenotransplantation into NOD.Prkdc^{scid}.Il2rg^{null} (NSG) mice. Each of the functionally defined 138 LSC+ and 89 LSC− fractions was subjected to gene expression (GE) analysis. By comparing GE profiles of LSC+ and LSC− fractions, a list of differentially expressed genes was obtained; 104 genes exhibited ≥2-fold expression level differences (P<0.01). An LSC+ reference profile was defined as the average expression levels of these 104 genes in the LSC+ fractions.

To extract the core transcriptional components of stemness that relate to clinical outcomes across a broad spectrum of AML patient subtypes, a large data set of 495 patients was interrogated (Gene Expression Omnibus (GEO) accession GSE6891 (Verhaak, R. G. et al. Haematologica 94, 131-134 (2009)), in which 89 of the 104 DE LSC genes were captured. Among the 89 LSC genes, 43 genes were more highly expressed in LSC+ fractions.

A statistical regression algorithm was applied based on the least absolute shrinkage and selection operator (LASSO) (Friedman, J., et al. J. Stat. Softw. 33, 1-22 (2010); Simon, N., et al. Stat. Softw. 39, 1-13 (2011)) to relate GE to patient survival in this training cohort, using either the full list of 89 LSC genes or the subset of 43 genes more highly expressed in LSC+ fractions.

Analysis of the latter subset yielded an optimal 17-gene signature (LSC17 score), which could be calculated for each patient as the weighted sum of expression of the 17 genes as shown in Table 2 and the algorithm below:

LSC17 signature score=(DNMT3B×0.0874)+(ZBTB46×−0.0347)+(NYNRIN×0.00865)+(ARHGAP22×−0.0138)+(LAPTM4B×0.00582)+(MMRN1×0.0258)+(DPYSL3×0.0284)+(KIAA0125×0.0196)+(CDK6×−0.0704)+(CPXM1×−0.0258)+(SOCS2×0.0271)+(SMIM24×−0.0226)+(EMP1×0.0146)+(NGFRAP1×0.0465)+(CD34×0.0338)+(AKR1C3×−0.0402)+(GPR56×0.0501).

As above- and below-median scores in the training cohort were associated with adverse and favorable cytogenetic risk, respectively, a median threshold was used to discretize scores into high and low groups.

TABLE 2
LSC Signature Genes and Corresponding
Weights in the LSC17 Score

| LSC signature gene | Weight |
|---|---|
| DNMT3B | 0.0874 |
| ZBTB46 | −0.0347 |
| NYNRIN | 0.00865 |
| ARHGAP22 | −0.0138 |
| LAPTM4B | 0.00582 |
| MMRN1 | 0.0258 |
| DPYSL3 | 0.0284 |
| KIAA0125 | 0.0196 |
| CDK6 | −0.0704 |
| CPXM1 | −0.0258 |
| SOCS2 | 0.0271 |
| SMIM24 | −0.0226 |
| EMP1 | 0.0146 |
| NGFRAP1 | 0.0465 |
| CD34 | 0.0338 |
| AKR1C3 | −0.0402 |
| GPR56 | 0.0501 |

To calculate a LSC17 score, leukemia cells can be collected from peripheral blood of patients. RNA-Seq can be performed on the patient cells to characterize gene expression profiles. In parallel with RNA-Seq, RNA extracted from patient samples can be evaluated in NanoString analysis to determine the LSC17 score. In certain embodiments, patient samples having an LSC17 score that was higher than the median threshold is classified in the group of high LSC17 score, whereas patient samples having an LSC17 score that is lower than the median threshold is classified in the group of low LSC17 score.

### 6.2. Efficacy of Compound D on Primary Acute Myeloid Leukemia Samples with Varying Leukemic Stem Cell Signature Scores

The following are examples of assays that can be used to i) assess efficacy and mechanism of action of Compound D on preclinical models from primary patient-derived AML samples using in vitro and in vivo methods; ii) evaluate LSC17 score correlation with Compound D efficacy; and iii) evaluate the differential effect on leukemic stem cell (LSC) versus normal hematopoietic stem cell (HSC) through secondary engraftment models.

6.2.1. Materials and Methods

6.2.1.1. Test Animals

The NOD/SCID mice used in this study were 10-week old females with an average body weight of 20 grams at the start of dosing.

6.2.1.2. Cell Lines/Cell Culture

All samples were tested for engraftment ability in NOD/SCID mice prior to use in the efficacy studies.

Materials used for in vitro assays of AML cells included X-VIVO 10 medium supplemented with 15% BIT, and growth factors including 100 ng/mL of stem cell factor, 20 ng/mL of interleukin (IL)-6, 20 ng/mL of granulocyte colony-stimulating factor (G-CSF), 20 ng/mL of IL-3, 100 ng/mL of fms-like tyrosine kinase (Flt 3) ligand (each provided by Amgen, USA), 20 ng/mL of granulocyte-monocyte colony-stimulating factor (GM-CSF; R&D Systems, USA) and 50 ng/mL of thrombopoietin (Kirin Brewery, Japan).

For the AML-colony forming unit (CFU) assay, 0.9% methylcellulose semi-solid culture was used, containing 15% fetal calf serum (FCS), 15% pretested human plasma, 50 μM β mercaptoethanol, and cytokines at concentrations of 100 ng/mL stem cell factor, 100 ng/mL Flt-3 ligand, 20 ng/mL IL-6, 20 ng/mL GM-CSF, 20 ng/mL IL-3, and 3 U/mL of erythropoietin (Amgen, USA).

6.2.1.3. Assay Materials and Reagents

Annexin V-PE apoptosis detection kit (BD Pharmingen, BD Bioscience, USA) was used to assess apoptosis.

The following mouse anti-human antibodies were used to assess the efficacy of Compound D in xenograft models of AML (all from BD Biosciences, USA, unless otherwise stated): mouse anti-human CD45-APC, CD33-PC5.5 (Beckman Coulter, USA), CD19-V450, CD14-PE, CD15-FITC, CD34 APC-Cy7, and CD38 PE Cy7. Propidium iodide (BD, USA) was used to identify the dead cells in the analysis.

6.2.2. Experimental Study Design

In this study, leukemia cells were collected from peripheral blood of patients at the Princess Margaret Leukemia Bank and subjected to Ficoll gradient centrifugation to obtain mononuclear cells for viable cryopreservation. All samples were tested for engraftment ability in NOD/SCID mice prior to use in the studies. Acute myeloid leukemia cells were used for short term in vitro suspension culture (4 and 24 hours) to assess the effect of Compound D on the GSPT1 degradation and apoptosis, AML-CFU assay to assess the effect of Compound D on colony forming progenitors, and xenograft transplantation into NOD/SCID mice to assess in vivo effect of Compound D against AML. Upon completion of dosing, all animals were euthanized as scheduled the day after the last Compound D dose and bone marrow was collected from the injected right femur and non injected left femur for flow cytometric analysis using human specific antibodies to evaluate engraftment. Secondary transplant was also performed with limiting dilution assay (LDA) to investigate whether Compound D targeted leukemia stem cells with self-renewal ability.

6.2.3. Experimental Procedures

6.2.3.1. In Vitro Degradation of G1 to S Phase Transition Protein 1 (GSPT1) by Compound D and Apoptosis Assay

Preparation of Test Article Stock Solutions and Dilutions: Compound D was prepared in anhydrous DMSO to make a 1 M stock solution then further diluted to final concentrations of 3, 30, and 100 nM.

Cell Culture: Materials used for in vitro assays of AML cells included X-VIVO 10 medium supplemented with 15% BIT, and growth factors including 100 ng/mL of stem cell factor, 20 ng/mL of IL-6, 20 ng/mL of G-CSF, 20 ng/mL of IL-3, 100 ng/mL of Flt 3 ligand (each provided by Amgen, USA), 20 ng/mL of GM-CSF (R&D Systems, USA), and 50 ng/mL of thrombopoietin (Kirin Brewery, Japan).

Assay Procedure: After 4 and 24 hours of in vitro culture with DMSO or Compound D, cells were harvested for GSPT1 expression and apoptosis. Levels of GSPT1 were analyzed by flow cytometry using median fluorescence intensity (MFI) and normalized against DMSO controls. Apoptosis was also analyzed by flow cytometry and measured as percentage of cells which were positive for cleaved caspase 3/7.

6.2.3.2. Acute Myeloid Leukemia-Colony Forming Unit Assay

Preparation of Test Article Stock Solutions and Dilutions: Compound D was prepared as described below.

Cell Culture: Acute myeloid leukemia cells were plated in 0.9% methylcellulose containing 15% FCS, 15% pretested human plasma, 50 μM β-mercaptoethanol, and cytokines at concentrations of 100 ng/mL stem cell factor, 100 ng/mL Flt-3 ligand, 20 ng/mL IL-6, 20 ng/mL GM-CSF, 20 ng/mL IL-3 and 3 U/mL of erythropoietin (Amgen, USA). Acute myeloid leukemia cells were cultured with DMSO or Compound D (prepared as described below) during suspension and CFU assays.

Assay Procedure: After the AML cultures were incubated with DMSO or Compound D for 12 to 14 days at 37° C., plates were assigned scores for the presence of AML-CFU (CFU defined as >50 cells).

6.2.3.3. Xenograft Assay

Preparation of Test Article for Dosing: For in vivo Compound D dosing, Compound D was formulated immediately prior to each dose following the protocol below.

Formulation: 5% NMP/45% polyethylene glycol (PEG) 400/50% Saline; NMP—Catalogue no. 69118 (new no. M79603-1L), Fluka; PEG400—Catalogue no. 81172-1L, Fluka; Saline—0.9% sodium chloride.

Preparation: Weigh the desired amount of compound in glass vial. Add NMP and vortex. Make sure that entire compound is wet. Add PEG400 and vortex until clear solution without particulates. Add saline slowly and mix thoroughly for about a minute with hand held homogenizer with disposable tip. Use immediately as the compound is not stable in the formulation over time.

Compound Administration: Compound D is dosed via the intraperitoneal route. Vehicle and Compound D are dosed in a volume of 2.5 mL/kg for twice daily (BID) dosing (if testing once daily [QD] dosing, use a dose volume of 5 mL/kg). Recommend a twice daily protocol with a 3-hour separation between the doses. Make up fresh for each administration since the compound is not stable in the formulation over time. Do not exceed a dose level of 5 mg/kg BID as this will result in a maximum concentration Cmax that is likely not clinically relevant.

Intrafemoral Transplantation: One day prior to transplantation, NOD/SCID mice were preconditioned by sublethally irradiating (275 cGy) followed by injection with anti-CD122 antibody (200 μg/mouse) to deplete residual host natural killer (NK) cells. On the day of transplantation, viably frozen AML bulk cells (see Section 6.2.1.2) were thawed, counted, and transplanted intrafemorally into the preconditioned mice at a dose of 5×106 cells/mouse in a total volume of 30 μL phosphate buffered saline.

Treatment and Assay Procedure: At Day 21 post AML transplantation, mice were randomly grouped and dosed with either Compound D at 2.5 mg/kg or vehicle (5% N-methyl-2-pyrrolidone [NMP]/45% polyethylene glycol [PEG] 400/50% saline), intraperitoneally twice daily in a dose volume of 50 μL for 4 weeks. All animals were euthanized at scheduled termination (1 day after the last treatment) and bone marrow was collected from the right femur (injected bone marrow) and the left femur and left and right tibia (non-injected bone marrow). Cells isolated from injected or non-injected bone marrow were analyzed by flow cytometry to assess AML engraftment, and were viably frozen for future secondary engraftment analysis.

Cells harvested from injected and non-injected bone marrow were stained with mouse anti human antibodies as indicated in Section 6.2.1.3. After staining, washed cells were run on an LSRII flow cytometer (BD, USA) with 10,000 to 20,000 events collected for each sample. Collected data were analyzed by FlowJo software (TreeStar, USA) to assess AML engraftment levels in different tissues as determined by the percentage of human CD45+CD33+ cells.

Normal cord blood experiment was carried out similar to what has been described as above but using CD34+ cells isolated from normal cord blood. Two or three normal donors were pulled together to generate enough cells for each of the CB engraftments, termed CB1 and CB2.

Secondary xenograft limiting dilution assay: To determine whether Compound D targeted leukemia stem cells with self-renewal ability, which are considered to contribute to leukemia progression, therapy-resistance, and relapse, secondary transplantation was performed using LDA. Limiting dilution assays are designed to define an unknown frequency of LSCs in the total leukemia graft of primary mice. LDA analysis in secondary transplantation will allow quantitative determination whether Compound D targets LSCs with self-renewal ability in primary mice. For this, multiple cell doses were used for secondary transplant to achieve both a positive response (engrafted mice at high cell doses) and a negative response (non-engrafted mice at lowest cell dose). Four different AML cell doses for each treated group (1 million, 500,000, 50,000 and 2000 cells/mouse) with 5 mice per cell dose, totally 40 mice for each AML graft sample. For any sample that was considered aggressive, LDA was performed with lower cell doses. The frequency of LSCs was analyzed using the Walter and Eliza Hall Institute (WEHI) bioinformatics extreme limiting dilution analysis (ELDA) software (bioinf.wehi.edu.au).

For secondary transplant NOD/SCID mice were sublethally irradiated (275 cGy) and pretreated with anti-CD122 antibody (200 μg/mouse) to deplete residual host natural killer cells. On the day of transplantation, viably frozen cells harvested from the vehicle- or Compound D-treated primary mice were thawed, counted, mouse-cell depleted (Mouse Cell Depletion Kit, Miltenyi Biotec, USA), and transplanted intrafemorally into the pretreated secondary mice at limiting doses described above. For secondary transplant without LDA, thawed cells were not depleted of mouse cells before transplantation. At 12 weeks post secondary transplantation, mice were euthanized, and bone marrow was collected and analyzed.

6.2.3.4. Data Analysis

Engraftment of AML cells in the injected femur and non-injected femur and tibias was analyzed by flow cytometry. Graphs and statistical analysis were generated with GraphPad Prism software. Statistical significance was assessed using one-way analysis of variation (ANOVA) followed by Tukey's multiple comparison posttest.

6.2.4. Effect of Compound D on In Vitro GSPT1 Degradation in Acute Myeloid Leukemia Cells

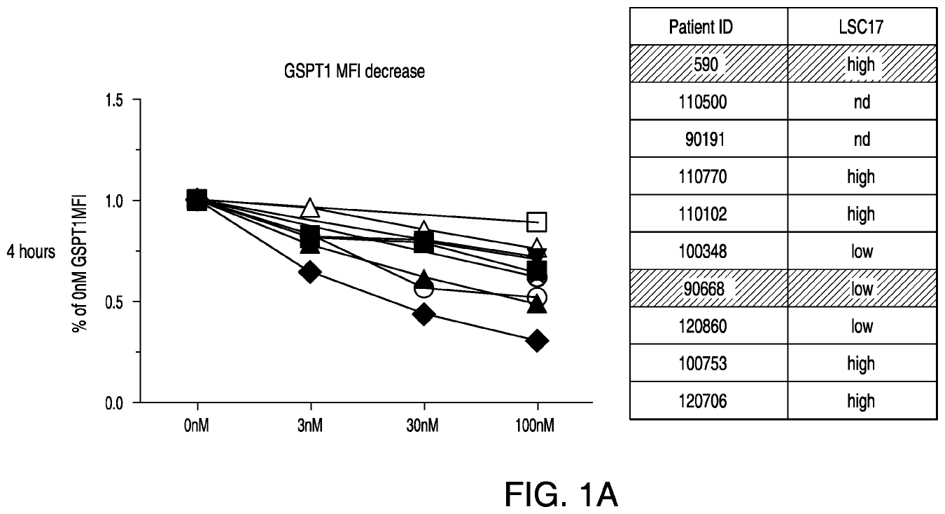

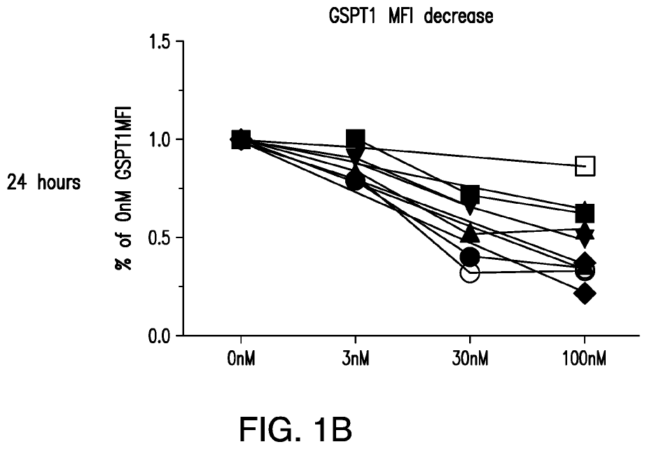

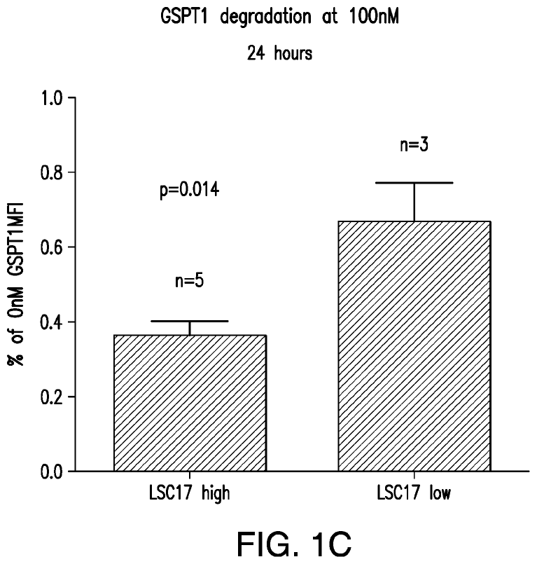

Ten primary AML patient samples were tested in vitro to investigate whether primary AML cells are sensitive to Compound D and whether the sensitivity to Compound D varies among AML samples. Level of GSPT1 in AML cells was first investigated because Compound D inhibits cell viability through GSPT1 degradation. Compound D reduced GSPT1 levels as early as 4 hours post treatment with GSPT1 levels remaining low at 24 hours, compared to control (FIGS. 1A-1B). The effect of Compound D on reduction of GSPT1 levels was concentration dependent. However, even at the highest Compound D concentration (100 nM), the levels of GSPT1 degradation varied between AML samples. AML samples treated in vitro with Compound D were grouped based on the LSC17 scores described above. Surprisingly, the results showed that samples with high LSC17 scores had significantly higher GSPT1 degradation compared to samples with low LSC17 scores (FIG. 1C).

Compound D inhibition of leukemia cell growth through apoptosis was evaluated by flow cytometry. Acute myeloid leukemia cells were cultured with Compound D for 24 hours. Representative flow cytometric analysis of apoptosis from 3 separate samples are shown in FIG. 2A. Apoptosis was not observed for all the samples at 4 hours, however at 24 hours, induction of apoptosis by Compound D was observed in 3 of the 10 samples tested in a concentration-dependent manner (FIG. 2B). Consistent with the GSPT1 degradation data, Compound D induced apoptosis at higher levels in samples with high LSC17 scores compared to samples with low LSC17 scores (FIG. 2B). Also consistent with apoptosis, cell count showed that with increased concentration, Compound D reduced cell numbers for most samples (FIG. 2C). Cells from the samples with higher LSC17 scores had a larger reduction in cell number compared to control than samples with the lower LSC17 scores (FIG. 2C).

Colony-forming assays were also performed to determine whether primary leukemia cells were also sensitive to Compound D. Among 10 samples tested, 7 samples formed colonies and all 7 samples had reduced colony formation with increased Compound D concentration (FIG. 3). Out of 7 samples, 3 had high LSC17 scores and 3 other samples had low LSC17 scores. Compound D reduced colony formation of the samples with high LSC17 scores more than the samples with low LSC17 samples (FIG. 3).

Together, these data indicate that Compound D has inhibitory effect on leukemia blasts and colony forming primary cells. Samples with high LSC17 scores were more responsive to Compound D than the samples with low scores, as assessed by degree of GSPT1 degradation, level of apoptosis induction, reduction in the number of blasts, and capacity of colony formation.

6.2.5. In Vivo Study to Determine the Dose of Compound D for Assessment of Effects on Acute Myeloid Leukemia Cells

A pilot study was first conducted to determine the potential dosage that can be used to target primary AML graft in NOD/SCID mice. Two AML patient samples (AML 110500 and AML 90191) were tested at 1.25 or 2.5 mg/kg Compound D once daily (QD) or twice daily (BID) intraperitoneally for a total of 4 different dose/schedule groups (FIG. 4). Following pretreatment with sublethal irradiation and anti-CD122 antibody, NOD/SCID mice were intrafemorally transplanted with AML cells that were previously collected from patients and viably frozen. Mice were dosed with Compound D for 2 weeks post transplantation starting on Day 21. As shown in FIG. 4 (upper left panel), Compound D reduced AML graft of patient sample AML 110500 cells in a dose-dependent manner relative to vehicle control. Acute myeloid lymphoma grafts in both the injected right femur (RF) and non-injected bone marrow (BM) were significantly decreased at 2.5 mg/kg QD and BID Compound D, with the highest reductions in AML graft at 2.5 mg/kg BID. Primitive leukemic cells positive for CD34+ were also decreased the most by Compound D in both RF and BM in the mice receiving 2.5 mg/kg BID Compound D (FIG. 4, middle left). Percentage of cells positive with myeloid cell marker CD15 were also elevated in both RF and BM of 2.5 mg/kg BID Compound D-dosed mice (FIG. 4, bottom left). Of note, grafted cells from patient sample AML 90191 were largely not affected by Compound D (FIG. 4, right panel). Based on this pilot study, Compound D was dosed at 2.5 mg/kg BID for the rest of the in vivo experiments.

6.2.6. Efficacy of Compound D on Acute Myeloid Leukemia Grafts in Xenografted Mice

Six AML samples were selected for the in vivo study based on LSC17 scores, with 3 samples each of high and low scores. Clinical characteristics and other information are summarized in Table 3 for the samples used for the studies. Two additional samples without LSC17 data were included in a subset of assays.

TABLE 3
Characterization of Acute Myeloid Leukemia Patient Samples for In Vivo Experiments

| | | | MRC | Additional | | | |
|---|---|---|---|---|---|---|---|
| | | Cytogenetics | cytogeneties | cytogeneties | | | |
| Sample | Diagnosis | at Dx | class | notes | NPM1 | FLT3-ITD | FLT3-TKD |
| 590 | unclassified, | 46, XY, | adverse | — | nd | nd | nd |
| | 2° MDS | t(3:3) | | | | | |
| | | (q21; q26.2) | | | | | |
| | | [20] | | | | | |
| 110500 | m5a, | 45, X, −Y, | intermediate | MLL-ENL | nd | nd | nd |
| | 2° chemo/ | t(11; 19) | (abnormal) | | | | |
| | irradiation | (q23; p13.1) | | | | | |
| | | [20] | | | | | |
| | | MLL-ENL | | | | | |
| 90191 | M1 | 46, XY, | adverse | — | nd | nd | nd |
| | | ider(7) (q10) | | | | | |
| | | del(7) | | | | | |
| | | (q21)[20] | | | | | |
| 110770 | unclassified | 46, XX[20] | intermediate | — | positive | positive | negative |
| | | | (normal) | | | | |
| 110102 | unclassified | 45, XX, | adverse | — | nd | nd | nd |
| | | inv(3) | | | | | |
| | | (q21q26), −7 | | | | | |
| | | [20] | | | | | |
| 100348 | unclassified, | 46, XX[17] | intermediate | — | negative | negative | negative |
| | 2° | | (normal) | | | | |
| | (chemo/rads) | | | | | | |
| 90668 | unclassified, | nd | nd | — | nd | nd | nd |
| | 2° chemo | | | | | | |
| 120860 | unclassified | 46, XX, | intermediate | MLL-AF9 | nd | nd | nd |
| | | t(9; 11) | | | | | |
| | | (p22; q23) | (abnormal) | | | | |
| | | [10] | | | | | |
| | | MLL-AF9 | | | | | |
| | | | Sorted | | LSC in | Estimated | |
| | | Engraftment | for LSC | | CD34+/ | LSC | ATF4 |
| | Sample | levels (%)^{a} | signature | LSC17 | CD38⁻ | frequency | results |
| | 590 | 8 × 10⁶ cells | yes | high | Yes | NE | — |
| | | 73/71/75/68/ | | | | | |
| | | 47 | | | | | |
| | | 1 × 10⁶ cells | | | | | |
| | | 0/11/11/2.5 | | | | | |
| | 110500 | yes | yes | nd | — | NE | CD34+ |
| | | | | | | | CD38+ |
| | | | | | | | and |
| | | | | | | | CD34+ |
| | | | | | | | CD38− |
| | | | | | | | are |
| | | | | | | | ATF4 |
| | | | | | | | positive |
| | 90191 | yes | yes | nd | +/− has | Only | CD34− |
| | | | | | high LSC | CD34+ | CD38− |
| | | | | | signature | CD38− | are |
| | | | | | but | repopulate | ATF4 |
| | | | | | double | mice at | positive |
| | | | | | positive | 1/12000 | |
| | | | | | had low | | |
| | 110770 | 57/5.4/60/37 | no | high | — | NE | — |
| | 110102 | 77/0 | yes | high | Yes | 1 in | — |
| | | | | | | 18034 | |
| | | | | | | for +/−; | |
| | | | | | | 1 in | |
| | | | | | | 53602 | |
| | | | | | | for −/− | |
| | 100348 | 32/31/70/ | yes | low | unclear | NE | — |
| | | 85/1.5 | | | | | |
| | 90668 | 52.6/20/ | yes | low | Yes | — | — |
| | | 70/78 | | | | | |
| | 120860 | 97/81/95/93 | no | low | — | nd | CD34+ |
| | | | | | | | CD38+ |
| | | | | | | | and |
| | | | | | | | CD34+ |
| | | | | | | | CD38− |
| | | | | | | | are |
| | | | | | | | ATF4 |
| | | | | | | | positive |

ATF4 = activating transcription factor 4;
MDS = myelodysplastic syndromes;
chemo = chemotherapy;
del = delete;
Dx = diagnosis;
FLT3-ITD = FMS-related tyrosine kinase 3-internal tandem duplication mutation;
FLT3-TKD = FMS-related tyrosine kinase 3-tyrosine kinase domain mutation;
inv = inverted;
LSC = leukemia stem cell;
MLL-AF9 = mixed lineage leukemia-acute lymphoid leukemia fused gene from chromosome 9;
MLL-ENL = mixed lineage leukemia-eleven nineteen leukemia;
MRC = Medical Research Council (MRC) cytogenetic classification system;
nd = no data;
NE = not evaluated;
NPM = nucleophosmin;
rads = radiation therapy;
t = translocation;
“—” = no data available.
^{a}Engraftment level lists percentage engraftment for individual mice from screening experiments. For Sample 590, engraftment was assessed at two cell dose levels.

Based on the pilot data (FIG. 4) in which one of the 2 samples responded to Compound D dosing at 2.5 mg/kg BID for 2 weeks, the duration of dosing was extended to 4 weeks to determine if the mice could tolerate longer Compound D dosing. Mice transplanted with 3 different AML samples were closely monitored for clinical conditions during treatment. Compound D-treated mice were not sick and did not lose weight when compared to vehicle-treated mice following 1, 2, or 3 weeks of Compound D treatment (Table 4). Only one Compound D-treated mouse was found paralyzed on the last day of treatment and was found dead the following morning prior to scheduled sacrifice. This mouse was transplanted with AML cells from Patient Sample 120860, which did not respond well to Compound D (FIG. 6) and likely died of high leukemia burden and profound infiltration given the paralyzation prior to death.

TABLE 4
Body Weight of Human Acute Myeloid Leukemia Xenograft
Mice Following Compound D Dosing

| Week 1 (g) | | Week 2 (g) | | Week 3 (g) | |
|---|---|---|---|---|---|
| | Compound | | Compound | | Compound |
| Vehicle | D | Vehicle | D | Vehicle | D |
| 19 | 22 | 19 | 20 | 20 | 20 |
| 22 | 21 | 23 | 21 | 20 | 22 |
| 24 | 21 | 24 | 22 | 23 | 23 |
| 19 | 23 | 20 | 20 | 23 | 21 |
| 24 | 20 | 24 | 20 | 24 | 19 |
| 20 | 20 | 19 | 22 | 21 | 22 |
| 20 | 21 | 20 | 24 | 22 | 24 |
| 21 | 21 | 21 | 23 | 21 | 22 |
| 24 | 22 | 23 | 24 | 21 | 24 |
| 24 | 22 | 23 | 23 | 22 | 21 |
| 20 | 21 | 20 | 19 | 19 | 20 |
| 22 | 23 | 21 | 22 | 20 | 21 |
| 22 | 24 | 22 | 23 | 21 | 22 |
| 21 | 24 | 22 | 21 | 23 | 21 |
| 22 | 22 | 22 | 21 | 23 | 22 |
| Avg = 21.6 | Avg = 21.8 | Avg = 21.5 | Avg = 21.7 | Avg = 21.5 | Avg = 21.6 |

Avg = average (mean).

Among 6 AML samples tested in vivo for Compound D efficacy, cells from AML Patient 90668 caused paralyzation of the transplanted mice (5 million cells per mouse) before Compound D treatment (Day 21) was initiated. Compound D treatment did not rescue these mice from paralyzation or death. Therefore, for AML Patient 90668, the experiment was repeated with fewer cells transplanted. Mice were still paralyzed around 4 weeks post-transplant even when 10 times fewer AML cells were transplanted (500,000 cells per mouse). Dosing Compound D from Day 21 did not improve survival in these mice engrafted with AML Patient 90668 likely because this sample was very aggressive in NOD/SCID mice, with high engraftment levels and quickly infiltrated to other organs, as evidenced by the rapid paralyzation and with a leukemia cell-intruded enlarged spleen.

For the other 5 AML samples tested, engraftment of the mice was sufficient for assessing the efficacy of Compound D. Compound D had significant effects on 4 out of 5 AML samples. Three of the 4 responder samples were scored high for LSC17 signature. Acute myeloid leukemia cells were completely eradicated to undetectable levels following Compound D administration, assessed by both percent of human CD45+ leukemia graft and absolute numbers of leukemia cells in RF and BM (FIG. 5, left). Because Compound D totally eradicated AML grafts of all 3 patient samples, the percentages of CD34+ primitive cells in the Compound D-treated mice were not reliable (nonspecific and autofluorescent events). The absolute numbers of primitive CD34+ cells were also at very low and undetectable levels in Compound D-treated mice (FIG. 5, right).

Patient Samples AML 120860 and AML 100348 had low LSC17 scores. Compound D did not reduce the numbers of AML cells from Patient 120860 in the injected femur. The number of AML cells were significantly reduced in the non-injected BM compared to vehicle control, but the effect was limited in comparison to reductions of LSC17 high grafts (see FIG. 6, top panel). Percent and number of CD34+ primitive cells in the leukemia graft of this sample were also not reduced by Compound D. Sample AML 100348 had significant response to Compound D treatment, determined by the reduction of human CD45+ leukemia graft in both RF and BM (FIG. 6, bottom panel), however, some level of residual blasts and CD34+ primitive leukemia cells were present in Compound D-treated mice. These results indicate that the samples with low LSC17 score may respond less to Compound D than the samples having high LSC17 scores.

6.2.7. Effect of Compound D on Leukemic Stem Cell Engraftment in Secondary Transplantation

To determine whether Compound D targeted AML leukemia stem cells with self-renewal, cells harvested from the RF and BM of the mice dosed with 2.5 mg/kg Compound D BID or vehicle were combined for transplantation into secondary mice. Following depletion of mouse cells, LDA was performed to determine the frequency of LSCs in the samples with residual AML cells following the Compound D dosing of the engrafted mice. For Patient Samples AML 90191 and 110500, LDA was performed on cells from the mice dosed with 2.5 mg/kg Compound D BID. When mice were sacrificed at approximately 12 weeks post transplantation, secondary mice that were transplanted with AML 90191 cells isolated from the Compound D primary-treated mice had no AML engraftment, indicating that no residual LSC were present in the sample isolated from the primary-treated mice. However, AML 110500 cells isolated from primary-dosed mice were successfully engrafted into secondary mice (FIG. 7A). Secondary mice transplanted with cells from Compound D-treated mice had much lower leukemia graft in comparison to the secondary mice that received vehicle-treated cells. A more than 13-fold decrease of LSC frequency was observed in the Compound D-treated primary mice by LDA analysis (FIG. 7A). Cells from mice transplanted with Patient Sample AML 120860, the sample with low LSC17 scores and showed no response to Compound D in the primary mice, also successfully repopulated secondary mice in the limiting dilution assay (LDA). Even at the lowest cell dose (20,000 AML cells per mouse) all the mice transplanted with cells from vehicle- or Compound D-treated primary mice were engrafted (FIG. 7B). Leukemic stem cell frequency was calculated using the data from the non-injected BM. There was no difference for LSC frequency (calculated using data from the non-injected BM) in vehicle- and Compound D-treated primary mice, indicating that as a non-responder, the LSCs of Patient Sample AML 120860 were not targeted by Compound D (FIG. 7B). Another LSC17-low sample, AML 100348, had a significant response to Compound D in primary-treated mice (FIG. 6). When cells from Patient Sample AML 100348 harvested from primary mice were injected into secondary mice, from 7500 to 200,000 per mouse, none of the transplanted mice were engrafted, even with the cells harvested from vehicle mice (FIG. 7C). Secondary mice were only repopulated when being injected with 1 million cells per mouse from vehicle-treated mice, indicating that the number of cells transplanted for LDA were too low. Limiting dilution assays were not carried out for the remaining 3 AML samples that had high LSC17 scores (AML 0590, 110102, and 110770) because leukemia cells were almost undetectable in the mouse RF and BM due to the high efficacy of Compound D on these patient samples. Thus, for each Patient Sample, combined BM cells without mouse cell depletion of each treated group were equally split into 5 mice per treated group. The percentage and total number of human CD45+ leukemia cells in the primary mice as well as the number of AML cells transplanted per mouse for each condition are summarized in FIG. 7D. Secondary mice transplanted with AML110770 cells either from vehicle or Compound D-dosed primary mice were not repopulated. This is likely because 1) secondary mice were transplanted with too few human leukemia cells (1.21 million cells for vehicle control and 0.04 million cells per mouse for Compound D treated), and 2) transplanted host mouse cells competed with human leukemia cells since mouse cell depletion was not performed for those patient samples.

In contrast, secondary mice transplanted with either AML 0590 or AML 110102 vehicle control cells had more AML cell engraftment compared to AML 110770 (FIGS. 7D-7E). However, cells harvested from the Compound D-treated primary mice did not repopulate the secondary mice, indicating that the residual leukemia cells in the Compound D-treated mice were not enriched with enough LSCs with self-renewal capacity. These results suggest that Compound D also targeted the LSCs of both AML 0590 and AML 110102.

6.2.8. Effect of Compound D on Normal Cord Blood-Derived Human Graft

Compound D toxicity on normal hematopoietic cells was investigated using CB samples. Mice were transplanted with two different CB samples (CB1 and CB2) and dosed with 2.5 mg/kg Compound D or vehicle control BID intraperitoneally. Flow cytometric analyses from a representative mouse from the vehicle- and Compound D-treated groups are shown in FIG. 8A and FIG. 8B, respectively. The quantitative summary is shown in FIG. 9.

As shown in FIG. 9A, while Compound D significantly reduced CB engraftment of both samples, CB cells remained engrafted in most of Compound D-treated mice. Thus, Compound D had a less inhibitory effect on normal CB grafts in comparison to its effect on AML responders, specifically in comparison to the samples with high LSC17 scores which were completely eradicated following Compound D dosing.

The cell population most affected by Compound D in the CB graft was investigated. Compound D treatment caused a significant reduction of human graft, however, 5% to 10% of the graft remained after treatment (FIG. 9A). This contrasts with the near complete elimination of graft in sensitive AML samples (FIG. 5). The number of CD19+ lymphocytes, which are usually the main cell population developed in immune-deficient NOD/SCID mice, were significantly decreased by Compound D (FIG. 9B, top panel). In contrast, the proportion of CD33+ myeloid cells increased following Compound D dosing. The absolute number of CD33+ cells was not reflective of the increased frequency given that the total number of total CB grafts were dramatically decreased (FIG. 9B). Similar results were observed for CD15+ and CD14+ differentiated cells (FIG. 9C). Compound D did not decrease glycophorin A (GlyA)+CD45− erythroid cells in CB1-engrafted mice but did cause a non-significant decrease of GlyA+CD45− erythroid cells in CB2-engrafted mice (FIG. 9D).

Primitive hematopoietic cells (CD34+) in CB graft were also analyzed. In contrast to results with AML responders, Compound D did not decrease the percentage of CD34+ cells while the absolute number of CD34+ cells were significantly reduced (FIG. 10A), due to the dramatic decrease of total CB graft with Compound D treatment. Similar to CD34+ cells, the percentages of CD34+CD38− primary cells, a population enriched for normal hematopoietic stem cells, were not specifically targeted by Compound D (FIG. 10B). In the CD34+ population, only CD34+CD19+ primitive lymphoid cells were significantly decreased by Compound D (FIG. 10C). CD34+CD33+ primitive myeloid cells were not targeted by Compound D (FIG. 10D), indicating that Compound D specifically targeted CD34+CD19+ primitive lymphoid cells and resulted in decrease of lymphocytes.

6.2.9. Conclusions

Compound D induced dose-dependent apoptosis of primary AML patient samples in vitro through degradation of GSPT1. Compound D decreased colony-forming AML progenitors in vitro. Overall, Compound D was well tolerated by NOD/SCID mice transplanted with different primary AML samples. There were no clinical indications of illness, including body weight loss, during 4 weeks of treatment. Among 7 AML samples (including 2 AML samples used in the pilot with 2 weeks treatment duration) that had full treatment schedule completed, 5 samples were responders to Compound D in the mouse xenograft model of human AML. Two samples were not responsive to Compound D, indicating various sensitivities to Compound D between AML samples. These data showed a largely direct relationship between LSC17 score and sensitivity to Compound D. Acute myeloid leukemia samples with high LSC17 scores were more sensitive to Compound D treatment in comparison to samples with low LSC17 scores. This was determined by multiple parameters including the level of GSPT1 degradation, cell growth inhibition with induction of apoptosis, decrease of colony-forming progenitors, and in vivo eradication of AML, grafts. Secondary transplantation showed that the LSCs in the leukemia graft of responders were also targeted. LSCs from sample AML 120860, which was scored low for LSC17 gene signature and a non-responder, were not targeted. Serial transplant may be carried out with more patient samples in order to corroborate the effect of Compound D on LSCs with self-renewal capacity. Compound D also decreased normal cord blood hematopoietic graft in the mice, but to a lesser extent compared to AML responders. The CD19+ lymphoid progenitors and lymphocytes in the CB graft were mainly targeted. In contrast, other types of human cells in CB graft were much less sensitive to Compound D.

Data generated from both in vitro and in vivo treatment clearly showed that Compound D inhibited primary AML cell growth through GSPT1 degradation. There were various sensitivities to Compound D among AML samples. The results indicated that samples with high LSC17 scores are more sensitive to Compound D toxicity than samples with low LSC scores. As discussed, the LSC17 score was previously found highly prognostic and accurately predicates initial therapy resistance of AML, i.e., patients with high LSC17 scores have poor outcomes with current treatments including allogeneic stem cell transplantation (Ng S W et al. Nature. 2016; 540(7633): 433-37). Thus, the present finding that samples with high LSC17 scores are more sensitive to Compound D indicates Compound D may target the refractory AML resistant to current chemotherapies. Furthermore, the observation that Compound D had less effect on normal hematopoietic graft than AML responders supports Compound D efficacy in AML patients with high LSC17 scores.

### 6.3. Responsiveness of Acute Myeloid Leukemia to Compound D and Discovery of Potential Predictive Biomarkers for Efficacy of Compound D

The following are examples of assays that can be used to i) determine the ratio of efficacy and resistance of AML to Compound D by performing experiments on a larger number of AML samples; ii) identify potential biomarkers that can predict AML response/resistance to Compound D through RNA Seq analysis on AML patient samples.

6.3.1. Materials and Methods

Details on test animals, cell lines/cell culture, and assay materials and reagents are provided in Section 6.2.1.

6.3.2. Experimental Procedures

6.3.2.1. RNA-Seq

Ribonucleic acid (RNA) was extracted from primary leukemia cells, quantified and qualified using bioanalyzer, and run for RNA-Seq. Totally 33 patients diagnosed with AML were used for RNA-Seq analysis, including 2 samples (110500 and 90191) that were tested for the effect of Compound D in the study described in Section 6.1.

6.3.2.2. Nano String for LSC17 Score

Ribonucleic acid extracted for RNA-Seq was also sent for Nano String analysis to determine LSC17 scores. Analysis was performed with 150 ng RNA in 5 μL for each sample for NanoString using elements chemistry assays. Twenty samples with known LSC17 high and low scores were submitted for NanoString analysis to serve as a control.

6.3.2.3. Preparation of Stock Solutions and Dilutions of Compound D

The procedure followed for preparation of solutions of Compound D for dosing animals is described in Section 6.2.3.3.

Stock solutions and dilutions for in vitro experiments were prepared as follows: Compound D was first dissolved in an hydrated dimethyl sulfoxide (DMSO) to reach 1M concentration and then further diluted serially to different concentrations (10 mM, 10 μM, 1 μM) in the completed medium for cell culture. The final concentrations of Compound D for in vitro culture were 3 nM, 30 nM, and 100 nM.

6.3.2.4. In Vivo Compound D Efficacy Against AML

Immune-deficient NOD/SCID mice were sublethally irradiated (225cGy) and treated with anti-CD122 antibody to eradicate residual mouse NK cells the day before AML implantation. Primary AML cells from each patient were intrafemorally injected into the mouse right femur at the cell dose 5×10⁶ per mouse, with 10 mice transplanted per sample. Compound D and vehicle treatment was initiated at day 21 post transplantation. Compound D was administered at 2.5 mg/kg, intraperitoneally (IP) twice a day at 3 hours apart for 4 weeks. Before each treatment, Compound D was freshly dissolved into the solution. Vehicle was the same solution without Compound D compound and given to the control-treated mice at same volume (50 μL/mouse) with the same therapeutic schedule as Compound D treatment. For each patient sample, each treated group had 5 mice.

After treatment finished, cells were harvested from both injected right femur (RF) and non-injected bone marrow (BM, including left femur, 2 tibias), and stained with human antibodies to assess the engraftment levels of AML. Antibodies used for staining included: Mouse anti-human CD45-APC, CD15-FITC, CD34-APC7, CD38-PC7 (BD Biosciences, USA), CD14-PE, CD33-PC5 (Beckman Coulter, USA), CD19-V450, CD19-AF700, CD11b-APC7, CD34-BV421 (BD Biosciences, USA), and propidium iodide (PI; Invitrogen, USA).

6.3.2.5. In Vitro Assays of the Effects of Compound D on GSPT1 Expression, Apoptosis, and Colony Growth of Primary Leukemia Cells

Viably frozen primary leukemia cells were thawed and plated in suspension culture in the Iscove's Modified Dulbecco's Medium plus 15% BIT Serum Substitute (Stem Cell Technology, Canada), supplemented with multiple human growth factors. Compound D was added to the culture at indicated concentrations.

For GSPT1 expression, intracellular flow cytometry (FACS) was performed at 24 hours in culture by staining cells with GSPT1 conjugated with Alexa Fluor 647. For apoptosis, cells were harvested at 24 hours in culture and stained with Annexin V-PE and 7-aminoactinomycin D (7AAD) (BD Biosciences, USA).

Colony assays were performed in semisolid culture supplemented with growth factors, in the presence of Compound D, or DMSO for control. Colonies were counted at Day 14.

6.3.2.6. In Vivo Effects of Compound D on GSPT1 Expression in Xenograft AML Model

After 4 weeks of transplantation with AML cells, mice were treated with Compound D at 2.5 mg/kg twice a day for 3 doses totally. Four hours after the last treatment, cells were harvested from both injected RF and non-injected BM of each mouse and were stained with CD45-FITC (BD Biosciences, USA), fixed and permeabilized. Cells were then stained with GSPT1-Alexa Fluor 647 for intracellular FACS to detect the expression of GSPT1 in engrafted leukemic cells.

6.3.2.7. Data Analysis

Engraftment of AML cells in the injected femur and non-injected femur was analyzed by flow cytometry. Graphs and statistical analysis were generated with GraphPad Prism software. Statistical significance was assessed using one-way analysis of variation (ANOVA) followed by Tukey's multiple comparison posttest.

6.3.3. Heterogeneous Responses of Acute Myeloid Leukemia Samples to Compound D

A total of 31 patient samples with clinical characterization (Table 5) were tested in xenograft assays to determine the efficacy of Compound D against AML in the mice. Leukemic engraftment was assessed by the percent of CD45+CD33+ population in the injected RF and non-injected BM. Some samples only repopulated the mouse RF at low levels with very low or undetectable leukemia cells in the BM (120347, 130311, 5786, and 141104). Patient 90156 did not repopulate either the mouse RF or BM at the time of analyzing. Other AML samples engrafted both injected RF and non-injected BM (FIG. 11). The majority of engrafted samples (24 out of 28) had significant and dramatic responses to Compound D, consistent with the previous pilot study for Compound D. Compound D reduced AML burden in both injected RF and non-injected BM in those responsive samples while leukemic cells in the BM had more profound responses to Compound D (FIG. 11). Some samples responded less and a few samples were resistant to Compound D, indicating that, while Compound D was potent against AML, the responsiveness varied among AML samples.

TABLE 5
Molecular Characterization of Samples from 31 Patients with Acute Myeloid Leukemia

| | | | MRC | | | |
|---|---|---|---|---|---|---|
| Patient | | | Cytogenetics | | Flt3- | Flt3- |
| ID | Diagnosis | Cytogenetics at Dx | Class | NPM1 | ITD | TKD |
| 120846 | secondary | 46, XY, t(1; 3)(q32; q26~27), | adverse | nd | nd | nd |
| | (MDS) | del(20)(q13.1)[11] | | | | |
| 110625 | De novo AML, | 46, XY | intermediate | negative | positive | negative |
| | M0 | | (normal) | | | |
| 110555 | De novo AML | 45, XX, inv(3)(q21q26), −7[20] | adverse | nd | nd | nd |
| 5786 | De novo AML, | 46, XY, −3, +del(6)(q21) | intermediate | nd | nd | nd |
| | M2 | | (abnormal) | | | |
| 90240 | De novo AML, | 52 XX +2 +9 +10 +13 +14 +15 | adverse | nd | nd | nd |
| | M1, relapse3, | | | | | |
| | peritoneal fluid | | | | | |
| 90543 | De novo AML, | 46, XY, inv(3)(q21q26.2), t(9; 22)(q34; | adverse | nd | nd | nd |
| | M2 | q11.2)[9]/46, XY[1] | | | | |
| 90156 | secondary | 48, XX, +8, +der(9)t(1; 9)(q21; p24)[8], | intermediate | nd | nd | nd |
| | (MPN), M6 | 48, XX, +8, +der(9)t(1; 9)(q21; q22)[7], | (abnormal) | | | |
| | | 48, XX, +8, +der(9)t(1; 9)(q21; q24), | | | | |
| | | +der(9)t(1; 9)[4] | | | | |
| 100474 | secondary M5a | 47, XY, +8[11] | intermediate | nd | nd | nd |
| | (MDS, | | (abnormal) | | | |
| | myeloma, low | | | | | |
| | grade LPD all | | | | | |
| | preceded AML) | | | | | |
| 110120 | De novo AML | 46, XX[20] | intermediate | positive | high | negative |
| | | | (normal) | | | |
| 110484 | De novo AML, | 45, XY, inv(3)(q21q26.2), −7, t(9; 22)(q34; | adverse | nd | nd | nd |
| | M1 | 11.2)[10] | | | | |
| 120093 | secondary | nd | nd | nd | nd | nd |
| | (NHL treated | | | | | |
| | with | | | | | |
| | chemotherapy) | | | | | |
| 120791 | De novo AML, | 46 XY | intermediate | nd | nd | nd |
| | M5 | | (normal) | | | |
| 120899 | De novo AML, | 46, XY | intermediate | positive | positive | negative |
| | M5a | | (normal) | | | |
| 130262 | De novo AML, | 46, XX[20] | intermediate | negative | low | negative |
| | M5a | | (normal) | | | |
| 130578 | De novo AML, | 46, XY | intermediate | negative | negative | negative |
| | M4 | | (normal) | | | |
| 130695 | De novo AML, | 46, XX[12] | intermediate | positive | high | negative |
| | M5a | | (normal) | | | |
| 130712 | De novo AML | 46, XX, (9; 11)(p22; q23)[10] | intermediate | negative | negative | negative |
| | | | (abnormal) | | | |
| 120858 | De novo AML, | 46, XX[20] | intermediate | positive | low | positive |
| | M5a, relapse | | (normal) | | | |
| 120347 | De novo AML | 46, XX, t(1; 14)(q21; q11.2) | intermediate | negative | low | positive |
| | | | (abnormal) | | | |
| 121020 | De novo AML, | 46, XY | intermediate | positive | positive | negative |
| | M4 | | (normal) | | | |
| 130607 | De novo AML, | 46, XX[20] | intermediate | nd | nd | nd |
| | M5b | | (normal) | | | |
| 130926 | De novo AML, | 46, XY | intermediate | negative | negative | negative |
| | M5a | | (normal) | | | |
| 598 | De novo AML, | 46, XY | intermediate | positive | positive | negative |
| | M5b | | (normal) | | | |
| 120287 | De novo AML, | 46, XY | intermediate | negative | negative | negative |
| | Ml | | (normal) | | | |
| 130311 | De novo AML, | 43, XX, der(2)ins(2; ?)(q11.2; ?),add(3)(q27), | adverse | nd | nd | nd |
| | M4 | add(4)(q12), del(5)(q13q33), der(6)t(4; 6)(q12; | | | | |
| | | q13), −7, +8, −10, del(12)(q15q24.1), | | | | |
| | | idic(13)(p11.2), −14, −15, −16, add(17)(p11.2), | | | | |
| | | −21, +3mar[11] | | | | |
| 130826 | De novo AML, | 46, XY[20] | intermediate | positive | high | negative |
| | M4 | | (normal) | | | |
| 140005 | De novo AML, | 46, XX[20] | intermediate | positive | intermed | negative |
| | M5a | | (normal) | | iate | |
| 140171 | De novo AML | 46, XY[20] | intermediate | negative | negative | negative |
| | | | (normal) | | | |
| 141104 | De novo AML | 46, XX[10] | intermediate | nd | intermed | nd |
| | | | (normal) | | iate | |
| 150238 | secondary | 47, XY, −7, +21, +21[10] | adverse | nd | nd | nd |
| | (MDS) | | | | | |
| 150250 | De novo AML | 46, XX[9] | intermediate | positive | negative | negative |
| | | | (normal) | | | |

AML = acute myeloid leukemia;
Dx = diagnosis;
Flt3-ITD = fms like tyrosine kinase 3-internal tandem duplication;
Flt3-TKD = fms related tyrosine kinase 3-tyrosine kinase domain;
ID = identification;
LPD = lymphoproliferative disorder;
MDS = myelodysplastic syndrome;
MPN = myeloproliferative neoplasm;
MRC = myelodysplasia-related changes;
NHL = non-Hodgkin lymphoma;
NPM1 = nucleophosmin 1.

6.3.4. Compound D Induces Differentiation of Primitive Acute Myeloid Leukemia Cells

With the observation that Compound D has dramatic effect against AML in mice, the question of whether Compound D targets primitive leukemic cells and induces differentiation was investigated next because AML cells are immature blasts with differentiation and maturation blockage in patients. Samples that were focused on were the samples that still had clear residual leukemia cells in the mice following Compound D treatment. As shown in FIG. 12A, 3 representative samples had clear increased expression of myeloid differentiation marker CD15 following Compound D treatment. Compound D also induced expression of monocytic cell marker CD14 in the graft of patient 120287. The majority of cells harvested from the mice transplanted with patient sample 120093 were CD34+ primitive cells lacking in CD15 expression. Compound D treatment induced CD15 expression and, in parallel, reduced CD34+ cell population, indicating that, in this sample, Compound D targeted and differentiated CD34+ primitive cells. Compound D also reduced CD34+ cells in the graft of patient samples 100348 and 130826, with reduction of CD14+ or CD11b+ cells (FIG. 12B). Different from those two samples, Compound D only eliminated CD14+ cells of 100474 with more residual CD34+ primitive cells in the remaining graft. CD11b is also a myeloid differentiation marker and was increased on patient 150250 grafted cells in parallel with increased expression of another myeloid differentiation marker CD15. However, Compound D decreased CD11b+ leukemia cells with the reduction of CD34+ cells of patient 130826 (FIG. 12B). The changes of CD15, CD14 and CD34 positive populations are summarized in FIGS. 12C-12E and 3 different patterns after Compound D treatment are apparent: increase, decrease, or no change of the populations in AML, graft. The samples with reduction of CD34+ primitive cells and increase of CD15+ and/or CD14+ cells are the samples responding well to Compound D (such as patient samples 110555, 110500, and 120093). Samples that did not respond or responded poorly to Compound D had no change or even an increase in CD34+ primitive cells, while some responsive samples to Compound D also had increased CD34+ cells in their graft. Increased expression of myeloid differentiation markers CD15 and CD14 were not observed in the samples that were resistant or poorly responsive to Compound D. The current studies indicate that, while Compound D dramatically eliminated total AML graft and thus the absolute number of both primitive and differentiated leukemia cells were significantly decreased, Compound D targeted primitive leukemia cells and resulted in myeloid differentiation at least in some samples. Secondary transplantation may be performed to determine whether Compound D targeted the LSCs with self-renewal capacity.

6.3.5. Identification of Biomarkers for Responses to Compound for Treating Acute Myeloid Leukemia

6.3.5.1. Compound D Responsiveness and Relation to Scores by Nano String

Acute myeloid leukemia is a group of malignant hematological diseases, phenotypic and genetically heterogeneous, and their responsiveness to clinical induction therapies varies between patients. In order to determine which kind of patients respond to Compound D better and whether Compound D has an effect on the samples that are resistant to clinical therapies, RNA-Seq was next performed on patient cells to characterize gene expression profiles of the samples used in the studies. In parallel with RNA-Seq, RNA extracted from patient samples was also sent for NanoString analysis to determine the LSC17 score described above. In total, 33 AML samples (including 31 samples used in current SRA Amendment and 2 samples from previous pilot SRA studies) were submitted for NanoString analysis. Twenty samples previously determined for their LSC17 scores were run in parallel as a control. Out of the 33 AML samples analyzed, only 6 had low LSC17 scores (see Table 6), likely due to the use of samples that had the criteria needed to enable the study: high engraftment capacity in the xenografts and having large numbers of biobanked vials. Such samples are typically characterized to be from aggressive disease with poor outcome and thus have high LSC17 scores.

TABLE 6
Acute Myeloid Leukemia Samples-NanoString for LSC17 Scores

| | | LSC17 Scores by | LSC17 Scores by |
|---|---|---|---|
| | | NanoString | NanoString |
| Patient ID | Code for RNA | Raw Score | Classification |
| 120846 | CC01 | 1.271191073 | LSC17hi |
| 110625 | CC02 | 1.053440946 | LSC17hi |
| 110555 | CC03 | 1.151928168 | LSC17hi |
| 5786 ^{a} | CC04 | 1.021199215 | LSC17hi |
| 90240 | CC05 | 0.368560828 | LSC17lo |
| 90543 | CC06 | 0.736074529 | LSC17hi |
| 90156 ^{a} | CC07 | 1.044834979 | LSC17hi |
| 90191 ^{b} | CC08 | 1.382215202 | LSC17hi |
| 100474 | CC09 | 0.420008982 | LSC17lo |
| 110120 | CC10 | 1.042194461 | LSC17hi |
| 110484 | CC11 | 1.304486071 | LSC17hi |
| 110500 ^{b} | CC12 | 0.67196099 | LSC17hi |
| 120093 | CC13 | 0.266904123 | LSC17lo |
| 120791 | CC14 | 0.109565333 | LSC17lo |
| 120899 | CC15 | 0.874884102 | LSC17hi |
| 130262 | CC16 | 0.779455095 | LSC17hi |
| 130578 | CC17 | 0.709446834 | LSC17hi |
| 130695 | CC18 | 0.719425415 | LSC17hi |
| 130712 | CC19 | 0.80061435 | LSC17hi |
| 120858 | CC20 | 0.954879524 | LSC17hi |
| 120347 ^{a} | CC21 | 0.63621056 | LSC17hi |
| 121020 | CC22 | 0.870600597 | LSC17hi |
| 130607 | CC23 | 1.119176906 | LSC17hi |
| 130926 | CC24 | 0.53775983 | LSC17hi |
| 598 | CC25 | 0.644954458 | LSC17hi |
| 120287 | CC26 | 0.338451625 | LSC17lo |
| 130311 ^{a} | CC27 | 0.844231421 | LSC17hi |
| 130826 | CC28 | 0.902088587 | LSC17hi |
| 140005 | CC29 | 0.6012812 | LSC17hi |
| 140171 | CC30 | 0.875195998 | LSC17hi |
| 141104 ^{a} | CC31 | 0.943161718 | LSC17hi |
| 150238 | CC32 | 1.33104564 | LSC17hi |
| 150250 | CC33 | 0.170716767 | LSC17lo |

ID = identification; LSC = leukemic stem cell; RNA = ribonucleic acid.
^{a} Some samples (120347, 130311, 5786, and 141104) only repopulated the mouse RF (injected right femur) at low levels with very low or undetectable leukemia cells in the BM (non-injected bone marrow). Patient 90156 did not repopulate either the mouse RF or BM at the time of analyzing.
^{b} Samples studied in the pilot SRA study as described in Section 6.1.

Reference values for classification of high and low LSC17 scores are presented in Table 7.

TABLE 7
Reference Values for NanoString Classification

| Sample ID | Raw Score | Classification |
|---|---|---|
| 5004 | 1.42460806 | LSC17hi |
| 243 | 1.34838962 | LSC17hi |
| 5326 | 1.23703093 | LSC17hi |
| 110052 | 1.20989306 | LSC17hi |
| 5619 | 1.19767003 | LSC17hi |
| 8315 | 1.16072595 | LSC17hi |
| 110770 | 1.15114498 | LSC17hi |
| 100845 | 1.11452443 | LSC17hi |
| 9030 | 1.1405956 | LSC17hi |
| 8147 | 1.09154565 | LSC17hi |
| 5264 | 1.05329073 | LSC17hi |
| 5199 | −0.0391706 | LSC17lo |
| 110843 | −0.1467073 | LSC17lo |
| 5657 | 0.10159177 | LSC17lo |
| 110029 | −0.0768989 | LSC17lo |
| 100260 | −0.0119696 | LSC17lo |
| 443 | 0.13937079 | LSC17lo |
| 100087 | −0.1014738 | LSC17lo |
| 90335 | −0.0103532 | LSC17lo |
| 90520 | −0.1271686 | LSC17lo |
| 9571 | −0.04397 | LSC17lo |
| 110633 | −0.1648828 | LSC17lo |

hi = high score; ID = identification; lo = low score.

As shown in FIG. 12, there were heterogeneous responses between samples which can be grouped into 3 subgroups (FIG. 13). Samples that engrafted mice less than 10% in vehicle-treated RF and BM tissues were considered too low for efficacy analysis and were excluded. Seventeen samples had dramatic responses to Compound D in their RF as most of leukemia cells were eradicated, with 14 samples showing similar responses in their BM (>80% reduction, group 1). Another 10 samples had decent responses but less than group 1 in their RF, with similar responses for 6 samples in the BM (50 to 75% reduction, group 2). The remaining 9 samples had poor responses with less than 25% reduction in RF. 4 out of 9 samples did not respond to Compound D at all in the injected RF, and 3 out of 6 samples had no AML reduction in the BM (group 3). The efficacy of Compound D was next analyzed based on the classification of high and low LSC17 scores to see whether they associated with the responsiveness of AML samples to Compound D. While one sample with low LSC17 score was not responsive to Compound D in injected right femur and had less than 25% of reduction in the non-injected bone marrow, the other 7 samples with low LSC17 scores responded very well to Compound D (more than 50% reduction, FIG. 13). Eight out of 9 non-responders in RF had high LSC17 scores, and 5 samples out of those 8 non-responding samples also had poor responses in BM. Interestingly, the majority of samples with high LSC17 scores, which should have poorer prognosis with resistance to clinical chemotherapies, responded well to Compound D with more than 50% leukemia reduction. A large number of samples had very impressive responses with more than 80% leukemia reduction (13 in RF and 17 in BM, FIG. 13), indicating that Compound D is very potent against AML cells from most samples with high LSC17 scores.

6.3.5.2. Gene Expression Biomarkers to Predict the AML Response to Compound D

Gene expression profiles of patient samples generated from RNA-Seq were next analyzed to find biomarkers that can predict AML response to Compound D. Twenty-six samples were eligible for analysis. LSC17 score and the correlation to the average LSC+ and LSC− gene expression profiles were not significantly associated with the percentage of AML, reduction probably because most of the samples used in the current study had high LSC scores and high percentage of AML reduction by Compound D. Thus, increasing the number of samples with low LSC scores would greatly increase the chances of detecting significant trends.

Next, an optimized sub-score was sought that can predict response to Compound D by using percentage of AML reduction as the response to guide the selection of signature genes. Seventy five percent of samples (n=20) were used as training and remaining 25% of samples (n=6) were used for testing. When LSC17 and 43 LSC+ genes (see Section 6.1) were chosen for the training and testing, no signature was found to predict response with high accuracy.

However, a 4-gene score was identified out of 89 LSC genes (see Section 6.1) that can predict the percent reduction in AML with moderate accuracy (FIG. 14 panel A, r=0.77, p=0.10). Discretizing the predictions by a median of the scores from all samples used in this study to either “response” or “no response” (the cut-off for no response is 25% reduction) also showed an association between the scores and % reduction (FIG. 14 panel B, r=0.87, p=0.02). The four signature genes and their standardized weights are shown in Table 8 and the algorithm below:

4-gene score(LSC4 signature score)=(TNFRSF4×−1.13)+(SLC4A1×13.59)+(SLC7A7×−3.57)+(AIM2×−3.04).

A positive weight suggests that a higher expression of the associated gene would increase percent reduction in the experiments, while negative weights suggest that higher expression of the associated genes would decrease percent reduction. TNFRSF4 is highly expressed in LSC+ samples and the protein is significantly higher expressed in relapse compared to diagnosis samples, where it is often the case that higher LSC frequency is observed at relapse. The remaining 3 signature genes are expressed higher in LSC− samples.

TABLE 8
LSC Signature Genes and Corresponding
Weights in the 4-Gene Score

| 4 genes out of the 89 LSC genes | Weight |
|---|---|
| TNFRSF4 | −1.13 |
| SLC4A1 | 13.59 |
| SLC7A7 | −3.57 |
| AIM2 | −3.04 |

LSC = leukemic stem cell.

Because 3 out of 4 genes expressed higher in LSC− samples, the LSC− data was next analyzed for predictive sub-scores. Signature training on the 46 LSC− genes resulted in a 3-gene score (see below) that was predictive of AML reduction, with very similar results when compared to the aforementioned 4-gene score. Indeed, the 3 genes comprising the 3-gene sub-score were the 3 LSC− genes in the 4-gene sub-score: SLC4A1, SLC7A7, and AIM2 (FIG. 14 panel C), indicating that LSC− samples may respond better to Compound D. The 3-gene score is as follows:

3-gene score(LSC3 signature score)=(SLC4A1×13.59)+(SLC7A7×−3.57)+(AIM2×−3.04).

In sum, the 4-gene score and the 3-gene score correlate well with response to Compound D.

6.3.5.3. Clinical Characterization of Samples Related to Compound D Responses

The clinical characteristics of samples were investigated to determine whether any clinical profiles are related to Compound D response. Cells collected from secondary and relapsed AML patients had Compound D responses similar to the cells from de novo AML patients in both RF and BM (FIG. 15A). Patients with adverse prognosis had even better response than the samples with intermediate prognosis, based on the median percentage of reduction of AML graft in the mice while the difference was not significant (FIG. 15B). Patients with abnormal karyotypes which usually have adverse prognosis also had better response than the samples with normal karyotypes (FIG. 15C). Cytogenetically normal AML samples with Flt3-ITD had slightly less response to Compound D in injected RF but similar response in BM, in comparison to the samples with wild-type FLt3 that usually have better prognosis (FIG. 15D). Although the number of patient samples analyzed here are limited, samples of relapsed or secondary AML with abnormal cytogenetics and adverse prognosis are responsive to Compound D at similar level to the samples of de novo diagnosed AML with intermediate prognosis.

6.3.6. GSPT1 was Targeted and Degraded by Compound D In Vivo

The mechanism underlying the effect of Compound D against AML is that Compound D degrades translation terminator GSPT1 by recruiting GSPT1 to cereblon in the E3 ubiquitin ligase complex. Whether in vivo Compound D treatment reduced GSPT1 in AML cells in mice and whether GSPT1 degradation was responsible for AML graft reduction was investigated next. Some samples were in vitro tested to determine whether GSPT1 can be reduced in AML cells by Compound D prior to being used for in vivo treatment. Similar to the pilot experiments previously done, exposure of AML cells to Compound D decreased GSPT1 expression (FIG. 16A). Increased apoptosis was observed in 24 hours (FIG. 16B) with reduction of live cells (FIG. 16C). Colony-forming assays showed that Compound D inhibited colony forming leukemia progenitors (FIG. 16D), indicating that Compound D degrades GSPT1 in leukemia cells and inhibits proliferation of both leukemia cells and leukemia progenitors through induction of apoptosis.

Whether Compound D also induced apoptosis in the xenografts was investigated next. Following 4 weeks Compound D treatment, harvested cells were stained with propidium iodide (PI) for detecting apoptotic and dead leukemia cells. The number of PI+ events were significantly increased in the mouse bone marrow, indicating that Compound D administration resulted in induction of apoptosis and cell death (FIG. 17). To determine whether GSPT1 can also be degraded by Compound D administration in vivo, leukemia cells were harvested after 3 doses of Compound D treatment for intracellular flow cytometry to assess the levels of GSPT1 in both injected RF and non-injected BM. The levels of GSPT1 were found decreased in RF or BM, or in both RF and BM in the majority of 17 samples tested (FIG. 18A). It seems that Compound D degraded GSPT1 more profoundly in the injected RF than in non-injected BM. More samples had lesser GSPT1 reduction in the non-injected BM than in RF. Perhaps this reflects differences in the niche or blood supply between these sites; intrafemoral (IF) injection involves reaming out the femoral cavity prior to cell injection. The levels of GSPT1 reduction by Compound D varied between samples and was not correlated to the responsiveness of leukemia cells to Compound D FIG. 18B). While some samples that responded well to Compound D had clear GSPT1 reduction in both RF and BM (for example Pt120287 and 110555), other Compound D responders did not show dramatic GSPT1 reduction (like Pt130607 and 150250). In some samples the reductions of GSPT1 were different between RF and BM, for example, Pt 130826 and 150238 had opposite GSPT1 responses in RF and BM. The complex observations of GSPT1 reduction following short Compound D treatment may be due to the heterogeneous responses between AML samples. Different durations of Compound D treatment should be performed to precisely capture in vivo GSPT1 reduction for each AML sample. Nevertheless, results showed that for most of the samples, GSPT1 can be degraded by Compound D in vitro and in the mice. However, more samples need to be tested to conclude whether GSPT1 degradation can be a biomarker to predict AML response to Compound D.

6.3.7. Conclusion

Studies with 31 AML samples showed that the cereblon modulator Compound D is very potent against AML in the preclinical mouse model, including the samples with adverse prognosis and high LSC17 scores. Sub-scores in LSC-associated genes was found that predicted AML response to Compound D.

Patients with high LSC17 scores are usually resistant to standard leukemia chemotherapies with resultant poorer prognosis. Observations in the current study indicate that Compound D may be applicable for clinical trials as a new therapeutic agent for AML patients whose diseases are more aggressive in the context of primary induction therapy if such patients are rapidly identified, such as through using the LSC17 scoring method. Further analysis of LSC-associated genes from RNA-Seq generated a 4-gene set score that may predict leukemia responsiveness to Compound D. Three out of 4 genes are LSC⁻ genes with similar predicting function to 4-gene score.

In addition to the induction of apoptosis and cell death by Compound D, FACS analysis of the phenotypes of AML cells following Compound D treatment in the mice showed Compound D also changed the cell surface markers in some responding samples including myeloid differentiation markers CD15, CD14, CD11b, suggesting that at least part of treated patient samples responded to Compound D with induction of leukemia differentiation. In parallel, the proportion of CD34+ primitive cells in AML graft were also reduced by Compound D treatment. Secondary transplantation experiments with limiting dilution assays (LDAs) described in Section 6.4 may reveal whether Compound D also target the functional LSCs capable of reproducing AML in serial transplantation.

Together, through the studies on the effect of the cereblon modulator Compound D against AML, Compound D has been shown to have a strong inhibitory effect against AML in the preclinical mouse model for human AML. These observations provide important implications for Compound D in future clinical trials to treat patients with poorer prognosis and chemo-resistance and the results suggest that Compound D may reduce the possibility of AML relapse.

### 6.4. Investigating the Effects of Compound D on Acute Myeloid Leukemia Stem Cells with Secondary Transplantation Limiting Dilution Assay

6.4.1. Materials and Methods

6.4.1.1. Test Animals

The NOD/SCID mice used in this study were 10-week-old female mice with an average body weight of 20 grams at the start of treatment.

6.4.1.2. Cell Lines/Cells

All patient samples used in these studies were collected with informed consent by the Princess Margaret Leukemia Bank and subjected to Ficoll gradient centrifugation to obtain mononuclear cells for viable cryopreservation. All samples were tested for engraftment ability in NOD/SCID mice prior to use in studies. The day after the final Compound D treatment, mice treated with either vehicle or Compound D were sacrificed. Cells were harvested separately from the right femur (RF; AML cell injected) and non-injected bone marrow (BM: left femur plus left and right tibia) aliquoted for FACS analysis to assess Compound D efficacy against AML graft in the mice. Remaining cells from the same tissues (RF or BM) of each treated group were combined and viably frozen for future secondary transplantation. For secondary transplantation, frozen cells were carefully thawed, filtered to remove dead cells, and human leukemia cells were then purified through mouse cell depletion process (Mouse Cell Depletion Kit, catalogue number 130-104-694, Miltenyi Biotec). Purified cells were counted, diluted for LDA, and intrafemorally injection into irradiated secondary female NOD/SCID mice.

6.4.1.3. Assay Materials and Reagents

Human AML cells engrafted in NOD/SCID xenograft were identified through cell surface marker expression of human CD45 (dim levels) and CD33. A combination of the following anti-human antibodies was used to detect the human AML cells in the secondary xenograft: CD45-allophycyanin (APC; catalogue number 340943, BD, USA), CD33-phycoerythrin-cyanine 5 (PE-Cy5; catalogue number PN IM2647U, Beckman Coulter, USA), CD19-V450 (catalogue number 560353, BD Biosciences, USA), CD14-PE (catalogue number PN IM0650U, Beckman Coulter, USA), CD15-fluorescein isothiocyanate (FITC; catalogue number 347423, BD, USA), CD34-APC-Cy7 (catalogue number 624072, BD Biosciences, USA), and CD38-PE-Cy7 (catalogue number 335790, BD, USA).

6.4.2. Experimental Study Design

Secondary transplant was performed in this study including LDA to investigate whether Compound D targeted LSCs with self-renewal ability in the treated primary mice.

6.4.3. Experimental Procedures

6.4.3.1. Secondary Xenograft Limiting Dilution Assay

Limiting dilution assay: Limiting dilution assays were used to define the frequency of leukemia stem cells in the total leukemia graft of the primary mice. Therefore, analysis of LDA in secondary transplantation will allow quantitative determination whether Compound D targets leukemia stem cells with self-renewal ability in primary mice. For this, multiple cell doses were used for secondary transplant to achieve both a positive response (engrafted mice at high cell doses) and a negative response (non-engrafted mice at lowest cell dose). Four different AML cell doses for each treated group were used (1 million, 500,000, 50,000 and 2000 cells/mouse) with 5 mice per cell dose, totally 40 mice for each leukemia graft sample. For any sample that was considered aggressive, LDA was performed with lower cell doses. The frequency of LSCs was analyzed using the Walter and Eliza Hall Institute (WEHI) bioinformatics extreme limiting dilution analysis (ELDA) software (bioinfwehi.edu.au).

Intrafemoral transplantation: One day prior to transplantation, NOD/SCID mice were sublethally irradiated (275 cGy) and pretreated with anti-CD122 antibody (200m/mouse) to deplete residual host natural killer cells. On the day of transplantation, viably frozen cells harvested from combined vehicle- or Compound D-treated (2.5 mg/kg intraperitoneally twice daily for 14 days, Section 6.1) primary mice were thawed, counted, mouse cell depleted, and transplanted intrafemorally into the pretreated secondary mice at limiting doses in a total volume of 30 μl. For the Compound D-treated samples with low human AML engraftment 25%), cells from both RF and BM were combined and 2 rounds of mouse depletion were performed to ensure a high human AML cell purity. Flow cytometric assays were performed on an LSRII flow cytometer (BD, USA) following mouse cell depletion which showed the purity was more than 90%. For the samples/leukemia grafts that did not respond well (e.g., numbers were reduced) to Compound D, LDA was performed only using the cells from either RF or BM given the higher leukemia population. Mouse cell depletion was performed following the instructions provided by Miltenyi Biotec (Mouse Cell Depletion Kit, catalogue number 130-104-694, Miltenyi Biotec, Germany).

Treatment and Assay Procedure: At 10 to 12 weeks post-secondary transplantation, mice were euthanized and both injected (RF) and non-injected bone marrow (BM) were collected to be flushed for suspended bone marrow cells. Engraftment of AML of injected and non-injected bone marrow was analyzed by flow cytometry using human-specific antibodies. Harvested cells were pooled from injected and non-injected bone marrow of each treated group and viably frozen for future analysis.

Cells harvested from injected right femur and the non-injected femur were stained with mouse anti-human antibodies as described above. After staining, washed cells were run on an LSRII flow cytometer (BD, USA). A total of 10,000 to 20,000 events were collected for each sample. Collected data were analyzed by FlowJo software to assess AML engraftment levels in different tissues as determined by the percentage of human CD45+CD33+ cells. The frequency of LSCs was analyzed and compared between vehicle and Compound D treated groups using the WEHI bioinformatics ELDA software (bioinf.wehi.edu.au).

6.4.3.2. Data Analysis

Engraftment of AML in the injected femur and non-injected femur was analyzed by flow cytometry. Graphs were generated and statistical analyses were performed using GraphPad Prism software. Statistical significance was assessed using one-way analysis of variation (ANOVA) followed by Tukey's multiple comparison post test.

6.4.4. Effects of Compound D on Eradication of Acute Myeloid Leukemia Blasts in Xenografts Including Leukemic Stem Cells

All samples were grouped based on the 17-gene score described above. In both the injected RF and non-injected BM, the majority of samples were considered responders or partial-responders to Compound D (29 out of 35 samples), including the samples that had high LSC17 scores. Because AML samples that have high LSC17 scores are typically low responders to standard chemotherapies with poorer prognosis, this data suggested that Compound D can target leukemia with poor prognosis and these patients could be good candidates for clinical trials for treatment of patients with refractory and relapsed leukemia.

To determine if quiescent LSCs are resistant to Compound D, secondary transplantation of AML cells of high responders was performed. High responders were identified as samples in which leukemia cells were almost undetectable in the primary mouse bone marrow following Compound D treatment. If rare quiescent LSCs are maintained in the residual leukemia cells following Compound D dosing, then those quiescent LSCs may become active and repopulate secondary mice after undergoing serial transplantations. Four LSC17 high samples that had AML blasts eradicated by Compound D treatment were selected for secondary transplantation (see Table 9) by injecting total combined bone marrow cells collected from 5 primary mice treated with either vehicle or Compound D into 5 secondary mice. In this way cells collected from one primary mouse were injected into one secondary mouse without any cell dose dilution or losing any residual cells. Cells harvested from the primary mice transplanted with cells from AML patients 130311 and 130826 did not proliferate/repopulate any of the secondary mice (from both vehicle and Compound D-treated primary mice). Cells from AML Patient 150238 harvested from vehicle-treated primary mice repopulated only one of 5 secondary mice. Cells from AML Patient 150238 harvested from Compound D-treated primary mice did not repopulate any secondary mice and cells from this patient harvested from vehicle-treated primary mice only repopulated one of 5 mice. While cells from AML Patient 110625 harvested from vehicle-treated primary mice repopulated all 5 transplanted secondary mice, cells harvested from Compound D-treated primary mice repopulated the bone marrow of one secondary mouse. Further, leukemia cells from Compound D-treated mice were not found in the bone marrow of the transplanted secondary mice, indicating that in this sampling of Compound D responders, LSCs with self-renewal capacities were targeted by Compound D.

TABLE 9
Secondary Transplantation of Acute Myeloid Leukemia Cells from Vehicle
and Compound D treated Primary Xenografted Mice

| | | | In Vivo | | | | | |
|---|---|---|---|---|---|---|---|---|
| | | | Efficacy | | | | | |
| | | | (Primary | | | | | |
| | | | Mice) | | | | | |
| | | | % AML cell | | | | LSC Status | |
| | | | reduction | | No. mice | | in Secondary | LSC Status |
| | | | (following | | with LSC | | Mice | in Secondary |
| | | | Compound | | engrafted/ | | Injected | Mice |
| | | | D treatment, | | No. injected | | with | Injected with |
| | | | relative | | mice in | | Veh-Treated | Compound D |
| Patient | LSC17 | LSC17 | to Veh) | | the group | | Primary | Treated |
| ID | Score | Class | RE | BM | Veh | Compound D | Cells | Primary Cells |
| 130311 | 0.84 | LSC17hi | 99.8 | 99.6 | 0/5 | 0/4 | not engrafted | not engrafted |
| 110625 | 1.05 | LSC17hi | 99.9 | 99.9 | 5/5 | 1/5 (in BM | LSC | LSC in BM |
| | | | | | | only) | | only^{a} |
| 130826 | 0.90 | LSC17hi | 95.0 | 98.1 | 0/5 | 0/5 | not engrafted | not engrafted |
| 150238 | 1.33 | LSC17hi | 95.2 | 98.7 | 1/5 | 0/5 | LSC | no LSC |

AML = acute myeloid leukemia;
BM = bone marrow (non-injected);
ID = identification;
hi = high;
LDA = limiting dilution assay;
LSC = leukemia stem cell;
LSC17 = leukemia stem cell 17-gene score;
No. = number;
RF = right femur (injected);
Veh = vehicle.
^{a}In the one secondary mouse with LSC engraftment following AML cell injection from Compound D-treated primary mice, LSC were only detected in the non-injected bone marrow, not the RF (AML cell injected).
Acute myeloid leukemia cells were isolated from vehicle or Compound D-treated injected primary mice (both from the RF and BM) identified as responders and injected into secondary mice without performing LDA. Mice that had more than 1% CD45+CD33+ human leukemia cells of the total bone marrow cells in the injected right femur would be considered engrafted by AML LSCs.

6.4.5. Limiting Dilution Assays Performed to Demonstrate the Elimination of Leukemic Stem Cells by Compound D in the Responding Samples

Serial transplantation with limiting dilution assays was performed on the samples with residual human AML cells, to determine whether Compound D decreased the frequency of LSCs in the primary mice. Mouse bone marrow cells were first depleted from the harvested primary mouse injected right femur and non-injected bone marrow to purify human leukemia cells. Purified human leukemia cells were intrafemorally implanted into secondary NOD/SCID mice at identical cell numbers for the vehicle- and Compound D-treated groups. For each treated group of each patient sample, at least 4 different cell doses were used for secondary LDA assays. Secondary mice were not dosed with Compound D. Secondary mice were sacrificed around 12 weeks post transplantation to assess the engraftment levels of AML at each cell dose. Mice that had more than 1% CD45+CD33+ human leukemia cells of the total bone marrow cells in the injected right femur were considered engrafted by AML LSCs. The frequency of LSCs was analyzed and compared between vehicle and Compound D treated groups using the WEHI bioinformatics ELDA software (bioinf.wehi.edu.au). A representative secondary transplantation LDA is shown in Table 10, Table 11, and FIG. 19 to detail how LDAs were analyzed with ELDA software. Cells of AML Patient 130578 harvested from vehicle or Compound D-treated primary mice were transplanted into secondary mice at indicated cell doses (Table 10). Numbers of mice transplanted and engrafted at each cell dose for each treated group were summarized (see Table 10) and entered into ELDA software for calculation of LSC frequencies.

TABLE 10
Representative Secondary Transplantation Limiting Dilution Assay:
Injection and Engraftment Results

| | Treatment of | No. cells | No. mice engrafted/No. mice |
|---|---|---|---|
| Group | Primary Mice | transplanted/mouse | transplanted |
| 1 | Vehicle | 120000 | 7/7 |
| 2 | | 50000 | 4/5 |
| 3 | | 20000 | 5/5 |
| 4 | | 5000 | 2/5 |
| 5 | Compound D | 120000 | 2/3 |
| 6 | | 50000 | 2/5 |
| 7 | | 20000 | 0/5 |
| 8 | | 5000 | 0/5 |

No. = number.
Transplantation and engraftment of serially diluted acute myeloid leukemia cells from acute myeloid leukemia Patient 130578 isolated from primary vehicle or Compound D treated mice into secondary mice is shown.

Confidence intervals for group mean LSC frequency are shown in Table 11 with LSC frequencies estimated at 1 LSC in 14,536 cells in vehicle-treated primary mice and 1 LSC in 136,136 cells in Compound D-treated primary mice; a 9.4-fold significant decrease in LSC frequency following Compound D treatment compared to vehicle (p=6.2×10⁻⁵). In the representative confidence interval plot shown in FIG. 19, the red lines represent the estimated group mean LSC frequency in vehicle control and sold lines represent the estimated group mean LSC frequency in Compound D-treated primary mice. These data indicate that LSCs with self-renewal abilities in AML Patient 130578 were significantly eliminated by Compound D compared to vehicle control.

TABLE 11
Representative Secondary Transplantation Limiting Dilution Assay:
Confidence Intervals of Leukemic Stem Cell Frequencies
Isolated from Primary Mice

| | Lower Confidence | Estimated | |
|---|---|---|---|
| Group | Level | Frequency | Upper Confidence Level |
| Compound D | 361399 | 136136*** | 51281 |
| Vehicle | 30952 | 14536 | 6827 |

***= p < 0.001 relative to vehicle control group.
The estimated frequency with lower and upper confidence intervals of leukemic stem cells (LSC) from acute myeloid leukemia Patient 130578 from Compound D- or vehicle-treated mice was calculated using the Walter and Eliza Hall Institute extreme limiting dilution analysis software (available from bioinf.wehi.edu.au) and are shown as 1 LSC/total cells (e.g., for Compound D the estimate is 1 LSC for every 136,136 cells).

The results of secondary transplantation LDA from 16 AML patient samples (including 4 samples that had LDAs performed in the first pilot study [110500, 120846, 100348, and 09191; Section 6.1) are summarized in Table 12. Three samples were unable to repopulate secondary mice including one responder (120791) and two non-responders (90191 and 140171). The other 13 samples were able to repopulate secondary mice, including 8 samples that were responsive to Compound D in primary mice, 2 samples that were partially responsive to Compound D, and 3 non-responders. Six samples had reduced LSC frequencies following Compound D dosing, at reduction levels from 2.1- to 13.3-fold in primary mice. The LDA of the responder AML Patient 130926 showed Compound D did not reduce LSC frequency, probably because the initiating cell dose was too low. The highest cell dose of LDA for this patient was only 200,000 cells per mouse because Compound D eliminated most of the AML cells in the primary xenografted mouse, and only one mouse from each treated group was repopulated at this cell dose. In contrast to other responders, samples obtained from AML Patients 110484 and 130695 had LSC frequency increased following Compound D treatment in primary mice (1.6- and 12.6-fold increase, respectively). For the 3 non-responders, AML Patient 120858 was an aggressive sample and repopulated all the mice well at 2000 cells/mouse (the lowest LDA dose), so doses lower than 2000 cells would need to be transplanted to determine LSC frequencies in vehicle- and Compound D-treated mice. Compound D did not decrease the frequencies of LSCs in the other 2 non-responding samples (1.3-fold and 1.8-fold increase for samples from AML Patients 120860 and 120846, respectively).

TABLE 12

| | Summary of All Samples for Limiting Dilution Assay for Secondary Transplantation | | | | | | | | | |
|---|---|---|---|---|---|---|---|---|---|---|
| | | | | | | Tissues | | | Fold reduction | |
| | | | In vivo Efficacy | | | Used for | LSC frequency | | of LSC | |
| Patient | LSC17 | LSC17 | (% reduction) | | | Secondary | (1/total number) | | frequency by | |
| ID | Score | Class | RF | BM | Response | Transplant | Vehicle | Compound D | Compound D | P-value |
| 110500 | 0.67 | LSC17hi | 72 | 67 | R | RF | 26769 | 356239 | 13.3*** | 0.0006 |
| 598 | 0.64 | LSC17hi | 46 | 95 | R | RF | 315898 | 675569 | 2.1 | 0.31 |
| 100348 | <0.50 | LSC17lo | 86 | 90 | R | RF + BM | 230162 | 923814 | 4.0 | 0.053 |
| 120899 | 0.87 | LSC17hi | 13 | 67 | R | BM | 6820 | 27120 | 4.0 | 0.088 |
| 130695 | 0.72 | LSC17hi | 52 | 86 | R | RF | 461681 | 36696 | −12.6*** ^{a} | 0.0000058 |
| 110484 | 1.30 | LSC17hi | 73 | 96 | R | RF | 1894424 | 1153831 | −1.6 ^{a} | 0.62 |
| 130578 | 0.71 | LSC17hi | 61 | 73 | R | RF + BM | 14536 | 136136 | 9.4*** | 0.000062 |
| 130926 | 0.54 | LSC17hi | 70 | 85 | R | RF + BM | 1207241 | 1207241 | 1.0 | 0.87 |
| 130712 | 0.80 | LSC17hi | 17 | 56 | PR | BM | 282636 | 758064 | 2.7 | 0.078 |
| 121020 | 0.87 | LSC17hi | 42 | 52 | PR | RF+BM | 5124582 | 7562084 | 1.5 | 0.994 |
| 120860 | <0.50 | LSC17lo | 1 | 20 | NR | BM | 81993 | 65626 | −1.3 ^{a} | 0.681 |
| 120846 | 1.27 | LSC17hi | 9 | 26 | NR | RF | 65261 | 14523 | −1.8 ^{a} | 0.35 |
| 120858 | 0.95 | LSC17hi | 0 | 2 | NR | BM | NC | NC | NA^{b} | ND |
| 90191 | 1.38 | LSC17hi | −21 | −250 | NR | RF + BM | Not | Not engrafted | NA | ND |
| | | | | | | | engrafted | | | |
| 140171 | 0.88 | LSC17hi | 4 | 28 | NR | RF + BM | Not | Not engrafted | NA | ND |
| | | | | | | | engrafted | | | |
| 120791 | 0.11 | LSC17lo | 69 | 98 | R | RF + BM | Not | Not engrafted | NA | ND |
| | | | | | | | engrafted | | | |

BM = bone marrow (non-injected);
ID = identification;
LSC = leukemic stem cell;
LSC17 = leukemia stem cell 17-gene score;
NA = not applicable;
NC = not calculated;
ND = not determined;
NR = non-responder;
PR = partial responder;
R = responder;
RF = right femur (injected);
Veh = vehicle. *** = p < 0.001.
^{a} All mice transplanted with the lowest cell doses of both treated groups were engrafted, therefore the fold change of LSC frequency was not applicable.
^{b}Negative sign indicates a fold change increase.
Acute myeloid leukemia cells were isolated from vehicle- or Compound D-treated acute myeloid leukemia (AML)-cell injected primary mice and assessed for LSC17 score (LSC17 hi ≥ 0.50) and in vivo responsiveness to Compound D based on reduction in AML cells compared to vehicle control. Patient Samples were classified based on the percent reduction of AML cells in RF or BM following Compound D treatment relative to vehicle (R = >60%; PR = 30%-60%; NR = <30%). Cells (from RF, BM, or both) were diluted in an LDA and injected into irradiated secondary mice where LSC frequency was determined using the Walter and Eliza Hall Institute extreme limiting dilution analysis software (bioinf.wehi.edu.au) and are shown as 1 LSC/total cells. A fold change in frequency by Compound D was determined relative to vehicle control and evaluated by one-way analysis of variation. The P-value represent comparison of LSC frequency change of Compound D-treated versus control.

6.4.6. Conclusions

Following Compound D treatment in NOD/SCID mice bearing human AML, secondary transplantations were performed with LDA to determine whether Compound D was able to target LSCs. The majority of responsive leukemia samples had LSCs targeted by Compound D with varying reductions in frequencies (6 out of 10 samples). The frequencies of LSCs were not reduced in one responder and were increased in 2 of the responders to Compound D treatment. Compound D did not reduce LSC frequencies in the 3 non-responders that repopulated secondary mice. Overall, these results indicate that Compound D not only targets leukemia blasts of responders in the mouse model of human AML, but also reduces the LSCs that have self-renewal capacities. The observation that Compound D did not target LSCs in all AML samples reflects the resistance and response heterogeneity of LSCs in AML to Compound D.

From the foregoing, it will be appreciated that, although specific embodiments have been described herein for the purpose of illustration, various modifications may be made without deviating from the spirit and scope of what is provided herein. All of the references referred to above are incorporated herein by reference in their entireties.

What is claimed is:

1. A method of identifying a subject having acute myeloid leukemia (AML) who is likely to be responsive to a treatment comprising a compound or predicting the responsiveness of a subject having or suspected of having AML to a treatment comprising the compound, comprising:
i. providing a sample from the subject;
ii. measuring gene expression level of one or more genes in the sample;
iii. calculating a leukemic stem cell (LSC) signature score for the sample based on the gene expression level of the one or more genes; and
iv. identifying the subject as being likely to be responsive to the treatment comprising the compound if the level of the LSC signature score is higher than a reference level thereof,
wherein the compound is 2-(4-chlorophenyl)-N-((2-(2,6-dioxopiperidin-3-yl)-1-oxoisoindolin-5-yl)methyl)-2,2-difluoroacetamide (Compound D), which has the following structure: 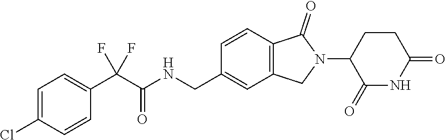 [SMILES: O=C1CCC(N2Cc3cc(CNC(=O)C(F)(F)c4ccc(Cl)cc4)ccc3C2=O)C(=O)N1]
or a stereoisomer or mixture of stereoisomers, isotopologue, pharmaceutically acceptable salt, tautomer, solvate, hydrate, co-crystal, clathrate, or polymorph thereof.

2. A method of treating a subject having AML with a compound, comprising:
(a) identifying the subject having AML that may be responsive to the treatment comprising the compound, comprising:
i. providing a sample from the subject;
ii. measuring gene expression level of one or more genes in the sample;
iii. calculating a leukemic stem cell (LSC) signature score for the sample based on the gene expression level of the one or more genes; and
iv. identifying the subject as being likely to be responsive to the treatment comprising the compound if the level of the LSC signature score is higher than a reference level thereof, and
(b) administering the subject a therapeutically effective amount of the compound if the subject is identified as being likely to be responsive to the treatment comprising the compound,
wherein the compound is Compound D, or a stereoisomer or mixture of stereoisomers, isotopologue, pharmaceutically acceptable salt, tautomer, solvate, hydrate, co-crystal, clathrate, or polymorph thereof.

3. The method of claim 1 or claim 2, wherein the LSC signature score is calculated as the weighted sum of the expression level of the one or more genes.

4. The method of any one of claims 1 to 3, wherein the reference level is the median LSC signature score in a population.

5. The method of any one of claims 1 to 3, wherein the reference level is a pre-determined LSC signature score level.

6. The method of any one of claims 1 to 5, wherein the LSC signature score that is higher than the reference level thereof suggests that the subject has resistant and/or refractory AML.

7. The method of any one of claims 1 to 6, wherein the one or more genes are selected from the group consisting of
(a) CD34, SPINK2, LAPTM48, HOXA5, GUCY1A3, SHANK3, ANGPT1, ARHGAP22, LOC284422, MYCN, MAMDC2, PRSSL1, KIAA0125, GPSM1, HOXA9, MMRN1, FSCN1, DNMT38, HOXA6, AIF1L, SOCS2, CDK6, FAM69B, NGFRAP1, C3orf54, CPXM1, TNFRSF4, ZBTB46, DPYSL3, NYNRIN, COL24A1, FAM30A, C10orf140, SPNS2, GPR56, AKR1C3, FLT3, TFPI, KCNK17, EPDR1, C1orf150, BIVM, H2AFY2, VWF, EMP1, RAGE, ATP8B4, GATA2, SLC25A37, SGK, LOC652694, ITPR3, LOC654103, CXCR4, FCRL3, RBM38, LILRA5, IL18RAP, CCDC109B, ISG20, MTSS1, CECR1, ADAM19, FCGR2A, AIM2, NPL, IL10RA, CTSL1, GNLY, CKAP4, ADM, KLRB1, SLC15A3, FGR, FCRLA, IL2RB, CXCL16, SLC4A1, GZMH, F1122662, LOC647506, GIMAP4, JAZF1, CTSH, GZMA, CHST15, AQP9, CD247, BCL6, SLC7A7, E2F2, LOC647450, GZMB, LOC652493, HBM, CD14, ALAS2, HBB, LOC642113, AHSP, FCN1, CD48, HBA2, and HBA1, or
(b) CD34, SPINK2, LAPTM48, HOXA5, GUCY1A3, SHANK3, ANGPT1, ARHGAP22, LOC284422, MYCN, MAMDC2, PRSSL1, KIAA0125, GPSM1, HOXA9, MMRN1, FSCN1, DNMT38, HOXA6, AIF1L, SOCS2, CDK6, FAM69B, NGFRAP1, C3orf54, CPXM1, TNFRSF4, ZBTB46, DPYSL3, NYNRIN, COL24A1, FAM30A, C10orf140, SPNS2, GPR56, AKR1C3, FLT3, TFPI, KCNK17, EPDR1, C1orf150, BIVM, H2AFY2, VWF, EMP1, RAGE, ATP8B4, and GATA2.

8. The method of any one of claims 1 to 6, wherein the one or more genes are selected from the group consisting of AKR1C3, ARHGAP22, CD34, CDK6, CPXM1, DNMT3B, DPYSL3, EMP1, GPR56, KIAA0125, LAPTM4B, MMRN1, NGFRAP1, NYNRIN, SMIM24, SOCS2, and ZBTB46.

9. The method of any one of claims 1 to 6, wherein the LSC signature score is based on the gene expression levels of AKR1C3, ARHGAP22, CD34, CDK6, CPXM1, DNMT3B, DPYSL3, EMP1, GPR56, KIAA0125, LAPTM4B, MMRN1, NGFRAP1, NYNRIN, SMIM24, SOCS2, and ZBTB46.

10. The method of any one of claims 1 to 6, wherein the LSC signature score is calculated as follows: (expression level of DNMT3B×weight of DNMTT3B)+(expression level of ZBTB46×weight of ZBTB46)+(expression level of NYNRIN×weight of NYNRIN)+(expression level of ARHGAP22×weight of ARHGAP22)+(expression level of LAPTM4B×weight of LAPTM4B)+(expression level of MMRN1×weight of MMRN1)+(expression level of DPYSL3×weight of DPYSL3)+(expression level of KIAA0125×weight of KIAA0125)+(expression level of CDK6×weight of CDK6)+(expression level of CPXM1×weight of CPXM1)+(expression level of SOCS2×weight of SOCS2)+(expression level of SMIM24×weight of SMIM24)+(expression level of EMP1×weight of EMP1)+(expression level of NGFRAP1×weight of NGFRAP1)+(expression level of CD34×weight of CD34)+(expression level of AKR1C3×weight of AKR1C3)+(expression level of GPR56×weight of GPR56); and
wherein the weight of DNMTT3B is in a range from 0.08 to 0.09, the weight of ZBTB46 is in a range from −0.03 to −0.04, the weight of NYNRIN is in a range from −0.008 to 0.009, the weight of ARHGAP22 is in a range from −0.015 to 0.01, the weight of LAPTM4B is in a range from −0.006 to 0.005, the weight of MMRN1 is in a range from 0.02 to 0.03, the weight of DPYSL3 is in a range from 0.02 to 0.03, the weight of KIAA0125 is in a range from 0.01 to 0.02, the weight of CDK6 is in a range from −0.08 to −0.07, the weight of CPXM1 is in a range from −0.02 to −0.03, the weight of SOCS2 is in a range from 0.02 to 0.03, the weight of SMIM24 is in a range from −0.02 to −0.03, the weight of EMP1 is in a range from 0.014 to 0.02, the weight of NGFRAP1 is in a range from 0.04 to 0.05, the weight of CD34 is in a range from 0.03 to 0.04, the weight of AKR1C3 is in a range from −0.04 to −0.05, and the weight of GPR56 is in a range from 0.04 to 0.055

11. The method of any one of claims 1 to 6, wherein the LSC signature score is calculated as follows: (expression level of DNMT3B×0.0874)+(expression level of ZBTB46×−0.0347)+(expression level of NYNRIN×0.00865)+(expression level of ARHGAP22×−0.0138)+(expression level of LAPTM4B×0.00582)+(expression level of MMRN1×0.0258)+(expression level of DPYSL3×0.0284)+(expression level of KIAA0125×0.0196)+(expression level of CDK6×−0.0704)+(expression level of CPXM1×−0.0258)+(expression level of SOCS2×0.0271)+(expression level of SMIM24×−0.0226)+(expression level of EMP1×0.0146)+(expression level of NGFRAP1×0.0465)+(expression level of CD34×0.0338)+(expression level of AKR1C3×−0.0402)+(expression level of GPR56×0.0501).

12. The method of claim 11, wherein the reference level is 0.1, 0.2, 0.3, 0.4, 0.5, 0.6, 0.7, 0.8. 0.9, 1.0, 1.1, 1.2, 1.3, 1.4, 1.5, 1.6, 1.7, 1.8, 1.9, or 2.

13. The method of any one of claims 1 to 6, wherein the LSC signature score is based on the gene expression levels of TNFRSF4, SLC4A1, SLC7A7, and AIM2.

14. The method of claim 13, wherein the LSC signature score is calculated as follows: (expression level of TNFRSF4×weight of TNFRSF4)+(expression level of SLC4A1×weight of SLC4A1)+(expression level of SLC7A7×weight of SLC7A7)+(expression level of AIM2×weight of AIM2); and wherein the weight of TNFRSF4 is in a range from −1.5 to −1, the weight of SLC4A1 is in a range from 13 to 14, the weight of SLC7A7 is in a range from −4 to −3, the weight of AIM2 is in a range from −3 to −4.

15. The method of claim 13, wherein the LSC signature score is calculated as follows: (expression level of TNFRSF4×−1.13)+(expression level of SLC4A1×13.59)+(expression level of SLC7A7×−3.57)+(expression level of AIM2×−3.04).

16. The method of claim 15, wherein the reference level is in a range from −50 to 115, from −45 to 110, from −40 to 105, from −37 to 100, from −30 to 95, from −25 to 90, from −20 to 85, from −15 to 80, from −10 to 75, from −5 to 70, from 0 to 65, from 5 to 60, from 10 to 55, from 15 to 50, from 20 to 45, from 25 to 40, or from 30 to 35.

17. The method of any one of claims 1 to 6, wherein the LSC signature score is based on the gene expression levels of SLC4A1, SLC7A7, and AIM2.

18. The method of claim 17, wherein the LSC signature score is calculated as follows: (expression level of SLC4A1×weight of SLC4A1)+(expression level of SLC7A7×weight of SLC7A7)+(expression level of AIM2×weight of AIM2); and wherein the weight of SLC4A1 is in a range from 11 to 15, the weight of SLC7A7 is in a range from −5.5 to −1.5, the weight of AIM2 is in a range from −5 to −1.

19. The method of claim 17, wherein the LSC signature score is calculated as follows: is calculated as follows: (expression level of SLC4A1×13.59)+(expression level of SLC7A7×−3.57)+(expression level of AIM2×−3.04).

20. The method of claim 19, wherein the reference level is in a range from −65 to 110, from −60 to 105, from −55 to 100, from −49 to 93, from −45 to 90, from −40 to 85, from −35 to 80, from −30 to 75, from −25 to 70, from −20 to 65, from −15 to 60, from −10 to 55, from −5 to 50, from 0 to 45, from 5 to 40, from 10 to 35, from 15 to 30, from 20 to 35, or from 25 to 30.

21. A method of identifying a subject having AML, who is likely to be responsive to a treatment comprising a compound or predicting the responsiveness of a subject having or suspected of having AML to a treatment comprising the compound, comprising:
i. providing a sample from the subject;
ii. administering the compound to the sample;
iii. measuring the proportion of one or more types of cells;
iv. identifying the subject as being likely to be responsive to the treatment comprising the compound if the proportion of the one or more types of cells differentiates from a reference proportion of the cells,
wherein the compound is Compound D, or a stereoisomer or mixture of stereoisomers, isotopologue, pharmaceutically acceptable salt, tautomer, solvate, hydrate, co-crystal, clathrate, or polymorph thereof.

22. A method of treating a subject having AML with a compound, comprising:
(a) identifying the subject having AML that may be responsive to the treatment comprising the compound, comprising:
i. providing a sample from the subject;
ii. administering the compound to the sample;
iii. measuring the proportion of one or more types of cells;
iv. identifying the subject as being likely to be responsive to the treatment comprising the compound if the proportion of the one or more types of cells differentiates from a reference proportion of the cells, and
(b) administering to the subject a therapeutically effective amount of the compound if the subject is identified as being likely to be responsive to the treatment comprising the compound,
wherein the compound is Compound D, or a stereoisomer or mixture of stereoisomers, isotopologue, pharmaceutically acceptable salt, tautomer, solvate, hydrate, co-crystal, clathrate, or polymorph thereof.

23. The method of claim 21 or claim 22, wherein the reference proportion of a type of cells is the proportion of the type of cells in the sample prior to administering the compound.

24. The method of claim 21 or claim 22, wherein the reference proportion of a type of cells is a pre-determined proportion.

25. The method of any one of claims 21 to 24, wherein the method comprising measuring the proportion of primitive cells and/or the proportion differentiated leukemia cells.

26. The method of claim 25, wherein a reduction of the proportion of primitive cells and/or an increase of the proportion of differentiated leukemia cells as compared to their respective proportions prior to administering the compound indicates that the subject is likely to be responsive to the treatment comprising the compound.

27. The method of any one of claims 21 to 26, wherein the method comprising measuring the proportion of CD34+, CD15+ cells, CD14+ cells, and/or CD11b+ cells.

28. The method of any one of claims 21 to 27, wherein the method comprising measuring the proportion of CD34+ cells, and wherein a reduction of the proportion of CD34+ cells as compared to the proportion of CD34+ cells prior to administering the compound indicates the subject is likely to be responsive to the treatment comprising the compound.

29. The method of any one of claims 21 to 27, wherein the method comprising measuring the proportion of CD15+ cells and/or CD14+ cells, and wherein an increase of the proportion of CD15+ cells and/or CD14+ cells as compared to the proportion of CD15+ cells and/or CD14+ cells prior to administering the compound indicates the subject is likely to be responsive to the treatment comprising the compound.

30. The method of any one of claims 1 to 29, wherein the AML is refractory or resistant.

31. The method of any one of claims 1 to 29, wherein the AML is resistant to treatment using one or more agents selected from the group consisting of daunorubicin, cytarabine (ara-C), and gemtuzumab ozogamicin, or resistant to chemotherapies.
